SEQUENCE LISTING (370 sequences)

SEQ ID NO: 1 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 2 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 3 - DNA, 345 bp, Homo sapiens
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccacc

SEQ ID NO: 4 - DNA, 1539 bp, Homo sapiens
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatctaa

SEQ ID NO: 5 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 21 aa, Apis sp.
MKFLVNVALVFMVVYISYIYA

SEQ ID NO: 8 - protein, 22 aa, Unknown
  Description of Unknown: Tissue plasminogen activator peptide
MDAMKRGLCCVLLLCGAVFVSP

SEQ ID NO: 9 - protein, 18 aa, Unknown
  Description of Unknown: Native peptide
MTAPWVALALLWGSLCAG

SEQ ID NO: 10 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 11 - protein, 6 aa, Artificial Sequence
  Description of Artificial Sequence: Synthetic peptide
GRGEAE

SEQ ID NO: 12 - protein, 335 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 16 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 17 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 19 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 20 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 22 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 228 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPFRPEVHLLPPSREEMTKNQVSLTCLARGFYPKDIAVEWESNGQPENNYKTTPSRQEPSQGTTTFAVTSKLTVDKSRWQQGNVFSCSVMHEALHNHYTQKTISLSPGK

SEQ ID NO: 25 - protein, 228 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPPSEELALNELVTLTCLVKGFYPSDIAVEWESNGQELPREKYLTWAPVLDSDGSFFLYSILRVAAEDWKKGDTFSCSVMHEALHNHYTQKSLDRSPGK

SEQ ID NO: 26 - protein, 261 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLEKELQALEKENAQLEWELQALEKELAQGAT

SEQ ID NO: 27 - protein, 261 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSAQLKKKLQALKKKNAQLKWKLQALKKKLAQGAT

SEQ ID NO: 28 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTENQVSLWCLVKGFYPSDIAVEWESNGQPENNYKTTPPVLDSDGSFFLYSKLTVDKSRWQQGNVFSCSVMHEALHNHYTQDSLSLSPGK

SEQ ID NO: 29 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESRGQPENNYKTTPPVLDSRGSFFLVSKLTVDKSRWQQGNVFSCSVMHEALHNHYTQKSLSLSPGK

SEQ ID NO: 30 - protein, 225 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 31 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDARLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1104 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacgcccggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 33 - protein, 343 aa, Artificial Sequence
  Description of Artificial Sequence: Synthetic polypeptide
GRGEAETRECIYYNANWELERTNQSGLERCEGEQDARLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDERLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1104 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacgagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 36 - protein, 343 aa, Artificial Sequence
  Description of Artificial Sequence: Synthetic polypeptide
GRGEAETRECIYYNANWELERTNQSGLERCEGEQDERLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDINCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 1104 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacatcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 39 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDINCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDKNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 1104 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacaagaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 42 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDKNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 367 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWEDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 44 - DNA, 1101 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggaagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacgacaccacgcctcccgtgctggactccgacggctccttcttcctctatagcgacctcaccgtggacaagagcaggtggcagcaggggaacgtcttctcatgctccgtgatgcatgaggctctgcacaaccactacacgcagaagagcctctccctgtctccgggt

SEQ ID NO: 45 - protein, 342 aa, Artificial Sequence
  Description of Artificial Sequence: Synthetic polypeptide
GRGEAETRECIYYNANWELERTNQSGLERCEGEQDKRLHCYASWRNSSGTIELVKKGCWEDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPG

SEQ ID NO: 46 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 47 - DNA, 1104 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaggagatgaccaagaaccaggtcagcctgacctgcctggtcaaaggcttctatcccagcgacatcgccgtggagtgggagagcaatgggcagccggagaacaactacaagaccacgcctcccgtgctgaagtccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 49 - protein, 367 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWEDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 342 aa, Artificial Sequence
  Description of Artificial Sequence: Synthetic polypeptide
GRGEAETRECIYYNANWELERTNQSGLERCEGEQDKRLHCYASWRNSSGTIELVKKGCWEDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 52 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 107 aa, Artificial Sequence
  Description of Artificial Sequence: Synthetic polypeptide
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPT

SEQ ID NO: 54 - protein, 329 aa, Artificial Sequence
  Description of Artificial Sequence: Synthetic polypeptide
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 344 aa, Artificial Sequence
  Description of Artificial Sequence: Synthetic polypeptide
SGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 357 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 57 - protein, 382 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 58 - DNA, 1149 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtgggggtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatga

SEQ ID NO: 59-260 - skipped (202 entries)

SEQ ID NO: 261 - protein, 3 aa, Artificial Sequence
  Description of Artificial Sequence: Synthetic peptide
GGG

SEQ ID NO: 262 - protein, 4 aa, Artificial Sequence
  Description of Artificial Sequence: Synthetic peptide
GGGG

SEQ ID NO: 263 - protein, 5 aa, Artificial Sequence
  Description of Artificial Sequence: Synthetic peptide
TGGGG

SEQ ID NO: 264 - protein, 5 aa, Artificial Sequence
  Description of Artificial Sequence: Synthetic peptide
SGGGG

SEQ ID NO: 265 - protein, 4 aa, Artificial Sequence
  Description of Artificial Sequence: Synthetic peptide
TGGG

SEQ ID NO: 266 - protein, 4 aa, Artificial Sequence
  Description of Artificial Sequence: Synthetic peptide
SGGG

SEQ ID NO: 267 - protein, 5 aa, Artificial Sequence
  Description of Artificial Sequence: Synthetic peptide
GGGGS

SEQ ID NO: 268 - protein, 10 aa, Artificial Sequence
  Description of Artificial Sequence: Synthetic peptide
GGGGSGGGGS

SEQ ID NO: 269 - protein, 11 aa, Artificial Sequence
  Description of Artificial Sequence: Synthetic peptide
TGGGGSGGGGS

SEQ ID NO: 270 - protein, 16 aa, Artificial Sequence
  Description of Artificial Sequence: Synthetic peptide
TGGGGSGGGGSGGGGS

SEQ ID NO: 271 - protein, 21 aa, Artificial Sequence
  Description of Artificial Sequence: Synthetic peptide
TGGGGSGGGGSGGGGSGGGGS

SEQ ID NO: 272 - protein, 26 aa, Artificial Sequence
  Description of Artificial Sequence: Synthetic peptide
TGGGGSGGGGSGGGGSGGGGSGGGGS

SEQ ID NO: 273 - protein, 31 aa, Artificial Sequence
  Description of Artificial Sequence: Synthetic polypeptide
TGGGGSGGGGSGGGGSGGGGSGGGGSGGGGS

SEQ ID NO: 274 - protein, 36 aa, Artificial Sequence
  Description of Artificial Sequence: Synthetic polypeptide
TGGGGSGGGGSGGGGSGGGGSGGGGSGGGGSGGGGS

SEQ ID NO: 275 - protein, 10 aa, Artificial Sequence
  Description of Artificial Sequence: Synthetic peptide
TGGGPKSCDK

SEQ ID NO: 276 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDIRCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacatccgttgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 278 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDIRCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDKRCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 4 aa, Artificial Sequence
  Description of Artificial Sequence: Synthetic peptide
GGGA

SEQ ID NO: 281 - protein, 4 aa, Artificial Sequence
  Description of Artificial Sequence: Synthetic peptide
GGGS

SEQ ID NO: 282 - protein, 5 aa, Artificial Sequence
  Description of Artificial Sequence: Synthetic peptide
GGGGA

SEQ ID NO: 283 - protein, 5 aa, Artificial Sequence
  Description of Artificial Sequence: Synthetic peptide
GGGGG

SEQ ID NO: 284 - protein, 4 aa, Artificial Sequence
  Description of Artificial Sequence: Synthetic peptide
GGAG

SEQ ID NO: 285 - protein, 4 aa, Artificial Sequence
  Description of Artificial Sequence: Synthetic peptide
GGSG

SEQ ID NO: 286 - protein, 4 aa, Artificial Sequence
  Description of Artificial Sequence: Synthetic peptide
AGGG

SEQ ID NO: 287 - protein, 4 aa, Artificial Sequence
  Description of Artificial Sequence: Synthetic peptide
GAGA

SEQ ID NO: 288 - protein, 4 aa, Artificial Sequence
  Description of Artificial Sequence: Synthetic peptide
GSGS

SEQ ID NO: 289 - protein, 6 aa, Artificial Sequence
  Description of Artificial Sequence: Synthetic peptide
GAGAGA

SEQ ID NO: 290 - protein, 6 aa, Artificial Sequence
  Description of Artificial Sequence: Synthetic peptide
GSGSGS

SEQ ID NO: 291 - protein, 8 aa, Artificial Sequence
  Description of Artificial Sequence: Synthetic peptide
GAGAGAGA

SEQ ID NO: 292 - protein, 8 aa, Artificial Sequence
  Description of Artificial Sequence: Synthetic peptide
GSGSGSGS

SEQ ID NO: 293 - protein, 10 aa, Artificial Sequence
  Description of Artificial Sequence: Synthetic peptide
GAGAGAGAGA

SEQ ID NO: 294 - protein, 10 aa, Artificial Sequence
  Description of Artificial Sequence: Synthetic peptide
GSGSGSGSGS

SEQ ID NO: 295 - protein, 12 aa, Artificial Sequence
  Description of Artificial Sequence: Synthetic peptide
GAGAGAGAGAGA

SEQ ID NO: 296 - protein, 12 aa, Artificial Sequence
  Description of Artificial Sequence: Synthetic peptide
GSGSGSGSGSGS

SEQ ID NO: 297 - protein, 6 aa, Artificial Sequence
  Description of Artificial Sequence: Synthetic peptide
GGAGGA

SEQ ID NO: 298 - protein, 6 aa, Artificial Sequence
  Description of Artificial Sequence: Synthetic peptide
GGSGGS

SEQ ID NO: 299 - protein, 9 aa, Artificial Sequence
  Description of Artificial Sequence: Synthetic peptide
GGAGGAGGA

SEQ ID NO: 300 - protein, 9 aa, Artificial Sequence
  Description of Artificial Sequence: Synthetic peptide
GGSGGSGGS

SEQ ID NO: 301 - protein, 12 aa, Artificial Sequence
  Description of Artificial Sequence: Synthetic peptide
GGAGGAGGAGGA

SEQ ID NO: 302 - protein, 12 aa, Artificial Sequence
  Description of Artificial Sequence: Synthetic peptide
GGSGGSGGSGGS

SEQ ID NO: 303 - protein, 4 aa, Artificial Sequence
  Description of Artificial Sequence: Synthetic peptide
GGAG

SEQ ID NO: 304 - protein, 4 aa, Artificial Sequence
  Description of Artificial Sequence: Synthetic peptide
GGSG

SEQ ID NO: 305 - protein, 8 aa, Artificial Sequence
  Description of Artificial Sequence: Synthetic peptide
GGAGGGAG

SEQ ID NO: 306 - protein, 8 aa, Artificial Sequence
  Description of Artificial Sequence: Synthetic peptide
GGSGGGSG

SEQ ID NO: 307 - protein, 12 aa, Artificial Sequence
  Description of Artificial Sequence: Synthetic peptide
GGAGGGAGGGAG

SEQ ID NO: 308 - protein, 12 aa, Artificial Sequence
  Description of Artificial Sequence: Synthetic peptide
GGSGGGSGGGSG

SEQ ID NO: 309 - protein, 5 aa, Artificial Sequence
  Description of Artificial Sequence: Synthetic peptide
GGGGA

SEQ ID NO: 310 - protein, 15 aa, Artificial Sequence
  Description of Artificial Sequence: Synthetic peptide
GGGGAGGGGAGGGGA

SEQ ID NO: 311 - protein, 15 aa, Artificial Sequence
  Description of Artificial Sequence: Synthetic peptide
GGGGSGGGGSGGGGS

SEQ ID NO: 312 - protein, 5 aa, Artificial Sequence
  Description of Artificial Sequence: Synthetic peptide
GGGAG

SEQ ID NO: 313 - protein, 6 aa, Artificial Sequence
  Description of Artificial Sequence: Synthetic peptide
GGGAGG

SEQ ID NO: 314 - protein, 7 aa, Artificial Sequence
  Description of Artificial Sequence: Synthetic peptide
GGGAGGG

SEQ ID NO: 315 - protein, 4 aa, Artificial Sequence
  Description of Artificial Sequence: Synthetic peptide
AAAL

SEQ ID NO: 316 - protein, 4 aa, Artificial Sequence
  Description of Artificial Sequence: Synthetic peptide
AAAK

SEQ ID NO: 317 - protein, 4 aa, Artificial Sequence
  Description of Artificial Sequence: Synthetic peptide
AAAR

SEQ ID NO: 318 - protein, 14 aa, Artificial Sequence
  Description of Artificial Sequence: Synthetic peptide
EGKSSGSGSESKST

SEQ ID NO: 319 - protein, 12 aa, Artificial Sequence
  Description of Artificial Sequence: Synthetic peptide
GSAGSAAGSGEF

SEQ ID NO: 320 - protein, 12 aa, Artificial Sequence
  Description of Artificial Sequence: Synthetic peptide
AEAAAKEAAAKA

SEQ ID NO: 321 - protein, 18 aa, Artificial Sequence
  Description of Artificial Sequence: Synthetic peptide
KESGSVSSEQLAQFRSLD

SEQ ID NO: 322 - protein, 10 aa, Artificial Sequence
  Description of Artificial Sequence: Synthetic peptide
GENLYFQSGG

SEQ ID NO: 323 - protein, 8 aa, Artificial Sequence
  Description of Artificial Sequence: Synthetic peptide
SACYCELS

SEQ ID NO: 324 - protein, 5 aa, Artificial Sequence
  Description of Artificial Sequence: Synthetic peptide
RSIAT

SEQ ID NO: 325 - protein, 17 aa, Artificial Sequence
  Description of Artificial Sequence: Synthetic peptide
RPACKIPNDLKQKVMNH

SEQ ID NO: 326 - protein, 36 aa, Artificial Sequence
  Description of Artificial Sequence: Synthetic polypeptide
GGSAGGSGSGSSGGSSGASGTGTAGGTGSGSGTGSG

SEQ ID NO: 327 - protein, 17 aa, Artificial Sequence
  Description of Artificial Sequence: Synthetic peptide
AAANSSIDLISVPVDSR

SEQ ID NO: 328 - protein, 36 aa, Artificial Sequence
  Description of Artificial Sequence: Synthetic polypeptide
GGSGGGSEGGGSEGGGSEGGGSEGGGSEGGGSGGGS

SEQ ID NO: 329 - protein, 5 aa, Artificial Sequence
  Description of Artificial Sequence: Synthetic peptide
EAAAK

SEQ ID NO: 330 - protein, 5 aa, Artificial Sequence
  Description of Artificial Sequence: Synthetic peptide
PAPAP

SEQ ID NO: 331 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacaagcgttgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 332 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDKRCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 333 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDTRCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 334 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacacccgttgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 335 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDTRCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 336 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDTNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 337 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacaccaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 338 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDTNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 339 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWHDDINCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 340 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggcacgatgacatcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 341 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWHDDINCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 342 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWHDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 343 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggcacgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 344 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWHDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWHDDKNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 346 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggcacgatgacaagaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 347 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWHDDKNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 348 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCLGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 349 - DNA, 1107 bp, Artificial Sequence
  Description of Artificial Sequence: Synthetic polynucleotide
atggatgcgatgaaacgcggcctgtgctgcgtgctgctgctgtgcggcgcggtgtttgtgagcccgggcgccagcggccgcggcgaagcggaaacccgcgaatgcatttattataacgcgaactgggaactggaacgcaccaaccagagcggcctggaacgctgcctgggcgaacaggataaacgcctgcattgctatgcgagctggcgcaacagcagcggcaccattgaactggtgaaaaaaggctgctggctggatgattttaactgctatgatcgccaggaatgcgtggcgaccgaagaaaacccgcaggtgtatttttgctgctgcgaaggcaacttttgcaacgaacgctttacccatctgccggaagcgggcggcccggaagtgacctatgaaccgccgccgaccgcgccgaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 350 - protein, 343 aa, Artificial Sequence
  Description of Artificial Sequence: Synthetic polypeptide
GRGEAETRECIYYNANWELERTNQSGLERCLGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 368 aa, Artificial Sequence
  Description of Artificial Sequence: Synthetic polypeptide
MDAMKRGLCCVLLLCGAVFVSPGASGRGEAETRECIYYNANWENERTNQSGLERCEGEQDKRLHCYASWRNSSGTIELVKKGCWRDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - DNA, 1107 bp, Artificial Sequence
  Description of Artificial Sequence: Synthetic polynucleotide
atggatgcaatgaagagagggctctgctgtgtgctgctgctgtgtggagcagtcttcgtttcgcccggcgcctctgggcgtggggaggctgagacacgggagtgcatctactacaacgccaactgggagaacgagcgcaccaaccagagcggcctggagcgctgcgaaggcgagcaggacaagcggctgcactgctacgcctcctggcgcaacagctctggcaccatcgagctcgtgaagaagggctgctggcgcgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 353 - protein, 343 aa, Artificial Sequence
  Description of Artificial Sequence: Synthetic polypeptide
GRGEAETRECIYYNANWENERTNQSGLERCEGEQDKRLHCYASWRNSSGTIELVKKGCWRDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 354 - protein, 368 aa, Artificial Sequence
  Description of Artificial Sequence: Synthetic polypeptide
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQGYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - DNA, 1107 bp, Artificial Sequence
  Description of Artificial Sequence: Synthetic polynucleotide
atggatgcgatgaaacgcggcctgtgctgcgtgctgctgctgtgcggcgcggtgtttgtgagcccgggcgccagcggccgcggcgaagcggaaacccgcgaatgcatttattataacgcgaactgggaactggaacgcaccaaccagagcggcctggaacgctgcgaaggcgaacaggataaacgcctgcattgctatgcgagctggcgcaacagcagcggcaccattgaactggtgaaaaaaggctgctggctggatgattttaactgctatgatcgccaggaatgcgtggcgaccgaagaaaacccgcagggctatttttgctgctgcgaaggcaacttttgcaacgaacgctttacccatctgccggaagcgggcggcccggaagtgacctatgaaccgccgccgaccgcgccgaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 356 - protein, 343 aa, Artificial Sequence
  Description of Artificial Sequence: Synthetic polypeptide
GRGEAETRECIYYNANWELERTNQSGLERCEGEQDKRLHCYASWRNSSGTIELVKKGCWLDDFNCYDRQECVATEENPQGYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 107 aa, Artificial Sequence; identical to SEQ ID NO: 53
  Description of Artificial Sequence: Synthetic polypeptide

SEQ ID NO: 358 - protein, 150 aa, Rattus sp.
MTAPWAALALLWGSLCAGSGRGEAETRECIYYNANWELERTNQSGLERCEGEQDKRLHCYASWPNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 359 - protein, 150 aa, Sus sp.
MTAPWAALALLWGSLCVGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 360 - protein, 150 aa, Mus sp.
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 361 - protein, 150 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 362 - protein, 150 aa, Bos taurus
MTAPWAALALLWGSLCAGSGRGEAETRECIYYNANWELERTNQSGLERCEGERDKRLHCYASWRNSSGTIELVKKGCWLDDFNCYDRQECVATEENPQVYFCCCEGNFCNERFTHLPEAGGPEVTYEPPPTAPTLLTVLAYSLLPVGGLS

SEQ ID NO: 363 - protein, 150 aa, Xenopus sp.
MGASVALTFLLLLATFRAGSGHDEVETRECIYYNANWELEKTNQSGVERLVEGKKDKRLHCYASWRNNSGFIELVKKGCWLDDFNCYDRQECIAKEENPQVFFCCCEGNYCNKKFTHLPEVETFDPKPQPSASVLNILIYSLLPIVGLSM

SEQ ID NO: 364 - protein, 150 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLI

SEQ ID NO: 365 - protein, 154 aa, Artificial Sequence
  Description of Artificial Sequence: Synthetic polypeptide
  MOD_RES (8)..(8): Ala, Thr, or absent
  MOD_RES (121)..(121): Pro, Ala, Val, or Met
MTAPWAAXLALLWGSLCAGSGRGEAETRECRYYNANWELERTNQSGLERLCEGEQDKRIHCYASWRNSSGTIEIVKKGCWLDDFNCYDRQECVATEENPQVYFCCCEGNFCNERFTHLPEXGGPEVTYEPKPPTAPTLLTVLAYSLLPIGGLSM

SEQ ID NO: 366 - protein, 6 aa, Artificial Sequence
  Description of Artificial Sequence: Synthetic 6xHis tag
HHHHHH

SEQ ID NO: 367 - protein, 10 aa, Artificial Sequence
  Description of Artificial Sequence: Synthetic peptide
  See specification as filed for detailed description of substitutions and preferred embodiments
GGGGSGGGGS

SEQ ID NO: 368 - protein, 20 aa, Artificial Sequence
  Description of Artificial Sequence: Synthetic peptide
  SITE (1)..(20): This sequence may encompass 1-4 “Gly Gly Gly Gly Ser” repeating units
GGGGSGGGGSGGGGSGGGGS

SEQ ID NO: 369 - protein, 10 aa, Artificial Sequence
  Description of Artificial Sequence: Synthetic peptide
  MOD_RES (1)..(1): Any amino acid
  MOD_RES (3)..(3): Any amino acid
  MOD_RES (5)..(5): Any amino acid
  MOD_RES (7)..(7): Any amino acid
  MOD_RES (9)..(9): Any amino acid
  SITE (1)..(10): This sequence may encompass 1-5 “Xaa Pro” repeating units
XPXPXPXPXP

SEQ ID NO: 370 - DNA, 48 bp, Artificial Sequence
  Description of Artificial Sequence: Synthetic oligonucleotide
cagacagacagacagacagacagacagacagacagacagacagacaga

(19) United States
(12) Patent Application Publication
(10) Pub. No.: US 2024/0181007 A1
(43) Pub. Date: Jun. 6, 2024

(54) COMPOSITIONS AND METHODS FOR TREATING PULMONARY HYPERTENSION

(71) Applicant: Acceleron Pharma Inc., Cambridge, MA (US)

(72) Inventors: Gang Li, Cambridge, MA (US); Ravindra Kumar, Cambridge, MA (US); Jonathan Toshi Lu, Cambridge, MA (US); Sachindra Joshi, Cambridge, MA (US); Patrick Andre, Cambridge, MA (US)

(21) Appl. No.: 18/271,258

(22) PCT Filed: Jan. 7, 2022
(86) PCT No.: PCT/US2022/011619

§ 371 Date: Jul. 7, 2023

Related U.S. Application Data
(60) Provisional application No. 63/135,267, filed on Jan. 8, 2021.
(60) Provisional application No. 63/213,052, filed on Jun. 21, 2021.

(51) Int. Cl. A61K 38/17; A61P 9/12

(52) U.S. Cl. CPC A61K 38/179; A61P 9/12

(57) ABSTRACT

In some aspects, the disclosure relates to compositions and methods comprising variant ActRIIB polypeptides or heteromultimers thereof to treat, prevent, or reduce the progression rate and/or severity of post-capillary pulmonary hypertension (PcPH), particularly treating, preventing or reducing the progression rate and/or severity of one or more PcPH-associated complications.

### CROSS-REFERENCE TO RELATED APPLICATIONS

This application claims the benefit of priority to U.S. provisional application Ser. No. 63/135,267, filed on Jan. 8, 2021 and 63/213,052, filed on Jun. 21, 2021. The disclosures of the foregoing applications are hereby incorporated by reference in their entirety.

### BACKGROUND OF THE INVENTION

Pulmonary hypertension (PH) is a disease characterized by high blood pressure in lung vasculature, including pulmonary arteries, pulmonary veins, and pulmonary capillaries. In general, PH is defined as a mean pulmonary arterial pressure (mPAP)≥20 mm Hg at rest or ≥30 mm Hg with exercise [Hill et al., Respiratory Care 54(7):958-68 (2009)]. One of the main PH symptoms is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches [Simonneau et al., JACC 54(1):S44-54 (2009)]. Clinical classification of PH was first proposed in 1973, and a recent updated clinical classification was endorsed by the World Health Organization (WHO) in 2018. According to the updated PH clinical classification, there are five main groups of PH: pulmonary arterial hypertension (PAH), characterized by a pulmonary arterial wedge pressure (PAWP)≤15 mm Hg; PH due to left heart disease (also known as pulmonary venous hypertension or congestive heart failure), characterized by a PAWP >15 mm Hg; PH due to lung diseases and/or hypoxia; PH due to pulmonary artery obstructions; and PH with unclear and/or multifactorial mechanisms [Simonneau (2019) Eur Respir J: 53:1801913]. PH due to left heart disease is further classified into PH due to heart failure with preserved left ventricular ejection fraction; PH due to heart failure with reduced left ventricular ejection fraction; valvular heart disease; and congenital/acquired cardiovascular conditions leading to post-capillary PH [Simonneau (2019) Eur Respir J: 53:1801913]. Diagnosis of various types of PH typically requires a series of tests.

In general, PH treatment depends on the cause or classification of PH. Where PH is caused by a known medicine or medical condition, it is known as a secondary PH, and its treatment is usually directed at the underlying disease. Treatment of Group 2 pulmonary hypertension (e.g., venous hypertension) generally involves optimizing left ventricular function by administering diuretics, beta blockers, angiotensin receptor-neprilysin inhibitors (ARNI), and ACE inhibitors, cardiac resynchronization therapy, or repairing or replacing a mitral valve or aortic valve.

There is a high, unmet need for effective therapies for treating pulmonary hypertension. Accordingly, it is an object of the present disclosure to provide methods for treating, preventing, or reducing the progression rate and/or severity of PH, particularly treating, preventing or reducing the progression rate and/or severity of one or more PH-associated complications.

### SUMMARY OF THE INVENTION

In certain aspects, the disclosure relates to methods of treating post-capillary pulmonary hypertension (PcPH). In certain aspects, the disclosure provides methods of treating post-capillary pulmonary hypertension (PcPH), comprising administering to a patient in need thereof an effective amount of a variant ActRIIB polypeptide comprising an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2 selected from the group consisting of A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, F108, E111, R112, A119, G120, E123, P129, P130, and A132. In other aspects, the disclosure provides methods of treating, preventing, or reducing the progression rate and/or severity of one or more complications of post-capillary pulmonary hypertension, comprising administering to a patient in need thereof an effective amount of a variant ActRIIB polypeptide comprising an amino acid sequence that is at least 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2 selected from the group consisting of: A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, F108, E111, R112, A119, G120, E123, P129, P130, and A132. In some embodiments, the one or more complications of post-capillary pulmonary hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, left ventricular hypertrophy, left atrium dilation, left ventricular fibrosis, right ventricular fibrosis, and pulmonary fibrosis. In some embodiments, the PcPH is isolated post-capillary pulmonary hypertension (IpcPH). In some embodiments, the PcPH is combined post- and pre-capillary PH (CpcPH). In some embodiments, the patient has Group 2 pulmonary hypertension as recognized by the World Health Organization (WHO). In some embodiments, the patient has pulmonary hypertension due to heart failure with preserved left ventricular ejection fraction (LVEF). In some embodiments, the patient has pulmonary hypertension due to heart failure with reduced left ventricular ejection fraction (LVEF). In some embodiments, the patient has valvular heart disease. In some embodiments, the patient has congenital/acquired cardiovascular conditions leading to post-capillary PH. In some embodiments, the patient has Group 5 pulmonary hypertension as recognized by the WHO. In some embodiments, the patient has pulmonary hypertension with unclear and/or multifactorial mechanisms. In some embodiments, the valvular heart disease is aortic regurgitation. In some embodiments, the valvular heart disease is aortic stenosis. In some embodiments, the valvular heart disease is mitral valve regurgitation. In some embodiments, the valvular heart disease is mitral valve stenosis. In some embodiments, the patient has a mean pulmonary arterial pressure (mPAP) selected from the group consisting of: an mPAP of at least 20 mmHg; an mPAP of at least 25 mmHg; an mPAP of at least 30 mmHg; an mPAP of at least 35 mmHg; an mPAP of at least 40 mmHg; an mPAP of at least 45 mmHg; and an mPAP of at least 50 mmHg. In some embodiments, the method reduces mPAP in the patient. In some embodiments, the method reduces the mPAP in the patient by at least 10% (e.g., 10%, 15%, 20%, 25%, 30%, 35%, 40%, 45%, or at least 50%). In some embodiments, the method reduces the mPAP by at least 3 mmHg (e.g., at least 3, 5, 7, 10, 12, 15, 20, or 25 mm Hg) in the patient. In some embodiments, the patient has a pulmonary arterial wedge pressure (PAWP) of greater than 15 mmHg. In some embodiments, the method decreases the PAWP in the patient. In some embodiments, the method reduces the PAWP in the patient by at least 10% (e.g., 10%, 15%, 20%, 25%, 30%, 35%, 40%, 45%, or at least 50%). In some embodiments, the patient has a left ventricular end diastolic pressure (LVEDP) of greater than 15 mmHg. In some embodiments, the method decreases the LVEDP in the patient. In some embodiments, the method reduces the LVEDP in the patient by at least 10% (e.g., 10%, 15%, 20%, 25%, 30%, 35%, 40%, 45%, or at least 50%). In some embodiments, the patient has a diastolic pressure gradient (DPG) of less than 7 mmHg. In some embodiments, the patient has a DPG of at least 7 mmHg. In some embodiments, the method decreases the DPG in the patient. In some embodiments, the method reduces the DPG in the patient by at least 10% (e.g., 10%, 15%, 20%, 25%, 30%, 35%, 40%, 45%, or at least 50%). In some embodiments, the patient has a transpulmonary pressure gradient (TPG) of less than or equal to 12 mm Hg. In some embodiments, the patient has a TPG of greater than 12 mm Hg. In some embodiments, the method decreases the TPG in the patient. In some embodiments, the method reduces the TPG in the patient by at least 10% (e.g., 10%, 15%, 20%, 25%, 30%, 35%, 40%, 45%, or at least 50%). In some embodiments, the patient has a pulmonary vascular resistance (PVR) greater than or equal to 3 Wood Units. In some embodiments, the method decreases the PVR in the patient. In some embodiments, the method reduces the PVR in the patient by at least 10% (e.g., 10%, 15%, 20%, 25%, 30%, 35%, 40%, 45%, or at least 50%).

In some embodiments, the method prevents the progression of IpcPH to CpcPH. In some embodiments, the method reduces the development of a pre-capillary component of PH. In some embodiments, the patient has preserved left ventricular ejection fraction. In some embodiments, the preserved left ventricular ejection fraction is greater than 45%. In some embodiments, the patient has reduced left ventricular ejection fraction. In some embodiments, the reduced left ventricular fraction is less than 45%. In some embodiments, the preserved left ventricular fraction is measured using echocardiography. In some embodiments, the patient has diastolic dysfunction of the left ventricle. In some embodiments, the patient has systolic dysfunction of the left ventricle. In some embodiments, the method decreases right ventricular hypertrophy in the patient. In some embodiments, the method decreases right ventricular hypertrophy in the patient by at least 10% (e.g., 10%, 15%, 20%, 25%, 30%, 35%, 40%, 45%, or at least 50%). In some embodiments, the method decreases left ventricular hypertrophy in the patient. In some embodiments, the method decreases left ventricular hypertrophy in the patient by at least 10% (e.g., 10%, 15%, 20%, 25%, 30%, 35%, 40%, 45%, or at least 50%). In some embodiments, the method decreases smooth muscle hypertrophy in the patient. In some embodiments, the method decreases smooth muscle hypertrophy in the patient by at least 10% (e.g., 10%, 15%, 20%, 25%, 30%, 35%, 40%, 45%, or at least 50%). In some embodiments, the method decreases pulmonary arteriole muscularity in the patient. In some embodiments, the method decreases pulmonary arteriole muscularity in the patient by at least 10% (e.g., 10%, 15%, 20%, 25%, 30%, 35%, 40%, 45%, or at least 50%). In some embodiments, the patient has a right ventricular systolic pressure (RVSP) of greater than 35 mmHg. In some embodiments, the method decreases the RVSP in the patient. In some embodiments, the method reduces the RVSP in the patient by at least 10% (e.g., 10%, 15%, 20%, 25%, 30%, 35%, 40%, 45%, or at least 50%). In some embodiments, the patient has left ventricular fibrosis. In some embodiments, the method decreases the left ventricular fibrosis in the patient. In some embodiments, the method reduces the left ventricular fibrosis in the patient by at least 10% (e.g., 10%, 15%, 20%, 25%, 30%, 35%, 40%, 45%, or at least 50%). In some embodiments, the patient has right ventricular fibrosis. In some embodiments, the method decreases the right ventricular fibrosis in the patient. In some embodiments, the method reduces the right ventricular fibrosis in the patient by at least 10% (e.g., 10%, 15%, 20%, 25%, 30%, 35%, 40%, 45%, or at least 50%). In some embodiments, the patient has pulmonary fibrosis. In some embodiments, the method decreases the pulmonary fibrosis in the patient. In some embodiments, the method reduces the pulmonary fibrosis in the patient by at least 10% (e.g., 10%, 15%, 20%, 25%, 30%, 35%, 40%, 45%, or at least 50%).

In some embodiments, the patient has a comorbidity selected from the group consisting of systemic hypertension, diabetes mellitus, obesity, coronary artery disease (CAD), heart failure, and anemia. In some embodiments, the method further comprises administering to the patient an additional active agent and/or supportive therapy. In some embodiments, the additional active agent and/or supportive therapy is selected from the group consisting of: beta-blockers, angiotensin-converting enzyme inhibitors (ACE inhibitors), angiotensin receptor blockers (ARBs), neprilysin inhibitors, angiotensin receptor-neprilysin inhibitors (ARNI), mineralocorticoid receptor antagonists (MRA), hyperpolarization-activated cyclic nucleotide-gated (HCN) channel blockers, diuretic agents, lipid-lowering medications, endothelin blockers, PDE5 inhibitors, prostacyclins, cardiac resynchronization therapy, valve replacement, valve repair, implantable cardioverter-defibrillator (ICD), or a left ventricular assist device (LVAD). In some embodiments, the additional active agent and/or supportive therapy is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1->3)-beta-D-glucopyranosyl] oleanolic acid 28-O-beta-D-glucopyranosyl ester; 3-O-[beta-D-glucopyranosyl-(1->2)-beta-D-glucopyranosyl] oleanolic acid 28-O-beta-D-glucopyranosyl ester; 3-O-[a-L-rhamnopyranosyl-(1->3)-beta-D-glucuronopyranosyl] oleanolic acid; 3-O-[alpha-L-rhamnopyranosyl-(1-->3)-beta-D-glucuronopyranosyl]oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1): oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); eplerenone, spironolactone, ivabradine, implantable cardioverter-defibrillator (ICD), a left ventricular assist device (LVAD), or lung and/or heart transplantation.

In some embodiments, the patient has elevated brain natriuretic peptide (BNP) levels as compared to a heathy patient. In some embodiments, the patient has a BNP level of at least 100 pg/mL (e.g., 100, 150, 200, 300, 400, 500, 1000, 3000, 5000, 10,000, 15,000, or 20,000 pg/mL). In some embodiments, the method decreases BNP levels in the patient by at least 10% (e.g., 10%, 15%, 20%, 25%, 30%, 35%, 40%, 45%, 50%, 55%, 60%, 65%, 70%, 75%, or at least 80%). In some embodiments, the method decreases BNP levels to normal levels (i.e., <100 pg/ml).

In some embodiments, the method decreases NT-proBNP levels in the patient. In some embodiments, the method decreases NT-proBNP levels in the patient by at least 10% (e.g., 10%, 15%, 20%, 25%, 30%, 35%, 40%, 45%, 50%, 55%, 60%, 65%, 70%, 75%, or at least 80%). In some embodiments, the method decreases NT-proBNP levels in the patient by at least 30%. In some embodiments, the method decreases NT-proBNP levels to normal levels. In some embodiments, the normal level of NT-proBNP is <100 pg/ml. In some embodiments, the method increases exercise capacity of the patient. In some embodiments, the patient has a 6-minute walk distance from 150 to 400 meters. In some embodiments, the patient has a 6-minute walk distance from 150 to 550 meters. In some embodiments, the method increases the patient's 6-minute walk distance. In some embodiments, the method increases the patient's 6-minute walk distance by at least 10 meters (e.g., at least 10, 20, 30, 40, 50, 60, 70, 80, 90, 100, 125, 150, 175, 200, 250, 300, or more than 400 meters). In some embodiments, the method reduces the patient's Borg dyspnea index (BDI). In some embodiments, the method reduces the patient's BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points). In some embodiments, the patient has decreased renal function. In some embodiments, the method further improves renal function.

In some embodiments, the patient has Functional Class II or Class III pulmonary hypertension in accordance with the World Health Organization's functional classification system for pulmonary hypertension. In some embodiments, the patient has Functional Class I, Class II, Class III, or Class IV pulmonary hypertension as recognized by the World Health Organization. In some embodiments, the method prevents or delays pulmonary hypertension Functional Class progression (e.g., prevents or delays progression from Functional Class 1 to Class II, Class II to Class III, or Class III to Class IV pulmonary hypertension as recognized by the World Health Organization). In some embodiments, the method promotes or increases pulmonary hypertension Functional Class regression (e.g., promotes or increases regression from Class IV to Class III, Class III to Class 11, or Class 11 to Class I pulmonary hypertension as recognized by the World Health Organization). In some embodiments, the patient has Functional Class II or Class III pulmonary hypertension in accordance with the New York Heart Association's functional classification system for pulmonary hypertension. In some embodiments, the patient has Functional Class I, Class II, Class III, or Class IV pulmonary hypertension as recognized by the New York Heart Association. In some embodiments, the method prevents or delays pulmonary hypertension Functional Class progression (e.g., prevents or delays progression from Functional Class I to Class II, Class II to Class III, or Class III to Class IV pulmonary hypertension as recognized by the New York Heart Association). In some embodiments, the method promotes or increases pulmonary hypertension Functional Class regression (e.g., promotes or increases regression from Class IV to Class III, Class III to Class II, or Class II to Class I pulmonary hypertension as recognized by the New York Heart Association). In some embodiments, the method delays clinical worsening of PcPH. In some embodiments, the method delays clinical worsening of PcPH in accordance with the World Health Organization's functional classification system for pulmonary hypertension. In some embodiments, the method delays clinical worsening of PcPH in accordance with the New York Heart Association's functional classification system for pulmonary hypertension.

In some embodiments, the method reduces the risk of hospitalization for one or more complications associated with PcPH. In some embodiments, the patient has a hemoglobin level from >8 and <15 g/dl. In some embodiments, the patient has been treated with one or more vasodilators. In some embodiments, the patient has been treated with one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, trcprostinil, iloprost, ambrisentan, and tadalafil. In some embodiments, the method further comprises administration of one or more vasodilators. In some embodiments, the method further comprises administration of one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil.

In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 25-131 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 53. In some embodiments, the variant ActRIIB polypeptide is a fusion polypeptide comprising an ActRIIB polypeptide domain and one or more heterologous domains. In some embodiments, the variant ActRIIB polypeptide is an ActRIIB-Fc fusion polypeptide. In some embodiments, the fusion polypeptide further comprises a linker domain positioned between the ActRIIB polypeptide domain and the one or more heterologous domains or Fe domain. In some embodiments, the linker domain is selected from: TGGG, TGGGG, SGGGG, GGGGS. GGG. GGGG. SGGG, and GGGGS. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 5. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 12. In some embodiments, the ActRIIB polypeptide comprises one or more amino acid substitution with respect to the amino acid sequence of SEQ ID NO: 2 selected from the group consisting of: L38N, E50L, E52N, L57E, L57I, L57R, L57T, L57V, Y60D, G68R. K74E, W78Y, L79F, L79S, L79T, L79W, F82D, F82E, F82L, F82S, F82T, F82Y, N83R, E94K, and V99G. In some embodiments, the ActRIIB polypeptide comprises one or more amino acid substitution with respect to the amino acid sequence of SEQ ID NO: 2 selected from the group consisting of: L38N, E50L, E52D. E52N, E52Y, L57E, L57I, L57R, L57T, L57V, Y60D, G68R, K74E, W78Y, L79E, L79F, L79H, L79R, L79S, L79T, L79W, F82D, F82E, F82I, F82K, F82L, F82S, F82T, F82Y, N83R, E94K, and V99G. In some embodiments, the ActRIIB polypeptide comprises one or more amino acid substitution with respect to the amino acid sequence of SEQ ID NO: 2 selected from the group consisting of: A24N, S26T, N35E, E37A, E37D, L38N, R40A, R40K, S44T, L46V, L461, L46F, L46A, E50K, E50P, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R. V99E, V99G, V99K, E105N, F1081, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N. In some embodiments, the ActRIIB polypeptide comprises an L substitution at the position corresponding to E50 of SEQ ID NO: 2. In some embodiments, the ActRIIB polypeptide comprises an N substitution at the position corresponding to L38 of SEQ ID NO: 2. In some embodiments, the ActRIIB polypeptide comprises a G substitution at the position corresponding to V99 of SEQ ID NO: 2. In some embodiments, the ActRIIB polypeptide comprises an R substitution at the position corresponding to N83 of SEQ ID NO: 2. In some embodiments, the ActRIIB polypeptide comprises a T substitution at the position corresponding to F82 of SEQ ID NO: 2. In some embodiments, the ActRIIB polypeptide comprises an I substitution at the position corresponding to F82 of SEQ ID NO: 2. In some embodiments, the ActRIIB polypeptide comprises an K substitution at the position corresponding to F82 of SEQ ID NO: 2. In some embodiments, the ActRIIB polypeptide comprises an H substitution at the position corresponding to L79 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 276. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 278. In some embodiments, the polypeptide comprises an I substitution at the position corresponding to F82 of SEQ ID NO: 2 and an R substitution at the position corresponding to N83. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 279. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 332. In some embodiments, the polypeptide comprises a K substitution at the position corresponding to F82 of SEQ ID NO: 2 and an R substitution at the position corresponding to N83. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 333. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 335. In some embodiments, the polypeptide comprises a T substitution at the position corresponding to F82 of SEQ ID NO: 2 and an R substitution at the position corresponding to N83. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 336. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 338. In some embodiments, the polypeptide comprises a T substitution at the position corresponding to F82 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 339. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 341. In some embodiments, the polypeptide comprises an H substitution at the position corresponding to L79 of SEQ ID NO: 2 and an I substitution at the position corresponding to F82. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 342. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 344. In some embodiments, the polypeptide comprises an H substitution at the position corresponding to L79 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 345. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 347. In some embodiments, the polypeptide comprises an H substitution at the position corresponding to L79 of SEQ ID NO: 2 and a K substitution at the position corresponding to F82. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 348. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 350. In some embodiments, the polypeptide comprises an L substitution at the position corresponding to E50 of SEQ ID NO: 2. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 351. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 353. In some embodiments, the polypeptide comprises an N substitution at the position corresponding to L38 of SEQ ID NO: 2 and an R substitution at the position corresponding to L79. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 354. In some embodiments, the polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 356. In some embodiments, the polypeptide comprises an G substitution at the position corresponding to V99 of SEQ ID NO: 2. In some embodiments, the polypeptide comprises an N substitution at the position corresponding to A24 of SEQ ID NO: 2. In some embodiments, the polypeptide comprises an N substitution at the position corresponding to A24 of SEQ ID NO: 2 and an A substitution at the position corresponding to K74. In some embodiments, the polypeptide comprises a P substitution at the position corresponding to L79 of SEQ ID NO: 2. In some embodiments, the polypeptide comprises a P substitution at the position corresponding to L79 of SEQ ID NO: 2 and an A substitution at the position corresponding to K74. In some embodiments, the polypeptide comprises a D substitution at the position corresponding to L79 of SEQ ID NO: 2. In some embodiments, the polypeptide comprises an E substitution at the position corresponding to L79 of SEQ ID NO: 2. In some embodiments, the polypeptide comprises an S substitution at the position corresponding to P129 of SEQ ID NO: 2 and an A substitution at the position corresponding to P130. In some embodiments, the polypeptide comprises an A substitution at the position corresponding to N65 of SEQ ID NO: 2 and an N substitution at the position corresponding to S67. In some embodiments, the polypeptide comprises an A substitution at the position corresponding to K55 of SEQ ID NO: 2 and an I substitution at the position corresponding to F82. In some embodiments, the polypeptide comprises an H substitution at the position corresponding to L79 of SEQ ID NO: 2 and an I substitution at the position corresponding to F82. In some embodiments, the polypeptide comprises a K substitution at the position corresponding to L79 of SEQ ID NO: 2 and a K substitution at the position corresponding to F82. In some embodiments, the polypeptide comprises a W substitution at the position corresponding to F82 of SEQ ID NO: 2 and an A substitution at the position corresponding to N83. In some embodiments, the ActRIIB polypeptide is a homodimer polypeptide. In some embodiments, the ActRIIB polypeptide is a heteromultimer polypeptide. In some embodiments, the ActRIIB polypeptide is a heterodimer polypeptide. In some embodiments, the variant ActRIIB polypeptide is lyophilized. In some embodiments, the variant ActRIIB polypeptide is soluble. In some embodiments, the variant ActRIIB polypeptide is administered using subcutaneous injection. In some embodiments, the variant ActRIIB polypeptide is administered every 4 weeks. In some embodiments, the variant ActRIIB polypeptide is glycosylated. In some embodiments, the variant ActRIIB polypeptide has a glycosylation pattern obtainable by expression in a Chinese hamster ovary cell.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1A and FIG. 1B show schematic examples of heteromeric polypeptide complexes comprising a first variant ActRIIB polypeptide (indicated as “X”) and a second variant ActRIIB polypeptide (indicated as “Y”). In the illustrated embodiments, the first variant ActRIIB polypeptide is part of a fusion polypeptide that comprises a first member of an interaction pair (“C₁”), and a second variant ActRIIB polypeptide is part of a fusion polypeptide that comprises a second member of an interaction pair (“C₂”). Suitable interaction pairs include, for example, heavy chain and/or light chain immunoglobulin interaction pairs, truncations, and variants thereof such as those described herein [e.g., Spiess et al (2015) Molecular Immunology 67(2A): 95-106]. In each fusion polypeptide, a linker may be positioned between the first variant ActRIIB polypeptide or second variant ActRIIB polypeptide and the corresponding member of the interaction pair. The first and second members of the interaction pair may be unguided, meaning that the members of the pair may associate with each other or self-associate without substantial preference, and they may have the same or different amino acid sequences. See FIG. 1A. Alternatively, the interaction pair may be a guided (asymmetric) pair, meaning that the members of the pair associate preferentially with each other rather than self-associate. See FIG. 1B.

FIG. 2 shows a multiple sequence alignment of various vertebrate ActRIIB precursor polypeptides without their intracellular domains (SEQ ID NOs: 358-363), human ActRIIA precursor polypeptide without its intracellular domain (SEQ ID NO: 364), and a consensus ActRII precursor polypeptide (SEQ ID NO: 365). Upper case letters in the consensus sequence indicate positions that are conserved. Lower case letters in the consensus sequence indicate an amino acid residue that is the predominant form, but not universal at that position.

FIG. 3 shows multiple sequence alignment of Fc domains from human IgG isotypes using Clustal 2.1. Hinge regions are indicated by dotted underline. Double underline indicates examples of positions engineered in IgG1 (SEQ ID NO: 13) Fc to promote asymmetric chain pairing and the corresponding positions with respect to other isotypes IgG4 (SEQ ID NO: 17), IgG2 (SEQ ID NO: 14), and IgG3 (SEQ ID NO: 15).

FIG. 4 shows the amino acid sequence of human ActRIIB precursor polypeptide (SEQ ID NO: 2); NCBI Reference Sequence NP_001097.2). The signal peptide is underlined, the extracellular domain is in bold (also referred to as SEQ ID NO: 1), and the potential N-linked glycosylation sites are boxed. SEQ ID NO: 2 is used as the wild-type reference sequence for human ActRIIB in this disclosure, and the numbering for the variants described herein are based on the numbering in SEQ ID NO: 2

FIG. 5 shows the amino acid sequence of a human ActRIIB extracellular domain polypeptide (SEQ ID NO: 1) in which numbering is based on the native human ActRIIB precursor sequence (see SEQ ID NO: 2).

FIG. 6 shows a nucleic acid sequence encoding human ActRIIB precursor polypeptide. SEQ ID NO: 4 consists of nucleotides 434-1972 of NCBI Reference Sequence NM_001106.4.

FIG. 7 shows a nucleic acid sequence (SEQ ID NO: 3) encoding a human ActRIIB(20-134) extracellular domain polypeptide.

FIG. 8A and FIG. 8B show values for ligand binding kinetics of homodimeric Fc-fusion polypeptides comprising variant or unmodified ActRIIB domains, as determined by surface plasmon resonance at 37° C. Amino acid numbering is based on SEQ ID NO: 2. ND # indicates that the value is not detectable over concentration range tested. Transient* indicates that the value is indeterminate due to transient nature of interaction. Control sample is ActRIIB-G1Fc (SEQ ID NO: 5).

FIG. 9 shows values for ligand binding kinetics of homodimeric Fc-fusion polypeptides comprising variant or unmodified ActRIIB domains, as determined by surface plasmon resonance at 37° C. Amino acid numbering is based on SEQ ID NO: 2. ND # indicates that the value is not detectable over concentration range tested. Transient binding* indicates that the value is indeterminate due to transient nature of interaction. Control sample is ActRIIB-G1Fc (SEQ ID NO: 5).

FIG. 10 shows values for ligand binding kinetics of homodimeric Fc-fusion polypeptides comprising variant or unmodified ActRIIB domains, as determined by surface plasmon resonance at 25° C. ND # indicates that the value is not detectable over concentration range tested. Amino acid numbering is based on SEQ ID NO: 2.

FIG. 11 shows a schematic image of a linearized version of cardiopulmonary circulation and the regions associated with various types of PH. The difference between pre-capillary pulmonary hypertension, isolated post-capillary pulmonary hypertension, and combined post- and pre-capillary pulmonary hypertension are based on pulmonary hemodynamic parameters and the involvement of various regions of the cardiopulmonary system (pre and/or post capillary regions). Abbreviations are as follows: VC—vena cava; RA—right atrium; RV—right ventricle; PA—pulmonary artery; PC—pulmonary capillaries; PV—pulmonary ventricles; LA—left atrium; LV—left ventricle; AO—Aorta. See, e.g., Aras M A, et al. Curr Cardiol Rep. 2019; 21(7):62 and Galiè N. et al. Eur Heart J. 2018:39(15):1265-1268.

FIG. 12 shows a schematic image of a linearized version of cardiopulmonary circulation and the hemodynamic parameters associated with pre-capillary PH. Abbreviations are as follows: VC—vena cava; RA—right atrium; RV—right ventricle; PA—pulmonary artery; PC—pulmonary capillaries; PV—pulmonary ventricles; LA—left atrium; LV—left ventricle; AO—Aorta; mPAP—mean pulmonary arterial pressure; PAWP—pulmonary arterial wedge pressure; PVR—pulmonary vascular resistance. Id.

FIG. 13 shows a schematic image of a linearized version of cardiopulmonary circulation and the hemodynamic parameters associated with isolated post-capillary PH (IpcPH). Abbreviations are as follows: VC—vena cava; RA—right atrium; RV—right ventricle; PA—pulmonary artery; PC—pulmonary capillaries; PV—pulmonary ventricles; LA—left atrium; LV—left ventricle; AO—Aorta; mPAP—mean pulmonary arterial pressure; PAWP—pulmonary arterial wedge pressure; PVR—pulmonary vascular resistance. Id.

FIG. 14 shows a schematic image of a linearized version of cardiopulmonary circulation and the hemodynamic parameters associated with combined post- and pre-capillary PH (CpcPH). Abbreviations are as follows: VC—vena cava; RA—right atrium; RV—right ventricle; PA—pulmonary artery; PC—pulmonary capillaries; PV—pulmonary ventricles; LA—left atrium; LV—left ventricle; AO—Aorta; mPAP—mean pulmonary arterial pressure; PAWP—pulmonary arterial wedge pressure; PVR—pulmonary vascular resistance. Id.

FIGS. 15-19 shows the therapeutic effect of ActRIIA-mFc in a TAC-PH model based on endpoints for left ventricle function. Twenty-six C57/B6 male mice (10 wks old) underwent TAC pulmonary hypertension surgery (TAC-PH) and ten age-matched animals underwent a mock surgical procedure (Sham) at day 0. Two weeks after the surgery, TAC-PH mice were randomized into two groups. i) fourteen mice were injected subcutaneously with vehicle control (phosphate buffered saline (PBS)), twice weekly for 4 weeks starting from day 14 after surgery, “TAC-PH/PBS”; and a ii) twelve mice were injected subcutaneously with ActRIIA-mFc at a dose of 10 mg/kg twice weekly for 4 weeks starting from day 14 after TAC surgery. “TAC-PH/ActRIIA-mFc”. FIGS. 15-19 show endpoints for left ventricle function, including changes in cardiac hypertrophy heart weight/body weight (HW/BW) (FIG. 15), LV function parameters fractional shorting (FIG. 16) and LV ejection fraction (FIG. 17); and LV diastolic function parameters E/E′ [Ratio of mitrial inflow velocity (E) to mitrial annular velocity (E′)] (FIG. 18) and isovolumetric relaxation time (IVRT) (FIG. 19). Relative to “TAC-PH/PBS” treated mice, “TAC-PH/ActRIIA-mFc” treated mice demonstrated a significant effect of ActRIIA-mFc in reducing cardiac hypertrophy and improving cardiac function. Statistical significance (p value) is depicted as * p<0.05, **p<0.01, ***p<0.001, and ****p<0.0001 for comparison between “Sham” and sample “TAC-PH/PBS”. Statistical significance (p value) is depicted as #p<0.05, ##p<0.01. ###p<0.001, and ####p<0.0001 for comparison between “Sham” and sample “TAC-PH/ActRIIA-mFc”. Statistical significance (p value) is depicted as @p<0.05, @@p<0.01, @@@p<0.001, and @@@@<0.0001 for comparison between sample “TAC-PH/PBS” and sample “TAC-PH/ActRIIA-mFc”.

FIGS. 20-23 show the therapeutic effect of ActRIIA-mFc in a TAC-PH model based on endpoints for right ventricle function. Twenty-six C57/B6 male mice (10 wks old) underwent TAC pulmonary hypertension surgery (TAC-PH) and ten age-matched animals underwent a mock surgical procedure (Sham) at day 0. Two weeks after the surgery, TAC-PH mice were randomized into two groups. i) fourteen mice were injected subcutaneously with vehicle control (phosphate buffered saline (PBS)), twice weekly for 4 weeks starting from day 14 after surgery, “TAC-PH/PBS”; and a ii) twelve mice were injected subcutaneously with ActRIIA-mFc at a dose of 10 mg/kg twice weekly for 4 weeks starting from day 14 after TAC surgery, “TAC-PH/ActRIIA-mFc”. FIGS. 20-23 show endpoints for right ventricle function, including RV remodeling parameter right ventricular free wall thickness (RVFWT) (FIG. 20), RV remodeling and function parameter tricuspid annular plane systolic excursion (TAPSE) (FIG. 21), and RV function parameters RV stroke work (FIG. 22) and RV contractility (dP/dT) (FIG. 23). Relative to “TAC-PH/PBS” treated mice, “TAC-PH/ActRIIA-mFc” treated mice demonstrated a significant effect of ActRIIA-mFc in improving right heart remodeling and function. Statistical significance (p value) is depicted as * p<0.05, **p<0.01, ***p<0.001, and ****p<0.0001 for comparison between “Sham” and sample “TAC-PH/PBS”. Statistical significance (p value) is depicted as #p<0.05, ##p<0.01, ###p<0.001, and ####p<0.0001 for comparison between “Sham” and sample “TAC-PH/ActRIIA-mFc”. Statistical significance (p value) is depicted as @ p<0.05, @@p<0.01, @@@p<0.001, and @@@@p<0.0001 for comparison between sample “TAC-PH/PBS” and sample “TAC-PH/ActRIIA-mFc”.

FIGS. 24 and 25 show the therapeutic effect of ActRIIA-mFc in a TAC-PH model based on endpoints for lung remodeling. Twenty-six C57/B6 male mice (10 wks old) underwent TAC pulmonary hypertension surgery (TAC-PH) and ten age-matched animals underwent a mock surgical procedure (Sham) at day 0. Two weeks after the surgery, TAC-PH mice were randomized into two groups. i) fourteen mice were injected subcutaneously with vehicle control (phosphate buffered saline (PBS)), twice weekly for 4 weeks starting from day 14 after surgery, “TAC-PH/PBS”; and a ii) twelve mice were injected subcutaneously with ActRIIA-mFc at a dose of 10 mg/kg twice weekly for 4 weeks starting from day 14 after TAC surgery, “TAC-PH/ActRIIA-mFc”. FIGS. 24 and 25 show endpoints for lung remodeling, including ratio of lung weight to tibia length (LW/TL) (FIG. 24) and lung fibrosis percentage (FIG. 25). Relative to “TAC-PH/PBS” treated mice, “TAC-PH/ActRIIA-mFc” treated mice demonstrated a significant effect of ActRIIA-mFc in reducing pulmonary remodeling and fibrosis. Statistical significance (p value) is depicted as * p<0.05, **p<0.01, ***p<0.001, and ****p<0.0001 for comparison between “Sham” and sample “TAC-PH/PBS”. Statistical significance (p value) is depicted as #p<0.05, ##p<0.01, ###p<0.001, and ####p<0.0001 for comparison between “Sham” and sample “TAC-PH/ActRIIA-mFc”. Statistical significance (p value) is depicted as @p<0.05, @@p<0.01, @@@p<0.001, and @@@@p<0.0001 for comparison between sample “TAC-PH/PBS” and sample “TAC-PH/ActRIIA-mFc”.

FIG. 26 shows components of a kit comprising a lyophilized polypeptide and an injection device. A vial (1) holds lyophilized polypeptide, reconstituted sterile injectable solution, or sterile injectable solution. A prefilled syringe (2) containing a reconstitution solution is used to reconstitute lyophilized polypeptide from (1) into a sterile injectable solution. A vial adapter (3) couples the vial (1) to the pre-filled syringe (2) via attachment to the vial at one end, and attachment to the pre-filled syringe at an opposite end. A syringe (4) and needle (5) are provided for administration of sterile injectable solution. Swab wipes (6) are provided for sterilization of individual kit components.

FIGS. 27-30 show that treatment with an ActRIIA-mFc fusion protein improves diastolic dysfunction in a rat model of left ventricular diastolic dysfunction (also referred to as HEpEF) group 2 (subgroup 2.2) pulmonary hypertension (PH). The experimental strategy used to test the preventative effects of ActRIIA-mFc in the rat model of HEpEF is shown in FIG. 27. FIGS. 28-30 show endpoints for left ventricular function, including the left ventricular ejection fraction (FIG. 28); LV diastolic function parameters E/E′ [Ratio of mitrial inflow velocity (E) to mitrial annular velocity (E′)] (FIG. 29); and isovolumetric relaxation time (IVRT) (FIG. 30). Statistical significance (p value) is depicted as * p<0.05, **p<0.01, and ***p<0.001.

FIGS. 31-33 show that treatment with an ActRIIA-mFc fusion protein reduces left heart remodeling in a rat model of left ventricular diastolic dysfunction (also referred to as HEpEF) group 2 (subgroup 2.2) pulmonary hypertension (PH). FIGS. 31-33 show endpoints for left heart remodeling, including changes in ratio of heart weight to tibia length (HW/TL) (FIG. 31); interventricular septal dimension at diastole (IVSd) (FIG. 32); and left ventricular mass (LVM) (FIG. 33). Statistical significance (p value) is depicted as * p<0.05, **p<0.01, and ***p<0.001.

FIGS. 34-36 show that treatment with an ActRIIA-mFc fusion protein reduces right ventricular systolic pressure (RVSP) and improves right ventricular function in a rat model of left ventricular diastolic dysfunction (also referred to as HEpEF) group 2 (subgroup 2.2) pulmonary hypertension (PH). FIGS. 34-36 show endpoints for right ventricular function, including changes in right ventricular free wall thickness (FIG. 34); pulmonary artery acceleration time (PAAT) (FIG. 35); and right ventricular systolic pressure (RVSP) (FIG. 36). Statistical significance (p value) is depicted as * p<0.05 and **p<0.01.

FIGS. 37-39 show that treatment with an ActRIIA-mFc fusion protein significantly reduced the fibrosis in LV, RV and lung in a rat model of left ventricular diastolic dysfunction (also referred to as HEpEF) group 2 (subgroup 2.2) pulmonary hypertension (PH). FIGS. 37-39 show a reduction in fibrosis, including changes in left ventricular fibrosis (FIG. 37); right ventricular fibrosis (FIG. 38); and lung fibrosis (FIG. 39). Statistical significance (p value) is depicted as * p<0.05 and **p<0.01.

FIGS. 40-43 show that treatment with an ActRIIA-mFc fusion protein significantly improves hyperglycemia and glucose intolerance in a rat model of left ventricular diastolic dysfunction (also referred to as HEpEF) group 2 (subgroup 2.2) pulmonary hypertension (PH). FIGS. 40-43 show endpoints for hyperglycemia and glucose intolerance, including changes in body weight (FIG. 40); fasting glucose (FIG. 41); blood glucose (FIG. 42); and glucose/creatine ratio (FIG. 43). Statistical significance (p value) is depicted as * p<0.05, **p<0.01, and ***p<0.001.

FIGS. 44-48 show that treatment with an ActRIIA-mFc fusion protein inhibits cardiac remodeling and improves LV function in a mouse model of PH due to heart failure with reduced LVEF (also referred to as HErEF) group 2 (subgroup 2.1) pulmonary hypertension (PH) and valvular heart disease (subgroup 2.3). The experimental strategy used to test the preventative effects of ActRIIA-mFc in the rat model of HErEF is shown in FIG. 44. FIGS. 45-48 show endpoints for left ventricle function, including changes in cardiac hypertrophy heart weight/tibia length (HW/TL)(FIG. 46), LV function parameters such as LV ejection fraction (FIG. 45), LV diastolic function parameters E/E′ [Ratio of mitral inflow velocity (E) to mitral annular velocity (E′)] (FIG. 47) and isovolumetric relaxation time (IVRT) (FIG. 48). Relative to “TAC PBS” treated mice, “TAC ActRIIA-mFc” treated mice demonstrated a significant effect of ActRIIA-mFc in inhibiting cardiac remodeling and improving LV function. Statistical significance (p value) is depicted as * p<0.05, **p<0.01, ***p<0.001, and ****p<0.0001 for comparison between “TAC PBS” and sample “TAC ActRIIA-mFc”. Statistical significance (p value) is depicted as #p<0.05, ##p<0.01, ###p<0.001, and ####p<0.0001 for comparison between “Sham” and sample “TAC PBS.”

FIGS. 49-51 show the therapeutic effect of ActRIIA-mFc in a TAC-PH model based on endpoints for right ventricle function. FIGS. 49-51 show endpoints for right ventricle function including right ventricular systolic pressure (RVSP)(FIG. 49), right ventricular free wall thickness (RVFWT)(FIG. 50), and pulmonary artery acceleration time (PAAT)(FIG. 51). Relative to “TAC PBS” treated mice, “TAC ActRIIA-mFc” mice treated with either 3 mpk and 10 mpk demonstrated a significant effect of ActRIIA-mFc in reducing RVSP and improving RV function. Statistical significance (p value) is depicted as * p<0.05, **p<0.01, ***p<0.001, and ****p<0.0001 for comparison between “TAC PBS” and sample “TAC ActRIIA-mFc.” Statistical significance (p value) is depicted as #p<0.05, ##p<0.01, ###p<0.001, and ####p<0.0001 for comparison between “Sham” and sample “TAC PBS.”

FIGS. 52-54 show the therapeutic effect of ActRIIA-mFc in a TAC-PH model based on endpoints for fibrosis in the left ventricle (LV), right ventricle (RV), and lung. FIGS. 52-54 show endpoints for fibrosis in the left ventricle (LV) (FIG. 47), right ventricle (RV) (FIG. 53), and lung (FIG. 54). Relative to “TAC PBS” treated mice, “TAC ActRIIA-mFc” mice treated with either 3 mpk or 10 mpk demonstrated a significant effect of ActRIIA-mFc in reducing fibrosis in the LV (FIG. 52), RV (FIG. 53), and lung (FIG. 54). Statistical significance (p value) is depicted as * p<0.05, **p<0.01, ***p<0.001, and ****p<0.0001 for comparison between “TAC PBS” and sample “TAC ActRIIA-mFc.” Statistical significance (p value) is depicted as #p<0.05, ##p<0.01, ###p<0.001, and ####p<0.0001 for comparison between “Sham” and sample “TAC PBS.”

FIGS. 55-60 show that treatment with an ActRIIA-mFc fusion protein reduces right ventricular systolic pressure (RVSP) and improves cardiopulmonary function in a rat model of left ventricular diastolic dysfunction (also referred to as HEpEF) group 2 (subgroup 2.2) pulmonary hypertension (PH). The experimental strategy used to test the preventative effects of an ActRIIA-mFc fusion protein in the rat model of HEpEF is shown in FIG. 55. FIGS. 56-60 show endpoints for right ventricular function, including changes in pulmonary artery acceleration time (PAAT) (FIG. 56); right ventricular systolic pressure (RVSP) (FIG. 57); right ventricular wall thickness (RVWT) (FIG. 58); tricuspid annular plane systolic excursion (TAPSE) (FIG. 59); and Fulton index, calculated as the ratio of right ventricular weight (RV) to weight of the combined left ventricle and septum (LV+S) (FIG. 60). Statistical significance (p value) is depicted as * p<0.05, **p<0.01, ***p<0.001, and ****p<0.0001.

### DETAILED DESCRIPTION

### 1. Overview

The present disclosure relates to compositions and methods of treating post-capillary pulmonary hypertension (e.g., WHO Group 2 and/or Group 5 PH) comprising administering to a patient in need thereof an effective amount of a variant ActRIIB polypeptide as described herein. In certain embodiments, the present disclosure provides methods of treating or preventing post-capillary pulmonary hypertension (PcPH) in an individual in need thereof through administering to the individual a therapeutically effective amount of a variant ActRIIB polypeptide as described herein. In certain embodiments, the present disclosure provides methods of treating or preventing combined post- and pre-capillary PH in an individual in need thereof through administering to the individual a therapeutically effective amount of a variant ActRIIB polypeptide as described herein.

Pulmonary hypertension due to left heart disease (PH-LHD) (also known as WHO Group 2 PH) is a complex pathophenotype that, when present, may result in an increased susceptibility to adverse events and a worse clinical outcome. Among those patients with PH-LHD, two phenotypes have been described: 1) a group of isolated post-capillary (IpcPH) or “passive” PH in which elevated pulmonary pressures are reversible and in proportion to increases in left atrial pressure, and 2) a group with “pre-capillary” component [combined post-capillary and pre-capillary PH (CpcPH)] whose pulmonary hypertension is worse than can be fully explained by passive elevation secondary to elevated left atrial pressure. This latter group, CpcPH, may have comorbid pulmonary vascular remodeling and therefore may demonstrate persistent PH after interventions to lower left sided filling pressures.

PH-LHD is sometimes defined as patients having a pulmonary capillary wedge pressure (PCWP)>15 mmHg and a mean pulmonary arterial pressure (mPAP)≥25 mmHg (or a mean pulmonary arterial pressure (mPAP)≥20 mmHg under updated guidelines). PH-LHD occurs as a consequence of the backward transmission of high left sided filling pressures, mainly driven by LV diastolic function, directly to the post-capillary pulmonary vessels and, thereby, to the rest of the pulmonary circulation. In some embodiments, PH-LHD is driven by both systolic and diastolic dysfunction. PH-LHD may be associated with or caused by PH due to heart failure with preserved left ventricle ejection fraction (LVEF) [also known as HFpEF], PH due to heart failure with reduced LVEF (also known as HFrEF), valvular heart disease, or congenital/acquired cardiovascular conditions leading to post-capillary PH. Compared with PAH, patients with PH-LHD are often older, female, with a higher prevalence of cardiovascular co-morbidities and most, if not all, of the features of metabolic syndrome.

For WHO Group 2 (PH-LHD) and Group 5 PH patients, there are no approved specific therapies available beyond treatment of the underlying disease. Most PH-LHD therapies target the underlying condition (e.g., repair of valvular heart disease) rather than specifically treating PH. The lack of specific therapies is particularly problematic because PH-LHD is the most common cause of PH in western countries and its presence commonly results in adverse course of the disease. Specifically, the presence of PH-LHD can result in more severe symptoms in LHD, worse exercise tolerance, and a negative impact on outcome. Accordingly, there is a high unmet need for new treatments for post-capillary pulmonary hypertension (e.g., WHO Group 2 and/or Group 5 PH) and these treatments would have the potential to positively affect large numbers of patients.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters arc set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is +10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore. “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRIIB Polypeptides

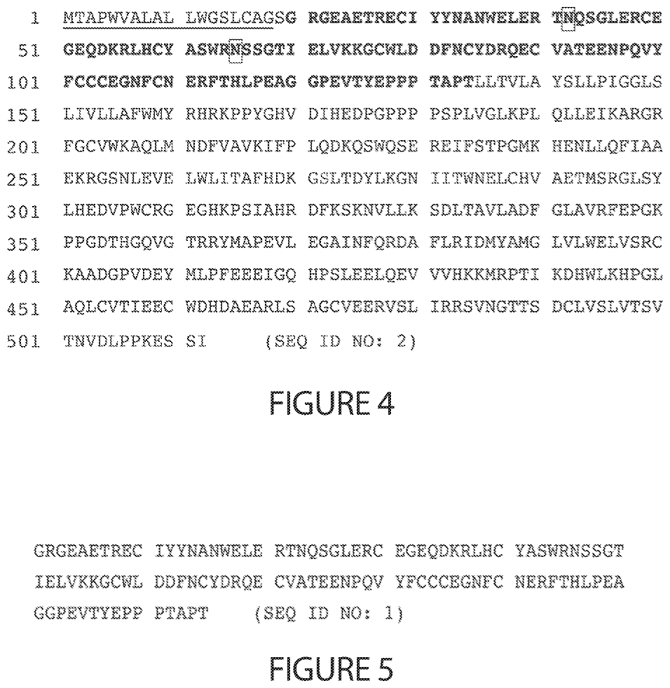

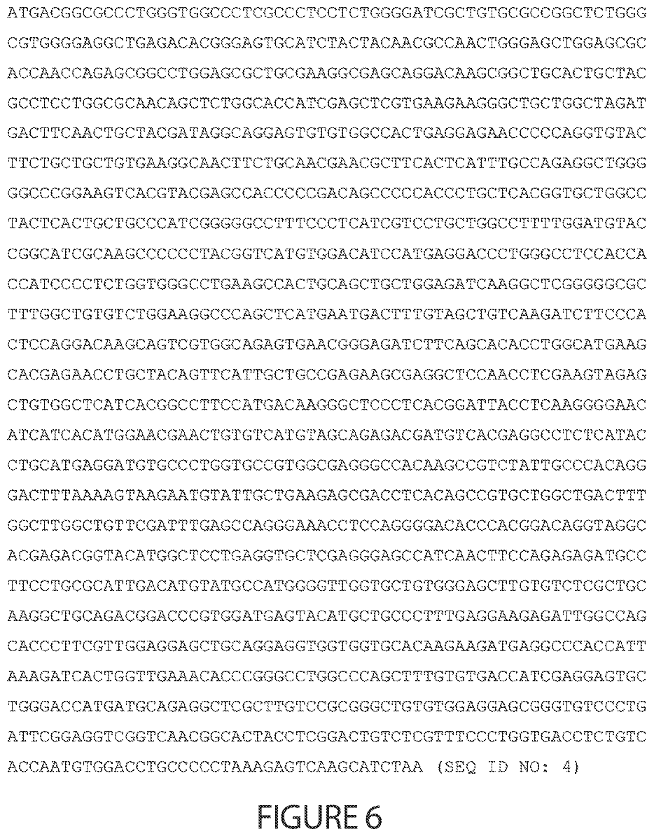

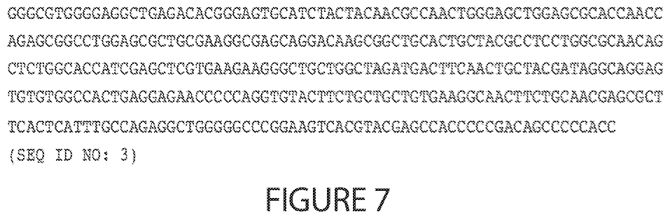

In certain aspects, the disclosure relates to variant ActRIIB polypeptides and uses thereof (e.g., of treating, preventing, or reducing the progression rate and/or severity of post-capillary pulmonary hypertension (PcPH) or one or more complications of PcPH). As used herein, the term “ActRIIB” refers to a family of activin receptor type IIB (ActRIIB) polypeptides and ActRIIB-related polypeptides, derived from any species. Members of the ActRIIB family are generally all transmembrane polypeptides, composed of a ligand-binding extracellular domain with cysteine-rich region, a transmembrane domain, and a cytoplasmic domain with predicted serine/threonine kinase specificity. The amino acid sequence of human ActRIIB precursor polypeptide is shown in FIG. 4 (SEQ ID NO: 2). Examples of variant ActRIIB polypeptides are provided throughout the present disclosure as well as in International Patent Application Publication Nos. WO 2006/012627, WO 2008/097541, WO 2010/151426, WO 2011/020045, WO 2018/009624, and WO 2018/067874 which are incorporated herein by reference in their entirety.

The term “ActRIIB polypeptide” is used to refer to polypeptides comprising any naturally occurring polypeptide of an ActRIIB family member as well as any variants thereof (including mutants, fragments, fusions, and peptidomimetic forms) that retain a useful activity. For example, ActRIIB polypeptides include polypeptides derived from the sequence of any known ActRIIB having a sequence at least about 80% identical to the sequence of an ActRIIB polypeptide, and preferably at least 85%, 90%, 91%, 92%, 93%, 94%, 95%, 96%, 97%, 98%, 99% or greater identity.

In a specific embodiment, the disclosure relates to soluble ActRIIB polypeptides. As described herein, the term “soluble ActRIIB polypeptide” generally refers to polypeptides comprising an extracellular domain of an ActRIIB polypeptide. The term “soluble ActRIIB polypeptide,” as used herein, includes any naturally occurring extracellular domain of an ActRIIB polypeptide as well as any variants thereof (including mutants, fragments and peptidomimetic forms) that retain a useful activity. For example, the extracellular domain of an ActRIIB polypeptide binds to a ligand and is generally soluble. Examples of soluble ActRIIB polypeptides include an ActRIIB extracellular domain (SEQ ID NO: 1) shown in FIG. 5 as well as SEQ ID NO: 53. This truncated ActRIIB extracellular domain (SEQ ID NO: 53) is denoted ActRIIB(25-131) based on numbering in SEQ ID NO: 2. Other examples of soluble ActRIIB polypeptides comprise a signal sequence in addition to the extracellular domain of an ActRIIB polypeptide (see Example 1). The signal sequence can be a native signal sequence of an ActRIIB, or a signal sequence from another polypeptide, such as a tissue plasminogen activator (TPA) signal sequence or a honey bee melatin signal sequence.

TGF-β signals are mediated by heteromeric complexes of type I and type II serine/threonine kinase receptors, which phosphorylate and activate downstream Smad proteins upon ligand stimulation (Massagud, 2000, Nat. Rev. Mol. Cell Biol. 1:169-178). These type I and type II receptors are all transmembrane polypeptides, composed of a ligand-binding extracellular domain with cysteine-rich region, a transmembrane domain, and a cytoplasmic domain with predicted serine/threonine specificity. Type I receptors are essential for signaling, and type II receptors are required for binding ligands. Type I and type II activin receptors form a stable complex after ligand binding, resulting in phosphorylation of type I receptors by type II receptors.

Two related type II receptors, ActRIIA and ActRIIB, have been identified as the type II receptors for activins (Mathews and Vale, 1991. Cell 65:973-982, Attisano et al., 1992, Cell 68: 97-108). Besides activins, ActRIIA and ActRIIB can biochemically interact with several other TGF-β family proteins, including BMP7, Nodal, GDF8, and GDF11 (Yamashita et al., 1995, J. Cell Biol. 130:217-226; Lee and McPherron, 2001, Proc. Natl. Acad. Sci. 98:9306-9311; Yeo and Whitman, 2001, Mol. Cell 7: 949-957; Oh et al., 2002, Genes Dev. 16:2749-54). Applicants have found that soluble ActRIIA-Fc fusion polypeptides and ActRIIB-Fc fusion polypeptides have substantially different effects in vivo, with ActRIIA-Fc having primary effects on bone and ActRIIB-Fc having primary effects on skeletal muscle.

In certain embodiments, the present disclosure relates to antagonizing a ligand of ActRIIB receptors (also referred to as an ActRIIB ligand) with a subject ActRIIB polypeptide (e.g., a variant ActRIIB polypeptide). In some embodiments, the variant ActRIIB polypeptide is a member of a homomultimer (e.g., homodimer). In some embodiments, the variant ActRIIB polypeptide is a member of a heteromultimer (e.g., a heterodimer). In some embodiments, the variant ActRIIB polypeptide heteromultimerizes with any of the other soluble receptors disclosed herein. In some embodiments, the variant ActRIIB polypeptide is fused to any of the polypeptides disclosed herein (any of the soluble receptors disclosed herein). Thus, compositions and methods of the present disclosure are useful for treating, preventing, or reducing the progression rate and/or severity of PcPH or one or more complications of PcPH. Exemplary ligands of ActRIIB receptors include some TGF-β family members, such as activin A, activin B, GDF3, GDF8, GDF11, BMP6, BMP9 and BMP10. In some embodiments, any of the heteromultimers disclosed herein have a different binding profile as compared to any of the ActRIIB homomultimers (e.g., homodimers) disclosed herein.

Activins are dimeric polypeptide growth factors and belong to the TGF-beta superfamily. There are three activins (A, B, and AB) that are homo/heterodimers of two closely related p subunits (β_{A}β_{A}, β_{B}β_{B}, and β_{A}β_{B}). In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos (DePaolo et al., 1991, Proc SocEp Biol Med. 198:500-512; Dyson et al., 1997, Curr Biol. 7:81-84; Woodruff, 1998, Biochem Pharmacol. 55:953-963). Moreover, erythroid differentiation factor (EDF) isolated from the stimulated human monocytic leukemic cells was found to be identical to activin A (Murata et al., 1988, PNAS, 85:2434). It was suggested that activin A acts as a natural regulator of erythropoiesis in the bone marrow. In several tissues, activin signaling is antagonized by its related heterodimer, inhibin. For example, during the release of follicle-stimulating hormone (FSH) from the pituitary, activin promotes FSH secretion and synthesis, while inhibin prevents FSH secretion and synthesis. Other polypeptides that may regulate activin bioactivity and/or bind to activin include follistatin (FS), follistatin-related polypeptide (FSRP). α₂-macroglobulin. Cerberus, and endoglin.

Growth and differentiation factor-8 (GDF8) is also known as myostatin. GDF8 is a negative regulator of skeletal muscle mass. GDF8 is highly expressed in the developing and adult skeletal muscle. The GDF8 null mutation in transgenic mice is characterized by a marked hypertrophy and hyperplasia of the skeletal muscle (McPherron et al., Nature, 1997, 387:83-90). Similar increases in skeletal muscle mass are evident in naturally occurring mutations of GDF8 in cattle (Ashmore et al., 1974, Growth, 38:501-507; Swatland and Kieffer, J. Anim. Sci., 1994, 38:752-757; McPherron and Lee, Proc. Natl. Acad. Sci. USA, 1997, 94:12457-12461; and Kambadur et al., Genome Res., 1997, 7:910-915) and, strikingly, in humans (Schuelke et al., N Engl J Med 2004; 350:2682-8). Studies have also shown that muscle wasting associated with HIV-infection in humans is accompanied by increases in GDF8 polypeptide expression (Gonzalez-Cadavid et al., PNAS, 1998, 95:14938-43). In addition, GDF8 can modulate the production of muscle-specific enzymes (e.g., creatine kinase) and modulate myoblast cell proliferation (WO 00/43781). The GDF8 propeptide can noncovalently bind to the mature GDF8 domain dimer, inactivating its biological activity (Miyazono et al. (1988) J. Biol. Chem., 263: 6407-6415; Wakefield et al. (1988) J. Biol. Chem., 263: 7646-7654; and Brown et al. (1990) Growth Factors, 3: 35-43). Other polypeptides which bind to GDF8 or structurally related polypeptides and inhibit their biological activity include follistatin, and potentially, follistatin-related polypeptides (Gamer et al. (1999) Dev. Biol., 208: 222-232).

Growth and differentiation factor-11 (GDF11), also known as BMP11, is a secreted protein (McPherron et al., 1999, Nat. Genet. 22: 260-264). GDF11 is expressed in the tail bud, limb bud, maxillary and mandibular arches, and dorsal root ganglia during mouse development (Nakashima et al., 1999, Mech. Dev. 80: 185-189). GDF11 plays a unique role in patterning both mesodermal and neural tissues (Gamer et al., 1999, Dev Biol., 208:222-32). GDF11 was shown to be a negative regulator of chondrogenesis and myogenesis in developing chick limb (Gamer et al., 2001, Dev Biol. 229:407-20). The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium (Wu et al., 2003, Neuron. 37:197-207).

In certain aspects, the present disclosure relates to the use of certain ActRIIB polypeptides (e.g., soluble ActRIIB polypeptides) to antagonize the signaling of ActRIIB ligands generally, in any process associated with PcPH (e.g., WHO Group 2 and/or Group 5 PH). Optionally. ActRIIB polypeptides of the disclosure may antagonize one or more ligands of ActRIIB receptors, such as activin A, activin B, GDF8, GDF11, or BMP10, and may therefore be useful in treating, preventing, or reducing the progression rate and/or severity of PcPH (e.g., WHO Group 2 and/or Group 5 PH) or one or more complications of PcPH (e.g., smooth muscle and/or endothelial cell proliferation in the pulmonary artery, angiogenesis in the pulmonary artery, dyspnea, chest pain, pulmonary vascular remodeling, right ventricular hypertrophy, left ventricular hypertrophy, left atrium dilation, and pulmonary fibrosis). In particular, the disclosure provides variant ActRIIB polypeptides with reduced binding affinity to BMP9 while retaining binding affinity to one or more of activin A, activin B, GDF8, GDF11, and BMP10. Accordingly, these variant ActRIIB polypeptides may be more useful than an unmodified ActRIIB polypeptide in certain applications where such selective antagonism is advantageous (e.g., PcPH). Examples include therapeutic applications where it is desirable to retain antagonisms of one or more of activin A, activin B, GDF8, GDF11, and BMP10, while reducing antagonism of BMP9. In some embodiments, any of the ActRIIB polypeptides disclosed herein may be combined with any of the other polypeptides disclosed herein. In some embodiments, the ActRIIB polypeptide is a member of a heteromultimer (e.g., a heterodimer) with any of the polypeptides disclosed herein.

### Variant ActRIIB Polypeptides

In certain specific embodiments, the present disclosure contemplates making mutations in the extracellular domain (also referred to as ligand-binding domain) of an ActRIIB polypeptide such that the variant (or mutant) ActRIIB polypeptide has altered ligand-binding activities (e.g., binding affinity or binding selectivity). In certain cases, such variant ActRIIB polypeptides have altered (elevated or reduced) binding affinity for a specific ligand. In other cases, the variant ActRIIB polypeptides have altered binding selectivity for their ligands. For example, the disclosure provides a number of variant ActRIIB polypeptides that have reduced binding affinity to BMP9, compared to a non-modified ActRIIB polypeptide, but retain binding affinity for one or more of activin A, activin B. GDF8, GDF11, and BMP10. Optionally, the variant ActRIIB polypeptides have similar or the same biological activities of their corresponding wild-type ActRIIB polypeptides. For example, a variant ActRIIB polypeptide of the disclosure may bind to and inhibit function of an ActRIIB ligand (e.g., activin A, activin B, GDF8, GDF11 or BMP10). In some embodiments, a variant ActRIIB polypeptide of the disclosure treats, prevents, or reduces the progression rate and/or severity of PcPH or one or more complications of PcPH. Examples of ActRIIB polypeptides include human ActRIIB precursor polypeptide (SEQ ID NO: 2), and soluble human ActRIIB polypeptides (e.g., SEQ ID NOs: 1, 5, 6, 12, 276, 278, 279, 332, 333, 335, 336, 338, 339, 341, 342, 344, 345, 347, 348, 350, 351, 353, 354, 356, and 357). In some embodiments, the variant ActRIIB polypeptide is a member of a homomultimer (e.g., homodimer). In some embodiments, the variant ActRIIB polypeptide is a member of a heteromultimer (e.g., a heterodimer). In some embodiments, any of the variant ActRIIB polypeptides may be combined (e.g., heteromultimerized with and/or fused to) with any of polypeptides disclosed herein.

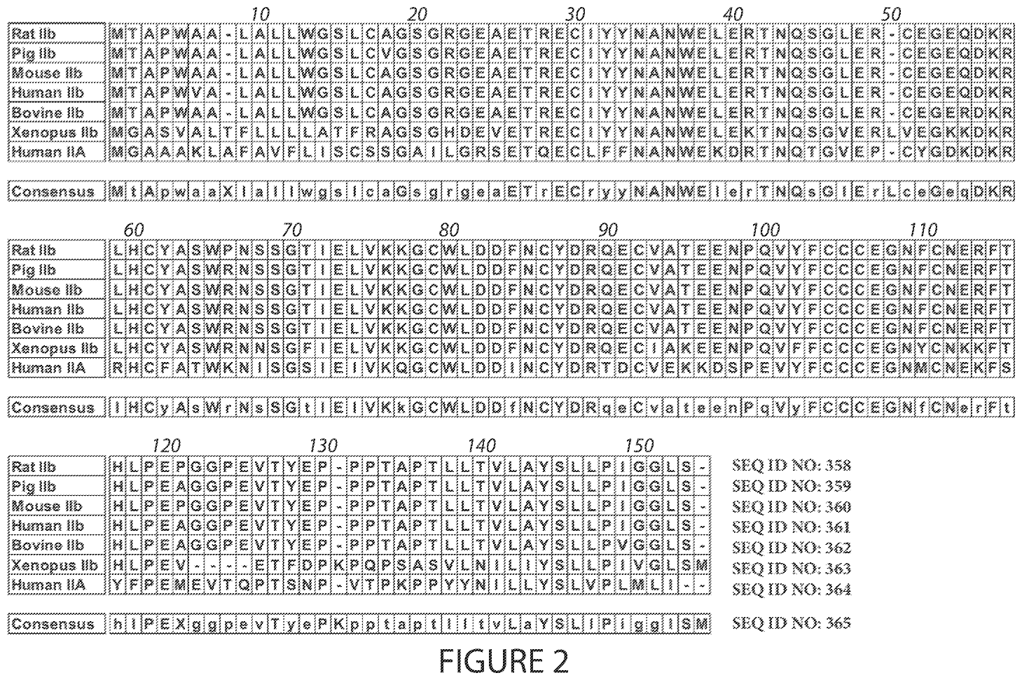

ActRIIB is well-conserved across nearly all vertebrates, with large stretches of the extracellular domain conserved completely. See, e.g., FIG. 2. Many of the ligands that bind to ActRIIB are also highly conserved. Accordingly, comparisons of ActRIIB sequences from various vertebrate organisms provide insights into residues that may be altered. Therefore, an active, human ActRIIB variant may include one or more amino acids at corresponding positions from the sequence of another vertebrate ActRIIB, or may include a residue that is similar to that in the human or other vertebrate sequence.

The disclosure identifies functionally active portions and variants of ActRIIB. Applicant has previously ascertained that an Fc fusion polypeptide having the sequence disclosed by Hilden et al. (Blood. 1994 Apr. 15; 83(8):2163-70), which has an alanine at the position corresponding to amino acid 64 of SEQ ID NO: 2 (A64), has a relatively low affinity for activin and GDF11. By contrast, the same Fc fusion polypeptide with an arginine at position 64 (R64) has an affinity for activin and GDF-11 in the low nanomolar to high picomolar range. Therefore, a sequence with an R64 (SEQ ID NO: 2) is used as the wild-type reference sequence for human ActRIIB in this disclosure, and the numbering for the variants described herein are based on the numbering in SEQ ID NO: 2. Additionally, one of skill in the art can make any of the ActRIIB variants described herein in the A64 background.

A processed extracellular ActRIIB polypeptide sequence is shown in SEQ ID NO: 1 (see, e.g., FIG. 5). In some embodiments, a processed ActRIIB polypeptide may be produced with an “SGR . . . ” sequence at the N-terminus. In some embodiments, a processed ActRIIB polypeptide may be produced with a “GRG . . . ” sequence at the N-terminus. For example, it is expected that some constructs, if expressed with a TPA leader, will lack the N-terminal serine. Accordingly, mature ActRIIB sequences described herein may begin with either an N-terminal serine or an N-terminal glycine (lacking the N-terminal serine).

Attisano et al. (Cell. 1992 Jan. 10; 68(1):97-108) showed that a deletion of the proline knot at the C-terminus of the extracellular domain of ActRIIB reduced the affinity of the receptor for activin. Data disclosed in WO2008097541 show that an ActRIIB-Fc fusion polypeptide containing amino acids 20-119 of SEQ ID NO: 2, “ActRIIB(20-119)-Fc” has reduced binding to GDF11 and activin relative to an ActRIIB(20-134)-Fc, which includes the proline knot region and the complete juxtamembrane domain. However, an ActRIIB(20-129)-Fc polypeptide retains similar but somewhat reduced activity relative to the wild type, even though the proline knot region is disrupted. Thus, ActRIIB extracellular domains that stop at amino acid 134, 133, 132, 131, 130 and 129 are all expected to be active, but constructs stopping at 134 or 133 may be most active. Similarly, mutations at any of residues 129-134 are not expected to alter ligand binding affinity by large margins. In support of this, mutations of P129 and P130 do not substantially decrease ligand binding. Therefore, an ActRIIB-Fc fusion polypeptide may end as early as amino acid 109 (the final cysteine), however, forms ending at or between 109 and 119 are expected to have reduced ligand binding. Amino acid 119 is poorly conserved and so is readily altered or truncated. Forms ending at 128 or later retain ligand binding activity. Forms ending at or between 119 and 127 will have an intermediate binding ability. Any of these forms may be desirable to use, depending on the clinical or experimental setting.

At the N-terminus of ActRIIB, it is expected that a polypeptide beginning at amino acid 29 or before will retain ligand binding activity. Amino acid 29 represents the initial cysteine. An alanine-to-asparagine mutation at position 24 introduces an N-linked glycosylation sequence without substantially affecting ligand binding. This confirms that mutations in the region between the signal cleavage peptide and the cysteine cross-linked region, corresponding to amino acids 20-29, are well tolerated. In particular, constructs beginning at position 20, 21, 22, 23 and 24 will retain activity, and constructs beginning at positions 25, 26, 27, 28 and 29 are also expected to retain activity. Data shown in WO2008097541 demonstrate that, surprisingly, a construct beginning at 22, 23, 24 or 25 will have the most activity.

Taken together, an active portion of ActRIIB comprises amino acids 29-109 of SEQ ID NO: 2, and constructs may, for example, begin at a residue corresponding to amino acids 20-29 and end at a position corresponding to amino acids 109-134. Other examples include constructs that begin at a position from 20-29 or 21-29 and end at a position from 119-134, 119-133 or 129-134, 129-133. Other examples include constructs that begin at a position from 20-24 (or 21-24, or 22-25) and end at a position from 109-134 (or 109-133), 119-134 (or 119-133) or 129-134 (or 129-133). Variants within these ranges are also contemplated, particularly those having at least 80%, 85%, 90%, 91%, 92%, 93%, 94%, 95%, 96%, 97%, 98%, or 99% identity to the corresponding portion of SEQ ID NO: 1.

In certain embodiments, a variant ActRIIB polypeptide has an amino acid sequence that is at least 75% identical to an amino acid sequence selected from SEQ ID NOs: 1, 2, and 53. In certain cases, the variant ActRIIB polypeptide has an amino acid sequence at least 80%, 85%, 90%, 91%, 92%, 93%, 94%, 95%, 96%, 97%, 98%, 99%, or 100% identical to an amino acid sequence selected from SEQ ID NOs: 1, 2, and 53. In certain cases, the variant ActRIIB polypeptide has an amino acid sequence at least 80%, 85%, 90%, 91%, 92%, 93%, 94%, 95%, 96%, 97%, 98%, 99%, or 100% identical to SEQ ID NO: 1. In certain cases, the variant ActRIIB polypeptide has an amino acid sequence at least 80%, 85%, 90%, 91%, 92%, 93%, 94%, 95%, 96%, 97%, 98%, 99%, or 100% identical to SEQ ID NO: 2. In certain cases, the variant ActRIIB polypeptide has an amino acid sequence at least 80%, 85%, 90%, 910, 92%, 93%, 94%, 95%, 96%, 97%, 98%, 99%, or 100% identical to SEQ ID NO: 53.

In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID NOs: 1, 2, 5, 6, 12, 31, 33, 34, 36, 37, 39, 40, 42, 43, 45, 46, 48, 49, 50, 51, 52, 53, 276, 278, 279, 332, 333, 335, 336, 338, 339, 341, 342, 344, 345, 347, 348, 350, 351, 353, 354, 356, and 357. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 1. The amino acid sequence of SEQ ID NO: 1 may optionally be provided with the lysine removed from the C-terminus. In some embodiments, variant ActRIIB poly peptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 2. The amino acid sequence of SEQ ID NO: 2 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 5. The amino acid sequence of SEQ ID NO: 5 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 6. The amino acid sequence of SEQ ID NO: 6 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 12. The amino acid sequence of SEQ ID NO: 12 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 31. The amino acid sequence of SEQ ID NO: 31 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 33. The amino acid sequence of SEQ ID NO: 33 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 34. The amino acid sequence of SEQ ID NO: 34 may optionally be provided with the lysine removed from the C-terminus. In some embodiments, variant ActRIIB poly peptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 36. The amino acid sequence of SEQ ID NO: 36 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 37. The amino acid sequence of SEQ ID NO: 37 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 39. The amino acid sequence of SEQ ID NO: 39 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 40. The amino acid sequence of SEQ ID NO: 40 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 42. The amino acid sequence of SEQ ID NO: 42 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 910, 92%, 93%, 94%, 95%, 96%, 97%, 98%, 99%, or 100% identical to the amino acid sequence of SEQ ID NO: 43. The amino acid sequence of SEQ ID NO: 43 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 45. The amino acid sequence of SEQ ID NO: 45 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 46. The amino acid sequence of SEQ ID NO: 46 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 48. The amino acid sequence of SEQ ID NO: 48 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 49. The amino acid sequence of SEQ ID NO: 49 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 50. The amino acid sequence of SEQ ID NO: 50 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 51. The amino acid sequence of SEQ ID NO: 51 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 52. The amino acid sequence of SEQ ID NO: 52 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 53. The amino acid sequence of SEQ ID NO: 53 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 276. The amino acid sequence of SEQ ID NO: 276 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 278. The amino acid sequence of SEQ ID NO: 278 may optionally be provided with the lysine removed from the C-terminus.

In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 279. The amino acid sequence of SEQ ID NO: 279 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 332. The amino acid sequence of SEQ ID NO: 332 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 333. The amino acid sequence of SEQ ID NO: 333 may optionally be provided with the lysine removed from the C-terminus.

In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 335. The amino acid sequence of SEQ ID NO: 335 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 336. The amino acid sequence of SEQ ID NO: 336 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 338. The amino acid sequence of SEQ ID NO: 338 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 339. The amino acid sequence of SEQ ID NO: 339 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 341. The amino acid sequence of SEQ ID NO: 341 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 342. The amino acid sequence of SEQ ID NO: 342 may optionally be provided with the lysine removed from the C-terminus.

In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 344. The amino acid sequence of SEQ ID NO: 344 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 345. The amino acid sequence of SEQ ID NO: 345 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 347. The amino acid sequence of SEQ ID NO: 347 may optionally be provided with the lysine removed from the C-terminus.

In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 348. The amino acid sequence of SEQ ID NO: 348 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 350. The amino acid sequence of SEQ ID NO: 350 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 351. The amino acid sequence of SEQ ID NO: 351 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 353. The amino acid sequence of SEQ ID NO: 353 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 354. The amino acid sequence of SEQ ID NO: 354 may optionally be provided with the lysine removed from the C-terminus. In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 356. The amino acid sequence of SEQ ID NO: 356 may optionally be provided with the lysine removed from the C-terminus.

In some embodiments, variant ActRIIB polypeptides or variant ActRIIB-Fc fusion polypeptides of the disclosure comprise, consist, or consist essentially of an amino acid sequence that is at least 70%, 75%, 80%, 85%, 86%, 87%, 88%, 89%, 90%, 91%, 920, 93%, 94%, 95%, 96%, 97%, 98%, 99%, or 100% identical to the amino acid sequence of SEQ ID NO: 357. The amino acid sequence of SEQ ID NO: 357 may optionally be provided with the lysine removed from the C-terminus.

In certain aspects, the disclosure relates to variant ActRIIB polypeptides comprising an amino acid sequence that is at least 70%, 75%, 80%, 85%, 90%, 91%, 92%, 93%, 94%, 95%, 96%, 97%, 98%, 99%, or 100% identical to an amino acid sequence that begins at any one of amino acids 20-29 (e.g., amino acid residues 20, 21, 22, 23, 24, 25, 26, 27, 28, or 29) of SEQ ID NO: 2 and ends at any one of amino acids 109-134 (e.g., amino acid residues 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 2, and wherein the polypeptide comprises one or more amino acid substitutions at a position of SEQ ID NO: 2 selected from the group consisting of: K55, F82, L79, A24, K74, R64, P129, P130, E37, R40, D54, R56, W78, D80, and F82 as well as heteromultimer complexes comprising one or more such variant ActRIIB polypeptides. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 29-109 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 25-131 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to amino acids 20-134 of SEQ ID NO: 2. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 53. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 12. In some embodiments, the variant ActRIIB polypeptide comprises an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 5. In some embodiments, the polypeptide comprises an amino acid substitution at the amino acid position corresponding to A24 of SEQ ID NO: 2. For example, in some embodiments, the substitution is A24N. In some embodiments, the polypeptide comprises an amino acid substitution at the amino acid position corresponding to S26 of SEQ ID NO: 2. For example, in some embodiments, the substitution is S26T. In some embodiments, the polypeptide comprises an amino acid substitution at the amino acid position corresponding to N35 of SEQ ID NO: 2. For example, in some embodiments, the substitution is N35E. In some embodiments, the polypeptide comprises an amino acid substitution at the amino acid position corresponding to E37 of SEQ ID NO: 2. For example, in some embodiments, the substitution is E37A. In some embodiments, the substitution is E37D. In some embodiments, the polypeptide comprises an amino acid substitution at the amino acid position corresponding to L38 of SEQ ID NO: 2. For example, in some embodiments, the substitution is L38N. In some embodiments, the polypeptide comprises an amino acid substitution at the amino acid position corresponding to R40 of SEQ ID NO: 2. For example, in some embodiments, the substitution is R40A. In some embodiments, the substitution is R40K. In some embodiments, the polypeptide comprises an amino acid substitution at the amino acid position corresponding to S44 of SEQ ID NO: 2. For example, in some embodiments, the substitution is S44T. In some embodiments, the polypeptide comprises an amino acid substitution at the amino acid position corresponding to L46 of SEQ ID NO: 2. For example, in some embodiments, the substitution is L46A. For example, in some embodiments, the substitution is L461. For example, in some embodiments, the substitution is L46F. For example, in some embodiments, the substitution is L46V. In some embodiments, the polypeptide comprises an amino acid substitution at the amino acid position corresponding to E50 of SEQ ID NO: 2. For example, in some embodiments, the substitution is E50K. In some embodiments, the substitution is E50L. In some embodiments, the substitution is E50P. In some embodiments, the polypeptide comprises an amino acid substitution at the amino acid position corresponding to E52 of SEQ ID NO: 2. For example, in some embodiments, the substitution is E52A. In some embodiments, the substitution is E52D. In some embodiments, the substitution is E52G. In some embodiments, the substitution is E52H. In some embodiments, the substitution is E52K. In some embodiments, the substitution is E52N. In some embodiments, the substitution is E52P. In some embodiments, the substitution is E52R. In some embodiments, the substitution is E52S. In some embodiments, the substitution is E52T. In some embodiments, the substitution is E52Y. In some embodiments, the polypeptide comprises an amino acid substitution at the amino acid position corresponding to Q53 of SEQ ID NO: 2. For example, in some embodiments, the substitution is Q53R. For example, in some embodiments, the substitution is Q53K. For example, in some embodiments, the substitution is Q53N. For example, in some embodiments, the substitution is Q53H. In some embodiments, the polypeptide comprises an amino acid substitution at the amino acid position corresponding to D54 of SEQ ID NO: 2. For example, in some embodiments, the substitution is D54A. In some embodiments, the polypeptide comprises an amino acid substitution at the amino acid position corresponding to K55 of SEQ ID NO: 2. For example, in some embodiments, the substitution is K55A. In some embodiments, the substitution is K55E. In some embodiments, the substitution is K55D. In some embodiments, the substitution is K55R. In some embodiments, the polypeptide comprises an amino acid substitution at the amino acid position corresponding to R56 of SEQ ID NO: 2. For example, in some embodiments, the substitution is R56A. In some embodiments, the polypeptide comprises an amino acid substitution at the amino acid position corresponding to L57 of SEQ ID NO: 2. For example, in some embodiments, the substitution is L57R. In some embodiments, the substitution is L57E. In some embodiments, the substitution is L57I. In some embodiments, the substitution is L57T. In some embodiments, the substitution is L57V. In some embodiments, the polypeptide comprises an amino acid substitution at the amino acid position corresponding to Y60 of SEQ ID NO: 2. For example, in some embodiments, the substitution is Y60F. In some embodiments, the substitution is Y60D. In some embodiments, the substitution is Y60K. In some embodiments, the substitution is Y60P. In some embodiments, the polypeptide comprises an amino acid substitution at the amino acid position corresponding to R64 of SEQ ID NO: 2. For example, in some embodiments, the substitution is R64K. In some embodiments, the substitution is R64N. In some embodiments, the substitution is R64A. In some embodiments, the substitution is R64H. In some embodiments, the polypeptide comprises an amino acid substitution at the amino acid position corresponding to N65 of SEQ ID NO: 2. For example, in some embodiments, the substitution is N65A. In some embodiments, the polypeptide comprises an amino acid substitution at the amino acid position corresponding to S67 of SEQ ID NO: 2. For example, in some embodiments, the substitution is S67N. In some embodiments, the substitution is S67T. In some embodiments, the polypeptide comprises an amino acid substitution at the amino acid position corresponding to G68 of SEQ ID NO: 2. For example, in some embodiments, the substitution is G68R. In some embodiments, the polypeptide comprises an amino acid substitution at the amino acid position corresponding to K74 of SEQ ID NO: 2. For example, in some embodiments, the substitution is K74A. In some embodiments, the substitution is K74E. In some embodiments, the substitution is K74F. In some embodiments, the substitution is K74I. In some embodiments, the substitution is K74Y. In some embodiments, the substitution is K74R. In some embodiments, the polypeptide comprises an amino acid substitution at the amino acid position corresponding to W78 of SEQ ID NO: 2. For example, in some embodiments, the substitution is W78A. In some embodiments, the substitution is W78Y. In some embodiments, the polypeptide comprises an amino acid substitution at the amino acid position corresponding to L79 of SEQ ID NO: 2. For example, in some embodiments, the substitution is L79D. In some embodiments, the substitution does not comprise an acidic amino acid at the position corresponding to L79 of SEQ ID NO: 2. In some embodiments, the substitution does not comprise an aspartic acid (D) at the position corresponding to L79 of SEQ ID NO: 2. In some embodiments, the substitution is L79A. In some embodiments, the substitution is L79E. In some embodiments, the substitution is L79F. In some embodiments, the substitution is L79H. In some embodiments, the substitution is L79K. In some embodiments, the substitution is L79P. In some embodiments, the substitution is L79R. In some embodiments, the substitution is L79S. In some embodiments, the substitution is L79T. In some embodiments, the substitution is L79W. In some embodiments, the polypeptide comprises an amino acid substitution at the amino acid position corresponding to D80 of SEQ ID NO: 2. For example, in some embodiments, the substitution is D80A. In some embodiments, the substitution is D80F. In some embodiments, the substitution is D80K. In some embodiments, the substitution is D80G. In some embodiments, the substitution is D80M. In some embodiments, the substitution is D80I. In some embodiments, the substitution is D80N. In some embodiments, the substitution is D80R. In some embodiments, the polypeptide comprises an amino acid substitution at the amino acid position corresponding to F82 of SEQ ID NO: 2. For example, in some embodiments, the substitution is F82I. In some embodiments, the substitution is F82K. In some embodiments, the substitution is F82A. In some embodiments, the substitution is F82W. In some embodiments, the substitution is F82D. In some embodiments, the substitution is F82Y. In some embodiments, the substitution is F82E. In some embodiments, the substitution is F82L. In some embodiments, the substitution is F82T. In some embodiments, the substitution is F82S. In some embodiments, the polypeptide comprises an amino acid substitution at the amino acid position corresponding to N83 of SEQ ID NO: 2. For example, in some embodiments, the substitution is N83A. In some embodiments, the substitution is N83R. In some embodiments, the polypeptide comprises an amino acid substitution at the amino acid position corresponding to 193 of SEQ ID NO: 2. For example, in some embodiments, the substitution is T93D. In some embodiments, the substitution is T93E. In some embodiments, the substitution is T93H. In some embodiments, the substitution is T93G. In some embodiments, the substitution is T93K. In some embodiments, the substitution is T93P. In some embodiments, the substitution is T93R. In some embodiments, the substitution is T93S. In some embodiments, the substitution is T93Y. In some embodiments, the polypeptide comprises an amino acid substitution at the amino acid position corresponding to E94 of SEQ ID NO: 2. For example, in some embodiments, the substitution is E94K. In some embodiments, the polypeptide comprises an amino acid substitution at the amino acid position corresponding to Q98 of SEQ ID NO: 2. For example, in some embodiments, the substitution is Q98D. In some embodiments, the substitution is Q98E. In some embodiments, the substitution is Q98K. In some embodiments, the substitution is Q98R. In some embodiments, the polypeptide comprises an amino acid substitution at the amino acid position corresponding to V99 of SEQ ID NO: 2. For example, in some embodiments, the substitution is V99E. In some embodiments, the substitution is V99G. In some embodiments, the substitution is V99K. In some embodiments, the polypeptide comprises an amino acid substitution at the amino acid position corresponding to E105 of SEQ ID NO: 2. For example, in some embodiments, the substitution is E105N. In some embodiments, the polypeptide comprises an amino acid substitution at the amino acid position corresponding to E106 of SEQ ID NO: 2. For example, in some embodiments, the substitution is E106N. In some embodiments, the polypeptide comprises an amino acid substitution at the amino acid position corresponding to F108 of SEQ ID NO: 2. For example, in some embodiments, the substitution is F1081. In some embodiments, the substitution is F108L. In some embodiments, the substitution is F108V. In some embodiments, the substitution is F108Y. In some embodiments, the polypeptide comprises an amino acid substitution at the amino acid position corresponding to E111 of SEQ ID NO: 2. For example, in some embodiments, the substitution is E111K. In some embodiments, the substitution is E111D. In some embodiments, the substitution is E111R. In some embodiments, the substitution is E111H. In some embodiments, the substitution is E111Q. In some embodiments, the substitution is E111N. In some embodiments, the polypeptide comprises an amino acid substitution at the amino acid position corresponding to R112 of SEQ ID NO: 2. For example, in some embodiments, the substitution is R112H. In some embodiments, the substitution is R112K. In some embodiments, the substitution is R112N. In some embodiments, the substitution is R112S. In some embodiments, the substitution is R112T. In some embodiments, the polypeptide comprises an amino acid substitution at the amino acid position corresponding to A119 of SEQ ID NO: 2. For example, in some embodiments, the substitution is A119P. In some embodiments, the substitution is A119V. In some embodiments, the polypeptide comprises an amino acid substitution at the amino acid position corresponding to G120 of SEQ ID NO: 2. For example, in some embodiments, the substitution is G120N. In some embodiments, the polypeptide comprises an amino acid substitution at the amino acid position corresponding to E123 of SEQ ID NO: 2. For example, in some embodiments, the substitution is E123N. In some embodiments, the polypeptide comprises an amino acid substitution at the amino acid position corresponding to P129 of SEQ ID NO: 2. For example, in some embodiments, the substitution is P129S. In some embodiments, the substitution is P129N. In some embodiments, the polypeptide comprises an amino acid substitution at the amino acid position corresponding to P130 of SEQ ID NO: 2. For example, in some embodiments, the substitution is P130A. In some embodiments, the substitution is P130R. In some embodiments, the polypeptide comprises an amino acid substitution at the amino acid position corresponding to A132 of SEQ ID NO: 2. For example, in some embodiments, the substitution is A132N.

In some embodiments, any of the variant ActRIIB polypeptides disclosed herein comprises a substitution at a position of SEQ ID NO: 2 selected from the group consisting of: A24, E37, R40, D54, K55, R56, R64, K74, W78, L79, D80, F82, P129, and P130. In some embodiments, the variant ActRIIB polypeptide comprises a substitution at position A24 with respect to SEQ ID NO: 2. In some embodiments, the variant ActRIIB polypeptide comprises a substitution at position E37 with respect to SEQ ID NO: 2. In some embodiments, the variant ActRIIB polypeptide comprises a substitution at position R40 with respect to SEQ ID NO: 2. In some embodiments, the variant ActRIIB polypeptide comprises a substitution at position D54 with respect to SEQ ID NO: 2. In some embodiments, the variant ActRIIB polypeptide comprises a substitution at position K55 with respect to SEQ ID NO: 2. In some embodiments, the variant ActRIIB polypeptide comprises a substitution at position R56 with respect to SEQ ID NO: 2. In some embodiments, the variant ActRIIB polypeptide comprises a substitution at position R64 with respect to SEQ ID NO: 2. In some embodiments, the variant ActRIIB polypeptide comprises a substitution at position K74 with respect to SEQ ID NO: 2. In some embodiments, the variant ActRIIB polypeptide comprises a substitution at position W78 with respect to SEQ ID NO: 2. In some embodiments, the variant ActRIIB polypeptide comprises a substitution at position L79 with respect to SEQ ID NO: 2. In some embodiments, the variant ActRIIB polypeptide comprises a substitution at position D80 with respect to SEQ ID NO: 2. In some embodiments, the variant ActRIIB polypeptide comprises a substitution at position F82 with respect to SEQ ID NO: 2. In some embodiments, the variant ActRIIB polypeptide comprises a substitution at position P129 with respect to SEQ ID NO: 2. In some embodiments, the variant ActRIIB polypeptide comprises a substitution at position P130 with respect to SEQ ID NO: 2.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 31. In some embodiments, the variant ActRIIB polypeptide comprises an alanine at the position corresponding to K55 of SEQ ID NO: 2. In some embodiments, the amino acid sequence of SEQ ID NO: 31 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 33. In some embodiments, the variant ActRIIB polypeptide comprises an alanine at the position corresponding to K55 of SEQ ID NO: 2. In some embodiments, the amino acid sequence of SEQ ID NO: 33 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 34. In some embodiments, the variant ActRIIB polypeptide comprises a glutamic acid at the position corresponding to K55 of SEQ ID NO: 2. In some embodiments, the amino acid sequence of SEQ ID NO: 34 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 36. In some embodiments, the variant ActRIIB polypeptide comprises a glutamic acid at the position corresponding to K55 of SEQ ID NO: 2. In some embodiments, the amino acid sequence of SEQ ID NO: 36 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 37. In some embodiments, the variant ActRIIB polypeptide comprises an isoleucine at the position corresponding to F82 of SEQ ID NO: 2. In some embodiments, the amino acid sequence of SEQ ID NO: 37 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 39. In some embodiments, the variant ActRIIB polypeptide comprises an isoleucine at the position corresponding to F82 of SEQ ID NO: 2. In some embodiments, the amino acid sequence of SEQ ID NO: 39 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 40. In some embodiments, the variant ActRIIB polypeptide comprises a lysine at the position corresponding to F82 of SEQ ID NO: 2. In some embodiments, the amino acid sequence of SEQ ID NO: 40 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 42. In some embodiments, the variant ActRIIB polypeptide comprises a lysine at the position corresponding to F82 of SEQ ID NO: 2. In some embodiments, the amino acid sequence of SEQ ID NO: 42 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 43. In some embodiments, the variant ActRIIB polypeptide comprises a glutamic acid at the position corresponding to L79 of SEQ ID NO: 2. In some embodiments, the amino acid sequence of SEQ ID NO: 43 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 45. In some embodiments, the variant ActRIIB polypeptide comprises a glutamic acid at the position corresponding to L79 of SEQ ID NO: 2. In some embodiments, the amino acid sequence of SEQ ID NO: 45 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 336. In some embodiments, the variant ActRIIB polypeptide comprises a threonine at the position corresponding to F82 of SEQ ID NO: 2. In some embodiments, the amino acid sequence of SEQ ID NO: 336 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 338. In some embodiments, the variant ActRIIB polypeptide comprises a threonine at the position corresponding to F82 of SEQ ID NO: 2. In some embodiments, the amino acid sequence of SEQ ID NO: 338 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 342. In some embodiments, the variant ActRIIB polypeptide comprises a histidine at the position corresponding to L79 of SEQ ID NO: 2. In some embodiments, the amino acid sequence of SEQ ID NO: 342 may optionally be provided with the lysine removed from the C-terminus.

In certain aspects, the disclosure relates to a variant ActRIIB polypeptide comprising an amino acid sequence that is at least 75%, 80%,o, 85%, 90%, 91%, 92%, 93%, 94%, 95%, 96%, 97%, 98%, 99%, or 100% identical to the amino acid sequence of SEQ ID NO: 344. In some embodiments, the variant ActRIIB polypeptide comprises a histidine at the position corresponding to L79 of SEQ ID NO: 2. In some embodiments, the amino acid sequence of SEQ ID NO: 344 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 348. In some embodiments, the variant ActRIIB polypeptide comprises a leucine at the position corresponding to E50 of SEQ ID NO: 2. In some embodiments, the amino acid sequence of SEQ ID NO: 348 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 350. In some embodiments, the variant ActRIIB polypeptide comprises a leucine at the position corresponding to E50 of SEQ ID NO: 2. In some embodiments, the amino acid sequence of SEQ ID NO: 350 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 354. In some embodiments, the variant ActRIIB polypeptide comprises a glycine at the position corresponding to V99 of SEQ ID NO: 2. In some embodiments, the amino acid sequence of SEQ ID NO: 354 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 356. In some embodiments, the variant ActRIIB polypeptide comprises a glycine at the position corresponding to V99 of SEQ ID NO: 2. In some embodiments, the amino acid sequence of SEQ ID NO: 356 may optionally be provided with the lysine removed from the C-terminus.

In some embodiments, any of the variant ActRIIB polypeptides disclosed herein comprises at least 2, 3, 4, 5, 6, 7, 8, 9, or 10 of any of the amino acid substitutions disclosed herein. In some embodiments, any of the variant ActRIIB polypeptides disclosed herein comprises 2 of any of the amino acid substitutions disclosed herein. In some embodiments, any of the variant ActRIIB polypeptides disclosed herein comprises 3 of any of the amino acid substitutions disclosed herein. In some embodiments, any of the variant ActRIIB polypeptides disclosed herein comprises 4 of any of the amino acid substitutions disclosed herein. In some embodiments, any of the variant ActRIIB polypeptides disclosed herein comprises 5 of any of the amino acid substitutions disclosed herein. In some embodiments, any of the variant ActRIIB polypeptides disclosed herein comprises 6 of any of the amino acid substitutions disclosed herein. In some embodiments, any of the variant ActRIIB polypeptides disclosed herein comprises 7 of any of the amino acid substitutions disclosed herein. In some embodiments, any of the variant ActRIIB polypeptides disclosed herein comprises 8 of any of the amino acid substitutions disclosed herein. In some embodiments, any of the variant ActRIIB polypeptides disclosed herein comprises 9 of any of the amino acid substitutions disclosed herein. In some embodiments, any of the variant ActRIIB polypeptides disclosed herein comprises 10 of any of the amino acid substitutions disclosed herein.

In certain aspects, the disclosure relates to a variant ActRIIB polypeptide comprising two or more amino acid substitutions as compared to the reference amino acid sequence of SEQ ID NO: 2. For example, in some embodiments, the variant ActRIIB polypeptide comprises an A24N substitution and a K74A substitution. In some embodiments, the variant ActRIIB polypeptide comprises a L79P substitution and a K74A substitution. In some embodiments, the variant ActRIIB polypeptide comprises a P129S substitution and a P130A substitution. In some embodiments, the variant ActRIIB polypeptide comprises a L38N substitution and a L79R substitution. In some embodiments, the variant ActRIIB polypeptide comprises a F82I substitution and a N83R substitution. In some embodiments, the variant ActRIIB polypeptide comprises a F82K substitution and a N83R substitution. In some embodiments, the variant ActRIIB polypeptide comprises a F82T substitution and a N83R substitution. In some embodiments, the variant ActRIIB polypeptide comprises a L79H substitution and a F82K substitution. In some embodiments, the variant ActRIIB polypeptide comprises a L79H substitution and a F82I substitution. In some embodiments, the variant ActRIIB polypeptide comprises a F82D substitution and a N83R substitution. In some embodiments, the variant ActRIIB polypeptide comprises a F82E substitution and a N83R substitution. In some embodiments, the variant ActRIIB polypeptide comprises a L79F substitution and a F82D substitution. In some embodiments, the variant ActRIIB polypeptide comprises a L79F substitution and a F82T substitution. In some embodiments, the variant ActRIIB polypeptide comprises a E52D substitution and a F82D substitution. In some embodiments, the variant ActRIIB polypeptide comprises an E52D substitution and a F82T substitution. In some embodiments, the variant ActRIIB polypeptide comprises a L57R substitution and a F82D substitution. In some embodiments, the variant ActRIIB polypeptide comprises a L57R substitution and a F82T substitution. In some embodiments, the variant ActRIIB polypeptide comprises a F82I substitution and an E94K substitution. In some embodiments, the variant ActRIIB polypeptide comprises a F82S substitution and a N83R substitution. In some embodiments, the variant ActRIIB polypeptide comprises a L57R substitution and a F82S substitution. In some embodiments, the variant ActRIIB polypeptide comprises a K74A substitution and a L79P substitution. In some embodiments, the variant ActRIIB polypeptide comprises a K55A substitution and a F82I substitution. In some embodiments, the variant ActRIIB polypeptide comprises a L79K substitution and a F82K substitution. In some embodiments, the variant ActRIIB polypeptide comprises a F82W substitution and a N83A substitution. In some embodiments, the variant ActRIIB polypeptide comprises an A24N substitution and a K74A substitution. In some embodiments, the variant ActRIIB polypeptide comprises a L79P substitution and a K74A substitution. In some embodiments, the variant ActRIIB polypeptide comprises a P129S substitution and a P130A substitution. In some embodiments, the variant ActRIIB polypeptide comprises a N65A substitution and a S67N substitution. In some embodiments, the variant ActRIIB polypeptide comprises a K55A substitution and a F82I substitution. In some embodiments, the variant ActRIIB polypeptide comprises a L79H substitution and a F82I substitution. In some embodiments, the variant ActRIIB polypeptide comprises a L79K substitution and a F82K substitution. In some embodiments, the variant ActRIIB polypeptide comprises a F82W substitution and a N83A substitution.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 276. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to F82 of SEQ ID NO: 2 and an arginine at the position corresponding to N83 of SEQ ID NO: 2. In some embodiments, the amino acid sequence of SEQ ID NO: 276 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 278. In some embodiments, the variant ActRIIB polypeptide comprises an isoleucine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises an isoleucine at the position corresponding to F82 of SEQ ID NO: 2 and an arginine at the position corresponding to N83 of SEQ ID NO: 2. In some embodiments, the amino acid sequence of SEQ ID NO: 278 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 279. In some embodiments, the variant ActRIIB polypeptide comprises an lysine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a lysine at the position corresponding to F82 of SEQ ID NO: 2 and an arginine at the position corresponding to N83 of SEQ ID NO: 2. In some embodiments, the amino acid sequence of SEQ ID NO: 279 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 332. In some embodiments, the variant ActRIIB polypeptide comprises an lysine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a lysine at the position corresponding to F82 of SEQ ID NO: 2 and an arginine at the position corresponding to N83 of SEQ ID NO: 2. In some embodiments, the amino acid sequence of SEQ ID NO: 332 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 333. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to F82 of SEQ ID NO: 2 and an arginine at the position corresponding to N83 of SEQ ID NO: 2. In some embodiments, the amino acid sequence of SEQ ID NO: 333 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 335. In some embodiments, the variant ActRIIB polypeptide comprises a threonine at the position corresponding to F82 of SEQ ID NO: 2. In some embodiments, the variant ActRIIB polypeptide comprises an arginine at the position corresponding to N83 of SEQ ID NO: 2. In some embodiments, the variant ActRIIB polypeptide comprises a threonine at the position corresponding to F82 of SEQ ID NO: 2 and an arginine at the position corresponding to N83 of SEQ ID NO: 2. In some embodiments, the amino acid sequence of SEQ ID NO: 335 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 339. In some embodiments, the variant ActRIIB polypeptide comprises a histidine at the position corresponding to L79 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises a histidine at the position corresponding to L79 of SEQ ID NO: 2 and an isoleucine at the position corresponding to F82 of SEQ ID NO: 2. In some embodiments, the amino acid sequence of SEQ ID NO: 339 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 341. In some embodiments, the variant ActRIIB polypeptide comprises a histidine at the position corresponding to L79 of SEQ ID NO: 2. In some embodiments, the variant ActRIIB polypeptide comprises an isoleucine at the position corresponding to F82 of SEQ ID NO: 2. In some embodiments, the variant ActRIIB polypeptide comprises a histidine at the position corresponding to L79 of SEQ ID NO: 2 and an isoleucine at the position corresponding to F82 of SEQ ID NO: 2. In some embodiments, the amino acid sequence of SEQ ID NO: 341 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 345. In some embodiments, the variant ActRIIB polypeptide comprises a histidine at the position corresponding to L79 of SEQ ID NO: 2. In some embodiments, the variant ActRIIB polypeptide comprises a lysine at the position corresponding to F82 of SEQ ID NO: 2. In some embodiments, the variant ActRIIB polypeptide comprises a histidine at the position corresponding to L79 of SEQ ID NO: 2, and a lysine at the position corresponding to F82 of SEQ ID NO: 2. In some embodiments, the amino acid sequence of SEQ ID NO: 345 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 347. In some embodiments, the variant ActRIIB polypeptide comprises a histidine at the position corresponding to L79 of SEQ ID NO: 2. In some embodiments, the variant ActRIIB polypeptide comprises a lysine at the position corresponding to F82 of SEQ ID NO: 2. In some embodiments, the variant ActRIIB polypeptide comprises a histidine at the position corresponding to L79 of SEQ ID NO: 2, and a lysine at the position corresponding to F82 of SEQ ID NO: 2. In some embodiments, the amino acid sequence of SEQ ID NO: 347 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 351. In some embodiments, the variant ActRIIB polypeptide comprises an asparagine at the position corresponding to L38 of SEQ ID NO: 2. In some embodiments, the variant ActRIIB polypeptide comprises an arginine at the position corresponding to L79 of SEQ ID NO: 2. In some embodiments, the variant ActRIIB polypeptide comprises an asparagine at the position corresponding to L38 of SEQ ID NO: 2, and an arginine at the position corresponding to L79 of SEQ ID NO: 2. In some embodiments, the amino acid sequence of SEQ ID NO: 351 may optionally be provided with the lysine removed from the C-terminus.

In certain aspects, the disclosure relates to a variant ActRIIB polypeptide comprising an amino acid sequence that is at least 75%, 80%, 85%, 90%, 91%, 92%, 93%, 94%, 95%, 96%, 97%, 98%, 99%, or 100% identical to the amino acid sequence of SEQ ID NO: 353. In some embodiments, the variant ActRIIB polypeptide comprises an asparagine at the position corresponding to L38 of SEQ ID NO: 2. In some embodiments, the variant ActRIIB polypeptide comprises an arginine at the position corresponding to L79 of SEQ ID NO: 2. In some embodiments, the variant ActRIIB polypeptide comprises an asparagine at the position corresponding to L38 of SEQ ID NO: 2, and an arginine at the position corresponding to L79 of SEQ ID NO: 2. In some embodiments, the amino acid sequence of SEQ ID NO: 353 may optionally be provided with the lysine removed from the C-terminus.

In certain aspects, the disclosure relates to a variant ActRIIB polypeptide comprising three or more amino acid substitutions as compared to the reference amino acid sequence of SEQ ID NO: 2. In some embodiments, the variant ActRIIB polypeptide comprises a G68R substitution, a F82S substitution, and a N83R substitution. In some embodiments, the variant ActRIIB polypeptide comprises a G68R substitution, a W78Y substitution, and a F82Y substitution. In some embodiments, the variant ActRIIB polypeptide comprises a E52D substitution, a F82D substitution, and a N83R substitution. In some embodiments, the variant ActRIIB polypeptide comprises an E52Y substitution, a F82D substitution, and a N83R substitution. In some embodiments, the variant ActRIIB polypeptide comprises an E52D substitution, a F82E substitution, and a N83R substitution. In some embodiments, the variant ActRIIB polypeptide comprises an E52D substitution, a F82T substitution, and a N83R substitution. In some embodiments, the variant ActRIIB polypeptide comprises an E52N substitution, a F82I substitution, and a N83R substitution. In some embodiments, the variant ActRIIB polypeptide comprises an E52N substitution, a F82Y substitution, and a N83R substitution. In some embodiments, the variant ActRIIB polypeptide comprises an E50L substitution, a F82D substitution, and a N83R substitution. In some embodiments, the variant ActRIIB polypeptide comprises a L57I substitution, a F82D substitution, and a N83R substitution. In some embodiments, the variant ActRIIB polypeptide comprises a L57V substitution, a F82D substitution, and a N83R substitution. In some embodiments, the variant ActRIIB polypeptide comprises a L57R substitution, a F82D substitution, and a N83R substitution. In some embodiments, the variant ActRIIB polypeptide comprises a L57E substitution, a F82E substitution, and a N83R substitution. In some embodiments, the variant ActRIIB polypeptide comprises a L57R substitution, a F82E substitution, and a N83R substitution. In some embodiments, the variant ActRIIB polypeptide comprises a L57I substitution, a F82E substitution, and a N83R substitution. In some embodiments, the variant ActRIIB polypeptide comprises a L57R substitution, a F82L substitution, and a N83R substitution. In some embodiments, the variant ActRIIB polypeptide comprises a L57T substitution, a F82Y substitution, and a N83R substitution. In some embodiments, the variant ActRIIB polypeptide comprises a L57V substitution, a F82Y substitution, and a N83R substitution. In some embodiments, the variant ActRIIB polypeptide may comprise at least two of the amino acid substitutions described in any of the variant ActRIIB polypeptides above.

In certain aspects, the disclosure relates to a variant ActRIIB polypeptide comprising four or more amino acid substitutions as compared to the reference amino acid sequence of SEQ ID NO: 2. For example, in some embodiments, the variant ActRIIB polypeptide comprises a G68R substitution, a L79E substitution, a F82Y substitution, and a N83R substitution. In some embodiments, the variant ActRIIB polypeptide comprises a G68R substitution, a L79E substitution, a F82T substitution, and a N83R substitution. In some embodiments, the variant ActRIIB polypeptide comprises a G68R substitution, a L79T substitution, a F82T substitution, and a N83R substitution. In some embodiments, the variant ActRIIB polypeptide comprises an E52N substitution, a G68R substitution, a F82Y substitution, and a N83R substitution. In some embodiments, the variant ActRIIB polypeptide may comprise at least two of the amino acid substitutions described in any of the variant ActRIIB polypeptides above. In some embodiments, the variant ActRIIB polypeptide may comprise at least three of the amino acid substitutions described in any of the variant ActRIIB polypeptides above.

In certain embodiments, the present disclosure contemplates further mutations of the variant ActRIIB polypeptides so as to alter the glycosylation of the polypeptide. Exemplary glycosylation sites in variant ActRIIB polypeptides are illustrated in FIG. 4. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine (where “X” is any amino acid) which is specifically recognized by appropriate cellular glycosylation enzymes. The alteration may also be made by the addition of, or substitution by, one or more serine or threonine residues to the sequence of the wild-type ActRIIB polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a variant ActRIIB polypeptide is by chemical or enzymatic coupling of glycosides to the variant ActRIIB polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. These methods are described in WO 87/05330 published Sep. 11, 1987, and in Aplin and Wriston (1981) CRC Crit. Rev. Biochem., pp. 259-306, incorporated by reference herein. Removal of one or more carbohydrate moieties present on a variant ActRIIB polypeptide may be accomplished chemically and/or enzymatically. Chemical deglycosylation may involve, for example, exposure of the variant ActRIIB polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Chemical deglycosylation is further described by Hakimuddin et al. (1987) Arch. Biochem. Biophys. 259:52 and by Edge et al. (1981) Anal. Biochem. 118:131. Enzymatic cleavage of carbohydrate moieties on variant ActRIIB polypeptides can be achieved by the use of a variety of endo- and exo-glycosidases as described by Thotakura et al. (1987) Meth. Enzymol. 138:350. The sequence of a variant ActRIIB polypeptide may be adjusted, as appropriate, depending on the type of expression system used, as mammalian, yeast, insect and plant cells may all introduce differing glycosylation patterns that can be affected by the amino acid sequence of the peptide. In general, variant ActRIIB polypeptides for use in humans will be expressed in a mammalian cell line that provides proper glycosylation, such as HEK293 or CHO cell lines, although other mammalian expression cell lines are expected to be useful as well.

This disclosure further contemplates a method of generating variants, particularly sets of combinatorial variants of an ActRIIB polypeptide, including, optionally, truncation variants; pools of combinatorial mutants are especially useful for identifying functional variant sequences. The purpose of screening such combinatorial libraries may be to generate, for example, variant ActRIIB polypeptides which have altered properties, such as altered pharmacokinetics, or altered ligand binding. A variety of screening assays are provided below, and such assays may be used to evaluate variants. For example, a variant ActRIIB polypeptide may be screened for ability to bind to an ActRIIB polypeptide, to prevent binding of an ActRIIB ligand to an ActRIIB polypeptide.

The activity of an ActRIIB polypeptide or its variants may also be tested in a cell-based or in vivo assay. For example, the effect of a variant ActRIIB polypeptide on the expression of genes involved in bone production in an osteoblast or precursor may be assessed. This may, as needed, be performed in the presence of one or more recombinant ActRIIB ligand polypeptide (e.g., BMP7), and cells may be transfected so as to produce an ActRIIB polypeptide and/or variants thereof, and optionally, an ActRIIB ligand. Likewise, an ActRIIB polypeptide may be administered to a mouse or other animal, and one or more bone properties, such as density or volume may be assessed. The healing rate for bone fractures may also be evaluated. Similarly, the activity of an ActRIIB polypeptide or its variants may be tested in cardiac or pulmonary cells for any effect on growth of these cells, for example, by the assays as described below. Such assays are well known and routine in the art. A SMAD-responsive reporter gene may be used in such cell lines to monitor effects on downstream signaling.

Combinatorially-derived variants can be generated which have a selective potency relative to a naturally occurring ActRIIB polypeptide. Such variant polypeptides, when expressed from recombinant DNA constructs, can be used in gene therapy protocols. Likewise, mutagenesis can give rise to variants which have intracellular half-lives dramatically different than the corresponding a wild-type ActRIIB polypeptide. For example, the altered polypeptide can be rendered either more stable or less stable to proteolytic degradation or other processes which result in destruction of, or otherwise inactivation of a native ActRIIB polypeptide. Such variants, and the genes which encode them, can be utilized to alter ActRIIB polypeptide levels by modulating the half-life of the ActRIIB polypeptides. For instance, a short half-life can give rise to more transient biological effects and, when part of an inducible expression system, can allow tighter control of recombinant ActRIIB polypeptide levels within the cell.

In certain embodiments, the variant ActRIIB polypeptides of the disclosure may further comprise post-translational modifications in addition to any that are naturally present in the variant ActRIIB polypeptides. Such modifications include, but are not limited to, acetylation, carboxylation, glycosylation, phosphorylation, lipidation, and acylation. As a result, the modified variant ActRIIB polypeptides may contain non-amino acid elements, such as polyethylene glycols, lipids, poly- or mono-saccharide, and phosphates. Effects of such non-amino acid elements on the functionality of a variant ActRIIB polypeptide may be tested as described herein for other variant ActRIIB polypeptides. When a variant ActRIIB polypeptide is produced in cells by cleaving a nascent form of the variant ActRIIB polypeptide, post-translational processing may also be important for correct folding and/or function of the polypeptide. Different cells (such as CHO, HeLa. MDCK, 293, WI38, NIH-3T3 or HEK293) have specific cellular machinery and characteristic mechanisms for such post-translational activities and may be chosen to ensure the correct modification and processing of the variant ActRIIB polypeptides.

In certain aspects, variant ActRIIB polypeptides include fusion polypeptides having at least a portion of the variant ActRIIB polypeptides and one or more fusion domains. Well known examples of such fusion domains include, but are not limited to, polyhistidine, Glu-Glu, glutathione S transferase (GST), thioredoxin, protein A, protein G, an immunoglobulin heavy chain constant region (e.g., an Fc), maltose binding protein (MBP), or human serum albumin. A fusion domain may be selected so as to confer a desired property. For example, some fusion domains are particularly useful for isolation of the fusion polypeptides by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the variant ActRIIB polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for factor Xa or thrombin, which allows the relevant protease to partially digest the fusion polypeptides and thereby liberate the recombinant polypeptides therefrom. The liberated polypeptides can then be isolated from the fusion domain by subsequent chromatographic separation. In certain preferred embodiments, a variant ActRIIB polypeptide is fused with a domain that stabilizes the variant ActRIIB polypeptide in vivo (a “stabilizer” domain). By “stabilizing” is meant anything that increases serum half life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. Likewise, fusions to human serum albumin can confer desirable properties. Other types of fusion domains that may be selected include multimerizing (e.g., dimerizing, tetramerizing) domains and functional domains (that confer an additional biological function, such as further stimulation of muscle growth).

In certain aspects the polypeptides disclosed herein may form homomeric variant ActRIIB polypeptides. In some embodiments, each fusion polypeptide chain in the polypeptide complex comprises the same variant ActRIIB polypeptide as any other such chain in the complex. In certain aspects, the polypeptides disclosed herein may form heteromultimers comprising at least one variant ActRIIB polypeptide associated, covalently or non-covalently, with at least one unmodified ActRIIB polypeptide or at least one variant ActRIIB polypeptide different from the first variant ActRIIB polypeptide. For example, in some embodiments the disclosure provides for an ActRIIB heteromultimer (e.g., dimer), wherein the heteromultimer comprises: a) a first variant ActRIIB polypeptide comprising one or more of any of the amino acid substitutions disclosed herein multimerizes (e.g., dimerizes), and b) a second variant ActRIIB polypeptide having a different amino acid substitution or a different combination of amino acid substitutions as the first ActRIIB polypeptide. In some embodiments, heteromeric polypeptides disclosed herein form heterodimers, although higher order heteromultimers are also included such as, but not limited to, heterotrimers, heterotetramers, and further oligomeric structures.

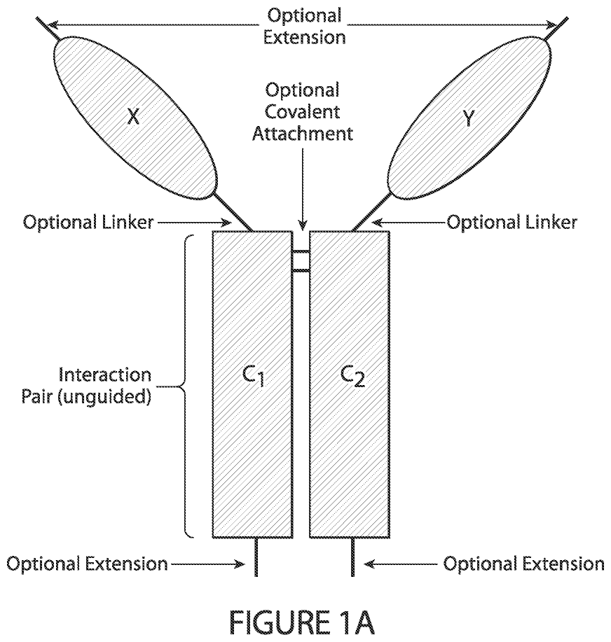

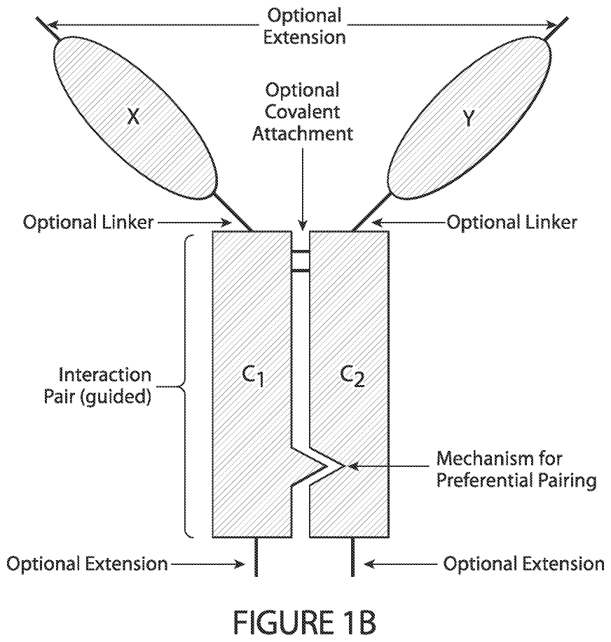

In some embodiments, variant ActRIIB polypeptides of the present disclosure comprise at least one multimerization domain. As disclosed herein, the term “multimerization domain” refers to an amino acid or sequence of amino acids that promote covalent or non-covalent interaction between at least a first polypeptide and at least a second polypeptide. Variant ActRIIB polypeptides disclosed herein may be joined covalently or non-covalently to a multimerization domain. Preferably, a multimerization domain promotes interaction between a first polypeptide (e.g., a first variant ActRIIB polypeptide) and a second polypeptide (e.g., a second variant ActRIIB polypeptide) to promote heteromultimer formation (e.g., heterodimer formation), and optionally hinders or otherwise disfavors homomultimer formation (e.g., homodimer formation), thereby increasing the yield of desired heteromultimer (see, e.g., FIG. 1B). In some embodiments, variant ActRIIB polypeptide of the disclosure form homodimers. In some embodiments, variant ActRIIB polypeptides may from heterodimers through covalent interactions. In some embodiments, variant ActRIIB polypeptides may from heterodimers through non-covalent interactions. In some embodiments, variant ActRIIB polypeptides may from heterodimers through both covalent and non-covalent interactions.

In certain aspects, a variant ActRIIB polypeptide, including homomultimers thereof (e.g., homodimers), binds to one or more TGF-beta superfamily ligands. In some embodiments, variant ActRIIB polypeptide, including homomultimers thereof, binds to one or more TGF-beta superfamily ligands with a KL of at least 1×10⁻⁷ M. In some embodiments, the one or more TGF-beta superfamily ligands is selected from the group consisting of: activin A, activin B, GDF8, GDF11, and BMP10.

In certain aspects, a variant ActRIIB polypeptide, including homomultimers thereof (e.g., homodimers), inhibits one or more TGF-beta super family ligands. In some embodiments, variant ActRIIB polypeptide, including homomultimers thereof, inhibits signaling of one or more TGF-beta super family ligands. In some embodiments, variant ActRIIB polypeptide, including homomultimers thereof, inhibits Smad signaling of one or more TGF-beta super family ligands. In some embodiments, variant ActRIIB polypeptide, including homomultimers thereof, inhibits signaling of one or more TGF-beta super family ligands in a cell-based assay. In some embodiments, variant ActRIIB polypeptide, including homomultimers thereof, inhibits one or more TGF-beta super family ligands selected from the group consisting of: activin A, activin B, GDF8, GDF11, and BMP10.

In certain embodiments, the disclosure relates to a heteromultimer comprising a first variant ActRIIB-Fc fusion polypeptide and a second variant ActRIIB-Fc fusion polypeptide, wherein the first variant ActRIIB polypeptide does not comprise the amino acid sequence of the second variant ActRIIB polypeptide. In some embodiments, an ActRIIB-Fc:ActRIIB-Fc heteromultimer binds to one or more TGF-beta superfamily ligands such as those described herein. In some embodiments, an ActRIIB-Fc:ActRIIB-Fc heteromultimer inhibits signaling of one or more TGF-beta superfamily ligands such as those described herein. In some embodiments, an ActRIIB-Fc:ActRIIB-Fc heteromultimer is a heterodimer.

In some embodiments, the first ActRIIB polypeptide comprises one or more amino acid substitutions at the amino acid positions corresponding to any one of F82, L79, A24, K74, R64, P129, P130, E37, R40, D54, R56, W78, D80, and F82 of SEQ ID NO: 2. In some embodiments, the first ActRIIB polypeptide comprises one or more amino acid substitutions at the amino acid positions corresponding to any one of L38N, E50L, E52N, L57E, L57I, L57R, L57T, L57V, Y60D, G68R, K74E. W78Y, L79F, L79S, L79T, L79W, F82D, F82E, F82L, F82S, F82T, F82Y, N83R, E94K, and V99G of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the one or more amino acid substitutions is selected from the group consisting of: L38N, E50L, E52N, L57E, L57I, L57R, L57T, L57V, Y60D, G68R, K74E, W78Y, L79F, L79S, L79T, L79W, F82D, F82E, F82L, F82S, F82T, F82Y, N83R, E94K, and V99G. In some embodiments, the second ActRIIB polypeptide comprises one or more amino acid substitutions at the amino acid positions corresponding to any one of F82, L79, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of L38N, E50L, E52N, L57E, L57I, L57R, L57T, L57V, Y60D. G68R, K74E, W78Y, L79F, L79S, L79T, L79W, F82D, F82E, F82L, F82S, F82T, F82Y, N83R, E94K, and V99G of SEQ ID NO: 2. In some embodiments, the one or more amino acid substitutions is selected from the group consisting of: L38N, E50L, E52N, L57E, L57I, L57R, L57T, L57V, Y60D, G68R. K74E, W78Y, L79F, L79S, L79T, L79W, F82D, F82E, F82L, F82S, F82T, F82Y, N83R, E94K, and V99G. In some embodiments, the first ActRIIB polypeptide and/or the second ActRIIB polypeptide comprise one or more amino acid modification that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 36, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, the first ActRIIB polypeptide comprises a glutamic acid at the amino acid position corresponding to 55 of SEQ ID NO: 2. In some embodiments, the second ActRIIB polypeptide does not comprise a glutamic acid at the amino acid position corresponding to 55 of SEQ ID NO: 2. In some embodiments, the second ActRIIB polypeptide comprises a lysine at the amino acid position corresponding to 55 of SEQ ID NO: 2. In some embodiments, the first ActRIIB polypeptide comprises one or more amino acid substitutions at the amino acid positions corresponding to any one of F82, L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of F82, L79. A24, K74. R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the first ActRIIB polypeptide and/or the second ActRIIB polypeptide comprise one or more amino acid modification that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 39, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB poly peptide does not comprise the amino acid sequence of the second ActRIIB polypeptide. In some embodiments, the first ActRIIB polypeptide comprises an isoleucine at the amino acid position corresponding to 82 of SEQ ID NO: 2. In some embodiments, the second ActRIIB polypeptide does not comprise an isoleucine acid at the amino acid position corresponding to 82 of SEQ ID NO: 2. In some embodiments, the second ActRIIB polypeptide comprises a phenylalanine at the amino acid position corresponding to 82 of SEQ ID NO: 2. In some embodiments, the first ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A. R40A, D54A, R56A, K74F, K74I, K74Y, W78A, D80A, D80F, D80G, D80I, D80K, D80M, D80M, D80N, and D80R. In some embodiments, the second ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modification that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 42, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 5, wherein the first ActRIIB polypeptide does not comprise the amino acid sequence of the second ActRIIB polypeptide. In some embodiments, first ActRIIB polypeptide comprises a lysine at the amino acid position corresponding to 82 of SEQ ID NO: 2. In some embodiments, the second ActRIIB polypeptide does not comprise a lysine acid at the amino acid position corresponding to 82 of SEQ ID NO: 2. In some embodiments, the second ActRIIB polypeptide comprises a phenylalanine at the amino acid position corresponding to 82 of SEQ ID NO: 2. In some embodiments, the first ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S, P130A, P130R, E37A, R40A, D54A, R56A, K74F, K74I, K74Y, W78A, D80A, D80F, D80G, D80I, D80K, D80M, D80M, D80N, and D80R. In some embodiments, the second ActRIIB polypeptide comprises one or more amino acid substitutions at the amino acid positions corresponding to any one of L79, A24, K74, R64, P129, P130, E37, R40, D54, R56, W78, and D80 of SEQ ID NO: 2. In some embodiments, the one or more amino acid substitutions is selected from the group consisting of: A24N, K74A, R64K, R64N, K74A, L79A, L79D, L79E, L79P, P129S. P130A, P130R, E37A. R40A, D54A, R56A, K74F, K74I, K74Y, W78A, D80A, D80F, D80G, D80I, D80K, D80M, D80M, D80N, and D80R. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modifications that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 45, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 48, wherein the first ActRIIB polypeptide does not comprise the amino acid sequence of the second ActRIIB polypeptide. In some embodiments, the first ActRIIB polypeptide comprises an acidic amino acid position corresponding to 79 of SEQ ID NO: 2. In some embodiments, the acidic amino acid is an aspartic acid. In some embodiments, the acidic amino acid is a glutamic acid. In some embodiments, the second ActRIIB polypeptide does not comprise an acidic acid (e.g., aspartic acid or glutamic acid) at the amino acid position corresponding to 79 of SEQ ID NO: 2. In some embodiments, the second ActRIIB polypeptide comprises a leucine at the amino acid position corresponding to 79 of SEQ ID NO: 2. In some embodiments, the first ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79P, P129S, P130A, P130R. E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of F82, A24, K74, R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, P129S. P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the first ActRIIB polypeptide and/or the second ActRIIB polypeptide comprise one or more amino acid modifications that promote heteromultimer formation. In some embodiments, the first ActRIIB polypeptide and/or the second ActRIIB polypeptide comprise one or more amino acid modifications that inhibit heteromultimer formation. In some embodiments, the heteromultimer is a heterodimer.

In certain aspects, the disclosure relates to a heteromultimer comprising a first ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 50, and second ActRIIB polypeptide that is at least 75%, 80%, 85%, 90%, 91%, 92%, 93%, 94%, 95%, 96%, 97%, 98%, 99%, or 100% identical to the amino acid sequence of SEQ ID NO: 52, wherein the first ActRIIB polypeptide does not comprise the amino acid sequence of the second ActRIIB polypeptide. In some embodiments, the first ActRIIB polypeptide comprises an acidic amino acid position corresponding to 79 of SEQ ID NO: 2. In some embodiments, the acidic amino acid is an aspartic acid. In some embodiments, the acidic amino acid is a glutamic acid. In some embodiments, the second ActRIIB polypeptide does not comprise an acidic acid (e.g., aspartic acid or glutamic acid) at the amino acid position corresponding to 79 of SEQ ID NO: 2. In some embodiments, the second ActRIIB polypeptide comprises a leucine at the amino acid position corresponding to 79 of SEQ ID NO: 2. In some embodiments, the first ActRIIB polypeptide comprises one or more amino acid substitutions at the amino acid positions corresponding to any one of F82, A24, K74. R64, P129, P130, E37, R40, D54, R56, W78, D80, and F82 of SEQ ID NO: 2. In some embodiments, the one or more amino acid substitutions is selected from the group consisting of: A24N, K74A, R64K, R64N, K74A, L79P, P129S, P130A, P130R, E37A, R40A, D54A, R56A, K74F, K74I, K74Y, W78A, D80A, D80F, D80G, D80I, D80K, D80M, D80M, D80N, D80R, and F82A. In some embodiments, the second ActRIIB polypeptide comprises one or more amino acid substitutions at the amino acid positions corresponding to any one of F82, A24, K74, R64, P129, P130, E37, R40, D54, R56, W78. D80, and F82 of SEQ ID NO: 2. In some embodiments, the one or more amino acid substitutions is selected from the group consisting of: A24N, K74A, R64K, R64N, K74A, P129S, P130A, P130R, E37A, R40A, D54A, R56A, K74F, K74I, K74Y, W78A, D80A, D80F, D80G, D80I, D80K, D80M, D80M, D80N, D80R, and F82A.

In certain embodiments, the disclosure relates to a heteromultimer comprising a first variant ActRIIB-Fc fusion polypeptide and a second variant ActRIIB-Fc fusion polypeptide, wherein the second variant ActRIIB-Fc fusion polypeptide differs from that present in the first polypeptide. In some embodiments, an ActRIIB-Fc:ActRIIB-Fc heteromultimers binds to one or more TGF-beta superfamily ligands such as those described herein. In some embodiments, an ActRIIB-Fc:ActRIIB-Fc heteromultimers inhibit signaling of one or more TGF-beta superfamily ligands such as those described herein. In some embodiments, an ActRIIB-Fc:ActRIIB-Fc heteromultimers is a heterodimer.

In some embodiments, the present disclosure contemplates making functional variants by modifying the structure of a variant ActRIIB polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more TGF-beta superfamily ligands including, for example, activin A, activin B, GDF8, GDF11 and BMP10.

In some embodiments, the present disclosure contemplates making functional variants by modifying the structure of a variant ActRIIB polypeptide for such purposes as enhancing therapeutic efficacy or stability (e.g., increased shelf-life and/or increased resistance to proteolytic degradation).

In certain embodiments, the present disclosure contemplates specific mutations of a variant ActRIIB polypeptide of the disclosure so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence.

Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, a variant ActRIIB polypeptide complex of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of a variant ActRIIB polypeptide of the disclosure, as well as truncation mutants. Pools of combinatorial mutants are especially useful for identifying functionally active (e.g., ligand binding) variant ActRIIB polypeptide sequences.

The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, variant ActRIIB polypeptide sequences may be screened for ability to bind to a TGF-beta superfamily ligand (e.g., activin A, activin B, GDF8, GDF11, and BMP10), to prevent binding of a TGF-beta superfamily ligand to a TGF-beta superfamily receptor, and/or to interfere with signaling caused by an TGF-beta superfamily ligand.

The activity of a variant ActRIIB polypeptide heteromultimer of the disclosure also may be tested, for example in a cell-based or in vivo assay. For example, the effect of a heteromultimer complex on the expression of genes or the activity of proteins involved in BMP signaling in a pulmonary cell may be assessed. This may, as needed, be performed in the presence of one or more recombinant TGF-beta superfamily ligand proteins (e.g., activin A, activin B. GDF8, GDF11, and BMP10), and cells may be transfected so as to produce a variant ActRIIB polypeptide complex, and optionally, a TGF-beta superfamily ligand. Likewise, a heteromultimer complex of the disclosure may be administered to a mouse or other animal, and one or more measurements, such as decreasing muscularization of pulmonary arteries may be assessed using art-recognized methods. Similarly, the activity of a heteromultimer, or variants thereof, may be tested in cardiac or pulmonary cells for any effect on growth of these cells, for example, by the assays as described herein and those of common knowledge in the art. A SMAD-responsive reporter gene may be used in such cell lines to monitor effects on downstream signaling.

In certain aspects, heteromultimers of the disclosure bind to one or more TGF-beta superfamily ligands. In some embodiments, heteromultimers of the disclosure bind to one or more TGF-beta superfamily ligands with a Ku of at least 1×10V M. In some embodiments, the one or more TGF-beta superfamily ligands is selected from the group consisting of: activin A, activin B, GDF8, GDF11, and BMP10.

In certain aspects, heteromultimers of the disclosure inhibits one or more TGF-beta super family ligands. In some embodiments, heteromultimers of the disclosure inhibits signaling of one or more TGF-beta super family ligands. In some embodiments, heteromultimers of the disclosure inhibits Smad signaling of one or more TGF-beta super family ligands. In some embodiments, heteromultimers of the disclosure inhibits signaling of one or more TGF-beta super family ligands in a cell-based assay. In some embodiments, heteromultimers of the disclosure inhibits one or more TGF-beta super family ligands selected from the group consisting of: activin A, activin B. GDF8, GDF11, and BMP10.

Combinatorial-derived variants can be generated which have increased selectivity or generally increased potency relative to a reference variant ActRIIB polypeptide heteromultimer. Such variants, when expressed from recombinant DNA constructs, can be used in gene therapy protocols. Likewise, mutagenesis can give rise to variants which have intracellular half-lives dramatically different than the corresponding unmodified variant ActRIIB polypeptide heteromultimer. For example, the altered polypeptide can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the poly peptide. For instance, a short half-life can give rise to more transient biological effects and, when part of an inducible expression system, can allow tighter control of recombinant polypeptide complex levels within the cell. In an Fc fusion polypeptide, mutations may be made in the linker (if any) and/or the Fc portion to alter one or more activities of the variant ActRIIB polypeptide heteromultimer complex including, for example, immunogenicity, half-life, and solubility.

Many methods known in the art can be used to generate heteromultimers of the disclosure. For example, non-naturally occurring disulfide bonds may be constructed by replacing on a first polypeptide (e.g., a first variant ActRIIB polypeptide) a naturally occurring amino acid with a free thiol-containing residue, such as cysteine, such that the free thiol interacts with another free thiol-containing residue on a second polypeptide (e.g., a second variant ActRIIB polypeptide) such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S. Pat. No. 8,592,562; coiled-coil interactions such as described in Christensen et al., U.S. 20120302737; leucine zippers such as described in Pack & Plueckthun, (1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form polypeptide complexes comprising a first polypeptide covalently or non-covalently associated with a second polypeptide, wherein the first polypeptide comprises the amino acid sequence of a first variant ActRIIB polypeptide and the amino acid sequence of a first member of an interaction pair; and the second polypeptide comprises the amino acid sequence of a second variant ActRIIB polypeptide, and the amino acid sequence of a second member of an interaction pair. The interaction pair may be any two polypeptide sequences that interact to form a complex, particularly a heterodimeric complex although operative embodiments may also employ an interaction pair that can form a homodimeric complex. An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric complex (see, e.g., FIG. 1B). Alternatively, the interaction pair may be unguided, meaning that the members of the pair may associate with each other or self-associate without substantial preference and thus may have the same or different amino acid sequences (see, e.g., FIG. 1A). Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair. As specific examples, the present disclosure provides fusion polypeptides comprising a variant ActRIIB polypeptide or an unmodified ActRIIB polypeptide fused to a polypeptide comprising a constant domain of an immunoglobulin, such as a CH1, CH2, or CH3 domain of an immunoglobulin or an Fc domain. Fc domains derived from human IgG1, IgG2, IgG3, and IgG4 are provided herein. Other mutations are known that decrease either CDC or ADCC activity, and collectively, any of these variants are included in the disclosure and may be used as advantageous components of a heteromultimers of the disclosure. Optionally, the IgG1 Fc domain of SEQ ID NO: 13 has one or more mutations at residues such as Asp-265, Lys-322, and Asn-434 (numbered in accordance with the corresponding full-length IgG1). In certain cases, the variant Fe domain having one or more of these mutations (e.g., Asp-265 mutation) has reduced ability of binding to the Fcγ receptor relative to a wildtype Fc domain. In other cases, the variant Fc domain having one or more of these mutations (e.g., Asn-434 mutation) has increased ability of binding to the MHC class I-related Fc-receptor (FcRN) relative to a wild-type Fc domain.

An example of a native amino acid sequence that may be used for the Fc portion of human IgG1 (G1Fc) is shown below (SEQ ID NO: 13). Dotted underline indicates the hinge region, and solid underline indicates positions with naturally occurring variants. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 13. In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 13, and wherein the second variant ActRIIB-Fc fusion polypeptide comprises an Fe domain that is at least 75%, 80%, 85%, 90%, 91%, 92%, 93%, 94%, 95%, 96%, 97%, 98%, 99%, or 100% identical to the amino acid sequence of SEQ ID NO: 13. Naturally occurring variants in G1Fc would include E134D and M136L according to the numbering system used in SEQ ID NO: 13 (see Uniprot P01857).

| (SEQ ID NO: 13) | |
|---|---|
| 1 | 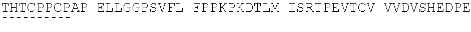 |
| 51 | VKFNWYVDGV EVHNAKTKPR EEQYNSTYRV VSVLTVLHQD WLNGKEYKCK |
| 101 | 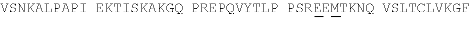 |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

An example of a native amino acid sequence that may be used for the Fc portion of human IgG2 (G2Fc) is shown below (SEQ ID NO: 14). Dotted underline indicates the hinge region and double underline indicates positions where there are data base conflicts in the sequence (according to UniProt P01859). In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 14. In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14, and wherein the second variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 14.

| (SEQ ID NO: 14) | |
|---|---|
| 1 | 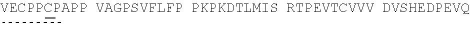 |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | 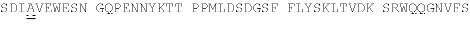 |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 15) contains a short hinge region consisting of a single 15-residue segment, whereas the second G3Fc sequence (SEQ ID NO: 16) contains a full-length hinge region. In each case, dotted underline indicates the hinge region, and solid underline indicates positions with naturally occurring variants according to UniProt P01859. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NOs: 15 and 16. In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15, and wherein the second variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 15. In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 16, and wherein the second variant ActRIIB-Fc fusion polypeptide comprises an Fc domain that is at least 75%, 80%, 85%, 90%, 91%, 92%, 93% 94%, 95%, 96%, 97%, 98%, 99%, or 100% identical to the amino acid sequence of SEQ ID NO: 16.

| (SEQ ID NO: 15) | |
|---|---|
| 1 | 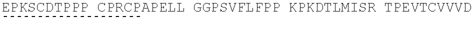 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| (SEQ ID NO: 16) | |
| 1 | 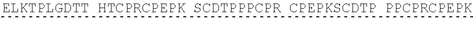 |
| 51 | 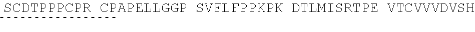 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del. F221Y when converted to the numbering system used in SEQ ID NO: 15, and the present disclosure provides fusion polypeptides comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion polypeptides comprising G3Fc domains containing one or more of these variants. An example of a native amino acid sequence that may be used for the Fc portion of human IgG4 (G4Fc) is shown below (SEQ ID NO: 17). Dotted underline indicates the hinge region. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 17. In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 17, and wherein the second variant ActRIIB-Fc fusion polypeptide comprises an Fe domain that is at least 75%, 80%, 85%, 90%, 91%, 92%, 93%, 94%, 95%, 96%, 97%, 98%, 99%, or 100% identical to the amino acid sequence of SEQ ID NO: 17.

| (SEQ ID NO: 17) | |
|---|---|
| 1 | 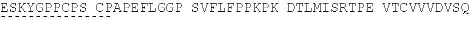 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

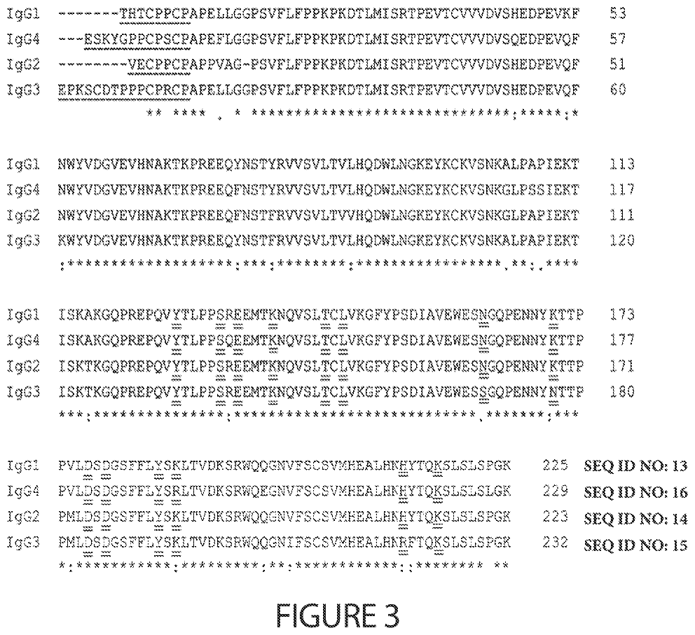

A variety of engineered mutations in the Fe domain are presented herein with respect to the G1Fc sequence (SEQ ID NO: 13), and analogous mutations in G2Fc, G3Fc, and G4Fc can be derived from their alignment with G1Fc in FIG. 3. Due to unequal hinge lengths, analogous Fc positions based on isotype alignment (FIG. 3) possess different amino acid numbers in SEQ ID NOs: 13, 14, 15, and 17. It can also be appreciated that a given amino acid position in an immunoglobulin sequence consisting of hinge, CH2, and CH3 regions (e.g., SEQ ID NOs: 13, 14, 15, 16, or 17) will be identified by a different number than the same position when numbering encompasses the entire IgG1 heavy-chain constant domain (consisting of the CH1, hinge, CH2, and CH3 regions) as in the Uniprot database. For example, correspondence between selected CH3 positions in a human G1Fc sequence (SEQ ID NO: 13), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | IgG1 heavy chain |
|---|---|---|
| (Numbering begins | constant domain | (EU numbering |
| at first threonine | (Numbering begins | scheme of |
| in hinge region) | at C_{H}1) | Kabat et al., 1991*) |
| Y127 | Y232 | Y349 |
| S132 | S237 | S354 |
| E134 | E239 | E356 |
| K138 | K243 | K360 |
| T144 | T249 | T366 |
| L146 | L251 | L368 |
| N162 | N267 | N384 |
| K170 | K275 | K392 |
| D177 | D282 | D399 |
| D179 | D284 | D401 |
| Y185 | Y290 | Y407 |
| K187 | K292 | K409 |
| H213 | H318 | H435 |
| K217 | K322 | K439 |

*Kabat et al. (eds) 1991; pp. 688-696 in Sequences of Proteins of Immunological Interest, 5^{th} ed., Vol. 1, NIH, Bethesda, MD.

A problem that arises in large-scale production of asymmetric immunoglobulin-based proteins from a single cell line is known as the “chain association issue”. As confronted prominently in the production of bispecific antibodies, the chain-association issue concerns the challenge of efficiently producing a desired multichain protein from among the multiple combinations that inherently result when different heavy chains and/or light chains are produced in a single cell line [see, for example. Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

Various methods are known in the art that increase desired pairing of Fc-containing fusion polypeptide chains in a single cell line to produce a preferred asymmetric fusion protein at acceptable yields [see, for example, Klein et al (2012) mAbs 4:653-663; and Spiess et al (2015) Molecular Immunology 67(2A): 95-106]. Methods to obtain desired pairing of Fe-containing chains include, but are not limited to, charge-based pairing (electrostatic steering). “knobs-into-holes” steric pairing, SEEDbody pairing, and leucine zipper-based pairing. See, for example, Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605. As described herein, these methods may be used to generate heterodimers comprising a variant ActRIIB polypeptide and another, optionally different, variant ActRIIB polypeptide or an unmodified ActRIIB polypeptide.

For example, one means by which interaction between specific polypeptides may be promoted is by engineering protuberance-into-cavity (knob-into-holes) complementary regions such as described in Arathoon et al., U.S. Pat. No. 7,183,076 and Carter et al., U.S. Pat. No. 5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged, and lysine, arginine, and histidine are positively charged. These charged residues can be used to promote heterodimer formation and at the same time hinder homodimer formation. Attractive interactions take place between opposite charges and repulsive interactions occur between like charges. In part, polypeptide complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will be represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation of the heteromultimers disclosed herein.

Examples of Pair-Wise Charged Residue Mutations
to Enhance Heterodimer Formation

| | | Interacting | Corresponding |
|---|---|---|---|
| Position in | Mutation in | position in | mutation in second |
| first chain | first chain | second chain | chain |
| Lys409 | Asp or Glu | Asp399′ | Lys, Arg, or His |
| Lys392 | Asp or Glu | Asp399′ | Lys, Arg, or His |
| Lys439 | Asp or Glu | Asp356′ | Lys, Arg, or His |
| Lys370 | Asp or Glu | Glu357′ | Lys, Arg, or His |
| Asp399 | Lys, Arg, or His | Lys409′ | Asp or Glu |
| Asp399 | Lys, Arg, or His | Lys392′ | Asp or Glu |
| Asp356 | Lys, Arg, or His | Lys439′ | Asp or Glu |
| Glu357 | Lys, Arg, or His | Lys370′ | Asp or Glu |

In some embodiments, one or more residues that make up the CH3-CH3 interface in a fusion polypeptide of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to a variant ActRIIB polypeptide of the construct, with or without an optional linker, to generate a variant ActRIIB-Fc fusion polypeptide. This single chain can be coexpressed in a cell of choice along with the Fc sequence complementary to the first Fc sequence to favor generation of the desired multichain construct (e.g., a variant ActRIIB-Fc:variant ActRIIB-Fc heteromultimer). In this example based on electrostatic steering, SEQ ID NO: 18 [human G1Fc(E134K/D177K)] and SEQ ID NO: 19 [human G1Fc(K170D/K187D)] are examples of complementary Fc sequences in which the engineered amino acid substitutions are double underlined, and a first variant ActRIIB polypeptide, second variant ActRIIB polypeptide, or unmodified ActRIIB polypeptide of the construct can be fused to either SEQ ID NO: 18 or SEQ ID NO: 19, but not both. Given the high degree of amino acid sequence identity between native hG1Fe, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair below (SEQ ID NOs: 18 and 19).

| (SEQ ID NO: 18) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSRKEMTKNQ VSLTCLVKGF |
| 151 | YPSDIAVEWE SNGQPENNYK TTPPVLKSDG SFFLYSKLTV DKSRWQQGNV |
| 201 | FSCSVMHEAL HNHYTQKSLS LSPGK |
| (SEQ ID NO: 19) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYD TTPPVLDSDG SFFLYSDLTV DKSRWQQGNV |
| 201 | FSCSVMHEAL HNHYTQKSLS LSPGK |

In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19, and the second variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18. In some embodiments, the disclosure relates to ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 18, and wherein the second variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 19.

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to a variant ActRIIB polypeptide of the construct, with or without an optional linker, to generate a variant ActRIIB-Fc fusion polypeptide. This single chain can be coexpressed in a cell of choice along with the Fc sequence complementary to the first Fc sequence to favor generation of the desired multichain construct. In this example based on knobs-into-holes pairing. SEQ ID NO: 20 [human G1Fc(T144Y)] and SEQ ID NO: 21 [human G1Fc(Y185T)] are examples of complementary Fc sequences in which the engineered amino acid substitutions are double underlined, and a first variant ActRIIB polypeptide, second variant ActRIIB polypeptide, or unmodified ActRIIB polypeptide of the construct can be fused to either SEQ ID NO: 20 or SEQ ID NO: 21, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair below (SEQ ID NOs: 20 and 21).

| (SEQ ID NO: 20) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLYCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 21) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLTSKLTV DKSRWQQGNV |
| 201 | FSCSVMHEAL HNHYTQKSLS LSPGK |

In some embodiments, the disclosure relates to ActRIIB:ActRIIB heteromultimer polypeptides comprising a first variant ActRIIB-Fc fusion polypeptide and second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21, and wherein the second variant ActRIIB-Fc fusion polypeptide comprises an Fe domain that is at least 75%, 80%, 85%, 90%, 91%, 92%, 93%, 94%, 95%, 96%, 97%, 98%, 99%, or 100% identical to the amino acid sequence of SEQ ID NO: 20. In some embodiments, the disclosure relates to variant ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 20, and wherein the second variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 21.

An example of Fc complementarity based on knobs-into-holes pairing combined with an engineered disulfide bond is disclosed in SEQ ID NO: 22 [hG1Fc(S132C/T144W)] and SEQ ID NO: 23 [hG1Fc(Y127C/T144S/L146A/Y185V)]. The engineered amino acid substitutions in these sequences are double underlined, and a first variant ActRIIB polypeptide, second variant ActRIIB polypeptide, or unmodified ActRIIB polypeptide of the construct can be fused to either SEQ ID NO: 22 or SEQ ID NO: 23, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair

| (SEQ ID NO: 22) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 23) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SNGQPENNYK TTPPVLDSDG SFFLVSKLTV DKSRWQQGNV |
| 201 | FSCSVMHEAL HNHYTQKSLS LSPGK |

In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23, and wherein the second variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 22.

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion polypeptides [see, for example, Davis et al (2010) Protein Eng Design Sel 23:195-202]. One of a pair of Fc sequences with SEEDbody complementarity can be arbitrarily fused to a first variant ActRIIB polypeptide, second variant ActRIIB polypeptide, or unmodified ActRIIB polypeptide of the construct, with or without an optional linker, to generate a variant ActRIIB-Fc or unmodified ActRIIB-Fc fusion polypeptide. This single chain can be coexpressed in a cell of choice along with the Fc sequence complementary to the first Fc sequence to favor generation of the desired multichain construct. In this example based on SEEDbody (Sb) pairing. SEQ ID NO: 24 [hG1Fc(Sb_{AG})] and SEQ ID NO: [hG1Fc(Sb_{GA})] are examples of complementary IgG Fc sequences in which the engineered amino acid substitutions from IgA Fc are double underlined, and a first variant ActRIIB polypeptide, second variant ActRIIB polypeptide, or unmodified ActRIIB polypeptide of the construct can be fused to either SEQ ID NO: 24 or SEQ ID NO: 25, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc, or hG4Fc (see FIG. 3) will generate an Fc monomer which may be used in the complementary IgG-IgA pair below (SEQ ID NOs: 24 and 25).

| (SEQ ID NO: 24) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PFRPEVHLLP PSREEMTKNQ VSLTCLARGF |
| 151 | YPKDIAVEWE SNGQPENNYK TTPSRQEPSQ GTTTFAVTSK LTVDKSRWQQ |
| 201 | GNVFSCSVMH EALHNHYTQK TISLSPGK |
| (SEQ ID NO: 25) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PPSEELALNE LVTLTCLVKG |
| 151 | FYPSDIAVEW ESNGQELPRE KYLTWAPVLD SDGSFFLYSI LRVAAEDWKK |
| 201 | GDTFSCSVMH EALHNHYTQK SLDRSPGK |

In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 25, and wherein the second variant ActRIIB-Fc fusion polypeptide comprises an Fe domain that is at least 75%, 80%, 85%, 90%, 91%, 92%, 93%, 94%, 95%, 96%, 97%, 98%, 99%, or 100% identical to the amino acid sequence of SEQ ID NO: 24.

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains. See, e.g., Wranik et al (2012) J Biol Chem 287:43331-43339. As disclosed herein, one of a pair of Fc sequences attached to a leucine zipper-forming strand can be arbitrarily fused to a first variant ActRIIB polypeptide, second variant ActRIIB polypeptide, or unmodified ActRIIB polypeptide of the construct, with or without an optional linker, to generate a variant ActRIIB-Fc or unmodified ActRIIB-Fc fusion polypeptide. This single chain can be coexpressed in a cell of choice along with the Fc sequence attached to a complementary leucine zipper-forming strand to favor generation of the desired multichain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 26 [hG1Fc-Ap1 (acidic)] and SEQ ID NO: 27 [hG1Fc-Bp1 (basic)] are examples of complementary IgG Fc sequences in which the engineered complimentary leucine zipper sequences are underlined, and a first variant ActRIIB polypeptide, second variant ActRIIB polypeptide, or wild-type ActRIIB polypeptide of the construct can be fused to either SEQ ID NO: 26 or SEQ ID NO: 27, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fc, hG2Fc, hG3Fc, or hG4Fc (see FIG. 3) will generate an Fc monomer which may be used in the complementary leucine zipper-forming pair below (SEQ ID NOs: 26 and 27).

| (SEQ ID NO: 26) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LEKELQALEK ENAQLEWELQ |
| 251 | ALEKELAQGA T |
| (SEQ ID NO: 27) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LKKKLQALKK KNAQLKWKLQ |
| 251 | ALKKKLAQGA T |

In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 27, and wherein the second variant ActRIIB-Fc fusion polypeptide comprises an Fe domain that is at least 75%, 80%, 85%, 90%, 91%, 92%, 93%, 94%, 95%, 96%, 97%, 98%, 99%, or 100% identical to the amino acid sequence of SEQ ID NO: 26.

In part, the disclosure provides desired pairing of asymmetric Fc-containing polypeptide chains by methods described above in combination with additional mutations in the Fc domain which facilitate purification of the desired heteromeric species. An example uses complementarity of Fc domains based on knobs-into-holes pairing combined with an engineered disulfide bond, as disclosed in SEQ ID NOs: 22 and 23, plus additional substitution of two negatively charged amino acids (aspartic acid or glutamic acid) in one Fc-containing polypeptide chain and two positively charged amino acids (e.g., arginine) in the complementary Fc-containing polypeptide chain (SEQ ID NOs: 28-29). These four amino acid substitutions facilitate selective purification of the desired heteromeric fusion polypeptide from a heterogencous polypeptide mixture based on differences in isoelectric point or net molecular charge. The engineered amino acid substitutions in these sequences are double underlined below, and a first variant ActRIIB polypeptide, second variant ActRIIB polypeptide, or unmodified ActRIIB polypeptide of the construct can be fused to either SEQ ID NO: 28 or SEQ ID NO: 29, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair below (SEQ ID NOs: 28-29).

| (SEQ ID NO: 28) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTENQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQDSLS LSPGK |
| (SEQ ID NO: 29) | |
| 1 | THTCPPCPAP ELLGGPSVFL FPPKPKDTEM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SRGQPENNYK TTPPVLDSRG SFFLVSKLTV DKSRWQQGNV |
| 201 | FSCSVMHEAL HNHYTQKSLS LSPGK |

In some embodiments, the disclosure relates to ActRIIB:ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fe domain that is at least 75%, 80%, 85%, 90%, 91%, 92%, 93%, 94%, 95%, 96%, 97%, 98%, 99%, or 100% identical to the amino acid sequence of SEQ ID NO: 28, and wherein the second variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 29. In some embodiments, the first variant ActRIIB-Fc fusion polypeptide Fc domain comprises a cysteine at amino acid position 132, glutamic acid at amino acid position 138, a tryptophan at amino acid position 144, and an aspartic acid at amino acid position 217. In some embodiments, the second variant ActRIIB-Fc fusion polypeptide Fc domain comprises a cysteine at amino acid position 127, a serine at amino acid position 144, an alanine at amino acid position 146, an arginine at amino acid position 162, an arginine at amino acid position 179, and a valine at amino acid position 185.

Another example involves complementarity of Fc domains based on knobs-into-holes pairing combined with an engineered disulfide bond, as disclosed in SEQ ID NOs: 22-23, plus a histidine-to-arginine substitution at position 213 in one Fc-containing polypeptide chain (SEQ ID NO: 30). This substitution (denoted H435R in the numbering system of Kabat et al.) facilitates separation of desired heteromer from undesirable homodimer based on differences in affinity for protein A. The engineered amino acid substitution is indicated by double underline, and a first variant ActRIIB polypeptide, second variant ActRIIB polypeptide, or unmodified ActRIIB polypeptide of the construct can be fused to either SEQ ID NO: 30 or SEQ ID NO: 23, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 3) will generate complementary Fc pairs which may be used instead of the complementary hG1Fc pair of SEQ ID NO: 30 (below) and SEQ ID NO: 23.

| (SEQ ID NO: 30) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNRYTQKSLS LSPGK |

In some embodiments, the disclosure relates to ActRIIB: ActRIIB heteromultimer polypeptides comprising a first variant ActRIIB-Fc fusion polypeptide and a second variant ActRIIB-Fc fusion polypeptide wherein the first variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 30, and wherein the second variant ActRIIB-Fc fusion polypeptide comprises an Fc domain that is at least 75%, 80%, 85%, 90%, 91%, 92%, 93%, 94%, 95%, 96%, 97%, 98%, 99%, or 100% identical to the amino acid sequence of SEQ ID NO: 23. In some embodiments, the first variant ActRIIB-Fc fusion polypeptide Fc domain comprises a cysteine at amino acid position 132, a tryptophan at amino acid position 144, and an arginine at amino acid position 435. In some embodiments, the second variant ActRIIB-Fc fusion polypeptide Fc domain comprises cysteine at amino acid position 127, a serine at amino acid position 144, an alanine at amino acid position 146, and a valine at amino acid position 185.

A variety of engineered mutations in the Fc domain are presented above with respect to the G1Fc sequence (SEQ ID NO: 13). Analogous mutations in G2Fc, G3Fc, and G4Fc can be derived from their alignment with G1Fc in FIG. 3. Due to unequal hinge lengths, analogous Fc positions based on isotype alignment (FIG. 3) possess different amino acid numbers in SEQ ID NOs: 13, 14, 15, 16, and 17 as summarized in the following table.

Correspondence between C_{H}3 Positions for Human Fc Isotypes*

| IgG1 | IgG4 | IgG2 | IgG3 |
|---|---|---|---|
| SEQ ID NO: 13 | SEQ ID NO: 17 | SEQ ID NO: 14 | SEQ ID NO: 15 |
| Numbering | Numbering | Numbering | Numbering |
| begins at | begins at | begins at | begins at |
| THT . . . | ESK . . . | VEC . . . | EPK . . . |
| Y127 | Y131 | Y125 | Y134 |
| S132 | S136 | S130 | S139 |
| E134 | E138 | E132 | E141 |
| K138 | K142 | K136 | K145 |
| T144 | T148 | T142 | T151 |
| L146 | L150 | L144 | L153 |
| N162 | N166 | N160 | S169 |
| K170 | K174 | K168 | N177 |
| D177 | D181 | D175 | D184 |
| D179 | D183 | D177 | D186 |
| Y185 | Y189 | Y183 | Y192 |
| K187 | R191 | K185 | K194 |
| H213 | H217 | H211 | R220 |
| K217 | K221 | K215 | K224 |

*Numbering based on multiple sequence alignment shown in FIG. 3

It is understood that different elements of the fusion polypeptides (e.g., immunoglobulin Fc fusion polypeptides) may be arranged in any manner that is consistent with desired functionality. For example, a variant ActRIIB polypeptide domain may be placed C-terminal to a heterologous domain, or alternatively, a heterologous domain may be placed C-terminal to a variant ActRIIB polypeptide domain. The variant ActRIIB polypeptide domain and the heterologous domain need not be adjacent in a fusion polypeptide, and additional domains or amino acid sequences may be included C- or N-terminal to either domain or between the domains.

For example, a variant ActRIIB polypeptide may comprise an amino acid sequence as set forth in the formula A-B-C. The B portion corresponds to a variant ActRIIB polypeptide domain. The A and C portions may be independently zero, one, or more than one amino acid, and both the A and C portions when present are heterologous to B. The A and/or C portions may be attached to the B portion via a linker sequence. In certain embodiments, a variant ActRIIB fusion polypeptide comprises an amino acid sequence as set forth in the formula A-B-C, wherein A is a leader (signal) sequence, B consists of a variant ActRIIB polypeptide domain, and C is a polypeptide portion that enhances one or more of in vivo stability, in vivo half-life, uptake/administration, tissue localization or distribution, formation of protein complexes, and/or purification. In certain embodiments, a variant ActRIIB fusion protein comprises an amino acid sequence as set forth in the formula A-B-C, wherein A is a TPA leader sequence, B consists of a variant ActRIIB polypeptide domain, and C is an immunoglobulin Fc domain.

In certain embodiments, the variant ActRIIB polypeptides of the present disclosure contain one or more modifications that are capable of stabilizing the variant ActRIIB polypeptides. For example, such modifications enhance the in vitro half life of the variant ActRIIB polypeptides, enhance circulatory half life of the variant ActRIIB polypeptides or reducing proteolytic degradation of the variant ActRIIB polypeptides. Such stabilizing modifications include, but are not limited to, fusion polypeptides (including, for example, fusion polypeptides comprising a variant ActRIIB polypeptide and a stabilizer domain), modifications of a glycosylation site (including, for example, addition of a glycosylation site to a variant ActRIIB polypeptide), and modifications of carbohydrate moiety (including, for example, removal of carbohydrate moieties from a variant ActRIIB polypeptide). In the case of fusion polypeptides, a variant ActRIIB polypeptide is fused to a stabilizer domain such as an IgG molecule (e.g., an Fc domain). As used herein, the term “stabilizer domain” not only refers to a fusion domain (e.g., Fc) as in the case of fusion polypeptides, but also includes nonproteinaceous modifications such as a carbohydrate moiety, or nonproteinaceous polymer, such as polyethylene glycol.

In certain embodiments, the present disclosure makes available isolated and/or purified forms of the variant ActRIIB polypeptides, which are isolated from, or otherwise substantially free of, other polypeptides.

In certain embodiments, variant ActRIIB polypeptides of the disclosure can be produced by a variety of art-known techniques. For example, such variant ActRIIB polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the variant ActRIIB polypeptides, fragments or variants thereof may be recombinantly produced using various expression systems (e.g., E. coli, Chinese Hamster Ovary cells, COS cells, baculovirus) as is well known in the art (also see below). In a further embodiment, the variant ActRIIB polypeptides may be produced by digestion of naturally occurring or recombinantly produced full-length variant ActRIIB polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites. Alternatively, such variant ActRIIB polypeptides may be produced from naturally occurring or recombinantly produced full-length variant ActRIIB polypeptides such as standard techniques known in the art, such as by chemical cleavage (e.g., cyanogen bromide, hydroxylamine).

In certain embodiments, variant ActRIIB polypeptides of the disclosure can include a purification subsequence, such as an epitope tag, a FLAG tag, a polyhistidine sequence, and a GST fusion. Optionally, a variant ActRIIB polypeptide includes one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent.

In some embodiments, the disclosure relates to variant ActRIIB polypeptides, including variant ActRIIB polypeptides as well as homomultimer and heteromultimers comprising the same, that comprise one or more amino acid modifications selected from the group consisting of: aglycosylated amino acid, a PEGylated amino acid, a farnesylated amino acid, an acetylated amino acid, a biotinylated amino acid, and an amino acid conjugated to a lipid moiety. In some embodiments, variant ActRIIB polypeptides of the disclosure are glycosylated and have a glycosylation pattern obtainable from of the polypeptide in a CHO cell.

### 3. Linkers

The disclosure provides for variant ActRIIB polypeptides that may be fused to any of the other polypeptides disclosed herein, or that may be fused to a heterologous portion (e.g., an Fc portion). In these embodiments, the polypeptide portion (e.g, a variant ActRIIB polypeptide) is connected to the other polypeptide (e.g., a TGFβRII polypeptide) and/or to the heterologous portion (e.g., Fc portion) by means of a linker. In some embodiments, the linkers are glycine and serine rich linkers. In some embodiments, the linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 261), GGGG (SEQ ID NO: 262), TGGGG (SEQ ID NO: 263), SGGGG (SEQ ID NO: 264). TGGG (SEQ ID NO: 265), or SGGG (SEQ ID NO: 266) singlets, or repeats. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In preferred embodiments, the linker comprises the amino acid sequence GlyGlyGlyGlySer (GGGGS) (SEQ ID NO: 267), or repetitions thereof (GGGGS)n, where n≥2. In particular embodiments n≥3, or n=3-10. In some embodiments, n≥4, or n=4-10. In some embodiments, n is not greater than 4 in a (GGGGS)n linker. In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS)n sequence also comprises an N-terminal threonine. In some embodiments, the linker is any one of the following:

(SEQ ID NO: 268)
GGGGSGGGGS
(SEQ ID NO: 269)
TGGGGSGGGGS
(SEQ ID NO: 270)
TGGGGSGGGGSGGGGS
(SEQ ID NO: 271)
TGGGGSGGGGSGGGGSGGGGS
(SEQ ID NO: 272)
TGGGGSGGGGSGGGGSGGGGSGGGGS
(SEQ ID NO: 273)
TGGGGSGGGGSGGGGSGGGGSGGGGSGGGGS
or
(SEQ ID NO: 274)
TGGGGSGGGGSGGGGSGGGGSGGGGSGGGGSGGGGS.

In some embodiments, the linker comprises the amino acid sequence of TGGGPKSCDK (SEQ ID NO: 275). In some embodiments, the linker is any one of SEQ ID NOs: 268-275 lacking the N-terminal threonine. In some embodiments, the linker does not comprise the amino acid sequence of SEQ ID NO: 273 or 274.

In some embodiments, a polypeptide described (e.g., variant ActRIIB polypeptides in either homomeric or heteromeric forms) herein may include a polypeptide fused to a moiety by way of a linker. In some embodiments, the moiety increases stability of the polypeptide. In some embodiments, the moity is selected from the group consisting of an Fc domain monomer, a wild-type Fc domain, an Fc domain with amino acid substitutions (e.g., one or more substitutions that reduce dimerization), an albumin-binding peptide, a fibronectin domain, or a human serum albumin. Suitable peptide linkers are known in the art, and include, for example, peptide linkers containing flexible amino acid residues such as glycine, alanine, and serine. In some embodiments, a linker can contain motifs, e.g., multiple or repeating motifs, of GA, GS, GG, GGA, GGS, GGG (SEQ ID NO: 261), GGGA (SEQ ID NO: 280), GGGS (SEQ ID NO: 281), GGGG (SEQ ID NO: 262), GGGGA (SEQ ID NO: 282), GGGGS (SEQ ID NO: 267), GGGGG (SEQ ID NO: 283), GGAG (SEQ ID NO: 284), GGSG (SEQ ID NO: 285), AGGG (SEQ ID NO: 286), or SGGG (SEQ ID NO: 266). In some embodiments, a linker can contain 2 to 12 amino acids including motifs of GA or GS, e.g., GA, GS, GAGA (SEQ ID NO: 287), GSGS (SEQ ID NO: 288), GAGAGA (SEQ ID NO: 289), GSGSGS (SEQ ID NO: 290), GAGAGAGA (SEQ ID NO: 291), GSGSGSGS (SEQ ID NO: 292), GAGAGAGAGA (SEQ ID NO: 293), GSGSGSGSGS (SEQ ID NO: 294), GAGAGAGAGAGA (SEQ ID NO: 295), and GSGSGSGSGSGS (SEQ ID NO: 296). In some embodiments, a linker can contain 3 to 12 amino acids including motifs of GGA or GGS, e.g., GGA, GGS, GGAGGA (SEQ ID NO: 297), GGSGGS (SEQ ID NO: 298), GGAGGAGGA (SEQ ID NO: 299), GGSGGSGGS (SEQ ID NO: 300), GGAGGAGGAGGA (SEQ ID NO: 301), and GGSGGSGGSGGS (SEQ ID NO: 302). In some embodiments, a linker can contain 4 to 12 amino acids including motifs of GGAG (SEQ ID NO: 303), GGSG (SEQ ID NO: 304), GGAGGGAG (SEQ ID NO: 305). GGSGGGSG (SEQ ID NO: 306), GGAGGGAGGGAG (SEQ ID NO: 307), and GGSGGGSGGGSG (SEQ ID NO: 308). In some embodiments, a linker can contain motifs of GGGGA (SEQ ID NO: 309) or GGGGS (SEQ ID NO: 267). e.g., GGGGAGGGGAGGGGA (SEQ ID NO: 310) and GGGGSGGGGSGGGGS (SEQ ID NO: 311). In some embodiments, an amino acid linker between a moiety (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a polypeptide (e.g., variant ActRIIB polypeptides in either homomeric or heteromeric forms) may be GGG, GGGA (SEQ ID NO: 280), GGGG (SEQ ID NO: 262), GGGAG (SEQ ID NO: 312), GGGAGG (SEQ ID NO: 313), or GGGAGGG (SEQ ID NO: 314).

In some embodiments, a linker can also contain amino acids other than glycine, alanine, and serine, e.g., AAAL (SEQ ID NO: 315), AAAK (SEQ ID NO: 316), AAAR (SEQ ID NO: 317), EGKSSGSGSESKST (SEQ ID NO: 318), GSAGSAAGSGEF (SEQ ID NO: 319), AEAAAKEAAAKA (SEQ ID NO: 320), KESGSVSSEQLAQFRSLD (SEQ ID NO: 321), GENLYFQSGG (SEQ ID NO: 322), SACYCELS (SEQ ID NO: 323), RSIAT (SEQ ID NO: 324), RPACKIPNDLKQKVMNH (SEQ ID NO: 325), GGSAGGSGSGSSGGSSGASGTGTAGGTGSGSGTGSG (SEQ ID NO: 326), AAANSSIDLISVPVDSR (SEQ ID NO: 327), or GGSGGGSEGGGSEGGGSEGGGSEGGGSEGGGSGGGS (SEQ ID NO: 328). In some embodiments, a linker can contain motifs, e.g., multiple or repeating motifs, of EAAAK (SEQ ID NO: 329). In some embodiments, a linker can contain motifs, e.g., multiple or repeating motifs, of praline-rich sequences such as (XP)n, in which X may be any amino acid (e.g., A, K, or E) and n is from 1-5, and PAPAP(SEQ ID NO: 330).

The length of the peptide linker and the amino acids used can be adjusted depending on the two polypeptides involved and the degree of flexibility desired in the final polypeptide fusion polypeptide. The length of the linker can be adjusted to ensure proper polypeptide folding and avoid aggregate formation.

### 4. Nucleic Acids Encoding ActRIIB Polypeptides

In certain aspects, the disclosure provides isolated and/or recombinant nucleic acids encoding any of the variant ActRIIB polypeptides (e.g., soluble ActRIIB polypeptides), including any of the variants disclosed herein. For example, SEQ ID NO: 4 encodes a naturally occurring ActRIIB precursor polypeptide, while SEQ ID NO: 3 encodes a soluble ActRIIB polypeptide. The subject nucleic acids may be single-stranded or double stranded. Such nucleic acids may be DNA or RNA molecules. These nucleic acids are may be used, for example, in methods for making ActRIIB polypeptides or as direct therapeutic agents (e.g., in a gene therapy approach).

In certain aspects, the disclosure relates to isolated and/or recombinant nucleic acids comprising a coding sequence for one or more of the variant ActRIIB polypeptide(s) as described herein. For example, in some embodiments, the disclosure relates to an isolated and/or recombinant nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, 47, 277, 331, 334, 337, 340, 343, 346, 349, 352, and 355. In some embodiments, an isolated and/or recombinant polynucleotide sequence of the disclosure comprises a promoter sequence operably linked to a coding sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, or 47). In some embodiments, the disclosure relates to vectors comprising an isolated and/or recombinant nucleic acid described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, or 47). In some embodiments, the disclosure relates to a cell comprising an isolated and/or recombinant polynucleotide sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 3, 4, 10, 32, 35, 38, 41, 44, or 47). In some embodiments, the cell is a CHO cell. In some embodiments, the cell is a COS cell.

In certain embodiments, nucleic acids encoding variant ActRIIB (or homomultimers or heteromultimers thereof) polypeptides of the disclosure are understood to include nucleic acids that are variants of any one of SEQ ID NOs: 3, 4, 10, 32, 35, 38, 41, 44, 47, 277, 331, 334, 337, 340, 343, 346, 349, 352, and 355. Variant nucleotide sequences include sequences that differ by one or more nucleotide substitutions, additions, or deletions including allelic variants, and therefore, will include coding sequence that differ from the nucleotide sequence designated in any one of SEQ ID NOs: 3, 4, 10, 32, 35, 38, 41, 44, 47, 277, 331, 334, 337, 340, 343, 346, 349, 352, and 355.

In certain embodiments, variant ActRIIB (or homomultimers or heteromultimers thereof), polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to any one of SEQ ID NOs: 3, 4, 10, 32, 35, 38, 41, 44, 47, 277, 331, 334, 337, 340, 343, 346, 349, 352, and 355. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 3. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 4. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 10. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 32. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 35. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90°/a, 91%, 92%, 93%, 94%, 95%, 96%, 97%, 98%, 99%, or 100% identical to SEQ ID NO: 38. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 41. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 44. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 47. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 277. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 331. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 334. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 337. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 340. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 343. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 346. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 349. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 352. In certain embodiments, variant ActRIIB polypeptides (or homomultimers or heteromultimers thereof) of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 355.

In certain aspects, the subject nucleic acids encoding variant ActRIIB polypeptides are further understood to include nucleic acids that are variants of SEQ ID NO: 3. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NO: 4.

In certain embodiments, the disclosure provides isolated or recombinant nucleic acid sequences that are at least 75%, 80%, 85%, 90%, 91%, 92%, 93%, 94%, 95%, 96%, 97%, 98%, 99%, or 100% identical to SEQ ID NO: 3. One of ordinary skill in the art will appreciate that nucleic acid sequences complementary to SEQ ID NO: 3, and variants of SEQ ID NO: 3 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to nucleic acids encoding variant ActRIIB polypeptides (e.g., in either homomeric or heteromeric forms) of the disclosure (e.g., SEQ ID NO: 3), the complement sequence, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NO: 3 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the polypeptide. However, it is expected that DNA sequence polymorphisms that do lead to changes in the amino acid sequences of the subject polypeptides will exist among mammalian cells. One skilled in the art will appreciate that these variations in one or more nucleotides (up to about 3-5% of the nucleotides) of the nucleic acids encoding a particular polypeptide may exist among individuals of a given species due to natural allelic variation. Any and all such nucleotide variations and resulting amino acid polymorphisms are within the scope of this disclosure.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

In certain aspects, the subject nucleic acid is provided in an expression vector comprising a nucleotide sequence encoding a variant ActRIIB polypeptide and operably linked to at least one regulatory sequence. Regulatory sequences are art-recognized and are selected to direct expression of the variant ActRIIB polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding a variant ActRIIB polypeptide. Such useful expression control sequences, include, for example, the early and late promoters of SV40, tet promoter, adenovirus or cytomegalovirus immediate early promoter, RSV promoters, the lac system, the trp system, the TAC or TRC system, 17 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of polypeptide desired to be expressed. Moreover, the vector's copy number, the ability to control that copy number and the expression of any other polypeptide encoded by the vector, such as antibiotic markers, should also be considered.

A recombinant nucleic acid of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant variant ActRIIB polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of polypeptides in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In a preferred embodiment, a vector will be designed for production of the subject variant ActRIIB polypeptides in CHO cells, such as a Pcmv-Script vector (Stratagene, La Jolla, Calif.), pcDNA4 vectors (Invitrogen, Carlsbad. Calif.) and pCI-neo vectors (Promega, Madison, Wisc.). As will be apparent, the subject gene constructs can be used to cause expression of the subject variant ActRIIB polypeptides in cells propagated in culture, e.g., to produce polypeptides, including fusion polypeptides or variant polypeptides, for purification.

In certain embodiments, the disclosure relates to methods of making ActRIIB polypeptides, including variant ActRIIB polypeptides as well as homomultimer and heteromultimers comprising the same, as described herein. Such a method may include expressing any of the nucleic acids disclosed herein in a suitable cell (e.g., a CHO cell or COS cell). Such a method may comprise: a) culturing a cell under conditions suitable for expression of the soluble ActRIIB polypeptide, wherein said cell comprise with an ActRIIB polypeptide expression construct. In some embodiments, the method further comprises recovering the expressed ActRIIB polypeptide. ActRIIB polypeptides may be recovered as crude, partially purified or highly purified fractions using any of the well-known techniques for obtaining protein from cell cultures.

This disclosure also pertains to a host cell transfected with a recombinant gene including a coding sequence (e.g., SEQ ID NO: 4) for one or more of the subject variant ActRIIB polypeptides. The host cell may be any prokaryotic or eukaryotic cell. For example, a variant ActRIIB polypeptide of the disclosure may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject variant ActRIIB polypeptides. For example, a host cell transfected with an expression vector encoding a variant ActRIIB polypeptide can be cultured under appropriate conditions to allow expression of the ActRIIB poly peptide to occur. The variant ActRIIB polypeptide may be secreted and isolated from a mixture of cells and medium containing the variant ActRIIB polypeptide. Alternatively, the variant ActRIIB polypeptide may be retained cytoplasmically or in a membrane fraction and the cells harvested, lysed and the protein isolated. A cell culture includes host cells, media and other byproducts. Suitable media for cell culture are well known in the art. The subject variant ActRIIB polypeptides can be isolated from cell culture medium, host cells, or both, using techniques known in the art for purifying polypeptides, including ion-exchange chromatography, gel filtration chromatography, ultrafiltration, electrophoresis, and immunoaffinity purification with antibodies specific for particular epitopes of the variant ActRIIB polypeptides. In a preferred embodiment, the variant ActRIIB polypeptide is a fusion polypeptide containing a domain which facilitates its purification.

In another embodiment, a fusion gene coding for a purification leader sequence, such as a poly-(His)/enterokinase cleavage site sequence at the N-terminus of the desired portion of the recombinant variant ActRIIB polypeptide, can allow purification of the expressed fusion polypeptide by affinity chromatography using a Ni²⁺ metal resin. The purification leader sequence can then be subsequently removed by treatment with enterokinase to provide the purified variant ActRIIB polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different poly peptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 5. Screening Assays

In certain aspects, the present disclosure relates to the use of the subject variant ActRIIB polypeptides (e.g., variant ActRIIB polypeptides) to identify compounds (agents) which are agonist or antagonists of the variant ActRIIB polypeptides. Compounds identified through this screening can be tested in tissues such as pulmonary or cardiac tissues to assess their ability to modulate tissue growth in vitro. Additionally, compounds identified through this screening can be tested for efficacy in treating, preventing, or reducing the progression rate and/or severity of PcPH (e.g., WHO Group 2 and/or Group 5 PH) or one or more complications of PcPH (e.g., smooth muscle and/or endothelial cell proliferation in the pulmonary artery, angiogenesis in the pulmonary artery, dyspnea, chest pain, pulmonary vascular remodeling, right ventricular hypertrophy, left ventricular hypertrophy, left atrium dilation, and pulmonary fibrosis). Optionally, these compounds can further be tested in animal models to assess their ability to modulate tissue growth in vivo.

There are numerous approaches to screening for therapeutic agents for modulating tissue growth by targeting the variant ActRIIB polypeptides. In certain embodiments, high-throughput screening of compounds can be carried out to identify agents that perturb ActRIIB-mediated effects on growth of tissues (e.g., pulmonary tissue or cardiac tissue). In certain embodiments, the assay is carried out to screen and identify compounds that specifically inhibit or reduce binding of a variant ActRIIB polypeptide to its binding partner, such as a ligand of wild type ActRIIB (e.g., activin A, activin B, GDF8, GDF11, and BMP10). Alternatively, the assay can be used to identify compounds that enhance binding of a variant ActRIIB polypeptide to its binding partner such as an ActRIIB ligand. In a further embodiment, the compounds can be identified by their ability to interact with a variant ActRIIB polypeptide.

A variety of assay formats will suffice, and, in light of the present disclosure, those not expressly described herein will nevertheless be comprehended by one of ordinary skill in the art. As described herein, the test compounds (agents) of the disclosure may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present disclosure include non-peptidyl organic molecules, peptides, polypeptides, peptidomimetics, sugars, hormones, and nucleic acid molecules. In a specific embodiment, the test agent is a small organic molecule having a molecular weight of less than about 2,000 daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S transferase (GST), photoactivatible crosslinkers or any combinations thereof.

In many drug screening programs that test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a variant ActRIIB polypeptide and its binding polypeptide (e.g., an ActRIIB ligand).

Merely to illustrate, in an exemplary screening assay of the present disclosure, the compound of interest is contacted with an isolated and purified ActRIIB polypeptide which is ordinarily capable of binding to an ActRIIB ligand, as appropriate for the intention of the assay.

To the mixture of the compound and ActRIIB polypeptide is then added a composition containing an ActRIIB ligand. Detection and quantification of ActRIIB/ActRIIB ligand complexes provides a means for determining the compound's efficacy at inhibiting (or potentiating) complex formation between the ActRIIB polypeptide and its binding polypeptide. The efficacy of the compound can be assessed by generating dose response curves from data obtained using various concentrations of the test compound. Moreover, a control assay can also be performed to provide a baseline for comparison. For example, in a control assay, isolated and purified ActRIIB ligand is added to a composition containing the ActRIIB polypeptide, and the formation of ActRIIB/ActRIIB ligand complex is quantitated in the absence of the test compound. It will be understood that, in general, the order in which the reactants may be admixed can be varied, and can be admixed simultaneously. Moreover, in place of purified polypeptides, cellular extracts and lysates may be used to render a suitable cell-free assay system.

Complex formation between the ActRIIB polypeptide and its binding polypeptide may be detected by a variety of techniques. For instance, modulation of the formation of complexes can be quantitated using, for example, detectably labeled polypeptides such as radiolabeled (e.g., ³²P, ³⁵S, ¹⁴C or ³H), fluorescently labeled (e.g., FITC), or enzymatically labeled ActRIIB polypeptide or its binding polypeptide, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between an ActRIIB polypeptide and its binding polypeptide. Further, other modes of detection, such as those based on optical waveguides (PCT Publication WO 96/26432 and U.S. Pat. No. 5,677,196), surface plasmon resonance (SPR), surface charge sensors, and surface force sensors, are compatible with many embodiments described herein.

Moreover, the present disclosure contemplates the use of an interaction trap assay, also known as the “two hybrid assay,” for identifying agents that disrupt or potentiate interaction between an ActRIIB polypeptide and its binding polypeptide. See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two hybrid systems to identify compounds (e.g., small molecules or peptides) that dissociate interactions between an ActRIIB polypeptide and its binding polypeptide. See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368.

In certain embodiments, the subject compounds are identified by their ability to interact with a variant ActRIIB polypeptide of the disclosure. The interaction between the compound and the variant ActRIIB polypeptide may be covalent or non-covalent. For example, such interaction can be identified at the polypeptide level using in vitro biochemical methods, including photo-crosslinking, radiolabeled ligand binding, and affinity chromatography (Jakoby W B et al., 1974, Methods in Enzymology 46: 1). In certain cases, the compounds may be screened in a mechanism based assay, such as an assay to detect compounds which bind to a variant ActRIIB polypeptide. This may include a solid phase or fluid phase binding event. Alternatively, the gene encoding a variant ActRIIB polypeptide can be transfected with a reporter system (e.g., β-galactosidase, luciferase, or green fluorescent protein) into a cell and screened against the library preferably by a high throughput screening or with individual members of the library. Other mechanism based binding assays may be used, for example, binding assays which detect changes in free energy. Binding assays can be performed with the target fixed to a well, bead or chip or captured by an immobilized antibody or resolved by capillary electrophoresis. The bound compounds may be detected usually using colorimetric or fluorescence or surface plasmon resonance.

In certain aspects, the present disclosure provides methods and agents for decreasing pulmonary hypertension and decreasing the pathogenic mechanism contributing to pulmonary hypertension, for example, by antagonizing functions of an ActRIIB polypeptide and/or an ActRIIB ligand. Therefore, any compound (e.g. variant ActRIIB polypeptides) identified can be tested in whole cells or tissues, in vitro or in vivo, to confirm their ability to decrease pulmonary hypertension. Various methods known in the art can be utilized for this purpose. For example, methods of the disclosure are performed such that the signal transduction through an ActRIIB polypeptide activated by binding to an ActRIIB ligand (e.g., activin A, activin B, GDF8, GDF11, or BMP10) has been reduced or inhibited.

The present disclosure also contemplates in vivo assays to measure the effects of any compound (e.g. variant ActRIIB polypeptides) described herein on pulmonary hypertension. Various animal models of pulmonary hypertension (e.g. pulmonary arterial hypertension) known in the art can be utilized for this purpose. These include animal models such as chronic hypoxia induced pulmonary hypertension (e.g. Sugen Hypoxia model) and monocrotaline induced pulmonary hypertension. The effects of any compound on these animal models can assessed by measuring various parameters such as vessel muscularity, pulmonary artery cross sectional area, right ventricular stroke volume, right ventricular hypertrophy, right ventricular systolic pressure, and survival. These parameters can be assessed, in part, by removing and measuring (e.g. weight and length) the left ventricle (LV), septum (S), and right ventricle (RV) of each animal. Hypertrophy can be assessed, in part, by calculating RV/LV+S. Histopathologic scoring can also be used to measure the vessel muscularity. In some embodiments, methods and agents of the present disclosure can be screened in combination with additional active agents and/or supportive therapies that are currently used in treating PcPH (e.g. LVAD). In some embodiments, methods and agents of the present disclosure can be compared to active agents and/or supportive therapies that are currently used in treating PcPH (e.g. LVAD).

In certain aspects, the present disclosure provides methods and agents for decreasing muscularization of the pulmonary arteries, for example, by antagonizing functions of an ActRIIB polypeptide and/or an ActRIIB ligand. Therefore, any compound identified can be tested in whole cells or tissues, in vitro or in vivo, to confirm their ability to modulate muscularization of the pulmonary arteries. Various methods known in the art can be utilized for this purpose. For example, methods of the disclosure are performed such that the signal transduction through an ActRIIB polypeptide activated by binding to an ActRIIB ligand (e.g., activin A, activin B, GDF8, GDF11, or BMP10) has been reduced or inhibited.

It is understood that the screening assays of the present disclosure apply to not only the subject variant ActRIIB polypeptides, but also any test compounds including agonists and antagonist of the ActRIIB polypeptides. Further, these screening assays are useful for drug target verification and quality control purposes.

### 6. Methods of Use

In part, the present disclosure relates to methods of treating post-capillary pulmonary hypertension (PcPH) (e.g., WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms). In some embodiments, the PcPH is combined post- and pre-capillary PH. In certain embodiments, the present disclosure provides methods of treating or preventing post-capillary pulmonary hypertension (PcPH) in an individual in need thereof through administering to the individual a therapeutically effective amount of one or more variant ActRIIB polypeptides as described herein. In some embodiments, the variant ActRIIB polypeptide is administered at a dosing range of 0.1 mg/kg to 2.0 mg/kg (e.g., 0.3 mg/kg or 0.7 mg/kg). These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated (e.g., WHO Group 2 and/or Group 5 PH). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure (e.g., WHO Group 2 and/or Group 5 PH) is achieved by administering one or more variant ActRIIB polypeptides of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of variant ActRIIB polypeptide, in combination with one or more additional active agents or other supportive therapy for treating or preventing a disease or condition (e.g., WHO Group 2 and/or Group 5 PH). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more variant ActRIIB polypeptides of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the variant ActRIIB polypeptide of the present disclosure with the additional active agent or therapy and/or the desired effect.

### WHO Classification Outline

A pulmonary hypertension condition treated by methods describe herein, can comprise any one or more of the conditions recognized according to the World Health Organization (WHO). See, e.g., Simonneau (2019) Eur Respir J: 53:1801913.

TABLE 1
Clinical Classification of Pulmonary Hypertension

```
Group 1: Pulmonary arterial hypertension (PAH)
      1.1 Idiopathic PAH
      1.2 Heritable PAH
          1.2.1 BMPR2
          1.2.2 ALK-1, ENG, SMAD9, CAV1, KCNK3
          1.2.3 Unknown
      1.3 Drug and toxin induced PAH
      1.4 Associated with:
          1.4.1 Connective tissue disease
          1.4.2 HIV infection
          1.4.3 Portal hypertension
          1.4.4 Congenital heart diseases
          1.4.5 Schistosomiasis
      1.5 PAH long-term responders to calcium channel blockers
      1.6 PAH with overt features of venous/capillaries (PVOD/PCH) involvement
      1.7 Persistent PH of the newborn syndrome
Group 2: Pulmonary hypertension due to left heart disease
      2.1 PH due to heart failure with preserved LVEF¹ (HFpEF)
      2.2 PH due to heart failure with reduced LVEF (HFrEF)
      2.3 Valvular heart disease
      2.4 Congenital/acquired cardiovascular conditions leading to post-capillary PH
Group 3: Pulmonary hypertension due to lung disease and/or hypoxia
      3.1 Obstructive lung disease
      3.2 Restrictive lung disease
      3.3 Other lung disease with mixed restrictive/obstructive pattern
      3.4 Hypoxia without lung disease
      3.5 Developmental lung disorders
Group 4: Pulmonary hypertension due to pulmonary artery obstructions
      4.1 Chronic thromboembolic PH
      4.2 Other pulmonary artery obstructions
          4.2.1 Sarcoma (high or intermediate grade) or angiosarcoma
          4.2.2 Other malignant tumours
            Renal carcinoma
            Uterine carcinoma
            Germ cell tumours of the testis
            Other tumours
          4.2.3 Non-malignant tumours
            Uterine leiomyoma
          4.2.4 Arteritis without connective tissue disease
          4.2.5 Congenital pulmonary artery stenoses
          4.2.6 Parasites
            Hydatidosis
Group 5: Pulmonary hypertension with unclear and/or multifactorial mechanisms.
      5.1 Hematological disorders (e.g., Chronic hemolytic anaemia and myeloproliferative
      disorders)
      5.2 Systemic and metabolic disorders (e.g., Pulmonary Langerhans cell histiocytosis,
      Gaucher disease, Glycogen storage disease, Neurofibromatosis, and Sarcoidosis)
      5.3 Others (e.g., Chronic renal failure with or without haemodialysis and Fibrosing
      mediastinitis)
      5.4 Complex congenital heart disease
¹Left ventricular ejection fraction
```

The clinical purpose of the classification of PH is to categorize clinical conditions associated with PH into five groups according to their pathophysiological mechanisms, clinical presentation, hemodynamic characteristics, and treatment strategy. This clinical classification may be updated when new data are available on the above features or when additional clinical entities are considered.

Pulmonary hypertension (PH) has been previously classified as primary or secondary PH. The term primary pulmonary hypertension has now been replaced by idiopathic PAH or familial PAH depending on the absence or presence of genetic information; the term secondary pulmonary hypertension has been abandoned.

As used herein, the term “pulmonary hemodynamic parameter” refers to any parameter used to describe or evaluate the blood flow through the heart and pulmonary vasculature. Examples of pulmonary hemodynamic parameters include, but are not limited to, mean pulmonary arterial pressure (mPAP), diastolic pulmonary arterial pressure (dPAP) [also known as pulmonary artery diastolic pressure (PADP)], systolic pulmonary arterial pressure (sPAP) [also known as pulmonary artery systolic pressure (PASP)], mean right atrial pressure (mRAP), pulmonary capillary wedge pressure (PCWP) [also known as pulmonary artery wedge pressure (PAWP)], left ventricular end-diastolic pressure (LVEDP), diastolic pressure gradient (DPG) [also known as diastolic pressure difference (DPD)], left atrial pressure (LAP), transpulmonary gradient (TPG), pulmonary vascular resistance (PVR), total pulmonary vascular resistance (TPR), cardiac index, and cardiac output (CO).

Many of the pulmonary hemodynamic parameters described above are interrelated. For example, PCWP is often used as a more convenient, less invasive approximation of LAP.

Additionally, PVR is related to mPAP, PCWP and CO according to the following equation:

PVR=(mPAP−PCWP)/CO[Woods Units]

The PVR measures the resistance to flow imposed by the pulmonary vasculature without the influence of the left-sided filling pressure. PVR can also be measured according to the following equations:

PVR=TPG×80/CO [unit: dynes-sec-cm⁻⁵] OR PVR=(mPAP−PCWP)×80/CO [unit: dynes-sec-cm⁻⁵]

In some embodiments, the total peripheral resistance (TPR) can be measured using the following equation: TPR=mPAP/CO.

In some embodiments, the cardiac index (CI) can be measured using the following equation: CI=CO/BSA. In some embodiments, body surface area (BSA) can be calculated using the Du Bois formula (defined as 0.007184×W^{0.425}×H^{0.725}).

According to some embodiments, a pre-capillary pulmonary arterial contribution to PH may be reflected by an elevated PVR In some embodiments, the normal PVR is 20-130 dynes-sec-cm⁻⁵ or 0.5-1.1 Wood units. According to some embodiments, an elevated PVR may refer to a PVR above 2 Wood units, above 2.5 Wood units, above 3 Wood units or above 3.5 Wood units.

As yet another example, TPG is the difference between mPAP and left atrial pressure (PLA; commonly estimated by pulmonary capillary wedge pressure: PCWP) as shown by the following equation: TPG=mPAP-PCWP

The TPG is influenced by all the determinants of mPAP, including flow, resistance and left heart filling pressure. A pre-capillary pulmonary arterial contribution to PH may be reflected by an increased trans-pulmonary gradient (TPG). According to some embodiments, an increased TPG may refer to an mPAP-PCWP that exceeds 12-15 mmHg.

DPG (defined as diastolic PAP−mean PAWP) appears to best approach the characteristics required to determine pulmonary vascular disease. In some embodiments, the DPG is synonymous with diastolic pressure difference (DPD). In normal subjects, DPG generally lies in the 1-3 mmHg range, and in patients evaluated for cardiac disease (excluding shunts), DPG remains≤5 mmHg in most cases.

As a further example, mPAP is related to dPAP and sPAP according to the following equation: mPAP=(⅔)dPAP+(⅓)sPAP

Furthermore, dPAP and sPAP can be used to calculate the pulse pressure (mmHg) using the following equation: pulse pressure=sPAP-dPAP

Pulse pressure can be used to calculate the pulmonary artery compliance using the following equation: pulmonary artery compliance (mI·mmHg⁻¹)=stroke volume/pulse pressure

In some embodiments, the pulmonary hemodynamic parameters are measured directly, such as during a right heart catheterization. In other embodiments, the pulmonary hemodynamic parameters are estimated and/or evaluated through other techniques such as magnetic resonance imaging (MRI) or echocardiography.

Exemplary pulmonary hemodynamic parameters include mPAP, PAWP TPG DPG, and PVR. The one or more pulmonary hemodynamic parameters may be measured by any appropriate procedures, such as by utilizing a right heart catheterization or echocardiography. Various hemodynamic types of GPH are shown in Table 2 together with their corresponding clinical classification (Table 1).

TABLE 2
Hemodynamic Types of Pulmonary Hypertension (PH)

| Hemodynamic | Hemodynamic | | WHO PH |
|---|---|---|---|
| Type | Subtype | Characteristics | Classification |
| Pulmonary | — | mPAP >20 mmHg | All (Groups 1-5) |
| Hypertension | | | |
| Pre-Capillary | — | mPAP >20 mmHg | Group 1: |
| PH | | PAWP ≤15 mmHg | Pulmonary |
| | | PVR ≥3 Wood units | arterial |
| | | | hypertension |
| | | | Group 3: |
| | | | PH due to |
| | | | lung disease |
| | | | and/or hypoxia |
| | | | Group 4: |
| | | | PH due to |
| | | | pulmonary artery |
| | | | obstructions |
| | | | Group 5: PH with |
| | | | unclear and/or |
| | | | multifactorial |
| | | | mechanisms. |
| Post-Capillary | Isolated Post- | mPAP >20 mmHg | Group 2: |
| PH | Capillary PH | PAWP >15 mmHg | PH due to |
| | | PVR <3 Wood units | left heart disease |
| | | DPG <7 mmHg | Group 5: PH with |
| | Combined Pre- | mPAP >20 mmHg | unclear and/or |
| | and Post- | PAWP >15 mmHg | multifactorial |
| | Capillary PH | PVR ≥3 Wood units | mechanisms. |
| | | DPG ≥7 mmHg | |

The types of PH and the difference between pre-capillary pulmonary hypertension and post-capillary pulmonary hypertension are based on pulmonary hemodynamic parameters. As used herein, the term “pre-capillary pulmonary hypertension” includes WHO clinical Groups 1, 3, 4, and 5. In general, pre-capillary pulmonary hypertension is characterized using the pulmonary hemodynamic parameters shown in Table 2 (i.e., an mPAP>20 mmHg or in some embodiments a mPAP>25 mmHg). As used herein, the term “post-capillary pulmonary hypertension” (PcPH) includes both isolated post-capillary pulmonary hypertension (IpcPH) and combined pre- and post-capillary pulmonary hypertension (CpcPH), both within WHO clinical Groups 2 and 5. In some embodiments, IpcPH is characterized using the pulmonary hemodynamic parameters shown in Table 2 (i.e., one or more of the following pulmonary hemodynamic parameters: mPAP>20 mmHg, PAWP>15 mmHg, PVR≤3 Wood units, and/or DPG≤7 mmHg). In some embodiments, CpcPH is characterized using the pulmonary hemodynamic parameters shown in Table 2 (i.e., one or more of the following pulmonary hemodynamic parameters: mPAP>20 mmHg, PAWP>15 mmHg, PVR≥3 Wood units, and/or DPG≥7 mmHg). In some embodiments, CpcPH is characterized as comprising one or more of the following hemodynamic parameters: mPAP≥25 mmHg: PAWP>15 mmHg; and PVR>3 WU.

The clinical classification or hemodynamic types of PH described herein and the associated diagnostic parameters may be updated or varied based on the availability of new or existing sources of data or when additional clinical entities are considered.

### Characteristics of PH

The diagnosis of PH including WHO PH class and functional group, can be determined based on symptoms and physical examination using a review of a comprehensive set of parameters to determine if the hemodynamic and other criteria are met. Some of the criteria which may considered include the patient's clinical presentation (e.g., shortness of breath, fatigue, weakness, angina, syncope, dry-couch, exercise-induced nausea and vomiting), electrocardiogram (ECG) results, chest radiograph results, pulmonary function tests, arterial blood gases, echocardiography results, ventilation/perfusion lung scan results, high-resolution computed tomography results, contrast-enhanced computed tomography results, pulmonary angiography results, cardiac magnetic resonance imaging, blood tests (e.g., biomarkers such as BNP or NT-proBNP), immunology, abdominal ultrasound scan, right heart catherization (RHC), vasoreactivity, and genetic testing. See, e.g., Galie N., et al Euro Heart J. (2016) 37, 67-119.

In some embodiments, a biomarker may be used to determine the diagnosis of PH. For instance, in some embodiments, the biomarker is a marker of vascular dysfunction (e.g., asymmetric dimethylarginine (ADMA), endothelin-1, angiopoeitins, or von Willebrand factor). In some embodiments, the biomarker is a marker of inflammation (C-reactive protein, interleukin 6, chemokines). In some embodiments, the biomarker is a marker of myocardial stress (e.g., (atrial natriuretic peptide, brain natriuretic peptide (BNP)/NT-proBNP, or troponins). In some embodiments, the biomarker is a marker of low CO and/or tissue hypoxia (e.g., pCO2, uric acid, growth differentiation factor 15 (GDF15), or osteopontin). In some embodiments, the biomarker is a marker of secondary organ damage (e.g., creatinine or bilirubin). See, e.g., Galie N., et al Euro Heart J. (2016) 37, 67-119.

### Group 1 PH

Pulmonary arterial hypertension (WHO Group 1 PH) is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in PVR and sustained elevations in mPAP, which ultimately lead to right ventricular failure and death. Patients diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of patients with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

PAH can be diagnosed based on a mean pulmonary arterial pressure of above 25 mmHg (or above 20 mmHg under updated guidelines) at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMP2, ALK1, ENG, SMAD9, CAV1, KCNK3, or EIF2AK4). In 70% of familial PAH cases, mutations are located in the BMPR2 gene. Risk factors for the development of PAH include family history of PAH, drug and toxin use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus). PAH may be associated with long term responders to calcium channel blockers, overt features of venous/capillaries (PVOD/PCH) involvement, and persistent PH of the newborn syndrome.

### Group 2 PH

Pulmonary hypertension due to left heart disease (PH-LHD) (WHO Group 2 PH) is a complex pathophenotype that, when present, may result in an increased susceptibility to adverse events and worse clinical outcome. PH-LHD is sometimes defined as patients having a pulmonary capillary wedge pressure (PCWP)>15 mmHg and a mean pulmonary arterial pressure (mPAP)≥25 mmHg (or a mean pulmonary arterial pressure (mPAP)≥20 mmHg under updated guidelines). PH-LHD occurs as a consequence of the backward transmission of high left sided filling pressures, mainly driven by LV diastolic function, directly to the post-capillary pulmonary vessels and, thereby, to the rest of the pulmonary circulation. PH-LHD may be associated with or caused by PH due to heart failure with preserved left ventricle ejection fraction (LVEF) [also known as HFpEF], PH due to heart failure with reduced LVEF (also known as HFrEF), valvular heart disease (VHD), or congenital/acquired cardiovascular conditions leading to post-capillary PH. Compared with PAH, patients with PH-LHD are often older, female, with a higher prevalence of cardiovascular co-morbidities and most, if not all, of the features of metabolic syndrome.

Valvular heart disease (VHD) associated with pulmonary hypertension may result from multiple mechanisms such as an increase in PVR pulmonary blood flow, or pulmonary venous pressure. The chronic rise in PAP frequently leads to RV pressure overload and subsequent RV failure. Clinical signs and symptoms of left-sided VHD with PH are orthopnea and paroxysmal nocturnal dyspnea. In advanced stages of diseases, signs of RV failure including peripheral edema, ascites, and syncope are frequently observed. There are four valvular heart disease subtypes which include mitral valve stenosis, mitral valve regurgitation, aortic stenosis, and aortic regurgitation.

Mitral valve stenosis occurs when the heart's mitral valve is narrowed due to the valve becoming stiff or scarred, or the valve flaps partially joining together. This results in the valve not opening as widely as it should, which causes poor blood flow and may result in blood backing up into the lungs. Left untreated, mitral valve stenosis can lead to serious heart complications. Common causes of mitral valve stenosis include rheumatic heart disease, radiation, and mitral annulus calcification. Typical interventions for mitral stenosis include balloon vavuloplasty, commisurrotomy, and surgical valve replacement.

Mitral valve regurgitation (also called mitral insufficiency) occurs when the flaps (leaflets) of the mitral valve do not close tightly, allowing blood to flow backward in the heart. As a result, blood can't move through the heart or to the rest of the body as efficiently, resulting in fatigue or shortness of breath. Additionally, the reduced flow increases pressure in the left atrium and lung vasculature. In moderate to severe cases, surgery may be recommended to either repair or replace the damaged valve. Left untreated, severe mitral valve regurgitation can cause heart failure or serious heart rhythm problems. Common causes of mitral valve regurgitation include degenerative mitral disease such as mitral valve prolapse and mitral annulus calcification. Typical interventions for mitral valve regurgitation include transcatheter mitral valve repair, surgical repair, or replacement.

In aortic stenosis, the aortic valve does not open fully. This decreases blood flow from the heart. As the aortic valve becomes more narrow, the pressure increases inside the left heart ventricle. This causes the left heart ventricle to become thicker, which decreases blood flow and can lead to chest pain. As the pressure continues to rise, blood may back up into the lungs causing dyspnea. Severe forms of aortic stenosis prevent enough blood from reaching the brain and rest of the body. Common causes of aortic stenosis include calcification of the aortic valve or the presence of a bicuspid aortic valve. Typical interventions include transcatheter aortic valve replacement (percutaneous valve replacement) and surgical valve replacement.

Aortic regurgitation (also known as aortic insufficiency) occurs when the aortic valve is unable to fully close. The valve leaks, resulting in reduced blood flow. As a result, the heart has to work harder to make up for the reduced blood flow, and over time it will weaken. Because of this, the amount of blood that flows from the heart to the rest of the body is reduced. Common causes of aortic regurgitation include aortic root dilatation and presence of a bicuspid aortic valve.

Among those patients with PH-LHD, two phenotypes have been described: 1) a group of isolated post-capillary (IpcPH) or “passive” PH in which elevated pulmonary pressures are reversible and in proportion to increases in left atrial pressure, and 2) a group with an added “pre-capillary” component [combined post-capillary and pre-capillary PH (CpcPH)]. This latter group, CpcPH, may have comorbid pulmonary vascular remodeling and therefore may demonstrate persistent PH after interventions to lower left sided filling pressures.

In some embodiments, a combination of mPAP, PAWP. PVR, or DPG may be used to define the different subtypes of PH-LHD, i.e., IpcPH and CpcPH (see, e.g., Table 2). In some embodiments, patients with CpcPH arc characterized as having a TPG>12-15 mmHg and a PVR>2.5-3 Wood units (WU). In some embodiments, CpcPH is distinguished from IpcPH using the DPG. In some embodiments, a patient with CpcPH has a DPG≥7 mmHg. In some embodiments, a patient with IpcPH has a DPG<7 mmHg. In some embodiments, a combination of DPG and PVR may be used to define the different types of PH-LHD. For instance, in some embodiments, IpcPH patients have a DPG<7 mmHg and/or a PVR of ≥3 WU. In some embodiments, CpcPH patients have a DPG≥7 mmHg and/or a PVR>3 WU.

The clinical classification or hematological classification described herein and the associated diagnostic parameters may be updated when new data are available or when additional clinical entities are considered. For instance, at the 5^{th} World Symposium on Pulmonary Hypertension (WSPH), a new terminology was adopted to distinguish IpcPH from CpcPH, based on the diastolic pressure difference/gradient (DPG) between the dPAP and PAWP. However, this definition was found to be too restrictive and exposed to interpretation, leading to controversies about whether the DPG would or would not predict outcome in patients with group 2 PH. Accordingly, at the 6^{th} WSPH, pulmonary vascular resistance (PVR) was subsequently reintroduced to better reflect the impact of the right ventricle on patient outcome. See, e.g., Vachiery J. L., et al. Eur Respir J 2019 Jan. 24; 53(1).

Therapies for treating PH-LHD primarily include treatment of the underlying condition (i.e., COPD, sleep apnea syndrome. CTEPH) prior to considering specific measures to treat the PH itself. Some therapies include repair of valvular heart disease (if indicated). Non-specific vasodilators such as nitrates and hydralazine may also be used. In some embodiments, an LV assist device (LVAD) may be used to lower pulmonary pressure. The lack of specific therapies is particularly problematic because PH-LHD is the most common cause of PH in western countries and its presence commonly results in adverse course of the disease. Specifically, the presence of PH-LHD can result in more severe symptoms in LHD, worse exercise tolerance, and a negative impact on outcome.

### Group 3PH

Pulmonary hypertension due to lung disease and/or hypoxia (WHO Group 3 PH) refers to a form of pulmonary hypertension that is due to lung disease or chronic hypoxia. This form of PH is also known as “hypoxic PH” or “hypoxic pulmonary hypertension.” Hypoxic PH may be associated with or caused by chronic obstructive pulmonary disease (e.g., emphysema), interstitial lung disease, sleep-disordered breathing (e.g., sleep apnca), lung disease (e.g., pulmonary fibrosis), alveolar hypoventilation disorders, chronic exposure to high altitude, or developmental abnormalities.

### Group 4 PH

Pulmonary hypertension due to pulmonary artery obstructions (WHO Group 4 PH) is a form of pulmonary hypertension that is related to chronic arterial obstruction (e.g., blood clots). There may be multiple pathophysiological mechanisms driving development of PH in Group 4 including chronic thromboembolic PH, sarcoma (high or intermediate grade) or angiosarcoma, other malignant tumors (e.g., renal carcinoma, uterine carcinoma, germ cell tumors of the testis, or other tumors), non-malignant tumors (e.g., uterine leiomyoma), arteritis without connective tissue disease, congenital pulmonary artery stenosis, or parasites (e.g., hydatidosis).

Various pulmonary hemodynamic parameters are associated with Group 4 PH. For instance, in patients with PH due to pulmonary artery obstructions, those with severe PH (>40 mmHg) often have a marked increase in PVR (around 10 WU); more often these patients may have a mild PH (mPAP 20-30 mmHg), associated with lower PVR but remaining generally >3 WU. See, e.g., Simonneau (2019) Eur Respir J: 53:1801913. In these different chronic lung diseases, even a modest elevation in mPAP (20-29 mmHg) can be associated with a poor prognosis. Furthermore, in chronic thromboembolism, patients may have severe pre-capillary PH with a mPAP of about 47 mmHg and a mean PVR of about 8.9 WU. Id. In this setting, even in patients with mild elevation of mPAP (20-24 mmHg), PVR is generally >3 WU.

### Group 5 PH

Pulmonary hypertension with unclear and/or multifactorial mechanisms (WHO Group 5 PH) is a group which contains less-studied forms of PH in comparison with the other groups. However, many of the PH forms currently in group 5 represent a significant part of the PH burden. The diseases within Group 5 PH are characterized by having no identified predominant mechanism driving the development of PH. There may be multiple pathophysiological mechanisms driving development of PH, including hematological disorders (e.g., chronic hemolytic anemia or mycloproliferative disorders), systemic and metabolic disorders (e.g., Pulmonary Langerhans cell histiocytosis, Gaucher disease, glycogen storage disease, neurofibromatosis, or sarcoidosis), others (e.g., chronic renal failure with or without hemodialysis or fibrosing mediastinitis), or complex congenital heart disease.

### Measurements of PH

In certain aspects, the disclosure relates to methods of treating, preventing, or reducing the progression rate and/or severity of PcPH (e.g., treating, preventing, or reducing the progression rate and/or severity of one or more complications of post-capillary pulmonary hypertension in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms). In some embodiments, the method relates to treating PcPH patients that have IpcPH. In some embodiments, the method relates to treating PcPH patients that have CpcPH. In some embodiments, the method relates to treating PcPH patients that have pulmonary hypertension due to left heart disease (PH-LHD). In some embodiments, the method relates to treating PcPH patients that have Group 2 PH as classified by the WHO. In some embodiments, the method relates to treating PcPH patients that have pulmonary hypertension due to heart failure with preserved LVEF (HFpEF). In some embodiments, the method relates to treating PcPH patients that have pulmonary hypertension due to heart failure with reduced LVEF (HFrEF). In some embodiments, the method relates to treating PcPH patients that have valvular heart disease. In some embodiments, the valvular heart disease is aortic regurgitation. In some embodiments, the valvular heart disease is aortic stenosis. In some embodiments, the valvular heart disease is mitral valve disease. In some embodiments, the valvular heart disease is mitral valve regurgitation. In some embodiments, the valvular heart disease is mitral valve stenosis. In some embodiments, the method relates to treating CpcPH patients who have PH due to valvular heart disease. In some embodiments, the method relates to treating IpcPH patients who have PH due to valvular heart disease. In some embodiments, the method relates to treating PcPH patients that have congenital/acquired cardiovascular conditions leading to post-capillary PH. In some embodiments, the method relates to treating PcPH patients that have pulmonary hypertension with unclear and/or multifactorial mechanisms. In some embodiments, the method relates to treating PcPH patients that have Group 5 PH as classified by the WHO.

In some embodiments, the disclosure relates to methods of treating, preventing, or reducing the progression rate and/or severity of pulmonary hypertension in combinations of certain patient populations. Each of the patient populations described herein can be combined and reorganized accordingly. For instance, in some embodiments, the method relates to treating CpcPH patients who have PH due to heart failure with preserved LVEF (HFpEF). In some embodiments, the method relates to treating CpcPH patients who have PH due to heart failure with reduced LVEF (HFrEF). In some embodiments, the method relates to treating CpcPH patients who have PH due to valvular heart disease. In some embodiments, the method relates to treating IpcPH patients who have PH due to heart failure with preserved LVEF (HFpEF). In some embodiments, the method relates to treating IpcPH patients who have PH due to heart failure with reduced LVEF (HFrEF). In some embodiments, the method relates to treating IpcPH patients who have PH due to valvular heart disease.

In some embodiments, the method relates to pulmonary hypertension patients that have pulmonary hypertension with unclear and/or multifactorial mechanisms. In some embodiments, the method relates to patients that have a hematological disorder (e.g., chronic hemolytic anemia and myeloproliferative disorders). In some embodiments, the method relates to patients that have a systemic and/or metabolic disorder (e.g., pulmonary langerhans cell histiocytosis, Gaucher disease, glycogen storage disease, neurofibromatosis, and sarcoidosis). In some embodiments, the method relates to pulmonary hypertension patients that have other disorders with unclear and/or multifactorial mechanisms (e.g., chronic renal failure with or without hemodialysis or fibrosing mediastinitis). In some embodiments, the method relates to patients that have complex congenital heart disease.

mPAP

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has resting mean pulmonary arterial pressure (mPAP) of at least 20 mmHg (e.g., at least 20, 25, 30, 35, 40, 45, or 50 mmHg). As used herein, the terms “mean pulmonary arterial pressure” and “mean pulmonary artery pressure” are used interchangeably. In some embodiments, the method relates to patients having a resting mPAP of at least 20 mmHg. In some embodiments, the method relates to patients having a resting mPAP of at least 25 mmHg. In some embodiments, the method relates to patients having a resting mPAP of at least 30 mmHg. In some embodiments, the method relates to patients having a resting mPAP of at least 35 mmHg. In some embodiments, the method relates to patients having a resting mPAP of at least 40 mmHg. In some embodiments, the method relates to patients having a resting mPAP of at least 45 mmHg. In some embodiments, the method relates to patients having a resting mPAP of at least 50 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving the pulmonary arterial pressure in the patient. In some embodiments, the improvement in pulmonary arterial pressure is a reduction in the mean pulmonary arterial pressure (mPAP). In some embodiments, the method relates to reducing mPAP. In some embodiments, the method relates to reducing the patient's mPAP by at least 1 mmHg. In some embodiments, the method relates to reducing the patient's mPAP by at least 2 mmHg. In some embodiments, the method relates to reducing the patient's mPAP by at least 3 mmHg. In certain embodiments, the method relates to reducing the patient's mPAP by at least 5 mmHg. In certain embodiments, the method relates to reducing the patient's mPAP by at least 7 mmHg. In certain embodiments, the method relates to reducing the patient's mPAP by at least 10 mmHg. In certain embodiments, the method relates to reducing the patient's mPAP by at least 12 mmHg. In certain embodiments, the method relates to reducing the patient's mPAP by at least 15 mmHg. In certain embodiments, the method relates to reducing the patient's mPAP by at least 20 mmHg. In certain embodiments, the method relates to reducing the patient's mPAP by at least 25 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's mPAP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's mPAP by at least 1%. In some embodiments, the method relates to decreasing the patient's mPAP by at least 5%. In some embodiments, the method relates to decreasing the patient's mPAP by at least 10%. In some embodiments, the method relates to decreasing the patient's mPAP by at least 15%. In some embodiments, the method relates to decreasing the patient's mPAP by at least 20%. In some embodiments, the method relates to decreasing the patient's mPAP by at least 25%. In some embodiments, the method relates to decreasing the patient's mPAP by at least 30%. In some embodiments, the method relates to decreasing the patient's mPAP by at least 35%. In some embodiments, the method relates to decreasing the patient's mPAP by at least 40%. In some embodiments, the method relates to decreasing the patient's mPAP by at least 45%. In some embodiments, the method relates to decreasing the patient's mPAP by at least 50%. In some embodiments, the method relates to decreasing the patient's mPAP by at least 55%. In some embodiments, the method relates to decreasing the patient's mPAP by at least 60%. In some embodiments, the method relates to decreasing the patient's mPAP by at least 65%. In some embodiments, the method relates to decreasing the patient's mPAP by at least 70%. In some embodiments, the method relates to decreasing the patient's mPAP by at least 75%. In some embodiments, the method relates to decreasing the patient's mPAP by at least 80%. In some embodiments, the method relates to decreasing the patient's mPAP by at least 85%. In some embodiments, the method relates to decreasing the patient's mPAP by at least 90%. In some embodiments, the method relates to decreasing the patient's mPAP by at least 95%. In some embodiments, the method relates to decreasing the patient's mPAP by at least 100%.

mRAP

In some patients, increased pulmonary vascular resistance to blood flow leads to increased right atrial pressure (RAP) and right heart failure. Patients with right heart failure typically have an increased ratio of RAP and pulmonary artery wedge pressure (PAWP). In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has resting mean right atrial pressure (mRAP) of at least 5 mmHg (e.g., at least 5, 6, 7, 8, 9, 10, 11, 12, 13, 14, 15, 16, 17, 18, 18, 20, 21, 22, 23, 24, or 25 mmHg). In some embodiments, the method relates to a patient having a resting mRAP of at least 5 mmHg. In some embodiments, the method relates to a patient having a resting mRAP of at least 6 mmHg. In some embodiments, the method relates to a patient having a resting mRAP of at least 7 mmHg. In some embodiments, the method relates to a patient having a resting mRAP of at least 8 mmHg. In some embodiments, the method relates to a patient having a resting mRAP of at least 9 mmHg. In some embodiments, the method relates to a patient having a resting mRAP of at least 10 mmHg. In some embodiments, the method relates to a patient having a resting mRAP of at least 11 mmHg. In some embodiments, the method relates to a patient having a resting mRAP of at least 12 mmHg. In some embodiments, the method relates to a patient having a resting mRAP of at least 13 mmHg. In some embodiments, the method relates to a patient having a resting mRAP of at least 14 mmHg. In some embodiments, the method relates to a patient having a resting mRAP of at least 15 mmHg. In some embodiments, the method relates to a patient having a resting mRAP of at least 16 mmHg. In some embodiments, the method relates to a patient having a resting mRAP of at least 17 mmHg. In some embodiments, the method relates to a patient having a resting mRAP of at least 18 mmHg. In some embodiments, the method relates to a patient having a resting mRAP of at least 19 mmHg. In some embodiments, the method relates to a patient having a resting mRAP of at least 20 mmHg. In some embodiments, the method relates to a patient having a resting mRAP of at least 21 mmHg. In some embodiments, the method relates to a patient having a resting mRAP of at least 22 mmHg. In some embodiments, the method relates to a patient having a resting mRAP of at least 23 mmHg. In some embodiments, the method relates to a patient having a resting mRAP of at least 24 mmHg. In some embodiments, the method relates to a patient having a resting mRAP of at least 25 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving the mean right atrial pressure in the patient. In some embodiments, the improvement in the mean right atrial pressure (mRAP) is a reduction in the mRAP. In some embodiments, the method relates to reducing mRAP. In some embodiments, the method relates to reducing the patient's mRAP by at least 1 mmHg. In some embodiments, the method relates to reducing the patient's mRAP by at least 2 mmHg. In some embodiments, the method relates to reducing the patient's mRAP by at least 3 mmHg. In some embodiments, the method relates to reducing the patient's mRAP by at least 4 mmHg. In certain embodiments, the method relates to reducing the patient's mRAP by at least 5 mmHg. In some embodiments, the method relates to reducing the patient's mRAP by at least 6 mmHg. In certain embodiments, the method relates to reducing the patient's mRAP by at least 7 mmHg. In some embodiments, the method relates to reducing the patient's mRAP by at least 8 mmHg. In some embodiments, the method relates to reducing the patient's mRAP by at least 9 mmHg. In certain embodiments, the method relates to reducing the patient's mRAP by at least 10 mmHg. In some embodiments, the method relates to reducing the patient's mRAP by at least 11 mmHg. In certain embodiments, the method relates to reducing the patient's mRAP by at least 12 mmHg. In some embodiments, the method relates to reducing the patient's mRAP by at least 13 mmHg. In some embodiments, the method relates to reducing the patient's mRAP by at least 14 mmHg. In certain embodiments, the method relates to reducing the patient's mRAP by at least 15 mmHg. In some embodiments, the method relates to reducing the patient's mRAP by at least 16 mmHg. In some embodiments, the method relates to reducing the patient's mRAP by at least 17 mmHg. In some embodiments, the method relates to reducing the patient's mRAP by at least 18 mmHg. In some embodiments, the method relates to reducing the patient's mRAP by at least 19 mmHg. In certain embodiments, the method relates to reducing the patient's mRAP by at least 20 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's mRAP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's mRAP by at least 1%. In some embodiments, the method relates to decreasing the patient's mRAP by at least 5%. In some embodiments, the method relates to decreasing the patient's mRAP by at least 10%. In some embodiments, the method relates to decreasing the patient's mRAP by at least 15%. In some embodiments, the method relates to decreasing the patient's mRAP by at least 20%. In some embodiments, the method relates to decreasing the patient's mRAP by at least 25%. In some embodiments, the method relates to decreasing the patient's mRAP by at least 30%. In some embodiments, the method relates to decreasing the patient's mRAP by at least 35%. In some embodiments, the method relates to decreasing the patient's mRAP by at least 40%. In some embodiments, the method relates to decreasing the patient's mRAP by at least 45%. In some embodiments, the method relates to decreasing the patient's mRAP by at least 50%. In some embodiments, the method relates to decreasing the patient's mRAP by at least 55%. In some embodiments, the method relates to decreasing the patient's mRAP by at least 60%. In some embodiments, the method relates to decreasing the patient's mRAP by at least 65%. In some embodiments, the method relates to decreasing the patient's mRAP by at least 70%. In some embodiments, the method relates to decreasing the patient's mRAP by at least 75%. In some embodiments, the method relates to decreasing the patient's mRAP by at least 80%. In some embodiments, the method relates to decreasing the patient's mRAP by at least 85%. In some embodiments, the method relates to decreasing the patient's mRAP by at least 90%. In some embodiments, the method relates to decreasing the patient's mRAP by at least 95%. In some embodiments, the method relates to decreasing the patient's mRAP by at least 100%.

### PVR

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has a pulmonary vascular resistance (PVR) of at least 2.5 Woods Units (e.g., at least 2.5, 3, 4, 5, 6, 7, 8, 9, 10, 12, 14, 16, 18, or 20 Woods Units). In some embodiments, the method relates to patients having a PVR of at least 2.5 Woods Units. In some embodiments, the method relates to patients having a PVR of at least 3 Woods Units. In some embodiments, the method relates to patients having a PVR of at least 4 Woods Units. In some embodiments, the method relates to patients having a PVR of at least 5 Woods Units. In some embodiments, the method relates to patients having a PVR of at least 6 Woods Units. In some embodiments, the method relates to patients having a PVR of at least 7 Woods Units. In some embodiments, the method relates to patients having a PVR of at least 8 Woods Units. In some embodiments, the method relates to patients having a PVR of at least 9 Woods Units. In some embodiments, the method relates to patients having a PVR of at least 10 Woods Units. In some embodiments, the method relates to patients having a PVR of at least 12 Woods Units. In some embodiments, the method relates to patients having a PVR of at least 14 Woods Units. In some embodiments, the method relates to patients having a PVR of at least 16 Woods Units. In some embodiments, the method relates to patients having a PVR of at least 18 Woods Units. In some embodiments, the method relates to patients having a PVR of at least 20 Woods Units.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's PVR. In some embodiments, the reduction in the patient's PVR is a result of a decrease in the patient's mean pulmonary arterial pressure (mPAP). In some embodiments, the method relates to reducing the patient's PVR by at least 0.5 Wood Units. In some embodiments, the method relates to reducing the patient's PVR by at least 1 Wood Units. In some embodiments, the method relates to reducing the patient's PVR by at least 2 Wood Units. In some embodiments, the method relates to reducing the patient's PVR by at least 4 Wood Units. In some embodiments, the method relates to reducing the patient's PVR by at least 6 Wood Units. In some embodiments, the method relates to reducing the patient's PVR by at least 8 Wood Units. In some embodiments, the method relates to reducing the patient's PVR by at least 10 Wood Units.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's PVR In some embodiments, the reduction in the patient's PVR is a result of a decrease in the patient's mean pulmonary arterial pressure (mPAP). In some embodiments, the method relates to decreasing the patient's PVR by least 1% (e.g., at least 1%, 5, 10, 15, 20, 25, 30, 35, 40, 45, 50, 55, 60, 65, 70, 75, 80, 85, 90, 95, or 100%). In some embodiments, the method relates to decreasing the patient's PVR. In some embodiments, the method relates to decreasing the patient's PVR by at least 1%. In some embodiments, the method relates to decreasing the patient's PVR by at least 5%. In some embodiments, the method relates to decreasing the patient's PVR by at least 10%. In some embodiments, the method relates to decreasing the patient's PVR by at least 15%. In some embodiments, the method relates to decreasing the patient's PVR by at least 20%. In some embodiments, the method relates to decreasing the patient's PVR by at least 25%. In some embodiments, the method relates to decreasing the patient's PVR by at least 30%. In some embodiments, the method relates to decreasing the patient's PVR by at least 35%. In some embodiments, the method relates to decreasing the patient's PVR by at least 40%. In some embodiments, the method relates to decreasing the patient's PVR by at least 45%. In some embodiments, the method relates to decreasing the patient's PVR by at least 50%. In some embodiments, the method relates to decreasing the patient's PVR by at least 55%. In some embodiments, the method relates to decreasing the patient's PVR by at least 60%. In some embodiments, the method relates to decreasing the patient's PVR by at least 65%. In some embodiments, the method relates to decreasing the patient's PVR by at least 70%. In some embodiments, the method relates to decreasing the patient's PVR by at least 75%. In some embodiments, the method relates to decreasing the patient's PVR by at least 80%. In some embodiments, the method relates to decreasing the patient's PVR by at least 85%. In some embodiments, the method relates to decreasing the patient's PVR by at least 90%. In some embodiments, the method relates to decreasing the patient's PVR by at least 95%. In some embodiments, the method relates to decreasing the patient's PVR by at least 100%.

In some embodiments, PVR is tested after the patient has received 4 weeks of treatment utilizing a variant ActRIIB polypeptide disclosed herein. In some embodiments, PVR is tested after the patient has received 8 weeks of treatment utilizing a variant ActRIIB polypeptide disclosed herein. In some embodiments, PVR is tested after the patient has received 12 weeks of treatment utilizing a variant ActRIIB polypeptide disclosed herein. In some embodiments, PVR is tested after the patient has received 16 weeks of treatment utilizing a variant ActRIIB polypeptide disclosed herein. In some embodiments, PVR is tested after the patient has received 20 weeks of treatment utilizing a variant ActRIIB polypeptide disclosed herein. In some embodiments, PVR is tested after the patient has received 22 weeks of treatment utilizing a variant ActRIIB polypeptide disclosed herein. In some embodiments, PVR is tested after the patient has received 24 weeks of treatment utilizing a variant ActRIIB polypeptide disclosed herein. In some embodiments, PVR is tested after the patient has received 26 weeks of treatment utilizing a variant ActRIIB polypeptide disclosed herein. In some embodiments, PVR is tested after the patient has received 28 weeks of treatment utilizing a variant ActRIIB polypeptide disclosed herein. In some embodiments, PVR is tested after the patient has received 48 weeks of treatment utilizing a variant ActRIIB polypeptide disclosed herein.

### PAWP

In certain aspects, the disclosure relates to methods of treating, preventing, or reducing the progression rate and/or severity of PcPH (e.g., treating, preventing, or reducing the progression rate and/or severity of one or more complications of PcPH WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has pulmonary arterial wedge pressure (PAWP) of at least 12 mmHg (e.g., at least 12, 15, 20, 25, 30, 35, 40, 45, or 50 mmHg). In some embodiments, the method relates to patients having a PAWP of at least 15 mmHg. In some embodiments, the method relates to patients having a PAWP of at least 20 mmHg. In some embodiments, the method relates to patients having a PAWP of at least 25 mmHg. In some embodiments, the method relates to patients having a PAWP of at least 30 mmHg. In some embodiments, the method relates to patients having a PAWP of at least 35 mmHg. In some embodiments, the method relates to patients having a PAWP of at least 40 mmHg. In some embodiments, the method relates to patients having a PAWP of at least 45 mmHg. In some embodiments, the method relates to patients having a PAWP of at least 50 mmHg. In some embodiments, the method relates to patients having a PCWP between to 30 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's PAWP by at least 1 mmHg. In some embodiments, the method relates to reducing the patient's PAWP by at least 2 mmHg. In some embodiments, the method relates to reducing the patient's PAWP by at least 4 mmHg. In some embodiments, the method relates to reducing the patient's PAWP by at least 6 mmHg. In some embodiments, the method relates to reducing the patient's PAWP by at least 10 mmHg. In some embodiments, the method relates to reducing the patient's PAWP by at least 15 mmHg. In some embodiments, the method relates to reducing the patient's PAWP by at least 20 mmHg. In some embodiments, the method relates to reducing the patient's PAWP by at least 25 mmHg. In some embodiments, the method relates to reducing the patient's PAWP by at least 30 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's PAWP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's PAWP by at least 1%. In some embodiments, the method relates to decreasing the patient's PAWP by at least 5%. In some embodiments, the method relates to decreasing the patient's PAWP by at least 10%. In some embodiments, the method relates to decreasing the patient's PAWP by at least 15%. In some embodiments, the method relates to decreasing the patient's PAWP by at least 20%. In some embodiments, the method relates to decreasing the patient's PAWP by at least 25%. In some embodiments, the method relates to decreasing the patient's PAWP by at least 30%. In some embodiments, the method relates to decreasing the patient's PAWP by at least 35%. In some embodiments, the method relates to decreasing the patient's PAWP by at least 40%. In some embodiments, the method relates to decreasing the patient's PAWP by at least 45%. In some embodiments, the method relates to decreasing the patient's PAWP by at least 50%. In some embodiments, the method relates to decreasing the patient's PAWP by at least 55%. In some embodiments, the method relates to decreasing the patient's PAWP by at least 60%. In some embodiments, the method relates to decreasing the patient's PAWP by at least 65%. In some embodiments, the method relates to decreasing the patient's PAWP by at least 70%. In some embodiments, the method relates to decreasing the patient's PAWP by at least 75%. In some embodiments, the method relates to decreasing the patient's PAWP by at least 80%. In some embodiments, the method relates to decreasing the patient's PAWP by at least 85%. In some embodiments, the method relates to decreasing the patient's PAWP by at least 90%. In some embodiments, the method relates to decreasing the patient's PAWP by at least 95%. In some embodiments, the method relates to decreasing the patient's PAWP by at least 100%.

### LVEDP

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has left ventricular end diastolic pressure (LVEDP) of at least 12 mmHg (e.g., at least 12, 15, 20, 25, 30, 35, 40, 45, or 50 mmHg). In some embodiments, the method relates to patients having a LVEDP of at least 15 mmHg. In some embodiments, the method relates to patients having a LVEDP of at least 20 mmHg. In some embodiments, the method relates to patients having a LVEDP of at least 25 mmHg. In some embodiments, the method relates to patients having a LVEDP of at least 30 mmHg. In some embodiments, the method relates to patients having a LVEDP of at least 35 mmHg. In some embodiments, the method relates to patients having a LVEDP of at least 40 mmHg. In some embodiments, the method relates to patients having a LVEDP of at least 45 mmHg. In some embodiments, the method relates to patients having a LVEDP of at least 50 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's LVEDP by at least 1 mmHg. In some embodiments, the method relates to reducing the patient's LVEDP by at least 2 mmHg. In some embodiments, the method relates to reducing the patient's LVEDP by at least 4 mmHg. In some embodiments, the method relates to reducing the patient's LVEDP by at least 6 mmHg. In some embodiments, the method relates to reducing the patient's LVEDP by at least 10 mmHg. In some embodiments, the method relates to reducing the patient's LVEDP by at least mmHg. In some embodiments, the method relates to reducing the patient's LVEDP by at least 20 mmHg. In some embodiments, the method relates to reducing the patient's LVEDP by at least 25 mmHg. In some embodiments, the method relates to reducing the patient's LVEDP by at least 30 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's LVEDP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's LVEDP by at least 1%. In some embodiments, the method relates to decreasing the patient's LVEDP by at least 5%. In some embodiments, the method relates to decreasing the patient's LVEDP by at least 10%. In some embodiments, the method relates to decreasing the patient's LVEDP by at least 15%. In some embodiments, the method relates to decreasing the patient's LVEDP by at least 20%. In some embodiments, the method relates to decreasing the patient's LVEDP by at least 25%. In some embodiments, the method relates to decreasing the patient's LVEDP by at least 30%. In some embodiments, the method relates to decreasing the patient's LVEDP by at least 35%. In some embodiments, the method relates to decreasing the patient's LVEDP by at least 40%. In some embodiments, the method relates to decreasing the patient's LVEDP by at least 45%. In some embodiments, the method relates to decreasing the patient's LVEDP by at least 50%. In some embodiments, the method relates to decreasing the patient's LVEDP by at least 55%. In some embodiments, the method relates to decreasing the patient's LVEDP by at least 60%. In some embodiments, the method relates to decreasing the patient's LVEDP by at least 65%. In some embodiments, the method relates to decreasing the patient's LVEDP by at least 70%. In some embodiments, the method relates to decreasing the patient's LVEDP by at least 75%. In some embodiments, the method relates to decreasing the patient's LVEDP by at least 80%. In some embodiments, the method relates to decreasing the patient's LVEDP by at least 85%. In some embodiments, the method relates to decreasing the patient's LVEDP by at least 90%. In some embodiments, the method relates to decreasing the patient's LVEDP by at least 95%. In some embodiments, the method relates to decreasing the patient's LVEDP by at least 100%.

### DPG

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has resting diastolic pressure gradient (DPG) of at least 5 mmHg (e.g., at least 5, 6, 7, 8, 9, 10, 15, 20, 25, 30, 35, 40, or 45 mmHg). In some embodiments, the method relates to patients having a DPG of at least 5 mmHg. In some embodiments, the method relates to patients having a DPG of at least 6 mmHg. In some embodiments, the method relates to patients having a DPG of at least 7 mmHg. In some embodiments, the method relates to patients having a DPG of at least 8 mmHg. In some embodiments, the method relates to patients having a DPG of at least 9 mmHg. In some embodiments, the method relates to patients having a DPG of at least 10 mmHg. In some embodiments, the method relates to patients having a DPG of at least 15 mmHg. In some embodiments, the method relates to patients having a DPG of at least 20 mmHg. In some embodiments, the method relates to patients having a DPG of at least 25 mmHg. In some embodiments, the method relates to patients having a DPG of at least 30 mmHg. In some embodiments, the method relates to patients having a DPG of at least 35 mmHg. In some embodiments, the method relates to patients having a DPG of at least 40 mmHg. In some embodiments, the method relates to patients having a DPG of at least 45 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's DPG by at least 1 mmHg. In some embodiments, the method relates to reducing the patient's DPG by at least 2 mmHg. In some embodiments, the method relates to reducing the patient's DPG by at least 4 mmHg. In some embodiments, the method relates to reducing the patient's DPG by at least 6 mmHg. In some embodiments, the method relates to reducing the patient's DPG by at least 10 mmHg. In some embodiments, the method relates to reducing the patient's DPG by at least 15 mmHg. In some embodiments, the method relates to reducing the patient's DPG by at least 20 mmHg. In some embodiments, the method relates to reducing the patient's DPG by at least 25 mmHg. In some embodiments, the method relates to reducing the patient's DPG by at least 30 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's DPG by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's DPG by at least 1%. In some embodiments, the method relates to decreasing the patient's DPG by at least 5%. In some embodiments, the method relates to decreasing the patient's DPG by at least 10%. In some embodiments, the method relates to decreasing the patient's DPG by at least 15%. In some embodiments, the method relates to decreasing the patient's DPG by at least 20%. In some embodiments, the method relates to decreasing the patient's DPG by at least 25%. In some embodiments, the method relates to decreasing the patient's DPG by at least 30%. In some embodiments, the method relates to decreasing the patient's DPG by at least 35%. In some embodiments, the method relates to decreasing the patient's DPG by at least 40%. In some embodiments, the method relates to decreasing the patient's DPG by at least 45%. In some embodiments, the method relates to decreasing the patient's DPG by at least 50%. In some embodiments, the method relates to decreasing the patient's DPG by at least 55%. In some embodiments, the method relates to decreasing the patient's DPG by at least 60%. In some embodiments, the method relates to decreasing the patient's DPG by at least 65%. In some embodiments, the method relates to decreasing the patient's DPG by at least 70%. In some embodiments, the method relates to decreasing the patient's DPG by at least 75%. In some embodiments, the method relates to decreasing the patient's DPG by at least 80%. In some embodiments, the method relates to decreasing the patient's DPG by at least 85%. In some embodiments, the method relates to decreasing the patient's DPG by at least 90%. In some embodiments, the method relates to decreasing the patient's DPG by at least 95%. In some embodiments, the method relates to decreasing the patient's DPG by at least 100%.

### TPG

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has a transpulmonary gradient (TPG) of at least 10 mmHg (e.g., at least 10, 11, 12, 13, 14, 15, 20, 25, 30, 35, 40, 45, or 50 mmHg). In some embodiments, the method relates to patients having a TPG of at least 10 mmHg. In some embodiments, the method relates to patients having a TPG of at least 11 mmHg. In some embodiments, the method relates to patients having a TPG of at least 12 mmHg. In some embodiments, the method relates to patients having a TPG of at least 13 mmHg. In some embodiments, the method relates to patients having a TPG of at least 14 mmHg. In some embodiments, the method relates to patients having a TPG of at least 15 mmHg. In some embodiments, the method relates to patients having a TPG of at least 20 mmHg. In some embodiments, the method relates to patients having a TPG of at least 25 mmHg. In some embodiments, the method relates to patients having a TPG of at least 30 mmHg. In some embodiments, the method relates to patients having a TPG of at least 35 mmHg. In some embodiments, the method relates to patients having a TPG of at least 40 mmHg. In some embodiments, the method relates to patients having a TPG of at least 45 mmHg. In some embodiments, the method relates to patients having a TPG of at least 50 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's TPG by at least 1 mmHg. In some embodiments, the method relates to reducing the patient's TPG by at least 2 mmHg. In some embodiments, the method relates to reducing the patient's TPG by at least 4 mmHg. In some embodiments, the method relates to reducing the patient's TPG by at least 6 mmHg. In some embodiments, the method relates to reducing the patient's TPG by at least 10 mmHg. In some embodiments, the method relates to reducing the patient's TPG by at least 15 mmHg. In some embodiments, the method relates to reducing the patient's TPG by at least 20 mmHg. In some embodiments, the method relates to reducing the patient's TPG by at least 25 mmHg. In some embodiments, the method relates to reducing the patient's TPG by at least 30 mmHg. In some embodiments, the method relates to reducing the patient's TPG by at least 40 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's TPG by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's TPG by at least 1%. In some embodiments, the method relates to decreasing the patient's TPG by at least 5%. In some embodiments, the method relates to decreasing the patient's TPG by at least 10%. In some embodiments, the method relates to decreasing the patient's TPG by at least 15%. In some embodiments, the method relates to decreasing the patient's TPG by at least 20%. In some embodiments, the method relates to decreasing the patient's TPG by at least 25%. In some embodiments, the method relates to decreasing the patient's TPG by at least 30%. In some embodiments, the method relates to decreasing the patient's TPG by at least 35%. In some embodiments, the method relates to decreasing the patient's TPG by at least 40%. In some embodiments, the method relates to decreasing the patient's TPG by at least 45%. In some embodiments, the method relates to decreasing the patient's TPG by at least 50%. In some embodiments, the method relates to decreasing the patient's TPG by at least 55%. In some embodiments, the method relates to decreasing the patient's TPG by at least 60%. In some embodiments, the method relates to decreasing the patient's TPG by at least 65%. In some embodiments, the method relates to decreasing the patient's TPG by at least 70%. In some embodiments, the method relates to decreasing the patient's TPG by at least 75%. In some embodiments, the method relates to decreasing the patient's TPG by at least 80%. In some embodiments, the method relates to decreasing the patient's TPG by at least 85%. In some embodiments, the method relates to decreasing the patient's TPG by at least 90%. In some embodiments, the method relates to decreasing the patient's TPG by at least 95%. In some embodiments, the method relates to decreasing the patient's TPG by at least 100%.

### BNP

Both BNP and NT-proBNP are markers of atrial and ventricular distension due to increased intracardiac pressure. The New York Heart Association (NYHA) developed a 4-stage functional classification system for congestive heart failure (CHF) based on the severity of symptoms. Studies have demonstrated that the measured concentrations of circulating BNP and NT-proBNP increase with the severity of CHF based on the NYHA classification. In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has a brain natriuretic peptide (BNP) level of at least 100 pg/mL (e.g., at least 100, 150, 200, 300, 400, 500, 600, 700, 800, 900, 1000, 3000, 5000, 10,000, 15,000, or 20,000 pg/mL). In some embodiments, the method relates to patient's having a BNP level of at least 100 pg/mL. In some embodiments, the method relates to patient's having a BNP level of at least 150 pg/mL. In some embodiments, the method relates to patient's having a BNP level of at least 200 pg/mL. In some embodiments, the method relates to patient's having a BNP level of at least 300 pg/mL. In some embodiments, the method relates to patient's having a BNP level of at least 400 pg/mL. In some embodiments, the method relates to patient's having a BNP level of at least 500 pg/mL. In some embodiments, the method relates to patient's having a BNP level of at least 600 pg/mL. In some embodiments, the method relates to patient's having a BNP level of at least 700 pg/mL. In some embodiments, the method relates to patient's having a BNP level of at least 800 pg/mL. In some embodiments, the method relates to patient's having a BNP level of at least 900 pg/mL. In some embodiments, the method relates to patient's having a BNP level of at least 1000 pg/mL. In some embodiments, the method relates to patient's having a BNP level of at least 5000 pg/mL. In some embodiments, the method relates to patient's having a BNP level of at least 10,000 pg/mL. In some embodiments, the method relates to patient's having a BNP level of at least 15,000 pg/mL. In some embodiments, the method relates to patient's having a BNP level of at least 20,000 pg/mL. In some embodiments, the method relates to treatment of a patient who has elevated BNP levels as compared to a healthy patient.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's BNP levels by at least 10 pg/mL. In some embodiments, the method relates to reducing the patient's BNP levels by at least 50 pg/mL. In some embodiments, the method relates to reducing the patient's BNP levels by at least 100 pg/mL. In some embodiments, the method relates to reducing the patient's BNP levels by at least 200 pg/mL. In some embodiments, the method relates to reducing the patient's BNP levels by at least 300 pg/mL. In some embodiments, the method relates to reducing the patient's BNP levels by at least 400 pg/mL. In some embodiments, the method relates to reducing the patient's BNP levels by at least 500 pg/mL. In some embodiments, the method relates to reducing the patient's BNP levels by at least 600 pg/mL. In some embodiments, the method relates to reducing the patient's BNP levels by at least 700 pg/mL. In some embodiments, the method relates to reducing the patient's BNP levels by at least 800 pg/mL. In some embodiments, the method relates to reducing the patient's BNP levels by at least 900 pg/mL. In some embodiments, the method relates to reducing the patient's BNP levels by at least 1000 pg/mL. In some embodiments, the method relates to reducing the patient's BNP levels by at least 5000 pg/mL. In some embodiments, the method relates to reducing the patient's BNP levels to normal levels. In some embodiments, normal levels correspond to levels of <100 pg/mL.

In some embodiments, the method relates to reducing the patient's BNP by at least 5% (e.g., at least 5, 10, 15, 20, 25, 30, 35, 40, 45, 50, 55, 60, 65, 70, 75, 80, 85, 90, 95, or 100%). In some embodiments, the method relates to reducing the patient's BNP by at least 5%. In some embodiments, the method relates to reducing the patient's BNP by at least 10%. In some embodiments, the method relates to reducing the patient's BNP by at least 15%. In some embodiments, the method relates to reducing the patient's BNP by at least 20%. In some embodiments, the method relates to reducing the patient's BNP by at least 25%. In some embodiments, the method relates to reducing the patient's BNP by at least 30%. In some embodiments, the method relates to reducing the patient's BNP by at least 35%. In some embodiments, the method relates to reducing the patient's BNP by at least 40%. In some embodiments, the method relates to reducing the patient's BNP by at least 45%. In some embodiments, the method relates to reducing the patient's BNP by at least 50%. In some embodiments, the method relates to reducing the patient's BNP by at least 55%. In some embodiments, the method relates to reducing the patient's BNP by at least 60%. In some embodiments, the method relates to reducing the patient's BNP by at least 65%. In some embodiments, the method relates to reducing the patient's BNP by at least 70%. In some embodiments, the method relates to reducing the patient's BNP by at least 75%. In some embodiments, the method relates to reducing the patient's BNP by at least 80%. In some embodiments, the method relates to reducing the patient's BNP by at least 85%. In some embodiments, the method relates to reducing the patient's BNP by at least 90%. In some embodiments, the method relates to reducing the patient's BNP by at least 95%. In some embodiments, the method relates to reducing the patient's BNP by at least 100%.

### NT-proBNP

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has a NT-proBNP level of at least 100 pg/mL (e.g., at least 100, 150, 200, 300, 400, 500, 600, 700, 800, 900, 1000, 3000, 5000, 10,000, 15,000, 20,000, 25,000, or 30,000 pg/mL). In some embodiments, the method relates to patient's having a NT-proBNP level of at least 100 pg/mL. In some embodiments, the method relates to patient's having a NT-proBNP level of at least 150 pg/mL. In some embodiments, the method relates to patient's having a NT-proBNP level of at least 200 pg/mL. In some embodiments, the method relates to patient's having a NT-proBNP level of at least 300 pg/mL. In some embodiments, the method relates to patient's having a NT-proBNP level of at least 400 pg/mL. In some embodiments, the method relates to patient's having a NT-proBNP level of at least 500 pg/mL. In some embodiments, the method relates to patient's having a NT-proBNP level of at least 600 pg/mL. In some embodiments, the method relates to patient's having a NT-proBNP level of at least 700 pg/mL. In some embodiments, the method relates to patient's having a NT-proBNP level of at least 800 pg/mL. In some embodiments, the method relates to patient's having a NT-proBNP level of at least 900 pg/mL. In some embodiments, the method relates to patient's having a NT-proBNP level of at least 1000 pg/mL. In some embodiments, the method relates to patient's having a NT-proBNP level of at least 5000 pg/mL. In some embodiments, the method relates to patient's having a NT-proBNP level of at least 10,000 pg/mL. In some embodiments, the method relates to patient's having a NT-proBNP level of at least 15,000 pg/mL. In some embodiments, the method relates to patient's having a NT-proBNP level of at least 20,000 pg/mL. In some embodiments, the method relates to patient's having a NT-proBNP level of at least 25,000 pg/mL. In some embodiments, the method relates to patient's having a NT-proBNP level of at least 30,000 pg/mL. In some embodiments, the method relates to treatment of a patient who has elevated NT-proBNP levels as compared to a healthy patient.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB poly peptides of the present disclosure (e.g., variant ActRIIB polypeptides in either homomeric or heteromeric forms). In some embodiments, the method relates to reducing the patient's NT-proBNP levels. In some embodiments, the method relates to reducing the patient's NT-proBNP by at least 10 pg/mL. In some embodiments, the method relates to reducing the patient's NT-proBNP by at least 50 pg/mL. In some embodiments, the method relates to reducing the patient's NT-proBNP by at least 100 pg/mL. In some embodiments, the method relates to reducing the patient's NT-proBNP by at least 200 pg/mL. In some embodiments, the method relates to reducing the patient's NT-proBNP by at least 300 pg/mL. In some embodiments, the method relates to reducing the patient's NT-proBNP by at least 400 pg/mL. In some embodiments, the method relates to reducing the patient's NT-proBNP by at least 500 pg/mL. In some embodiments, the method relates to reducing the patient's NT-proBNP by at least 600 pg/mL. In some embodiments, the method relates to reducing the patient's NT-proBNP by at least 700 pg/mL. In some embodiments, the method relates to reducing the patient's NT-proBNP by at least 800 pg/mL. In some embodiments, the method relates to reducing the patient's NT-proBNP by at least 900 pg/mL. In some embodiments, the method relates to reducing the patient's NT-proBNP by at least 1000 pg/mL. In some embodiments, the method relates to reducing the patient's NT-proBNP by at least 5000 pg/mL. In some embodiments, the method relates to reducing the patient's NT-proBNP by at least 10,000 pg/mL. In some embodiments, the method relates to reducing the patient's NT-proBNP by at least 15,000 pg/mL. In some embodiments, the method relates to reducing the patient's NT-proBNP by at least 20,000 pg/mL. In some embodiments, the method relates to reducing the patient's NT-proBNP by at least 25,000 pg/mL.

In some embodiments, the method relates to decreasing the patient's NT-proBNP levels to a normal level and maintaining their normal NT-proBNP levels. In some embodiments, the disclosure relates to methods of maintaining one or more hemodynamic parameters in the PcPH patient at a normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to maintaining the patient's NT-proBNP levels at a normal level. In some embodiments, the method relates to maintaining the patient's NT-proBNP level at less than 100 pg/mL. In some embodiments, the method relates to maintaining the patient's NT-proBNP level at less than 200 pg/mL. In some embodiments, the method relates to maintaining the patient's NT-proBNP level at less than 300 pg/mL. In some embodiments, the method relates to maintaining the patient's NT-proBNP level at less than 400 pg/mL.

In some embodiments, the method relates to reducing the patient's NT-proBNP by at least 5% (e.g., at least 5, 10, 15, 20, 25, 30, 35, 40, 45, 50, 55, 60, 65, 70, 75, 80, 85, 90, 95, or 100%). In some embodiments, the method relates to reducing the patient's NT-proBNP by at least 5%. In some embodiments, the method relates to reducing the patient's NT-proBNP by at least 10%. In some embodiments, the method relates to reducing the patient's NT-proBNP by at least 15%. In some embodiments, the method relates to reducing the patient's NT-proBNP by at least 20%. In some embodiments, the method relates to reducing the patient's NT-proBNP by at least 25%. In some embodiments, the method relates to reducing the patient's NT-proBNP by at least 30%. In some embodiments, the method relates to reducing the patient's NT-proBNP by at least 35%. In some embodiments, the method relates to reducing the patient's NT-proBNP by at least 40%. In some embodiments, the method relates to reducing the patient's NT-proBNP by at least 45%. In some embodiments, the method relates to reducing the patient's NT-proBNP by at least 50%. In some embodiments, the method relates to reducing the patient's NT-proBNP by at least 55%. In some embodiments, the method relates to reducing the patient's NT-proBNP by at least 60%. In some embodiments, the method relates to reducing the patient's NT-proBNP by at least 65%. In some embodiments, the method relates to reducing the patient's NT-proBNP by at least 70%. In some embodiments, the method relates to reducing the patient's NT-proBNP by at least 75%. In some embodiments, the method relates to reducing the patient's NT-proBNP by at least 80%. In some embodiments, the method relates to reducing the patient's NT-proBNP by at least 85%. In some embodiments, the method relates to reducing the patient's NT-proBNP by at least 90%. In some embodiments, the method relates to reducing the patient's NT-proBNP by at least 95%. In some embodiments, the method relates to reducing the patient's NT-proBNP by at least 100%. In some embodiments, the method relates to reducing the patient's NT-proBNP levels to normal levels. In some embodiments, normal levels of NT-proBNP is <100 pg/ml. In some embodiments, the method relates to reducing the patient's NT-proBNP levels to less than 300 ng/L.

### Smooth Muscle Hypertrophy

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has smooth muscle hypertrophy. In some embodiments, the disclosure relates to methods of adjusting one or more parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing smooth muscle hypertrophy in the patient. In some embodiments, the method relates to decreasing the patient's smooth muscle hypertrophy by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's smooth muscle hypertrophy by at least 1%. In some embodiments, the method relates to decreasing the patient's smooth muscle hypertrophy by at least 5%. In some embodiments, the method relates to decreasing the patient's smooth muscle hypertrophy by at least 10%. In some embodiments, the method relates to decreasing the patient's smooth muscle hypertrophy by at least 15%. In some embodiments, the method relates to decreasing the patient's smooth muscle hypertrophy by at least 20%. In some embodiments, the method relates to decreasing the patient's smooth muscle hypertrophy by at least 25%. In some embodiments, the method relates to decreasing the patient's smooth muscle hypertrophy by at least 30%. In some embodiments, the method relates to decreasing the patient's smooth muscle hypertrophy by at least 35%. In some embodiments, the method relates to decreasing the patient's smooth muscle hypertrophy by at least 40%. In some embodiments, the method relates to decreasing the patient's smooth muscle hypertrophy by at least 45%. In some embodiments, the method relates to decreasing the patient's smooth muscle hypertrophy by at least 50%. In some embodiments, the method relates to decreasing the patient's smooth muscle hypertrophy by at least 55%. In some embodiments, the method relates to decreasing the patient's smooth muscle hypertrophy by at least 60%. In some embodiments, the method relates to decreasing the patient's smooth muscle hypertrophy by at least 65%. In some embodiments, the method relates to decreasing the patient's smooth muscle hypertrophy by at least 70%. In some embodiments, the method relates to decreasing the patient's smooth muscle hypertrophy by at least 75%. In some embodiments, the method relates to decreasing the patient's smooth muscle hypertrophy by at least 80%. In some embodiments, the method relates to decreasing the patient's smooth muscle hypertrophy by at least 85%. In some embodiments, the method relates to decreasing the patient's smooth muscle hypertrophy by at least 90%. In some embodiments, the method relates to decreasing the patient's smooth muscle hypertrophy by at least 95%. In some embodiments, the method relates to decreasing the patient's smooth muscle hypertrophy by at least 100%.

### Pulmonary Arteriole Muscularity

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has increased pulmonary arteriole muscularity. In some embodiments, the disclosure relates to methods of adjusting one or more parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing pulmonary arteriole muscularity in the patient. In some embodiments, the method relates to decreasing the patient's pulmonary arteriole muscularity by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's pulmonary arteriole muscularity by at least 1%. In some embodiments, the method relates to decreasing the patient's pulmonary arteriole muscularity by at least 5%. In some embodiments, the method relates to decreasing the patient's pulmonary arteriole muscularity by at least 10%. In some embodiments, the method relates to decreasing the patient's pulmonary arteriole muscularity by at least 15%. In some embodiments, the method relates to decreasing the patient's pulmonary arteriole muscularity by at least 20%. In some embodiments, the method relates to decreasing the patient's pulmonary arteriole muscularity by at least 25%. In some embodiments, the method relates to decreasing the patient's pulmonary arteriole muscularity by at least 30%. In some embodiments, the method relates to decreasing the patient's pulmonary arteriole muscularity by at least 35%. In some embodiments, the method relates to decreasing the patient's pulmonary arteriole muscularity by at least 40%. In some embodiments, the method relates to decreasing the patient's pulmonary arteriole muscularity by at least 45%. In some embodiments, the method relates to decreasing the patient's pulmonary arteriole muscularity by at least 50%. In some embodiments, the method relates to decreasing the patient's pulmonary arteriole muscularity by at least 55%. In some embodiments, the method relates to decreasing the patient's pulmonary arteriole muscularity by at least 60%. In some embodiments, the method relates to decreasing the patient's pulmonary arteriole muscularity by at least 65%. In some embodiments, the method relates to decreasing the patient's pulmonary arteriole muscularity by at least 70%. In some embodiments, the method relates to decreasing the patient's pulmonary arteriole muscularity by at least 75%. In some embodiments, the method relates to decreasing the patient's pulmonary arteriole muscularity by at least 80%. In some embodiments, the method relates to decreasing the patient's pulmonary arteriole muscularity by at least 85%. In some embodiments, the method relates to decreasing the patient's pulmonary arteriole muscularity by at least 90%. In some embodiments, the method relates to decreasing the patient's pulmonary arteriole muscularity by at least 95%. In some embodiments, the method relates to decreasing the patient's pulmonary arteriole muscularity by at least 100%.

### Rate of Hospitalization

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the method reduces the patient's hospitalization rate by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, the method relates to reducing the patient's hospitalization rate by at least 1%. In some embodiments, the method relates to reducing the patient's hospitalization rate by at least 2%. In some embodiments, the method relates to reducing the patient's hospitalization rate by at least 3%. In some embodiments, the method relates to reducing the patient's hospitalization rate by at least 4%. In some embodiments, the method relates to reducing the patient's hospitalization rate by at least 5%. In some embodiments, the method relates to reducing the patient's hospitalization rate by at least 10%. In some embodiments, the method relates to reducing the patient's hospitalization rate by at least 15%. In some embodiments, the method relates to reducing the patient's hospitalization rate by at least 20%. In some embodiments, the method relates to reducing the patient's hospitalization rate by at least 25%. In some embodiments, the method relates to reducing the patient's hospitalization rate by at least 30%. In some embodiments, the method relates to reducing the patient's hospitalization rate by at least 35%. In some embodiments, the method relates to reducing the patient's hospitalization rate by at least 40%. In some embodiments, the method relates to reducing the patient's hospitalization rate by at least 45%. In some embodiments, the method relates to reducing the patient's hospitalization rate by at least 50%. In some embodiments, the method relates to reducing the patient's hospitalization rate by at least 55%. In some embodiments, the method relates to reducing the patient's hospitalization rate by at least 60%. In some embodiments, the method relates to reducing the patient's hospitalization rate by at least 65%. In some embodiments, the method relates to reducing the patient's hospitalization rate by at least 70%. In some embodiments, the method relates to reducing the patient's hospitalization rate by at least 75%. In some embodiments, the method relates to reducing the patient's hospitalization rate by at least 80%. In some embodiments, the method relates to reducing the patient's hospitalization rate by at least 85%. In some embodiments, the method relates to reducing the patient's hospitalization rate by at least 90%. In some embodiments, the method relates to reducing the patient's hospitalization rate by at least 95%. In some embodiments, the method relates to reducing the patient's hospitalization rate by at least 100%. In some embodiments, the method reduces the risk of hospitalization for one or more complications associated with PcPH.

### Quality of Life

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the method increases the patient's quality of life by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to increasing the patient's quality of life by at least 1%. In some embodiments, the method relates to increasing the patient's quality of life by at least 2%. In some embodiments, the method relates to increasing the patient's quality of life by at least 3%. In some embodiments, the method relates to increasing the patient's quality of life by at least 4%. In some embodiments, the method relates to increasing the patient's quality of life by at least 5%. In some embodiments, the method relates to increasing the patient's quality of life by at least 10%. In some embodiments, the method relates to increasing the patient's quality of life by at least 15%. In some embodiments, the method relates to increasing the patient's quality of life by at least 20%. In some embodiments, the method relates to increasing the patient's quality of life by at least 25%. In some embodiments, the method relates to increasing the patient's quality of life by at least 30%. In some embodiments, the method relates to increasing the patient's quality of life by at least 35%. In some embodiments, the method relates to increasing the patient's quality of life by at least 40%. In some embodiments, the method relates to increasing the patient's quality of life by at least 45%. In some embodiments, the method relates to increasing the patient's quality of life by at least 50%. In some embodiments, the method relates to increasing the patient's quality of life by at least 55%. In some embodiments, the method relates to increasing the patient's quality of life by at least 60%. In some embodiments, the method relates to increasing the patient's quality of life by at least 65%. In some embodiments, the method relates to increasing the patient's quality of life by at least 70%. In some embodiments, the method relates to increasing the patient's quality of life by at least 75%. In some embodiments, the method relates to increasing the patient's quality of life by at least 80%. In some embodiments, the method relates to increasing the patient's quality of life by at least 85%. In some embodiments, the method relates to increasing the patient's quality of life by at least 90%. In some embodiments, the method relates to increasing the patient's quality of life by at least 95%. In some embodiments, the method relates to increasing the patient's quality of life by at least 100%.

In some embodiments, the patient's quality of life is measured using the Cambridge Pulmonary Hypertension Outcome Review (CAMPHOR). In some embodiments, the patient's quality of life is measured using PAH-SYMPACTX®. In some embodiments, the patient's quality of life is measured using the Medical Outcomes Survey Short Form-36 (SF-36). In some embodiments, the patient's quality of life is measured using the Euro Quality of Life (EuroQol). In some embodiments, the patient's quality of life is measured using the Euro Quality of Life-5 dimensions (EQ-5D). In some embodiments, the patient's quality of life is measured using the Euro Quality of Life-5 dimensions 5-levels (EQ-5D-5L). In some embodiments, the patient's quality of life is measured using the Kansas City Cardiomyopathy Questionnaire (KCCQ).

### Diastolic Function

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the method increases the patient's LV diastolic function by at least 5% (e.g., at least 5, 10, 15, 20, 25, 30, 35, 40, 45, 50, 55, 60, 65, 70, 75, 80, 85, 90, 95, or 100%). In some embodiments, the method relates to increasing the patient's LV diastolic function by at least 5%. In some embodiments, the method relates to increasing the patient's LV diastolic function by at least 10%. In some embodiments, the method relates to increasing the patient's LV diastolic function by at least 15%. In some embodiments, the method relates to increasing the patient's LV diastolic function by at least 20%. In some embodiments, the method relates to increasing the patient's LV diastolic function by at least 25%. In some embodiments, the method relates to increasing the patient's LV diastolic function by at least 30%. In some embodiments, the method relates to increasing the patient's LV diastolic function by at least 35%. In some embodiments, the method relates to increasing the patient's LV diastolic function by at least 40%. In some embodiments, the method relates to increasing the patient's LV diastolic function by at least 45%. In some embodiments, the method relates to increasing the patient's LV diastolic function by at least 50%. In some embodiments, the method relates to increasing the patient's LV diastolic function by at least 55%. In some embodiments, the method relates to increasing the patient's LV diastolic function by at least 60%. In some embodiments, the method relates to increasing the patient's LV diastolic function by at least 65%. In some embodiments, the method relates to increasing the patient's LV diastolic function by at least 70%. In some embodiments, the method relates to increasing the patient's LV diastolic function by at least 75%. In some embodiments, the method relates to increasing the patient's LV diastolic function by at least 80%. In some embodiments, the method relates to increasing the patient's LV diastolic function by at least 85%. In some embodiments, the method relates to increasing the patient's LV diastolic function by at least 90%. In some embodiments, the method relates to increasing the patient's LV diastolic function by at least 95%. In some embodiments, the method relates to increasing the patient's LV diastolic function by at least 100%.

### Ejection Fraction

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has an ejection fraction of less than 10% (e.g., less than 10, 15, 20, 25, 30, 35, 40, or 45%). In some embodiments, the method relates to patient's having an ejection fraction of less than 10%. In some embodiments, the method relates to patient's having an ejection fraction of less than 15%. In some embodiments, the method relates to patient's having an ejection fraction of less than 20%. In some embodiments, the method relates to patient's having an ejection fraction of less than 25%. In some embodiments, the method relates to patient's having an ejection fraction of less than 30%. In some embodiments, the method relates to patient's having an ejection fraction of less than 35%. In some embodiments, the method relates to patient's having an ejection fraction of less than 40%. In some embodiments, the method relates to patient's having an ejection fraction of less than 45%. In some embodiments, the method relates to patient's having an ejection fraction of less than 50%. In some embodiments, the method relates to patient's having an ejection fraction of less than 55%. In some embodiments, the ejection fraction is the right ventricular ejection fraction. In some embodiments, the ejection fraction is the left ventricular ejection fraction.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has an ejection fraction of at least 35% (e.g., at least 35, 40, 45, 50, or 55%). In some embodiments, the method relates to patient's having an ejection fraction of at least 35%. In some embodiments, the method relates to patient's having an ejection fraction of at least 40%. In some embodiments, the method relates to patient's having an ejection fraction of at least 45%. In some embodiments, the method relates to patient's having an ejection fraction of at least 50%. In some embodiments, the method relates to patient's having an ejection fraction of at least 55%. In some embodiments, the ejection fraction is the right ventricular ejection fraction. In some embodiments, the ejection fraction is the left ventricular ejection fraction (LVEF). In some embodiments, the ejection fraction is measured using an echocardiogram. In some embodiments, the patient has a preserved left ventricular ejection fraction.

In some embodiments, the disclosure relates to methods of adjusting one or more hemodynamic parameters in the PcPH patient toward a more normal level (e.g., >50% ejection fraction), comprising administering to a patient in need thereof an effective amount of one or more variant ActRIIB polypeptides of the present disclosure (e.g., variant ActRIIB polypeptides in either homomeric or heteromeric forms). In some embodiments, the method relates to increasing the patient's ejection fraction by least 1%. In some embodiments, the method relates to increasing the patient's ejection fraction by at least 5%. In some embodiments, the method relates to increasing the patient's ejection fraction by at least 10%. In some embodiments, the method relates to increasing the patient's ejection fraction by at least 15%. In some embodiments, the method relates to increasing the patient's ejection fraction by at least 20%. In some embodiments, the method relates to increasing the patient's ejection fraction by at least 25%. In some embodiments, the method relates to increasing the patient's ejection fraction by at least 30%. In some embodiments, the method relates to increasing the patient's ejection fraction by at least 35%. In some embodiments, the method relates to increasing the patient's ejection fraction by at least 40%. In some embodiments, the method relates to increasing the patient's ejection fraction by at least 45%. In some embodiments, the method relates to increasing the patient's ejection fraction by at least 50%. In some embodiments, the method relates to increasing the patient's ejection fraction by at least 55%. In some embodiments, the method relates to increasing the patient's ejection fraction by at least 60%. In some embodiments, the method relates to increasing the patient's ejection fraction by at least 65%. In some embodiments, the method relates to increasing the patient's ejection fraction by at least 70%. In some embodiments, the method relates to increasing the patient's ejection fraction by at least 75%. In some embodiments, the method relates to increasing the patient's ejection fraction by at least 80%. In some embodiments, the method relates to increasing the patient's ejection fraction by at least 85%. In some embodiments, the method relates to increasing the patient's ejection fraction by at least 90%. In some embodiments, the method relates to increasing the patient's ejection fraction by at least 95%. In some embodiments, the method relates to increasing the patient's ejection fraction by at least 100%.

### Ventricular Function

In certain aspects, the disclosure relates to methods of improving or maintaining ventricular function (e.g., left ventricular function or right ventricular function) in PcPH comprising administering to a patient in need thereof an effective amount of one or more variant ActRIIB polypeptides of the present disclosure (e.g., variant ActRIIB polypeptides in either homomeric or heteromeric forms). Echocardiography is a useful noninvasive screening tool for determining the severity of pulmonary hypertension in a patient. Improvement or maintenance of ventricular function (e.g., left ventricular function or right ventricular function) can be assessed by many echocardiographic measurements. One such quantitative approach to assess ventricular function is the measurement of the tricuspid annular plane systolic excursion (TAPSE). The TAPSE estimates RV systolic function by measuring the level of systolic excursion of the lateral tricuspid valve annulus towards the apex. Other echocardiographic measurements that may be used to assess maintenance and/or improvements in ventricular function include, but are not limited to, right ventricular fractional area change (RVFAC), right ventricular end-diastolic area (RVEDA), right ventricular end-systolic area (RVESA), right ventricular free wall thickness (RVFWT), right ventricular ejection fraction (RVEF), right ventricular-pulmonary artery (RV-PA) coupling, pulmonary arterial systolic pressure (PASP), right ventricular systolic pressure (RVSP), pulmonary artery acceleration time (PAAT), tricuspid regurgitation velocity (TRV), left ventricular hypertrophy, and right ventricular hypertrophy.

### TAPSE

The tricuspid annular plane systolic excursion (TAPSE) can be obtained using echocardiography and represents a measure of RV longitudinal function. The TAPSE has previously been shown to have good correlations with parameters estimating RV global systolic function. A TAPSE<17 mm is highly suggestive of RV systolic dysfunction. In some embodiments, an improvement or maintenance of right ventricular function in a PcPH patient is measured as an increase in TAPSE. In some embodiments, a PcPH patient with an improvement or maintenance of right ventricular function has a TAPSE between 20 mm-28 mm. In some embodiments, a PcPH patient with an improvement or maintenance of right ventricular function has a TAPSE of at least 20 mm. In some embodiments, a PcPH patient with an improvement or maintenance of right ventricular function has a TAPSE of at least 22 mm. In some embodiments, a PcPH patient with an improvement or maintenance of right ventricular function has a TAPSE of at least 24 mm. In some embodiments, a PcPH patient with an improvement or maintenance of right ventricular function has a TAPSE of at least 26 mm. In some embodiments, a PcPH patient with an improvement or maintenance of right ventricular function has a TAPSE of at least 28 mm. In some embodiments, the TAPSE is measured using echocardiography.

In some embodiments, a PcPH patient with an improvement or maintenance of right ventricular function has a TAPSE between 16 mm-30 mm. In some embodiments, a PcPH patient with an improvement or maintenance of right ventricular function has a TAPSE between 18 mm-28 mm. In some embodiments, a PcPH patient with an improvement or maintenance of right ventricular function has a TAPSE of at least 18 mm. In some embodiments, the TAPSE is measured using echocardiography.

PASP and RVSP In certain aspects, the disclosure relates to methods of treating, preventing, or reducing the progression rate and/or severity of PcPH comprising administering to a patient in need thereof an effective amount of one or more variant ActRIIB polypeptides of the present disclosure (e.g., variant ActRIIB polypeptides in either homomeric or heteromeric forms), wherein the patient has a pulmonary arterial systolic pressure (PASP) of at least 30 mmHg (e.g., at least 30, 35, 40, 45, 50, 55, 60, 65, 70, 75, or 80 mmHg). In some embodiments, the method relates to patients having a PASP of at least 30 mmHg. In some embodiments, the method relates to patients having a PASP of at least 35 mmHg. In some embodiments, the method relates to patients having a PASP of at least 40 mmHg. In some embodiments, the method relates to patients having a PASP of at least 45 mmHg. In some embodiments, the method relates to patients having a PASP of at least 50 mmHg. In some embodiments, the method relates to patients having a PASP of at least 55 mmHg. In some embodiments, the method relates to patients having a PASP of at least 60 mmHg. In some embodiments, the method relates to patients having a PASP of at least 65 mmHg. In some embodiments, the method relates to patients having a PASP of at least 70 mmHg. In some embodiments, the method relates to patients having a PASP of at least 75 mmHg. In some embodiments, the method relates to patients having a PASP of at least 80 mmHg. In some embodiments, the PASP is a resting PASP. In some embodiments, the PASP is determined using the tricuspid regurgitation velocity (TRV) and right arterial (RA) pressure. In some embodiments, the PASP is determined using the following formula:

PASP=TRV²×4RA pressure

TRV has been shown to correlate with PASP at rest and with exercise. The pressure gradient between the right ventricle and the right atrium can be calculated using the modified Bernoulli equation (Δp=4V²).

In some embodiments, the right ventricular systolic pressure (RVSP) is equal to PASP. In some embodiments, the RVSP is measured in the absence of right ventricular outflow tract obstruction. In some embodiments, the RVSP is determined using the following formula:

RVSP4V²+RAP

In the above formula, V represents the peak tricuspid regurgitant jet velocity and RAP is the mean right atrial pressure. RVSP is frequently used for estimating PASP.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving the pulmonary arterial systolic pressure (PASP) in the patient. In some embodiments, the method relates to reducing PASP. In some embodiments, the method relates to reducing the patient's PASP by at least 1 mmHg (e.g., at least 1, 2, 3, 5, 7, 10, 12, 15, 20, 25, 30, or 35 mmHg). In some embodiments, the method relates to reducing the patient's PASP by at least 2 mmHg. In some embodiments, the method relates to reducing the patient's PASP by at least 3 mmHg. In certain embodiments, the method relates to reducing the patient's PASP by at least 5 mmHg. In certain embodiments, the method relates to reducing the patient's PASP by at least 7 mmHg. In certain embodiments, the method relates to reducing the patient's PASP by at least 10 mmHg. In certain embodiments, the method relates to reducing the patient's PASP by at least 12 mmHg. In certain embodiments, the method relates to reducing the patient's PASP by at least 15 mmHg. In certain embodiments, the method relates to reducing the patient's PASP by at least 20 mmHg. In certain embodiments, the method relates to reducing the patient's PASP by at least 25 mmHg. In certain embodiments, the method relates to reducing the patient's PASP by at least 30 mmHg. In certain embodiments, the method relates to reducing the patient's PASP by at least 35 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's PASP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to reducing the patient's PASP by at least 1%. In some embodiments, the method relates to reducing the patient's PASP by at least 5%. In some embodiments, the method relates to reducing the patient's PASP by at least 10%. In some embodiments, the method relates to reducing the patient's PASP by at least 15%. In some embodiments, the method relates to reducing the patient's PASP by at least 20%. In some embodiments, the method relates to reducing the patient's PASP by at least 25%. In some embodiments, the method relates to reducing the patient's PASP by at least 30%. In some embodiments, the method relates to reducing the patient's PASP by at least 35%. In some embodiments, the method relates to reducing the patient's PASP by at least 40%. In some embodiments, the method relates to reducing the patient's PASP by at least 45%. In some embodiments, the method relates to reducing the patient's PASP by at least 50%. In some embodiments, the method relates to reducing the patient's PASP by at least 55%. In some embodiments, the method relates to reducing the patient's PASP by at least 60%. In some embodiments, the method relates to reducing the patient's PASP by at least 65%. In some embodiments, the method relates to reducing the patient's PASP by at least 70%. In some embodiments, the method relates to reducing the patient's PASP by at least 75%. In some embodiments, the method relates to reducing the patient's PASP by at least 80%. In some embodiments, the method relates to reducing the patient's PASP by at least 85%. In some embodiments, the method relates to reducing the patient's PASP by at least 90%. In some embodiments, the method relates to reducing the patient's PASP by at least 95%. In some embodiments, the method relates to reducing the patient's PASP by at least 100%.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving the right ventricular systolic pressure (RVSP) in the patient. In some embodiments, the method relates to reducing RVSP. In some embodiments, the method relates to reducing the patient's RVSP by at least 1 mmHg (e.g., at least 1, 2, 3, 5, 7, 10, 12, 15, 20, 25, 30, or 35 mmHg). In some embodiments, the method relates to reducing the patient's RVSP by at least 2 mmHg. In some embodiments, the method relates to reducing the patient's RVSP by at least 3 mmHg. In certain embodiments, the method relates to reducing the patient's RVSP by at least 5 mmHg. In certain embodiments, the method relates to reducing the patient's RVSP by at least 7 mmHg. In certain embodiments, the method relates to reducing the patient's RVSP by at least 10 mmHg. In certain embodiments, the method relates to reducing the patient's RVSP by at least 12 mmHg. In certain embodiments, the method relates to reducing the patient's RVSP by at least 15 mmHg. In certain embodiments, the method relates to reducing the patient's RVSP by at least 20 mmHg. In certain embodiments, the method relates to reducing the patient's RVSP by at least 25 mmHg. In certain embodiments, the method relates to reducing the patient's RVSP by at least 30 mmHg. In certain embodiments, the method relates to reducing the patient's RVSP by at least 35 mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's RVSP by least 1% (e.g., at least 1, 5, 10, 15, 20, 25, 30, 35, 40, 45, 50, 55, 60, 65, 70, 75, 80, 85, 90, 95, or 100%). In some embodiments, the method relates to reducing the patient's RVSP by at least 5%. In some embodiments, the method relates to reducing the patient's RVSP by at least 10%. In some embodiments, the method relates to reducing the patient's RVSP by at least 15%. In some embodiments, the method relates to reducing the patient's RVSP by at least 20%. In some embodiments, the method relates to reducing the patient's RVSP by at least 25%. In some embodiments, the method relates to reducing the patient's RVSP by at least 30%. In some embodiments, the method relates to reducing the patient's RVSP by at least 35%. In some embodiments, the method relates to reducing the patient's RVSP by at least 40%. In some embodiments, the method relates to reducing the patient's RVSP by at least 45%. In some embodiments, the method relates to reducing the patient's RVSP by at least 50%. In some embodiments, the method relates to reducing the patient's RVSP by at least 55%. In some embodiments, the method relates to reducing the patient's RVSP by at least 60%. In some embodiments, the method relates to reducing the patient's RVSP by at least 65%. In some embodiments, the method relates to reducing the patient's RVSP by at least 70%. In some embodiments, the method relates to reducing the patient's RVSP by at least 75%. In some embodiments, the method relates to reducing the patient's RVSP by at least 80%. In some embodiments, the method relates to reducing the patient's RVSP by at least 85%. In some embodiments, the method relates to reducing the patient's RVSP by at least 90%. In some embodiments, the method relates to reducing the patient's RVSP by at least 95%. In some embodiments, the method relates to reducing the patient's RVSP by at least 100%.

### RV-PA Coupling

Right ventricular dysfunction can occur in PcPH and is a factor affecting prognosis. Energy transfer between ventricle contractility and arterial afterload is termed coupling. Energy transfer specifically between the right ventricle (RV) and pulmonary artery is termed right ventricle-pulmonary artery (RV-PA) coupling. In some embodiments, right ventricular dysfunction is due to a decrease in RV-PA coupling. RV-PA coupling can be estimated non-invasively as a ratio of TAPSE/PASP values. In some embodiments, a TAPSE/PASP ratio of ≥0.31 mm/mm Hg may be associated with a better prognosis and reduced risk of clinical worsening. In some embodiments, the improvement in RV-PA coupling is due to an improvement in PASP. In some embodiments, the calculation of RV-PA coupling is dependent upon paired results for three parameters (e.g., TRV, RAP, and TAPSE).

In certain aspects, the disclosure relates to methods of treating, preventing, or reducing the progression rate and/or severity of PcPH comprising administering to a patient in need thereof an effective amount of one or more variant ActRIIB polypeptides of the present disclosure (e.g., variant ActRIIB polypeptides in either homomeric or heteromeric forms), wherein the patient has a TAPSE/PASP ratio less than 0.31 mm/mmHg (e.g., less than 0.3, 0.25, 0.2, 0.15, or 0.1 mm/mmHg). In some embodiments, the method relates to patients having a TAPSE/PASP ratio less than 0.31 mm/mmHg. In some embodiments, the method relates to patients having a TAPSE/PASP ratio less than 0.3 mm/mmHg. In some embodiments, the method relates to patients having a TAPSE/PASP ratio less than 0.25 mm/mmHg. In some embodiments, the method relates to patients having a TAPSE/PASP ratio less than 0.2 mm/mmHg. In some embodiments, the method relates to patients having a TAPSE/PASP ratio less than 0.15 mm/mmHg. In some embodiments, the method relates to patients having a TAPSE/PASP ratio less than 0.1 mm/mmHg. In some embodiments, the method relates to patients having a decreased TAPSE/PASP ratio as compared to a normal TAPSE/PASP ratio.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving or maintaining the right ventricular function in the patient. In some embodiments, a PcPH patient with an improvement or maintenance of right ventricular function has a TAPSE/PASP ratio greater than 0.3 mm/mmHg (e.g., greater than 0.31, 0.32, 0.33, 0.34, or 0.35 mm/mmHg). In some embodiments, a PcPH patient with an improvement or maintenance of right ventricular function has a TAPSE/PASP ratio greater than 0.31 mm/mmHg. In some embodiments, a PcPH patient with an improvement or maintenance of right ventricular function has a TAPSE/PASP ratio greater than 0.32 mm/mmHg. In some embodiments, a PcPH patient with an improvement or maintenance of right ventricular function has a TAPSE/PASP ratio greater than 0.33 mm/mmHg. In some embodiments, a PcPH patient with an improvement or maintenance of right ventricular function has a TAPSE/PASP ratio greater than 0.34 mm/mmHg. In some embodiments, a PcPH patient with an improvement or maintenance of right ventricular function has a TAPSE/PASP ratio greater than 0.35 mm/mmHg. In some embodiments, the improvement in right ventricular function is an increase in TAPSE/PASP ratio. In some embodiments, the method relates to increasing the TAPSE/PASP ratio. In some embodiments, the method relates to increasing the patient's TAPSE/PASP ratio by at least 0.05 mm/mmHg. In some embodiments, the method relates to increasing the patient's TAPSE/PASP ratio by at least 0.07 mm/mmHg. In some embodiments, the method relates to increasing the patient's TAPSE/PASP ratio by at least 0.10 mm/mmHg. In some embodiments, the method relates to increasing the patient's TAPSE/PASP ratio by at least 0.12 mm/mmHg. In some embodiments, the method relates to increasing the patient's TAPSE/PASP ratio by at least 0.15 mm/mmHg. In some embodiments, the method relates to increasing the patient's TAPSE/PASP ratio by at least 0.18 mm/mmHg. In some embodiments, the method relates to increasing the patient's TAPSE/PASP ratio by at least 0.20 mm/mmHg.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to increasing the patient's TAPSE/PASP ratio by least 1% (e.g., at least 1, 5, 10, 15, 20, 25, 30, 35, 40, 45, 50, 55, 60, 65, 70, 75, 80, 85, 90, 95, or 100%). In some embodiments, the method relates to increasing the patient's TAPSE/PASP ratio by at least 5%. In some embodiments, the method relates to increasing the patient's TAPSE/PASP ratio by at least 10%. In some embodiments, the method relates to increasing the patient's TAPSE/PASP ratio by at least 15%. In some embodiments, the method relates to increasing the patient's TAPSE/PASP ratio by at least 20%. In some embodiments, the method relates to increasing the patient's TAPSE/PASP ratio by at least 25%. In some embodiments, the method relates to increasing the patient's TAPSE/PASP ratio by at least 30%. In some embodiments, the method relates to increasing the patient's TAPSE/PASP ratio by at least 35%. In some embodiments, the method relates to increasing the patient's TAPSE/PASP ratio by at least 40%. In some embodiments, the method relates to increasing the patient's TAPSE/PASP ratio by at least 45%. In some embodiments, the method relates to increasing the patient's TAPSE/PASP ratio by at least 50%. In some embodiments, the method relates to increasing the patient's TAPSE/PASP ratio by at least 55%. In some embodiments, the method relates to increasing the patient's TAPSE/PASP ratio by at least 60%. In some embodiments, the method relates to increasing the patient's TAPSE/PASP ratio by at least 65%. In some embodiments, the method relates to increasing the patient's TAPSE/PASP ratio by at least 70%. In some embodiments, the method relates to increasing the patient's TAPSE/PASP ratio by at least 75%. In some embodiments, the method relates to increasing the patient's TAPSE/PASP ratio by at least 80%. In some embodiments, the method relates to increasing the patient's TAPSE/PASP ratio by at least 85%. In some embodiments, the method relates to increasing the patient's TAPSE/PASP ratio by at least 90%. In some embodiments, the method relates to increasing the patient's TAPSE/PASP ratio by at least 100%.

RVFAC. RVEDA, and RVESA Right ventricular fractional area change (RVFAC) is a non-invasive quantitative measure of right ventricular function. RVFAC can be calculated using the formula [(RVEDA-RVESA)/RVEDA]*100. In some embodiments, the RVFAC is measured using echocardiography. In some embodiments, normal RVFAC is approximately 47.5 t 8.6% in men and approximately 50.9±8.0% in women. See, e.g., Kou S, et al. European Heart Journal—Cardiovascular Imaging. 2014 Jun. 1; 15(6):680-90. In some embodiments, PcPH patients have a decrease in RVFAC.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has a RVFAC of less than 20% (e.g., les than 20, 25, 30, 35, or 40%). In some embodiments, the method relates to patients having a RVFAC of less than 25%. In some embodiments, the method relates to patients having a RVFAC of less than 30%. In some embodiments, the method relates to patients having a RVFAC of less than 35%. In some embodiments, the method relates to patients having a RVFAC of less than 40%.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving or maintaining the right ventricular function in the patient. In some embodiments, the improvement or maintenance of right ventricular function is due to an increase in right ventricular fractional area change (RVFAC). In some embodiments, a PcPH patient with an improvement or maintenance of right ventricular function has a RVFAC between 32-56%. In some embodiments, a PcPH patient with an improvement or maintenance of right ventricular function has a RVFAC of at least 32%. In some embodiments, a PcPH patient with an improvement or maintenance of right ventricular function has a RVFAC of at least 34%. In some embodiments, a PcPH patient with an improvement or maintenance of right ventricular function has a RVFAC of at least 35%. In some embodiments, a PcPH patient with an improvement or maintenance of right ventricular function has a RVFAC of at least 36%. In some embodiments, a PcPH patient with an improvement or maintenance of right ventricular function has a RVFAC of at least 38%. In some embodiments, a PcPH patient with an improvement or maintenance of right ventricular function has a RVFAC of at least 40%. In some embodiments, a PcPH patient with an improvement or maintenance of right ventricular function has a RVFAC of at least 42%. In some embodiments, a PcPH patient with an improvement or maintenance of right ventricular function has a RVFAC of at least 44%. In some embodiments, a PcPH patient with an improvement or maintenance of right ventricular function has a RVFAC of at least 46%. In some embodiments, a PcPH patient with an improvement or maintenance of right ventricular function has a RVFAC of at least 48%. In some embodiments, a PcPH patient with an improvement or maintenance of right ventricular function has a RVFAC of at least 50%. In some embodiments, a PcPH patient with an improvement or maintenance of right ventricular function has a RVFAC of at least 52%. In some embodiments, a PcPH patient with an improvement or maintenance of right ventricular function has a RVFAC of at least 54%. In some embodiments, a PcPH patient with an improvement or maintenance of right ventricular function has a RVFAC of at least 56%.

In some embodiments, the disclosure relates to methods of adjusting one or more echocardiogram parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's RVEDA by least 1% (e.g., at least 1, 2, 3, 4, 5, 6, 7, 8, 9, 10, 12, 14, 16, 18, or 20%). In some embodiments, the method relates to increasing the patient's RVFAC by least 2%. In some embodiments, the method relates to increasing the patient's RVFAC by least 3%. In some embodiments, the method relates to increasing the patient's RVFAC by least 4%. In some embodiments, the method relates to increasing the patient's RVFAC by least 5%. In some embodiments, the method relates to increasing the patient's RVFAC by least 6%. In some embodiments, the method relates to increasing the patient's RVFAC by least 7%. In some embodiments, the method relates to increasing the patient's RVFAC by least 8%. In some embodiments, the method relates to increasing the patient's RVFAC by least 9%. In some embodiments, the method relates to increasing the patient's RVFAC by least 10%. In some embodiments, the method relates to increasing the patient's RVFAC by least 12%. In some embodiments, the method relates to increasing the patient's RVFAC by least 14%. In some embodiments, the method relates to increasing the patient's RVFAC by least 16%. In some embodiments, the method relates to increasing the patient's RVFAC by least 18%. In some embodiments, the method relates to increasing the patient's RVFAC by least 20%.

In some embodiments, the improvement in right ventricular function is due to an increase in ejection fraction. In some embodiments, the improvement in right ventricular function is due to an increase in ejection fraction and an increase in the patient's RVFAC.

The right ventricular end-diastolic area (RVEDA) can be measured using echocardiography. In some embodiments, normal RVEDA is approximately 18.2 f 4.3 cm² in men and approximately 14.8 f 3.5 cm² in women. See, e.g., Kou S, et al. European Heart Journal—Cardiovascular Imaging. 2014 Jun. 1; 15(6):680-90.

In certain aspects, the disclosure relates to methods of treating, preventing, or reducing the progression rate and/or severity of PcPH comprising administering to a patient in need thereof an effective amount of one or more variant ActRIIB polypeptides of the present disclosure (e.g., variant ActRIIB polypeptides in either homomeric or heteromeric forms), wherein the patient has a RVEDA of at least 22 cm² (e.g., at least 22, 24, 26, 28, 30, 32, or 34 cm²). In some embodiments, the method relates to patients having a RVEDA of at least 24 cm². In some embodiments, the method relates to patients having a RVEDA of at least 26 cm². In some embodiments, the method relates to patients having a RVEDA of at least 28 cm². In some embodiments, the method relates to patients having a RVEDA of at least 30 cm². In some embodiments, the method relates to patients having a RVEDA of at least 32 cm². In some embodiments, the method relates to patients having a RVEDA of at least 34 cm². In some embodiments, the method relates to patients having increased RVEDA as compared to normal RVEDA.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving or maintaining the right ventricular function in the patient. In some embodiments, a patient with an improvement or maintenance of right ventricular function has a RVEDA of 14-22 cm². In some embodiments, the improvement in right ventricular function is a reduction in RVEDA. In some embodiments, the method relates to reducing the RVEDA. In some embodiments, the method relates to reducing the patients RVEDA by at least 1 cm². In some embodiments, the method relates to reducing the patients RVEDA by at least 2 cm². In some embodiments, the method relates to reducing the patients RVEDA by at least 3 cm². In some embodiments, the method relates to reducing the patients RVEDA by at least 4 cm². In some embodiments, the method relates to reducing the patients RVEDA by at least 5 cm². In some embodiments, the method relates to reducing the patients RVEDA by at least 6 cm². In some embodiments, the method relates to reducing the patients RVEDA by at least 7 cm². In some embodiments, the method relates to reducing the patients RVEDA by at least 8 cm². In some embodiments, the method relates to reducing the patients RVEDA by at least 9 cm². In some embodiments, the method relates to reducing the patients RVEDA by at least 10 cm².

In some embodiments, the disclosure relates to methods of adjusting one or more echocardiogram parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's RVEDA by least 1% (e.g., at least 1, 5, 10, 15, 20, 25, 30, 35, or 40%). In some embodiments, the method relates to decreasing the patient's RVEDA by at least 5%. In some embodiments, the method relates to decreasing the patient's RVEDA by at least 10%. In some embodiments, the method relates to decreasing the patient's RVEDA by at least 15%. In some embodiments, the method relates to decreasing the patient's RVEDA by at least 20%. In some embodiments, the method relates to decreasing the patient's RVEDA by at least 25%. In some embodiments, the method relates to decreasing the patient's RVEDA by at least 30%. In some embodiments, the method relates to decreasing the patient's RVEDA by at least 35%. In some embodiments, the method relates to decreasing the patient's RVEDA by at least 40%.

The right ventricular end-systolic area (RVESA) can be measured using echocardiography. In some embodiments, normal RVESA is approximately 9.6±2.8 cm² in men and approximately 7.3±2.3 cm² in women. See, e.g., Kou S. et al. European Heart Journal—Cardiovascular Imaging. 2014 Jun. 1:15(6):680-90.

In certain aspects, the disclosure relates to methods of treating, preventing, or reducing the progression rate and/or severity of PcPH comprising administering to a patient in need thereof an effective amount of one or more variant ActRIIB polypeptides of the present disclosure (e.g., variant ActRIIB polypeptides in either homomeric or heteromeric forms), wherein the patient has a RVESA of at least 12 cm² (e.g., at least 12, 14, 16, 18, 20, 22, 24, 26, 28, 30, or 32 cm²). In some embodiments, the method relates to patients having a RVESA of at least 14 cm². In some embodiments, the method relates to patients having a RVESA of at least 16 cm². In some embodiments, the method relates to patients having a RVESA of at least 18 cm². In some embodiments, the method relates to patients having a RVESA of at least 20 cm². In some embodiments, the method relates to patients having a RVESA of at least 22 cm². In some embodiments, the method relates to patients having a RVESA of at least 24 cm². In some embodiments, the method relates to patients having a RVESA of at least 26 cm². In some embodiments, the method relates to patients having a RVESA of at least 28 cm². In some embodiments, the method relates to patients having a RVESA of at least 30 cm². In some embodiments, the method relates to patients having a RVESA of at least 32 cm². In some embodiments, the method relates to patients having increased RVESA as compared to normal RVESA.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving or maintaining the right ventricular function in the patient. In some embodiments, a patient with an improvement or maintenance of right ventricular function has a RVESA of 7-20 cm². In some embodiments, the improvement in right ventricular function is a reduction in RVESA. In some embodiments, the method relates to reducing the RVESA. In some embodiments, the method relates to reducing the patient's RVESA by at least 1 cm². In some embodiments, the method relates to reducing the patient's RVESA by at least 2 cm². In some embodiments, the method relates to reducing the patient's RVESA by at least 3 cm². In some embodiments, the method relates to reducing the patient's RVESA by at least 4 cm². In some embodiments, the method relates to reducing the patient's RVESA by at least 5 cm². In some embodiments, the method relates to reducing the patient's RVESA by at least 6 cm². In some embodiments, the method relates to reducing the patient's RVESA by at least 7 cm². In some embodiments, the method relates to reducing the patient's RVESA by at least 8 cm². In some embodiments, the method relates to reducing the patient's RVESA by at least 9 cm². In some embodiments, the method relates to reducing the patient's RVESA by at least 10 cm².

In some embodiments, the disclosure relates to methods of adjusting one or more echocardiogram parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's RVESA by least 1% (e.g., at least 1, 2, 3, 4, 5, 10, 15, 20, 25, 30, 35, or 40%). In some embodiments, the method relates to decreasing the patient's RVESA by at least 2%. In some embodiments, the method relates to decreasing the patient's RVESA by at least 3%. In some embodiments, the method relates to decreasing the patient's RVESA by at least 4%. In some embodiments, the method relates to decreasing the patient's RVESA by at least 5%. In some embodiments, the method relates to decreasing the patient's RVESA by at least 10%. In some embodiments, the method relates to decreasing the patient's RVESA by at least 15%. In some embodiments, the method relates to decreasing the patient's RVESA by at least 20%. In some embodiments, the method relates to decreasing the patient's RVESA by at least 25%. In some embodiments, the method relates to decreasing the patient's RVESA by at least 30%. In some embodiments, the method relates to decreasing the patient's RVESA by at least 35%. In some embodiments, the method relates to decreasing the patient's RVESA by at least 40%.

RVFWT In patients with pulmonary hypertension, the right ventricle dilates in response to increased PAP and right ventricular remodeling. As the disease progresses right ventricular hypertrophy develops, resulting in increased right ventricle free wall thickness. In some embodiments, the right ventricular free wall thickness (RVFWT) can be measured using echocardiography. In some embodiments, normal RVFWT is approximately 0.22-0.42 cm in women and approximately 0.24-0.42 cm in men. See, e.g., Lang R M, J Am Soc Echocardiogr. 2015; 28(1):1-39.e14.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has a RVFWT of at least 0.42 cm (e.g., at least 0.42, 0.44, 0.46, 0.48, 0.50, 0.52, 0.54, 0.56, 0.58, or 0.60 cm). In some embodiments, the method relates to patients having a RVFWT of at least 0.44 cm. In some embodiments, the method relates to patients having a RVFWT of at least 0.46 cm. In some embodiments, the method relates to patients having a RVFWT of at least 0.48 cm. In some embodiments, the method relates to patients having a RVFWT of at least 0.50 cm. In some embodiments, the method relates to patients having a RVFWT of at least 0.52 cm. In some embodiments, the method relates to patients having a RVFWT of at least 0.54 cm. In some embodiments, the method relates to patients having a RVFWT of at least 0.56 cm. In some embodiments, the method relates to patients having a RVFWT of at least 0.58 cm. In some embodiments, the method relates to patients having a RVFWT of at least 0.60 cm. In some embodiments, the method relates to patients having increased RVFWT as compared to normal RVFWT.

In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving or maintaining the right ventricular function in the patient. In some embodiments, a patient with an improvement or maintenance of right ventricular function has a RVFWT of between 0.22-0.42 cm. In some embodiments, the improvement in right ventricular function is a reduction in RVFWT. In some embodiments, the method relates to reducing the RVFWT. In some embodiments, the method relates to reducing the patients RVFWT by at least 0.05 cm (e.g., at least 0.05, 0.1, 0.15, 0.2, 0.25, 0.3, 0.35, or 0.4 cm). In some embodiments, the method relates to reducing the patients RVFWT by at least 0.1 cm. In some embodiments, the method relates to reducing the patients RVFWT by at least 0.15 cm. In some embodiments, the method relates to reducing the patients RVFWT by at least 0.2 cm. In some embodiments, the method relates to reducing the patients RVFWT by at least 0.25 cm. In some embodiments, the method relates to reducing the patients RVFWT by at least 0.3 cm. In some embodiments, the method relates to reducing the patients RVFWT by at least 0.35 cm. In some embodiments, the method relates to reducing the patients RVFWT by at least 0.4 cm.

In some embodiments, the disclosure relates to methods of adjusting the RVFWT in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's RVFWT by least 1% (e.g., at least 1, 5, 10, 15, 20, 25, 30, 35, 40, 45, 50, 55, 60, 65, 70, or 75%). In some embodiments, the method relates to decreasing the patient's RVFWT by at least 5%. In some embodiments, the method relates to decreasing the patient's RVFWT by at least 10%. In some embodiments, the method relates to decreasing the patient's RVFWT by at least 15%. In some embodiments, the method relates to decreasing the patient's RVFWT by at least 20%. In some embodiments, the method relates to decreasing the patient's RVFWT by at least 25%. In some embodiments, the method relates to decreasing the patient's RVFWT by at least 30%. In some embodiments, the method relates to decreasing the patient's RVFWT by at least 35%. In some embodiments, the method relates to decreasing the patient's RVFWT by at least 40%. In some embodiments, the method relates to decreasing the patient's RVFWT by at least 45%. In some embodiments, the method relates to decreasing the patient's RVFWT by at least 50%. In some embodiments, the method relates to decreasing the patient's RVFWT by at least 55%. In some embodiments, the method relates to decreasing the patient's RVFWT by at least 60%. In some embodiments, the method relates to decreasing the patient's RVFWT by at least 65%. In some embodiments, the method relates to decreasing the patient's RVFWT by at least 70%. In some embodiments, the method relates to decreasing the patient's RVFWT by at least 75%.

### RVEF

Right ventricular ejection fraction is a global measure of RV systolic performance. RVEF can be calculated using the RV end-diastolic volume (RVEDV) and RV end systolic volume (RVESV). Specifically, RVEF can be calculated using the following formula: RVEF (%)=((RVEDV−RVESV)/RVEDV)*100. Normal RVEF is approximately 56-65% in men and 60-71% in women. See, e.g., Lang R M, J Am Soc Echocardiogr. 2015; 28(1):1-39.e14. In some embodiments, the RVEF is measured using echocardiography. In some embodiments, the disclosure relates to methods of adjusting one or more hemodynamic parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to improving or maintaining the right ventricular function in the patient. In some embodiments, a patient with an improvement or maintenance of right ventricular function has a RVEF of 45-71%. In some embodiments, a patient with an improvement or maintenance of right ventricular function has a RVEF of at least 45%. In some embodiments, a patient with an improvement or maintenance of right ventricular function has a RVEF of at least 50%. In some embodiments, a patient with an improvement or maintenance of right ventricular function has a RVEF of at least 55%. In some embodiments, a patient with an improvement or maintenance of right ventricular function has a RVEF of at least 60%. In some embodiments, a patient with an improvement or maintenance of right ventricular function has a RVEF of at least 65%. In some embodiments, a patient with an improvement or maintenance of right ventricular function has a RVEF of at least 70%.

In some embodiments, the disclosure relates to methods of adjusting one or more echocardiogram parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to increasing the patient's RVEF by least 2%. In some embodiments, the method relates to increasing the patient's RVEF by least 3%. In some embodiments, the method relates to increasing the patient's RVEF by least 4%. In some embodiments, the method relates to increasing the patient's RVEF by least 5%. In some embodiments, the method relates to increasing the patient's RVEF by least 6%. In some embodiments, the method relates to increasing the patient's RVEF by least 7%. In some embodiments, the method relates to increasing the patient's RVEF by least 8%. In some embodiments, the method relates to increasing the patient's RVEF by least 9%. In some embodiments, the method relates to increasing the patient's RVEF by least 10%. In some embodiments, the method relates to increasing the patient's RVEF by least 11%. In some embodiments, the method relates to increasing the patient's RVEF by least 12%. In some embodiments, the method relates to increasing the patient's RVEF by least 13%. In some embodiments, the method relates to increasing the patient's RVEF by least 14%. In some embodiments, the method relates to increasing the patient's RVEF by least 15%.

### Right Ventricular Hypertrophy

In certain aspects, the improvement in right ventricular function is measured as a decrease in right ventricular hypertrophy. In some embodiment, the right ventricular hypertrophy is measured using the Fulton Index (RV/(LV+S)).

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has right ventricular hypertrophy. In some embodiments, the disclosure relates to methods of adjusting one or more parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the right ventricular hypertrophy is measured using the Fulton index (RV/(LV+S)). In some embodiments, the method relates to decreasing the patient's right ventricular hypertrophy by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's right ventricular hypertrophy by at least 1%. In some embodiments, the method relates to decreasing the patient's right ventricular hypertrophy by at least 5%. In some embodiments, the method relates to decreasing the patient's right ventricular hypertrophy by at least 10%. In some embodiments, the method relates to decreasing the patient's right ventricular hypertrophy by at least 15%. In some embodiments, the method relates to decreasing the patient's right ventricular hypertrophy by at least 20%. In some embodiments, the method relates to decreasing the patient's right ventricular hypertrophy by at least 25%. In some embodiments, the method relates to decreasing the patient's right ventricular hypertrophy by at least 30%. In some embodiments, the method relates to decreasing the patient's right ventricular hypertrophy by at least 35%. In some embodiments, the method relates to decreasing the patient's right ventricular hypertrophy by at least 40%. In some embodiments, the method relates to decreasing the patient's right ventricular hypertrophy by at least 45%. In some embodiments, the method relates to decreasing the patient's right ventricular hypertrophy by at least 50%. In some embodiments, the method relates to decreasing the patient's right ventricular hypertrophy by at least 55%. In some embodiments, the method relates to decreasing the patient's right ventricular hypertrophy by at least 60%. In some embodiments, the method relates to decreasing the patient's right ventricular hypertrophy by at least 65%. In some embodiments, the method relates to decreasing the patient's right ventricular hypertrophy by at least 70%. In some embodiments, the method relates to decreasing the patient's right ventricular hypertrophy by at least 75%. In some embodiments, the method relates to decreasing the patient's right ventricular hypertrophy by at least 80%. In some embodiments, the method relates to decreasing the patient's right ventricular hypertrophy by at least 85%. In some embodiments, the method relates to decreasing the patient's right ventricular hypertrophy by at least 90%. In some embodiments, the method relates to decreasing the patient's right ventricular hypertrophy by at least 95%. In some embodiments, the method relates to decreasing the patient's right ventricular hypertrophy by at least 100%.

### Left Ventricular Hypertrophy

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has left ventricular hypertrophy. In some embodiments, the disclosure relates to methods of adjusting one or more parameters in the PcPH patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to decreasing the patient's left ventricular hypertrophy by least 1% (e.g., at least 1, 5, 10, 15, 20, 25, 30, 35, 40, 45, 50, 55, 60, 65, 70, 75, 80, 85, 90, 95, or 100%). In some embodiments, the method relates to decreasing the patient's left ventricular hypertrophy by at least 1%. In some embodiments, the method relates to decreasing the patient's left ventricular hypertrophy by at least 5%. In some embodiments, the method relates to decreasing the patient's left ventricular hypertrophy by at least 10%. In some embodiments, the method relates to decreasing the patient's left ventricular hypertrophy by at least 15%. In some embodiments, the method relates to decreasing the patient's left ventricular hypertrophy by at least 20%. In some embodiments, the method relates to decreasing the patient's left ventricular hypertrophy by at least 25%. In some embodiments, the method relates to decreasing the patient's left ventricular hypertrophy by at least 30%. In some embodiments, the method relates to decreasing the patient's left ventricular hypertrophy by at least 35%. In some embodiments, the method relates to decreasing the patient's left ventricular hypertrophy by at least 40%. In some embodiments, the method relates to decreasing the patient's left ventricular hypertrophy by at least 45%. In some embodiments, the method relates to decreasing the patient's left ventricular hypertrophy by at least 50%. In some embodiments, the method relates to decreasing the patient's left ventricular hypertrophy by at least 55%. In some embodiments, the method relates to decreasing the patient's left ventricular hypertrophy by at least 60%. In some embodiments, the method relates to decreasing the patient's left ventricular hypertrophy by at least 65%. In some embodiments, the method relates to decreasing the patient's left ventricular hypertrophy by at least 70%. In some embodiments, the method relates to decreasing the patient's left ventricular hypertrophy by at least 75%. In some embodiments, the method relates to decreasing the patient's left ventricular hypertrophy by at least 80%. In some embodiments, the method relates to decreasing the patient's left ventricular hypertrophy by at least 85%. In some embodiments, the method relates to decreasing the patient's left ventricular hypertrophy by at least 90%. In some embodiments, the method relates to decreasing the patient's left ventricular hypertrophy by at least 95%. In some embodiments, the method relates to decreasing the patient's left ventricular hypertrophy by at least 100%.

Cardiac Output Cardiac output is the volume of blood the heart pumps per minute. Cardiac output is calculated by multiplying the stroke volume by the heart rate. In general, normal cardiac output at rest is about 4 to 8 L/min. The cardiac index is an assessment of the cardiac output value based on the patient's size. To find the cardiac index, the cardiac output is divided by the person's body surface area (BSA). The normal range for CI is 2.5 to 4 L/min/m². Cardiac output can decline by almost 40% without deviating from the normal limits. A low cardiac index of less than about 2.5 L/min/m2 usually indicates a disturbance in cardiovascular performance. The cardiac output can be utilized to calculate the cardiac index (e.g., cardiac index=cardiac output/body surface area). The cardiac output can also be utilized to calculate the stroke volume (e.g., stroke volume=CO/heart rate). In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the method increases the patient's cardiac output by at least 5% (e.g., at least 5, 10, 15, 20, 25, 30, 35, 40, 45, 50, 55, 60, 65, 70, 75, 80, 85, 90, 95, or 100%). In some embodiments, the method relates to increasing the patient's cardiac output by at least 5%. In some embodiments, the method relates to increasing the patient's cardiac output by at least 10%. In some embodiments, the method relates to increasing the patient's cardiac output by at least 15%. In some embodiments, the method relates to increasing the patient's cardiac output by at least 20%. In some embodiments, the method relates to increasing the patient's cardiac output by at least 25%. In some embodiments, the method relates to increasing the patient's cardiac output by at least 30%. In some embodiments, the method relates to increasing the patient's cardiac output by at least 35%. In some embodiments, the method relates to increasing the patient's cardiac output by at least 40%. In some embodiments, the method relates to increasing the patient's cardiac output by at least 45%. In some embodiments, the method relates to increasing the patient's cardiac output by at least 50%. In some embodiments, the method relates to increasing the patient's cardiac output by at least 55%. In some embodiments, the method relates to increasing the patient's cardiac output by at least 60%. In some embodiments, the method relates to increasing the patient's cardiac output by at least 65%. In some embodiments, the method relates to increasing the patient's cardiac output by at least 70%. In some embodiments, the method relates to increasing the patient's cardiac output by at least 75%. In some embodiments, the method relates to increasing the patient's cardiac output by at least 80%. In some embodiments, the method relates to increasing the patient's cardiac output by at least 85%. In some embodiments, the method relates to increasing the patient's cardiac output by at least 90%. In some embodiments, the method relates to increasing the patient's cardiac output by at least 95%. In some embodiments, the method relates to increasing the patient's cardiac output by at least 100%. In some embodiments, the method relates to increasing the patient's cardiac index to at least 4.2 L/min/m2. In some embodiments, the cardiac index is measured at rest. In some embodiments, the method relates to increasing the patient's cardiac output to at least 4 L/min.

In some embodiments, the cardiac output is measured at rest. In some embodiments, the cardiac output is using a right heart catheter. In some embodiments, cardiac output is measured by thermodilution. In some embodiments, cardiac output is measured using the Fick method.

### Progression of IpcPH to CpcPH

The predominant mechanism underlying PcPH (e.g., WHO Group 2 and/or Group 5 PH) is elevated left-side filling pressure (i.e., left atrial pressure). Sustained elevations in left atrial pressure may cause passive pulmonary venous congestion with elevation of pulmonary pressures. In some patients, transmission of venous congestion to the pulmonary capillaries results in leakage and damage, ultimately leading to the creation of an obstructive vasculopathy such that higher pulmonary pressures are needed to sustain forward flow. This is sometimes referred to as the development of a “pre-capillary” component of PH. In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the method reduces the development of a pre-capillary component of PH by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to reducing the development of a pre-capillary component of PH in a patient by at least 1%. In some embodiments, the method relates to reducing the development of a pre-capillary component of PH in a patient by at least 2%. In some embodiments, the method relates to reducing the development of a pre-capillary component of PH in a patient by at least 3%. In some embodiments, the method relates to reducing the development of a pre-capillary component of PH in a patient by at least 4%. In some embodiments, the method relates to reducing the development of a pre-capillary component of PH in a patient by at least 5%. In some embodiments, the method relates to reducing the development of a pre-capillary component of PH in a patient by at least 10%. In some embodiments, the method relates to reducing the development of a pre-capillary component of PH in a patient by at least 15%. In some embodiments, the method relates to reducing the development of a pre-capillary component of PH in a patient by at least 20%. In some embodiments, the method relates to reducing the development of a pre-capillary component of PH in a patient by at least 25%. In some embodiments, the method relates to reducing the development of a pre-capillary component of PH in a patient by at least 30%. In some embodiments, the method relates to reducing the development of a pre-capillary component of PH in a patient by at least 35%. In some embodiments, the method relates to reducing the development of a pre-capillary component of PH in a patient by at least 40%. In some embodiments, the method relates to reducing the development of a pre-capillary component of PH in a patient by at least 45%. In some embodiments, the method relates to reducing the development of a pre-capillary component of PH in a patient by at least 50%. In some embodiments, the method relates to reducing the development of a pre-capillary component of PH in a patient by at least 55%. In some embodiments, the method relates to reducing the development of a pre-capillary component of PH in a patient by at least 60%. In some embodiments, the method relates to reducing the development of a pre-capillary component of PH in a patient by at least 65%. In some embodiments, the method relates to reducing the development of a pre-capillary component of PH in a patient by at least 70%. In some embodiments, the method relates to reducing the development of a pre-capillary component of PH in a patient by at least 75%. In some embodiments, the method relates to reducing the development of a pre-capillary component of PH in a patient by at least 80%. In some embodiments, the method relates to reducing the development of a pre-capillary component of PH in a patient by at least 85%. In some embodiments, the method relates to reducing the development of a pre-capillary component of PH in a patient by at least 90%. In some embodiments, the method relates to reducing the development of a pre-capillary component of PH in a patient by at least 95%. In some embodiments, the method relates to reducing the development of a pre-capillary component of PH in a patient by at least 100%.

In some embodiments, sustained left atrial pressure in IpcPH has been shown to lead to the development of CpcPH. In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the method reduces the progression of IpcPH to CpcPH in a patient by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to reducing the progression of IpcPH to CpcPH in a patient by at least 1%. In some embodiments, the method relates to reducing the progression of IpcPH to CpcPH in a patient by at least 2%. In some embodiments, the method relates to reducing the progression of IpcPH to CpcPH in a patient by at least 3%. In some embodiments, the method relates to reducing the progression of IpcPH to CpcPH in a patient by at least 4%. In some embodiments, the method relates to reducing the progression of IpcPH to CpcPH in a patient by at least 5%. In some embodiments, the method relates to reducing the progression of IpcPH to CpcPH in a patient by at least 10%. In some embodiments, the method relates to reducing the progression of IpcPH to CpcPH in a patient by at least 15%. In some embodiments, the method relates to reducing the progression of IpcPH to CpcPH in a patient by at least 20%. In some embodiments, the method relates to reducing the progression of IpcPH to CpcPH in a patient by at least 25%. In some embodiments, the method relates to reducing the progression of IpcPH to CpcPH in a patient by at least 30%. In some embodiments, the method relates to reducing the progression of IpcPH to CpcPH in a patient by at least 35%. In some embodiments, the method relates to reducing the progression of IpcPH to CpcPH in a patient by at least 40%. In some embodiments, the method relates to reducing the progression of IpcPH to CpcPH in a patient by at least 45%. In some embodiments, the method relates to reducing the progression of IpcPH to CpcPH in a patient by at least 50%. In some embodiments, the method relates to reducing the progression of IpcPH to CpcPH in a patient by at least 55%. In some embodiments, the method relates to reducing the progression of IpcPH to CpcPH in a patient by at least 60%. In some embodiments, the method relates to reducing the progression of IpcPH to CpcPH in a patient by at least 65%. In some embodiments, the method relates to reducing the progression of IpcPH to CpcPH in a patient by at least 70%. In some embodiments, the method relates to reducing the progression of IpcPH to CpcPH in a patient by at least 75%. In some embodiments, the method relates to reducing the progression of IpcPH to CpcPH in a patient by at least 80%. In some embodiments, the method relates to reducing the progression of IpcPH to CpcPH in a patient by at least 85%. In some embodiments, the method relates to reducing the progression of IpcPH to CpcPH in a patient by at least 90%. In some embodiments, the method relates to reducing the progression of IpcPH to CpcPH in a patient by at least 95%. In some embodiments, the method relates to reducing the progression of IpcPH to CpcPH in a patient by at least 100%.

### Exercise Capacity (6MWD and BDI)

In certain aspects, the disclosure relates to methods of increasing exercise capacity in a patient having PcPH comprising administering to a patient in need thereof an effective amount of one or more variant ActRIIB polypeptides of the present disclosure (e.g., variant ActRIIB polypeptides in either homomeric or heteromeric forms). Any suitable measure of exercise capacity can be used. For example, exercise capacity in a 6-minute walk test (6MWT), which measures how far the subject can walk in 6 minutes, i.e., the 6-minute walk distance (6MWD), is frequently used to assess pulmonary hypertension severity and disease progression. In certain embodiments, the Borg dyspnea index (BDI) may be used to measure exercise capacity. The BDI is a numerical scale for assessing perceived dyspnea (breathing discomfort). It measures the degree of breathlessness, for example, after completion of the 6MWT, where a BDI of 0 indicates no breathlessness and 10 indicates maximum breathlessness. In some embodiments, the BDI is measured using the BORG CR10 scale.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has a 6MWD of less than 550 meters (e.g., a 6MWD of less than 550, 500, 450, 440, 400, 380, 350, 300, 250, 200, or 150 meters). In some embodiments, the method relates to patient's having a 6MWD of between 150 to 550 meters. In some embodiments, the method relates to patient's having a 6MWD of between 100 to 500 meters. In some embodiments, the method relates to patient's having a 6MWD of between 150 to 500 meters. In some embodiments, the method relates to patient's having a 6MWD of at least 100 meters. In some embodiments, the method relates to patient's having a 6MWD of greater than 150 meters. In some embodiments, the method relates to patient's having a 6MWD of less than 550 meters. In some embodiments, the method relates to patient's having a 6MWD of less than 500 meters. In some embodiments, the method relates to patient's having a 6MWD of less than 450 meters. In some embodiments, the method relates to patient's having a 6MWD of less than 440 meters. In some embodiments, the method relates to patient's having a 6MWD of less than 400 meters. In some embodiments, the method relates to patient's having a 6MWD of less than 380 meters. In some embodiments, the method relates to patient's having a 6MWD of less than 350 meters. In some embodiments, the method relates to patient's having a 6MWD of less than 300 meters. In some embodiments, the method relates to patient's having a 6MWD of less than 250 meters. In some embodiments, the method relates to patient's having a 6MWD of less than 200 meters. In some embodiments, the method relates to patient's having a 6MWD of less than 150 meters. In some embodiments, the method relates to increasing the patient's 6MWD to >380 meters. In some embodiments, the method relates to increasing the patient's 6MWD to >440 meters. In some embodiments, the method relates to increasing the patient's 6MWD to >500 meters. See, e.g., Galie N., et al Euro Heart J. (2016) 37, 67-119.

In some embodiments, the disclosure relates to methods of adjusting one or more measurements of exercise capacity in the PcPH (e.g., WHO Group 2 and/or Group 5 PH) patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to increasing the patient's 6MWD by at least 10 meters. In some embodiments, the method relates to increasing the patient's 6MWD by at least 20 meters. In some embodiments, the method relates to increasing the patient's 6MWD by at least 25 meters. In some embodiments, the method relates to increasing the patient's 6MWD by at least 30 meters. In some embodiments, the method relates to increasing the patient's 6MWD by at least 40 meters. In some embodiments, the method relates to increasing the patient's 6MWD by at least 50 meters. In some embodiments, the method relates to increasing the patient's 6MWD by at least 60 meters. In some embodiments, the method relates to increasing the patient's 6MWD by at least 70 meters. In some embodiments, the method relates to increasing the patient's 6MWD by at least 80 meters. In some embodiments, the method relates to increasing the patient's 6MWD by at least 90 meters. In some embodiments, the method relates to increasing the patient's 6MWD by at least 100. In some embodiments, the method relates to increasing the patient's 6MWD by at least 125. In some embodiments, the method relates to increasing the patient's 6MWD by at least 150 meters. In some embodiments, the method relates to increasing the patient's 6MWD by at least 175 meters. In some embodiments, the method relates to increasing the patient's 6MWD by at least 200 meters. In some embodiments, the method relates to increasing the patient's 6MWD by at least 250 meters. In some embodiments, the method relates to increasing the patient's 6MWD by at least 300 meters. In some embodiments, the method relates to increasing the patient's 6MWD by at least 400 meters. In some embodiments, the 6MWD is tested after the patient has received 4 weeks of treatment utilizing an ActRII polypeptide disclosed herein. In some embodiments, the 6MWD is tested after the patient has received 8 weeks of treatment utilizing an ActRII polypeptide disclosed herein. In some embodiments, the 6MWD is tested after the patient has received 12 weeks of treatment utilizing an ActRII polypeptide disclosed herein. In some embodiments, the 6MWD is tested after the patient has received 16 weeks of treatment utilizing an ActRII polypeptide disclosed herein. In some embodiments, the 6MWD is tested after the patient has received 20 weeks of treatment utilizing an ActRII polypeptide disclosed herein. In some embodiments, the 6MWD is tested after the patient has received 22 weeks of treatment utilizing an ActRII polypeptide disclosed herein. In some embodiments, the 6MWD is tested after the patient has received 24 weeks of treatment utilizing an ActRII polypeptide disclosed herein. In some embodiments, the 6MWD is tested after the patient has received 26 weeks of treatment utilizing an ActRII polypeptide disclosed herein. In some embodiments, the 6MWD is tested after the patient has received 28 weeks of treatment utilizing an ActRII polypeptide disclosed herein. In some embodiments, the 6MWD is tested after the patient has received 48 weeks of treatment utilizing an ActRII polypeptide disclosed herein.

In some embodiments, the disclosure relates to methods of adjusting one or more measurements of exercise capacity (e.g., BDI) in the PcPH (e.g., WHO Group 2 and/or Group 5 PH) patient toward a more normal level (e.g., normal as compared to healthy people of similar age and sex), comprising administering to a patient in need thereof an effective amount of one or more variant ActRIIB polypeptides of the present disclosure (e.g., variant ActRIIB polypeptides in either homomeric or heteromeric forms). In some embodiments, the method relates to reducing the patient's BDI. In some embodiments, the method relates to lowering the patient's BDI by at least 0.5 index points. In some embodiments, the method relates to lowering the patient's BDI by at least 1 index points. In some embodiments, the method relates to lowering the patient's BDI by at least 1.5 index points. In some embodiments, the method relates to lowering the patient's BDI by at least 2 index points. In some embodiments, the method relates to lowering the patient's BDI by at least 2.5 index points. In some embodiments, the method relates to lowering the patient's BDI by at least 3 index points. In some embodiments, the method relates to lowering the patient's BDI by at least 3.5 index points. In some embodiments, the method relates to lowering the patient's BDI by at least 4 index points. In some embodiments, the method relates to lowering the patient's BDI by at least 4.5 index points. In some embodiments, the method relates to lowering the patient's BDI by at least 5 index points. In some embodiments, the method relates to lowering the patient's BDI by at least 5.5 index points. In some embodiments, the method relates to lowering the patient's BDI by at least 6 index points. In some embodiments, the method relates to lowering the patient's BDI by at least 6.5 index points. In some embodiments, the method relates to lowering the patient's BDI by at least 7 index points. In some embodiments, the method relates to lowering the patient's BDI by at least 7.5 index points. In some embodiments, the method relates to lowering the patient's BDI by at least 8 index points. In some embodiments, the method relates to lowering the patient's BDI by at least 8.5 index points. In some embodiments, the method relates to lowering the patient's BDI by at least 9 index points. In some embodiments, the method relates to lowering the patient's BDI by at least 9.5 index points. In some embodiments, the method relates to lowering the patient's BDI by at least 3 index points. In some embodiments, the method relates to lowering the patient's BDI by at least 10 index points. Echocardiography There are numerous clinical presentation factors, echocardiography features, and other features that could be indicative of PcPH (e.g., WHO Group 2 and/or Group 5 PH). For instance, patients who are ≥65 years old are at higher risk for WHO Group 2 (also known as PH-LHD). In patients suspected of having PcPH (e.g., WHO Group 2 and/or Group 5 PH), an echocardiogram may be used to image the effects of PH on the heart and estimate the mPAP from continuous wave Doppler measurements. In some embodiments, an echocardiogram may be used to measure the chamber sizes (e.g., the right atrium area and the right ventricle area), the magnitude of tricuspid regurgitation, the left ventricle eccentricity index and right ventricle contractility. The right ventricle contractility can be determined using several variables, such as the right ventricle longitudinal systolic strain/strain rate and right ventricle fractional area change, Tei index, and tricuspid annular plane systolic excursion. See. e.g., Galie N., et al Euro Heart J. (2016) 37, 67-119.

In some embodiments, an echocardiogram performed on a patient shows structural left heart abnormalities. In some embodiments, the structural left heart abnormality is a disease of the left heart valves. In some embodiments, the structural left heart abnormality is left atrium enlargement (e.g., >4.2 cm). In some embodiments, an electrocardiogram performed on a patient shows left ventricular hypertrophy (LVH) and/or left atrial hypertrophy/dilation (LAH). In some embodiments, an electrocardiogram performed on a patient shows atrial flutter/atrial fibrillation (AF/Afib). In some embodiments, an electrocardiogram performed on a patient shows left bundle branch block (LBBB). In some embodiments, an electrocardiogram performed on a patient shows the presence of Q waves. Id.

In a patient that has symptoms of left heart failure, an echocardiogram may be performed to evaluate various parameters. For instance, in some embodiments, an echocardiogram using Doppler performed on a patient may show indices of increased filling pressures and/or diastolic dysfunction (e.g., increased E/E′ or >Type 2-3 mitral flow abnormality). In some embodiments, imaging (e.g. echocardiogram. CT scan, chest X-ray, or MRI) performed on a patient shows Kerley B lines. In some embodiments, imaging (e.g. echocardiogram, CT scan, chest X-ray, or MRI) performed on a patient shows pleural effusion.

In some embodiments, imaging (e.g. echocardiogram, CT scan, chest X-ray, or MRI) performed on a patient shows pulmonary edema. In some embodiments, imaging (e.g., echocardiogram, CT scan, chest X-ray, or MRI) performed on a patient shows left atrium enlargement. Id.

In some embodiments, an echocardiogram may be performed to evaluate various parameters. For instance, in some embodiments, an echocardiogram may be utilized to measure the tricuspid annular plane systolic excursion (TAPSE). In some embodiments, an echocardiogram may be utilized to measure the right ventricular fractional area change (RVFAC). In some embodiments, an echocardiogram may be utilized to measure the right ventricular end-systolic area (RVESA). In some embodiments, an echocardiogram may be utilized to measure the right ventricular end-diastolic area (RVEDA). In some embodiments, an echocardiogram may be utilized to measure the right ventricular ejection fraction (RVEF). In some embodiments, an echocardiogram may be utilized to measure the right ventricular stroke volume (RVSV). In some embodiments, an echocardiogram may be utilized to measure the left ventricular ejection fraction (LVEF).

Furthermore, in a patient that has features of metabolic syndrome, imaging (e.g, an echocardiogram) may be performed to evaluate various parameters. For instance, in some embodiments, an echocardiogram performed on a patient shows the absence of right ventricle dysfunction (e.g., IpcPH). In some embodiments, an echocardiogram performed on a patient shows the presence of right ventricle dysfunction (e.g., CpcPH). In some embodiments, an echocardiogram performed on a patient shows the absence of mid systolic notching of the pulmonary artery flow. In some embodiments, an echocardiogram performed on a patient shows the absence of pericardial effusion. In some embodiments, the patient has a history of heart disease (past or current). In some embodiments, the patient has persistent atrial fibrillation. Id. In some embodiments, an Echo Score or the TAPSE/systolic pulmonary arterial pressure ratio are used to differentiate Cpc-PH from Ipc-PH. In some embodiments, an integrative score of five echocardiographic parameters (RV/LV ratio, left ventricular eccentricity index (LVET), E/E′, RV forming apex, width and inspiratory collapse of IVC) as well as “notching” of the RV outflow tract Doppler envelope may be used to distinguish between precapillary PH (e.g., PAH) from post-capillary PH (e.g., WHO Group 2 and/or Group 5 PH).

In some embodiments, a patient has diastolic dysfunction. In some embodiments, the method improves diastolic dysfunction in the patient. In some embodiments, the improvement in diastolic dysfunction is an improvement in the E/E′ ratio (a ratio of mitral inflow velocity (E) to mitral annular velocity (E′). In some embodiments, the improvement in diastolic dysfunction is an improvement in the isovolumic relaxation time (IVRT). In some embodiments, the improvement in the diastolic dysfunction is a lower RVSP. In some embodiments, the diastolic dysfunction results from one or more conditions selected from the group consisting of hypertension, diabetes, and advanced age.

Complications of PH In certain aspects, the disclosure relates to methods of treating, preventing, or reducing the progression rate and/or severity of one or more complications of PcPH (e.g., WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms). In some embodiments, the method relates to treating, preventing, or reducing the progression rate and/or severity of cell proliferation in the pulmonary artery of a PcPH patient. In some embodiments, the method relates to treating, preventing, or reducing the progression rate and/or severity of smooth muscle and/or endothelial cells proliferation in the pulmonary artery of a PcPH patient. In some embodiments, the method relates to treating, preventing, or reducing the progression rate and/or severity of angiogenesis in the pulmonary artery of a PcPH patient. In some embodiments, the method relates to increasing physical activity of a patient having PcPH. In some embodiments, the method relates to treating, preventing, or reducing the progression rate and/or severity of dyspnea in a PcPH patient. In some embodiments, the method relates to treating, preventing, or reducing the progression rate and/or severity of chest pain in a PcPH patient. In some embodiments, the method relates to treating, preventing, or reducing the progression rate and/or severity of fatigue in a PcPH patient. In some embodiments, the method relates to treating, preventing, or reducing the progression rate and/or severity of fibrosis in a PcPH patient. In some embodiments, the fibrosis is selected from the group consisting of left ventricular fibrosis, right ventricular fibrosis, and pulmonary fibrosis. In some embodiments, the method relates to treating, preventing, or reducing the progression rate and/or severity of left ventricular fibrosis in a PcPH patient. In some embodiments, the method relates to treating, preventing, or reducing the progression rate and/or severity of right ventricular fibrosis in a PcPH patient. In some embodiments, the method relates to treating, preventing, or reducing the progression rate and/or severity of pulmonary fibrosis in a PcPH patient. In some embodiments, the method relates to decreasing the patient's fibrosis by least 1% (e.g., at least 1%, 5, 10, 15, 20, 25, 30, 35, 40, 45, 50, 55, 60, 65, 70, 75, 80, 85, 90, 95, or 100%). In some embodiments, the method relates to decreasing the patient's fibrosis by at least 1%. In some embodiments, the method relates to decreasing the patient's fibrosis by at least 5%. In some embodiments, the method relates to decreasing the patient's fibrosis by at least 10%. In some embodiments, the method relates to decreasing the patient's fibrosis by at least 15%. In some embodiments, the method relates to decreasing the patient's fibrosis by at least 20%. In some embodiments, the method relates to decreasing the patient's fibrosis by at least 25%. In some embodiments, the method relates to decreasing the patient's fibrosis by at least 30%. In some embodiments, the method relates to decreasing the patient's fibrosis by at least 35%. In some embodiments, the method relates to decreasing the patient's fibrosis by at least 40%. In some embodiments, the method relates to decreasing the patient's fibrosis by at least 45%. In some embodiments, the method relates to decreasing the patient's fibrosis by at least 50%. In some embodiments, the method relates to decreasing the patient's fibrosis by at least 55%. In some embodiments, the method relates to decreasing the patient's fibrosis by at least 60%. In some embodiments, the method relates to decreasing the patient's fibrosis by at least 65%. In some embodiments, the method relates to decreasing the patient's fibrosis by at least 70%. In some embodiments, the method relates to decreasing the patient's fibrosis by at least 75%. In some embodiments, the method relates to decreasing the patient's fibrosis by at least 80%. In some embodiments, the method relates to decreasing the patient's fibrosis by at least 85%. In some embodiments, the method relates to decreasing the patient's fibrosis by at least 90%. In some embodiments, the method relates to decreasing the patient's fibrosis by at least 95%. In some embodiments, the method relates to decreasing the patient's fibrosis by at least 100%.

In some embodiments, the method relates to treating, preventing, or reducing the progression rate and/or severity of pulmonary vascular remodeling in a PcPH patient. In some embodiments, the method relates to treating, preventing, or reducing the progression rate and/or severity of cardiac remodeling in a PcPH patient. In some embodiments, the method relates to treating, preventing, or reducing the progression rate and/or severity of left cardiac remodeling in a PcPH patient. In some embodiments, the method relates to treating, preventing, or reducing the progression rate and/or severity of right cardiac remodeling in a PcPH patient. In some embodiments, the method relates to treating, preventing, or reducing the progression rate and/or severity of right ventricular hypertrophy in a PcPH patient. In some embodiments, the method relates to treating, preventing, or reducing the progression rate and/or severity of left ventricular hypertrophy in a PcPH patient. In some embodiments, the method relates to treating, preventing, or reducing the progression rate and/or severity of metabolic syndrome in a PcPH patient. In some embodiments, the method relates to treating, preventing, or reducing the progression rate and/or severity of left atrium dilation in a PcPH patient. In some embodiments, the method relates to treating, preventing, or reducing the progression rate and/or severity of an underlying condition (e.g., COPD, sleep apnea syndrome, CTEPH) in a PcPH patient.

### Complications or Comorbidities and Combination Therapies

In some embodiments, the disclosure contemplates methods of treating one or more complications of PcPH (e.g., smooth muscle and/or endothelial cell proliferation in the pulmonary artery, angiogenesis in the pulmonary artery, dyspnea, chest pain, pulmonary vascular remodeling, cardiac remodeling, right ventricular hypertrophy, left ventricular hypertrophy, left atrium dilation, pulmonary fibrosis, need for lung and/or heart transplant, and need for atrial septostomy)) comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates methods of preventing one or more complications of PcPH comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates methods of reducing the progression rate of one or more complications of PcPH (e.g., WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates methods of reducing the severity of one or more complications of PcPH (e.g., WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms).

In some embodiments, the disclosure contemplates methods of treating one or more comorbidities of PcPH (e.g., systemic hypertension, decreased renal function, diabetes mellitus, obesity, coronary artery disease (CAD), heart failure, and anemia) comprising administering to a patient in need thereof an effective amount of one or more variant ActRIIB polypeptides of the present disclosure (e.g., variant ActRIIB polypeptides in either homomeric or heteromeric forms). In some embodiments, the method results in the improvement of one or more comorbidities of PcPH (e.g., systemic hypertension, decreased renal function, diabetes mellitus, obesity, coronary artery disease (CAD), heart failure, and anemia). In some embodiments, the one or more comorbidities of PcPH are improved indirectly (e.g., due to an improvement in the patient's PH).

In some embodiments, the disclosure contemplates methods of reducing the progression rate of PcPH (e.g., WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates methods of reducing the progression rate of one or more complications of PcPH (e.g., WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates methods of reducing the severity of PcPH (e.g., WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates methods of reducing the severity of one or more complications of PcPH (e.g., WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates method of reducing the need to initiate treatment with a known treatment for PcPH comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates a method of reducing the need to increase the dose of prostacyclin in a patient (e.g., increasing the dose by at least 10%) comprising administering to a patient in need thereof an effective amount of one or more variant ActRIIB polypeptides of the present disclosure (e.g., variant ActRIIB polypeptides in either homomeric or heteromeric forms). In some embodiments, the disclosure contemplates a method of reducing the need for PcPH-specific hospitalization comprising administering to a patient in need thereof an effective amount of one or more variant ActRIIB polypeptides of the present disclosure (e.g., variant ActRIIB polypeptides in either homomeric or heteromeric forms). In some embodiments, PcPH-specific hospitalization is hospitalization of patient for at least 24 hours. In some embodiments, the disclosure contemplates a method of reducing the deterioration of PcPH comprising administering to a patient in need thereof an effective amount of one or more variant ActRIIB polypeptides of the present disclosure (e.g., variant ActRIIB polypeptides in either homomeric or heteromeric forms). In some embodiments, deterioration of PcPH comprises worsening in WHO functional class and/or a decrease of at least 15% in the 6MWD of the patient.

Optionally, methods disclosed herein for treating, preventing, or reducing the progression rate and/or severity of PcPH (e.g., WHO Group 2 and/or Group 5 PH), particularly treating, preventing, or reducing the progression rate and/or severity of one or more complications of PcPH, may further comprise administering to the patient one or more supportive therapies or additional active agents for treating PcPH. For example, the patient also may be administered one or more supportive therapies or active agents selected from the group consisting of: nitrates, hydralazine, prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, darusentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat, vericiguat, and riociguat); ASK-1 inhibitors (e.g., CIIA; SCH79797; GS-4997; MSC2032964A; 3H-naphtho[1,2,3-de]quiniline-2,7-diones, NQD1-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1->2)-beta-D-glucopyranosyl] oleanolic acid 28-O-beta-D-glucopyranosyl ester, 3-O-[a-L-rhamnopyranosyl-(1-->3)-beta-D-glucuronopyranosyl]oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1): oleanolic acid 3-O-β-D-glucopyranosyl (1→3)-β-D-glucopyranosiduronic acid (CS2); methyl 3,11-dioxoolean-12-en-28-olate (DIOXOL), ZCVI₄-2, Benzyl 3-dehydr-oxy-1,2,5-oxadiazolo[3′,4′:2.3]oleanolate), an LV assist device (LVAD), implantable cardioverter-defibrillator (ICD), valve replacement, valve repair, lung and/or heart transplantation. In some embodiments, the methods described herein may further comprise administering to the patient parental prostacyclin. In some embodiments, the methods described herein may further comprise administering to the patient one additional supportive therapy or additional active agent (i.e., double therapy) for treating PcPH. In some embodiments, the methods described herein may further comprise administering to the patient two additional supportive therapies or additional active agents (i.e., triple therapy) for treating PcPH. In some embodiments, the methods described herein may further comprise administering to the patient three additional supportive therapies or additional active agents (i.e., quadruple therapy) for treating PcPH.

In some embodiments, the methods described herein may further comprise administering to the patient an angiotensin antagonist (e.g., angiotensin receptor blocker, ARB). In some embodiments, a patient is further administered one or more ARBs selected from the group consisting of losartan, irbesartan, olmesartan, candesartan, valsartan, fimasartan, azilsartan, salprisartan, and telmisartan. In some embodiments, a patient is administered losartan. In some embodiments, a patient is administered irbesartan. In some embodiments, a patient is administered olmesartan. In some embodiments, a patient is administered candesartan. In some embodiments, a patient is administered valsartan. In some embodiments, a patient is administered fimasartan. In some embodiments, a patient is administered azilsartan. In some embodiments, a patient is administered salprisartan. In some embodiments, a patient is administered telmisartan. In some embodiments, the methods described herein may further comprise administering to the patient a neprilysin inhibitor and an angiotensin antagonist (e.g., sacubitril/valsartan; also known as LCZ696).

In some embodiments, the methods described herein may further comprise administering to the patient one or more ACE inhibitors. In some embodiments, the one or more ACE inhibitors are selected from the group consisting of benazepril, captopril, enalapril, lisinopril, perindopril, ramipril (e.g., ramipen), trandolapril, and zofenopril. In some embodiments, a patient is administered benazepril. In some embodiments, a patient is administered captopril. In some embodiments, a patient is administered enalapril. In some embodiments, a patient is administered lisinopril. In some embodiments, a patient is administered perindopril. In some embodiments, a patient is administered ramipril. In some embodiments, a patient is administered trandolapril. In some embodiments, a patient is administered zofenopril. In some embodiments, the methods described herein may further comprise administering to the patient an ARB and an ACE inhibitor. In some embodiments, an alternative approach to angiotensin antagonism is to combine an ACE inhibitor and/or ARB with an aldosterone antagonist.

In some embodiments, the methods described herein may further comprise administering to the patient one or more neprilysin inhibitors. In some embodiments, the one or more neprilysin inhibitors are selected from the group consisting of thiorphan, phosphoramidon, candoxatrilat, candoxatril, ecadotril, omapatrilat, LBQ657, and sacubitril. In some embodiments, a patient is administered thiorphan. In some embodiments, a patient is administered phosphoramidon. In some embodiments, a patient is administered candoxatrilat. In some embodiments, a patient is administered candoxatril. In some embodiments, a patient is administered ecadotril. In some embodiments, a patient is administered omapatrilat. In some embodiments, a patient is administered LBQ657. In some embodiments, a patient is administered sacubitril. In some embodiments, the methods described herein may further comprise administering to the patient a neprilysin inhibitor and an ARB (e.g., sacubitril/valsartan; also known as LCZ696).

In some embodiments, the methods described herein may further comprise administering to the patient an angiotensin receptor-neprilysin inhibitor (ARNI). In some embodiments, the ARNI is sacubitril/valsartan (Entresto®). In some embodiments, a patient is administered sacubitril/valsartan (Entresto®).

In some embodiments, the methods described herein may further comprise administering to the patient one or more beta-blockers. In some embodiments, the one or more beta-blockers are selected from the group consisting of bisoprolol, carvedilol, metoprolol succinate (CR/XL), and nebivolol. In some embodiments, a patient is administered bisoprolol. In some embodiments, a patient is administered carvedilol. In some embodiments, a patient is administered metoprolol succinate (CR/XL). In some embodiments, a patient is administered nebivolol.

In some embodiments, the methods described herein may further comprise administering to the patient one or more mineralocorticoid receptor antagonists (MRA). In some embodiments, the one or more MRA are selected from the group consisting of eplerenone and spironolactone. In some embodiments, a patient is administered eplerenone. In some embodiments, a patient is administered spironolactone.

In some embodiments, the methods described herein may further comprise administering to the patient one or more hyperpolarization-activated cyclic nucleotide-gated (HCN) channel blockers. In some embodiments, the one or more HCN are selected from the group consisting of ivabradine. ZD7288, cilobradine, zatebradine, alinidine, clonidine, falipamil, TH92:20, and YM758. In some embodiments, a patient is administered ivabradine. In some embodiments, a patient is administered ZD7288. In some embodiments, a patient is administered cilobradine. In some embodiments, a patient is administered zatebradine. In some embodiments, a patient is administered alinidine. In some embodiments, a patient is administered clonidine. In some embodiments, a patient is administered falipamil. In some embodiments, a patient is administered TH92:20. In some embodiments, a patient is administered YM758.

In some embodiments, the one or more supportive therapies or additional active agents for treating PcPH are administered prior to administration of the variant ActRIIB polypeptide. In some embodiments, the one or more supportive therapies or additional active agents for treating PcPH are administered in combination with the variant ActRIIB polypeptide. In some embodiments, the one or more supportive therapies or additional active agents for treating PcPH are administered after the administration of the variant ActRIIB polypeptide. As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

Transplant free survival Lung and/or heart transplantation is a surgical treatment option for patients with PcPH, and is often recommended for patients who don't respond to less invasive therapies (e.g., vasodilator therapy). Generally, PcPH patients who receive lung and/or heart transplantation have Functional Class III or Class IV pulmonary hypertension in accordance with the World Health Organization's functional classification system for pulmonary hypertension.

In certain aspects, the disclosure relates to methods of treating, preventing, or reducing the progression rate and/or severity of PcPH comprising administering to a patient in need thereof an effective amount of one or more variant ActRIIB polypeptides of the present disclosure (e.g., variant ActRIIB polypeptides in either homomeric or heteromeric forms), wherein the method increases the patient's transplant free survival by at least 1% (e.g., at least 1%, 2%, 3%, 4%, 5%, 10%, 15%, 20%, 250, 30%, 35%, 40%, 45%, 50%, 55%, 60%, 65%, 70%, 75%, 80%, 85%, 90%, 95%, or 100%. In some embodiments, the method relates to increasing the patient's transplant free survival by at least 1%. In some embodiments, the method relates to increasing the patient's transplant free survival by at least 2%. In some embodiments, the method relates to increasing the patient's transplant free survival by at least 3%. In some embodiments, the method relates to increasing the patient's transplant free survival by at least 4%. In some embodiments, the method relates to increasing the patient's transplant free survival by at least 5%. In some embodiments, the method relates to increasing the patient's transplant free survival by at least 10%. In some embodiments, the method relates to increasing the patient's transplant free survival by at least 15%. In some embodiments, the method relates to increasing the patient's transplant free survival by at least 20%. In some embodiments, the method relates to increasing the patient's transplant free survival by at least 25%. In some embodiments, the method relates to increasing the patient's transplant free survival by at least 30%. In some embodiments, the method relates to increasing the patient's transplant free survival by at least 35%. In some embodiments, the method relates to increasing the patient's transplant free survival by at least 40%. In some embodiments, the method relates to increasing the patient's transplant free survival by at least 45%. In some embodiments, the method relates to increasing the patient's transplant free survival by at least 50%. In some embodiments, the method relates to increasing the patient's transplant free survival by at least 55%. In some embodiments, the method relates to increasing the patient's transplant free survival by at least 60%. In some embodiments, the method relates to increasing the patient's transplant free survival by at least 65%. In some embodiments, the method relates to increasing the patient's transplant free survival by at least 70%. In some embodiments, the method relates to increasing the patient's transplant free survival by at least 75%. In some embodiments, the method relates to increasing the patient's transplant free survival by at least 80%. In some embodiments, the method relates to increasing the patient's transplant free survival by at least 85%. In some embodiments, the method relates to increasing the patient's transplant free survival by at least 90%. In some embodiments, the method relates to increasing the patient's transplant free survival by at least 95%. In some embodiments, the method relates to increasing the patient's transplant free survival by at least 100%. In some embodiments, the method relates to increasing the patient's transplant free survival as compared to controls over 1 year. In some embodiments, the method relates to increasing the patient's transplant free survival as compared to controls over 2 years. In some embodiments, the method relates to increasing the patient's transplant free survival as compared to controls over 3 years. In some embodiments, the method relates to increasing the patient's transplant free survival as compared to controls over 4 years. In some embodiments, the method relates to increasing the patient's transplant free survival as compared to controls over 5 years. In some embodiments, the method relates to increasing the patient's transplant free survival as compared to controls over 6 years. In some embodiments, the method relates to increasing the patient's transplant free survival as compared to controls over 7 years.

### Death

In certain aspects, the disclosure relates to methods of reducing the risk of death in patients with PcPH comprising administering to a patient in need thereof an effective amount of one or more variant ActRIIB polypeptides of the present disclosure (e.g., variant ActRIIB polypeptides in either homomeric or heteromeric forms), wherein the method reduces the patient's risk of death by at least 1% (e.g., at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, the method relates to reducing the patient's risk of death by at least 1%. In some embodiments, the method relates to reducing the patient's risk of death by at least 2%. In some embodiments, the method relates to reducing the patient's risk of death by at least 3%. In some embodiments, the method relates to reducing the patient's risk of death by at least 4%. In some embodiments, the method relates to reducing the patient's risk of death by at least 5%. In some embodiments, the method relates to reducing the patient's risk of death by at least 10%. In some embodiments, the method relates to reducing the patient's risk of death by at least 15%. In some embodiments, the method relates to reducing the patient's risk of death by at least 20%. In some embodiments, the method relates to reducing the patient's risk of death by at least 25%. In some embodiments, the method relates to reducing the patient's risk of death by at least 30%. In some embodiments, the method relates to reducing the patient's risk of death by at least 35%. In some embodiments, the method relates to reducing the patient's risk of death by at least 40%. In some embodiments, the method relates to reducing the patient's risk of death by at least 45%. In some embodiments, the method relates to reducing the patient's risk of death by at least 50%. In some embodiments, the method relates to reducing the patient's risk of death by at least 55%. In some embodiments, the method relates to reducing the patient's risk of death by at least 60%. In some embodiments, the method relates to reducing the patient's risk of death by at least 65%. In some embodiments, the method relates to reducing the patient's risk of death by at least 70%. In some embodiments, the method relates to reducing the patient's risk of death by at least 75%. In some embodiments, the method relates to reducing the patient's risk of death by at least 80%. In some embodiments, the method relates to reducing the patient's risk of death by at least 85%. In some embodiments, the method relates to reducing the patient's risk of death by at least 90%. In some embodiments, the method relates to reducing the patient's risk of death by at least 95%. In some embodiments, the method relates to reducing the patient's risk of death by at least 100%. In some embodiments, the method reduces the risk of hospitalization for one or more complications associated with PcPH.

### Composite Clinical Endpoint

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the method reduces the patient's composite clinical endpoint by at least 1% (e.g., at least 1%, 2%, 3%, 4%, 50, 10%, 15%, 20%, 25%, 30%, 35%, 40%, 45%, 50%, 55%, 60%, 65%, 70%, 75%, 80%, 85%, 90%, 95%, or 100%). In some embodiments, the method relates to reducing the patient's composite clinical endpoint by at least 1%. In some embodiments, the method relates to reducing the patient's composite clinical endpoint by at least 2%. In some embodiments, the method relates to reducing the patient's composite clinical endpoint by at least 3%. In some embodiments, the method relates to reducing the patient's composite clinical endpoint by at least 4%. In some embodiments, the method relates to reducing the patient's composite clinical endpoint by at least 5%. In some embodiments, the method relates to reducing the patient's composite clinical endpoint by at least 10%. In some embodiments, the method relates to reducing the patient's composite clinical endpoint by at least 15%. In some embodiments, the method relates to reducing the patient's composite clinical endpoint by at least 20%. In some embodiments, the method relates to reducing the patient's composite clinical endpoint by at least 25%. In some embodiments, the method relates to reducing the patient's composite clinical endpoint by at least 30%. In some embodiments, the method relates to reducing the patient's composite clinical endpoint by at least 35%. In some embodiments, the method relates to reducing the patient's composite clinical endpoint by at least 40%. In some embodiments, the method relates to reducing the patient's composite clinical endpoint by at least 45%. In some embodiments, the method relates to reducing the patient's composite clinical endpoint by at least 50%. In some embodiments, the method relates to reducing the patient's composite clinical endpoint by at least 55%. In some embodiments, the method relates to reducing the patient's composite clinical endpoint by at least 60%. In some embodiments, the method relates to reducing the patient's composite clinical endpoint by at least 65%. In some embodiments, the method relates to reducing the patient's composite clinical endpoint by at least 70%. In some embodiments, the method relates to reducing the patient's composite clinical endpoint by at least 75%. In some embodiments, the method relates to reducing the patient's composite clinical endpoint by at least 80%. In some embodiments, the method relates to reducing the patient's composite clinical endpoint by at least 85%. In some embodiments, the method relates to reducing the patient's composite clinical endpoint by at least 90%. In some embodiments, the method relates to reducing the patient's composite clinical endpoint by at least 95%. In some embodiments, the method relates to reducing the patient's composite clinical endpoint by at least 100%. In some embodiments, the method reduces the risk of hospitalization for one or more complications associated with PcPH. In some embodiments, the composite clinical endpoint comprises one or more endpoints selected from the group consisting of clinical worsening, hospitalization, and death.

Functional Classes PcPH (e.g., WHO Group 2 and Group 5 PH) at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Functional Class I: pulmonary hypertension without resulting limitation of physical activity; ordinary physical activity does not cause undue dyspnea or fatigue, chest pain or near syncope; Functional Class II: pulmonary hypertension resulting in slight limitation of physical activity; patient comfortable at rest; ordinary physical activity causes undue dyspnea or fatigue, chest pain or near syncope; Functional Class III: pulmonary hypertension resulting in marked limitation of physical activity; patient comfortable at rest; less than ordinary activity causes undue dyspnea or fatigue, chest pain or near syncope; Functional Class IV: pulmonary hypertension resulting in inability to carry out any physical activity without symptoms; patient manifests signs of right-heart failure; dyspnea and/or fatigue may be present even at rest; discomfort is increased by any physical activity.

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has Functional Class I, Functional Class II, Functional Class III, or Functional Class IV pulmonary hypertension as recognized by the WHO. In some embodiments, the method relates to a patient that has Functional Class I pulmonary hypertension as recognized by the WHO. In some embodiments, the method relates to a patient that has Functional Class II pulmonary hypertension as recognized by the WHO. In some embodiments, the method relates to a patient that has Functional Class III pulmonary hypertension as recognized by the WHO. In some embodiments, the method relates to a patient that has Functional Class IV pulmonary hypertension as recognized by the WHO. In some embodiments, the method relates to patients having Functional Class II or Class III pulmonary hypertension as recognized by the WHO. In some embodiments, the method relates to patients having Functional Class II, Class Ill, or Class IV pulmonary hypertension as recognized by the WHO. In some embodiments, the method relates to patients having Functional Class I, Class II, Class III, or Class IV pulmonary hypertension as recognized by the WHO. In some embodiments, the method delays clinical worsening of PcPH. In some embodiments, the method delays clinical worsening of PcPH in accordance with the WHO's functional classification system for pulmonary hypertension.

In some embodiments, the disclosure relates to methods of preventing or reducing pulmonary hypertension Functional Class progression comprising administering to a patient in need thereof an effective amount of one or more variant ActRIIB polypeptides of the present disclosure (e.g., variant ActRIIB polypeptides in either homomeric or heteromeric forms). In some embodiments, the reduction in Functional Class progression is a delay in Functional Class progression. In some embodiments, the method relates to preventing or decreasing pulmonary hypertension functional class progression as recognized by the WHO. In some embodiments, the method relates to a patient that has Functional Class I pulmonary hypertension as recognized by the WHO. In some embodiments, the method relates to preventing or reducing patient progression from Functional Class I pulmonary hypertension to Functional Class II pulmonary hypertension as recognized by the WHO. In some embodiments, the method relates to a patient that has Functional Class II pulmonary hypertension as recognized by the WHO. In some embodiments, the method relates to preventing or reducing patient progression from Functional Class 11 pulmonary hypertension to Functional Class 111 pulmonary hypertension as recognized by the WHO. In some embodiments, the method relates to a patient that has Functional Class III pulmonary hypertension as recognized by the WHO. In some embodiments, the method relates to preventing or reducing patient progression from Functional Class 111 pulmonary hypertension to Functional Class IV pulmonary hypertension as recognized by the WHO.

In certain aspects, the disclosure relates to methods of promoting or increasing pulmonary hypertension Functional Class regression in a PcPH patient comprising administering to a patient in need thereof an effective amount of one or more variant ActRIIB polypeptides of the present disclosure (e.g., variant ActRIIB polypeptides in either homomeric or heteromeric forms), wherein the patient has Functional Class I, Functional Class 11, Functional Class III, or Functional Class IV pulmonary hypertension as recognized by the WHO. In some embodiments, the method relates to preventing or delaying patient progression from Functional Class I pulmonary hypertension to Functional Class II pulmonary hypertension as recognized by the WHO. In some embodiments, the method relates to promoting patient regression from Functional Class II pulmonary hypertension to Functional Class I pulmonary hypertension as recognized by the WHO. In some embodiments, the method relates to preventing or delaying patient progression from Functional Class II pulmonary hypertension to Functional Class III pulmonary hypertension as recognized by the WHO. In some embodiments, the method relates to promoting patient regression from Functional Class III pulmonary hypertension to Functional Class II pulmonary hypertension as recognized by the WHO. In some embodiments, the method relates to promoting patient regression from Functional Class III pulmonary hypertension to Functional Class I pulmonary hypertension as recognized by the WHO. In some embodiments, the method relates to preventing or delaying patient progression from Functional Class III pulmonary hypertension to Functional Class IV pulmonary hypertension as recognized by the WHO. In some embodiments, the method relates to promoting patient regression from Functional Class IV pulmonary hypertension to Functional Class III pulmonary hypertension as recognized by the WHO. In some embodiments, the method relates to promoting patient regression from Functional Class IV pulmonary hypertension to Functional Class II pulmonary hypertension as recognized by the WHO. In some embodiments, the method relates to promoting patient regression from Functional Class IV pulmonary hypertension to Functional Class I pulmonary hypertension as recognized by the WHO.

The New York Heart Association (NYHA) functional classification (Table 3) has been used to describe the severity of symptoms and exercise intolerance in patients with pulmonary hypertension. The NYHA functional classification system provides a rapid assessment of patients' functional status in everyday clinical practice and is a well-established means of predicting prognosis. The four functional classes recognized by the NYHA functional classification system are shown in Table 3.

TABLE 3
New York Heart Association (NYHA) functional classification
of pulmonary hypertension based on severity of
symptoms and physical activity

| | |
|---|---|
| Class I | No limitation of physical activity. Ordinary physical activity does |
| | not cause undue breathlessness, fatigue, or palpitations. |
| Class II | Slight limitation of physical activity. Comfortable at rest, but |
| | ordinary physical activity results in undue breathlessness, fatigue, |
| | or palpitations. |
| Class III | Marked limitation of physical activity. Comfortable at rest, but |
| | less than ordinary physical activity results in undue |
| | breathlessness, fatigue, or palpitations. |
| Class IV | Unable to carry on any physical activity without discomfort. |
| | Symptoms at rest can be present If any physical activity is |
| | undertaken, discomfort is increased. |

In certain aspects, the disclosure relates to methods of treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms), wherein the patient has Functional Class I, Functional Class II, Functional Class III, or Functional Class IV pulmonary hypertension as recognized by the NYHA.

In some embodiments, the method relates to a patient that has Functional Class 1 pulmonary hypertension as recognized by the NYHA. In some embodiments, a patient with Functional Class I pulmonary hypertension as recognized by the NYHA has no limitation of physical activity. In some embodiments, a patient with Functional Class I pulmonary hypertension as recognized by the NYHA experiences physical activity that does not cause undue breathlessness, fatigue, and/or palpitations. In some embodiments, the method relates to a patient that has Functional Class II pulmonary hypertension as recognized by the NYHA. In some embodiments, a patient with Functional Class II pulmonary hypertension as recognized by the NYHA has slight limitation of physical activity. In some embodiments, a patient with Functional Class II pulmonary hypertension as recognized by the NYHA experiences ordinary physical activity resulting in undue breathlessness, fatigue, or palpitations. In some embodiments, the method relates to a patient that has Functional Class III pulmonary hypertension as recognized by the NYHA. In some embodiments, a patient with Functional Class III pulmonary hypertension as recognized by the NYHA has marked limitation of physical activity. In some embodiments, a patient with Functional Class III pulmonary hypertension as recognized by the NYHA experiences less than ordinary physical activity resulting in undue breathlessness, fatigue, or palpitations. In some embodiments, the method relates to a patient that has Functional Class IV pulmonary hypertension as recognized by the NYHA. In some embodiments, a patient with Functional Class IV pulmonary hypertension as recognized by the NYHA is unable to carry on any physical activity without discomfort. In some embodiments, a patient with Functional Class IV pulmonary hypertension as recognized by the NYHA experiences symptoms at rest, as well as when any physical activity is undertaken, discomfort is increased. In some embodiments, the method relates to patients having Functional Class II or Class III pulmonary hypertension as recognized by the NYHA.

In some embodiments, the method relates to patients having Functional Class II, Class III, or Class IV pulmonary hypertension as recognized by the NYHA. In some embodiments, the method relates to patients having Functional Class I, Class II, Class III, or Class IV pulmonary hypertension as recognized by the NYHA. In some embodiments, the method delays clinical worsening of PcPH. In some embodiments, the method delays clinical worsening of PcPH in accordance with the NYHA's functional classification system for pulmonary hypertension.

In some embodiments, the disclosure relates to methods of preventing or reducing pulmonary hypertension Functional Class progression comprising administering to a patient in need thereof an effective amount of one or more variant ActRIIB polypeptides of the present disclosure (e.g., variant ActRIIB polypeptides in either homomeric or heteromeric forms). In some embodiments, the reduction in Functional Class progression is a delay in Functional Class progression. In some embodiments, the method relates to preventing or decreasing pulmonary hypertension functional class progression as recognized by the NYHA. In some embodiments, the disclosure relates to methods of promoting or increasing pulmonary hypertension Functional Class regression in a PcPH patient comprising administering to a patient in need thereof an effective amount of one or more variant ActRIIB polypeptides of the present disclosure (e.g., variant ActRIIB polypeptides in either homomeric or heteromeric forms), wherein the patient has Functional Class I, Functional Class II, Functional Class III, or Functional Class IV pulmonary hypertension as recognized by the NYHA. In some embodiments, the method relates to preventing or delaying patient progression from Functional Class I pulmonary hypertension to Functional Class 11 pulmonary hypertension as recognized by the NYHA. In some embodiments, the method relates to promoting patient regression from Functional Class II pulmonary hypertension to Functional Class I pulmonary hypertension as recognized by the NYHA. In some embodiments, the method relates to preventing or delaying patient progression from Functional Class II pulmonary hypertension to Functional Class III pulmonary hypertension as recognized by the NYHA. In some embodiments, the method relates to promoting patient regression from Functional Class III pulmonary hypertension to Functional Class 11 pulmonary hypertension as recognized by the NYHA. In some embodiments, the method relates to promoting patient regression from Functional Class III pulmonary hypertension to Functional Class I pulmonary hypertension as recognized by the NYHA. In some embodiments, the method relates to preventing or delaying patient progression from Functional Class III pulmonary hypertension to Functional Class IV pulmonary hypertension as recognized by the NYHA. In some embodiments, the method relates to promoting patient regression from Functional Class IV pulmonary hypertension to Functional Class III pulmonary hypertension as recognized by the NYHA. In some embodiments, the method relates to promoting patient regression from Functional Class IV pulmonary hypertension to Functional Class II pulmonary hypertension as recognized by the NYHA. In some embodiments, the method relates to promoting patient regression from Functional Class IV pulmonary hypertension to Functional Class I pulmonary hypertension as recognized by the NYHA.

In some embodiments, functional class regression is tested after the patient has received 4 weeks of treatment utilizing an ActRII polypeptide disclosed herein. In some embodiments, functional class regression is tested after the patient has received 8 weeks of treatment utilizing an ActRII polypeptide disclosed herein. In some embodiments, functional class regression is tested after the patient has received 12 weeks of treatment utilizing an ActRII polypeptide disclosed herein. In some embodiments, functional class regression is tested after the patient has received 16 weeks of treatment utilizing an ActRII polypeptide disclosed herein. In some embodiments, functional class regression is tested after the patient has received 20 weeks of treatment utilizing an ActRII polypeptide disclosed herein. In some embodiments, functional class regression is tested after the patient has received 22 weeks of treatment utilizing an ActRII polypeptide disclosed herein. In some embodiments, functional class regression is tested after the patient has received 24 weeks of treatment utilizing an ActRII polypeptide disclosed herein. In some embodiments, functional class regression is tested after the patient has received 26 weeks of treatment utilizing an ActRII polypeptide disclosed herein. In some embodiments, functional class regression is tested after the patient has received 28 weeks of treatment utilizing an ActRII polypeptide disclosed herein. In some embodiments, functional class regression is tested after the patient has received 48 weeks of treatment utilizing an ActRII polypeptide disclosed herein.

### Sustained Therapeutic Effect

In certain aspects, the disclosure relates to methods of treating, preventing, or reducing the progression rate and/or severity of PcPH in a sustained manner comprising administering to a patient in need thereof an effective amount of one or more variant ActRIIB polypeptides of the present disclosure (e.g., variant ActRIIB polypeptides in either homomeric or heteromeric forms). In some embodiments, the sustained manner comprises a persistent therapeutic effect following the reduction in administration of an ActRII polypeptide described herein. In some embodiments, the sustained manner comprises a persistent therapeutic effect following the withdrawal of administration of an ActRII polypeptide described herein. In some embodiments, the persistent therapeutic effect relates to maintaining functional or hematologic measurements over time. In some embodiments, the persistent therapeutic effect is measured as a sustained reduction in PVR In some embodiments, the patient's PVR level does not increase for at least 1 week to at least 12 weeks following withdrawal of an ActRII polypeptide treatment described herein. In some embodiments, the patient's PVR level does not increase for at least 1 week following withdrawal of an ActRII polypeptide treatment described herein. In some embodiments, the patient's PVR level does not increase for at least 2 weeks following withdrawal of an ActRII polypeptide treatment described herein. In some embodiments, the patient's PVR level does not increase for at least 3 weeks following withdrawal of an ActRII polypeptide treatment described herein. In some embodiments, the patient's PVR level does not increase for at least 4 weeks following withdrawal of an ActRII polypeptide treatment described herein. In some embodiments, the patient's PVR level does not increase for at least 5 weeks following withdrawal of an ActRII polypeptide treatment described herein. In some embodiments, the patient's PVR level does not increase for at least 6 weeks following withdrawal of an ActRII polypeptide treatment described herein. In some embodiments, the patient's PVR level does not increase for at least 1 month to at least 6 months following withdrawal of an ActRII polypeptide treatment described herein.

In certain aspects, the disclosure relates to methods of treating or preventing cardiopulmonary remodeling associated with PcPH in a patient, comprising administering to a patient in need thereof an effective amount of one or more variant ActRIIB polypeptides of the present disclosure (e.g., variant ActRIIB polypeptides in either homomeric or heteromeric forms), wherein said method slows down cardiac remodeling and/or reverses cardiac remodeling. In some embodiments, the reversal is a sustained reversal. In some embodiments, the cardiac remodeling is left cardiac remodeling. In some embodiments, the cardiac remodeling is right cardiac remodeling. In some embodiments, the cardiac remodeling is ventricle remodeling. In some embodiments, the ventricle remodeling is left ventricular remodeling. In some embodiments, the ventricle remodeling is right ventricular remodeling. In some embodiments, the cardiac remodeling is ventricular dilation. In some embodiments, the method decreases interventricular septal end diastole. In some embodiments, the method decreases posterior wall end diastole.

In some embodiments, echocardiographic measurements may be used to assess the persistent therapeutic effect. In some embodiments, the echocardiographic measurements include, but are not limited to, RV fractional area change (RVFAC), sPAP, tricuspid annular systolic velocity (TASV), and Tei index. In some embodiments, a patient treated with an ActRII polypeptide disclosed herein shows a persistent therapeutic effect. In some embodiments, the persistent therapeutic effect results in decreased intrusion of the ventral wall into the left ventricle. In some embodiments, the persistent therapeutic effect results in an increase in right ventricular fractional area change (RVFAC).

### Known Treatments for PcPH

There is no known cure for PcPH (e.g., WHO Group 2 and/or Group 5 PH); current methods of treatment focus on prolonging patient lifespan and enhancing patient quality of life. This is usually associated with good exercise capacity, good right ventricle function, and a low mortality risk (e.g., bringing the patient to and/or keeping the patient in WHO Functional Class 1 or Functional Class II). Current methods of treatment of PcPH may include administration of: vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of PcPH has also been carried out using oxygen therapy, atrial septostomy, pulmonary thromboendarterectomy, and lung and/or heart transplantation. Each of these methods, however, suffers from one or multiple drawbacks which may include lack of effectiveness, serious side effects, low patient compliance, and high cost. In certain aspects, the method relate to treating, preventing, or reducing the progression rate and/or severity of PcPH (e.g., treating, preventing, or reducing the progression rate and/or severity of one or more complications of PcPH in WHO Group 2 and/or Group 5 PH) comprising administering to a patient in need thereof an effective amount of one or more variant ActRIIB polypeptides of the present disclosure (e.g., variant ActRIIB polypeptides in either homomeric or heteromeric forms) in combination with one or more additional active agents and/or supportive therapies for treating PcPH (e.g., vasodilators such as prostacyclin, epoprostenol, and sildenafil; endothelin receptor antagonists such as bosentan; calcium channel blockers such as amlodipine, diltiazem, and nifedipine; anticoagulants such as warfarin; diuretics; oxygen therapy; atrial septostomy; pulmonary thromboendarterectomy: LVAD; implantable cardioverter-defibrillator (ICD); valve replacement; valve repair; and lung and/or heart transplantation); bardoxolone methyl or a derivative thereof; oleanolic acid or derivative thereof.

### Measuring Hematologic Parameters in a Patient

In certain embodiments, the present disclosure provides methods for managing a patient that has been treated with, or is a candidate to be treated with, one or more variant ActRIIB polypeptides of the present disclosure (e.g., variant ActRIIB polypeptides in either homomeric or heteromeric forms) by measuring one or more hematologic parameters in the patient. The hematologic parameters may be used to evaluate appropriate dosing for a patient who is a candidate to be treated with one or more variant ActRIIB polypeptides of the present disclosure, to monitor the hematologic parameters during treatment, to evaluate whether to adjust the dosage during treatment with one or more variant ActRIIB polypeptides of the disclosure, and/or to evaluate an appropriate maintenance dose of one or more variant ActRIIB polypeptides of the disclosure. If one or more of the hematologic parameters are outside the normal level, dosing with one or more variant ActRIIB polypeptides may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art recognized methods. In other embodiments, hematologic parameters such as white blood cell levels, platelet levels, and neutrophil levels may be measured using art recognized methods. Such parameters may be determined using a blood sample from a patient. Increases in red blood cell levels, hemoglobin levels, and/or hematocrit levels may cause increases in blood pressure. Decreases in white blood cell levels, platelet levels, and/or neutrophil levels may indicate a need to decrease, delay, or discontinue treatment of the administration of one or more variant ActRIIB polypeptides of the disclosure.

In one embodiment, if one or more hematologic parameters are outside the normal range or on the high side of normal in a patient who is a candidate to be treated with one or more variant ActRIIB polypeptides, then onset of administration of the one or more variant ActRIIB polypeptides of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. In some embodiments, administration of the one or more variant ActRIIB polypeptides of the disclosure may be delayed or reduced until the levels have returned to a normal or acceptable level if the patients have higher than normal red blood cell levels and/or hemoglobin levels (e.g., hemoglobin levels>16.0 g/dL or hemoglobin levels>18.0 g/dL), then administration of the one or more ActRII polypeptides of the disclosure may be delayed or reduced until the levels have returned to a normal or acceptable level. In some embodiments, a normal or acceptable level of hemoglobin includes patients with hemoglobin levels between 8-15 g/dl. In some embodiments, a normal or acceptable level of hemoglobin includes patients with hemoglobin levels of <18 g/dl. In some embodiments, a normal or acceptable level of hemoglobin increase overtime includes patients whose hemoglobin levels increase less than 2 g/dL over the first period of time in treatment. In some embodiments, the first period of time is 3 weeks. For patients having lower than normal white blood cell counts (e.g., leukopenia; white blood cell count<3000/mm³ or <3.0×10⁹/L (Grade 2), then administration of the one or more variant ActRIIB polypeptides of the disclosure may be delayed or reduced until the levels have returned to a normal or acceptable level for patients having lower than normal white blood cell counts (e.g., leukopenia; white blood cell count<2000/mm³ or <2.0×10⁹/L (Grade 3)), then administration of the one or more variant ActRIIB polypeptides of the disclosure may be delayed or reduced until the levels have returned to a normal or acceptable level. For patients having lower than normal platelet counts (e.g., thrombocytopenia; platelet count<75,000/mm³ or <75.0×10⁹/L (Grade 2)), then administration of the one or more variant ActRIIB polypeptides of the disclosure may be delayed or reduced until the levels have returned to a normal or acceptable level. For patients having lower than normal platelet counts (e.g., thrombocytopenia; platelet count<50,000/mm³ or <50.0×10⁹/L (Grade 3)), then administration of the one or more variant ActRIIB polypeptides of the disclosure may be delayed or reduced until the levels have returned to a normal or acceptable level. For patients having lower than normal neutrophil counts (e.g., neutropenia; neutrophil count<1500/mm³ or <1.5×10⁹/L (Grade 2)), then administration of the one or more variant ActRIIB polypeptides of the disclosure may be delayed or reduced until the levels have returned to a normal or acceptable level. For patients having lower than normal neutrophil counts (e.g., neutropenia; neutrophil count<1000/mm³ or <1.0×10⁹/L (Grade 3)), then administration of the one or more variant ActRIIB polypeptides of the disclosure may be delayed or reduced until the levels have returned to a normal or acceptable level.

In certain embodiments, if one or more hematologic parameters are outside the normal range or on the high side of normal in a patient who is a candidate to be treated with one or more variant ActRIIB polypeptides, then the onset of administration may not be delayed.

However, the dosage amount or frequency of dosing of the one or more variant ActRIIB polypeptides of the disclosure may be set at an amount that would reduce the risk of an unacceptable increase in the hematologic parameters arising upon administration of the one or more variant ActRIIB polypeptides of the disclosure. Alternatively, a therapeutic regimen may be developed for the patient that combines one or more variant ActRIIB polypeptides with a therapeutic agent that addresses the undesirable level of the hematologic parameter. For example, if the patient has elevated blood pressure, then a therapeutic regimen may be designed involving administration of one or more variant ActRIIB polypeptides and a blood pressure lowering agent. For a patient having lower than desired iron stores, a therapeutic regimen may be developed involving one or more variant ActRIIB polypeptides of the disclosure and iron supplementation.

In one embodiment, baseline parameter(s) for one or more hematologic parameters may be established for a patient who is a candidate to be treated with one or more variant ActRIIB polypeptides of the disclosure and an appropriate dosing regimen established for that patient based on the baseline value(s). Alternatively, established baseline parameters based on a patient's medical history could be used to inform an appropriate variant ActRIIB polypeptide dosing regimen for a patient. For example, if a healthy patient has an established baseline blood pressure reading that is above the defined normal range it may not be necessary to bring the patient's blood pressure into the range that is considered normal for the general population prior to treatment with the one or more variant ActRIIB polypeptides of the disclosure. A patient's baseline values for one or more hematologic parameters prior to treatment with one or more variant ActRIIB polypeptides of the disclosure may also be used as the relevant comparative values for monitoring any changes to the hematologic parameters during treatment with the one or more variant ActRIIB polypeptides of the disclosure.

In certain embodiments, one or more hematologic parameters are measured in patients who are being treated with one or more variant ActRIIB polypeptides. The hematologic parameters may be used to monitor the patient during treatment and permit adjustment or termination of the dosing with the one or more variant ActRIIB polypeptides of the disclosure or additional dosing with another therapeutic agent. For example, if administration of one or more variant ActRIIB polypeptides results in an increase in blood pressure, red blood cell level, or hemoglobin level, or a reduction in iron stores, white blood cell count, platelet count, or absolute neutrophil count, then the dose of the one or more variant ActRIIB polypeptides of the disclosure may be reduced in amount or frequency in order to decrease the effects of the one or more variant ActRIIB polypeptides of the disclosure on the one or more hematologic parameters. If administration of one or more variant ActRIIB polypeptides results in a change in one or more hematologic parameters that is adverse to the patient, then the dosing of the one or more variant ActRIIB polypeptides of the disclosure may be terminated either temporarily, until the hematologic parameter(s) return to an acceptable level, or permanently. Similarly, if one or more hematologic parameters are not brought within an acceptable range after reducing the dose or frequency of administration of the one or more variant ActRIIB polypeptides of the disclosure, then the dosing may be terminated. As an alternative, or in addition to, reducing or terminating the dosing with the one or more variant ActRIIB polypeptides of the disclosure, the patient may be dosed with an additional therapeutic agent that addresses the undesirable level in the hematologic parameter(s), such as, for example, a blood pressure lowering agent or an iron supplement. For example, if a patient being treated with one or more variant ActRIIB polypeptides has elevated blood pressure, then dosing with the one or more variant ActRIIB polypeptides of the disclosure may continue at the same level and a blood-pressure-lowering agent is added to the treatment regimen, dosing with the one or more antagonist of the disclosure may be reduced (e.g., in amount and/or frequency) and a blood-pressure-lowering agent is added to the treatment regimen, or dosing with the one or more antagonist of the disclosure may be terminated and the patient may be treated with a blood-pressure-lowering agent.

### Measuring Various Parameters Over Time

In certain embodiments, one or more of the measurements of PH (e.g., PcPH) described herein can be measured over various periods of treatment time. In some embodiments, one or more of the measurements of PH described herein is measured after the patient has received 4 weeks of treatment utilizing one or more variant ActRIIB polypeptides disclosed herein. In some embodiments, one or more of the measurements of PH described herein is measured after the patient has received 8 weeks of treatment utilizing one or more variant ActRIIB polypeptides disclosed herein. In some embodiments, one or more of the measurements of PH described herein is measured after the patient has received 12 weeks of treatment utilizing one or more variant ActRIIB polypeptides disclosed herein. In some embodiments, one or more of the measurements of PH described herein is measured after the patient has received 16 weeks of treatment utilizing one or more variant ActRIIB polypeptides disclosed herein. In some embodiments, one or more of the measurements of PH described herein is measured after the patient has received 20 weeks of treatment utilizing one or more variant ActRIIB polypeptides disclosed herein. In some embodiments, one or more of the measurements of PH described herein is measured after the patient has received 22 weeks of treatment utilizing one or more variant ActRIIB polypeptides disclosed herein. In some embodiments, one or more of the measurements of PH described herein is measured after the patient has received 24 weeks of treatment utilizing one or more variant ActRIIB polypeptides disclosed herein. In some embodiments, one or more of the measurements of PH described herein is measured after the patient has received 26 weeks of treatment utilizing one or more variant ActRIIB polypeptides disclosed herein. In some embodiments, one or more of the measurements of PH described herein is measured after the patient has received 28 weeks of treatment utilizing one or more variant ActRIIB polypeptides disclosed herein. In some embodiments, one or more of the measurements of PH described herein is measured after the patient has received 48 weeks of treatment utilizing one or more variant ActRIIB polypeptides disclosed herein.

### 7. Pharmaceutical Compositions & Modes of Administration

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the variant ActRIIB polypeptides which may also optionally be included in the composition as described above, may be administered simultaneously or sequentially with the subject compounds in the methods disclosed herein.

Typically, polypeptide therapeutic agents disclosed herein will be administered parentally, and particularly intravenously or subcutaneously. Pharmaceutical compositions suitable for parenteral administration may comprise one or more variant ActRIIB polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., variant ActRIIB polypeptides) to a target tissue site, providing a structure for the developing tissue and optimally capable of being resorbed into the body. For example, the matrix may provide slow release of the variant ActRIIB polypeptide. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure polypeptides or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., variant ActRIIB polypeptides). The various factors include, but are not limited to, the patient's age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling. In some embodiments, variant ActRIIB polypeptides of the disclosure are administered at a dosing range of 0.1 mg/kg to 2.0 mg/kg. In some embodiments, variant ActRIIB polypeptides of the disclosure are administered at 0.1 mg/kg. In some embodiments, variant ActRIIB polypeptides of the disclosure are administered at 0.2 mg/kg. In some embodiments, variant ActRIIB polypeptides of the disclosure are administered at 0.3 mg/kg. In some embodiments, variant ActRIIB polypeptides of the disclosure are administered at 0.4 mg/kg. In some embodiments, variant ActRIIB polypeptides of the disclosure are administered at 0.5 mg/kg. In some embodiments, variant ActRIIB polypeptides of the disclosure are administered at 0.6 mg/kg. In some embodiments, variant ActRIIB polypeptides of the disclosure are administered at 0.7 mg/kg. In some embodiments, variant ActRIIB polypeptides of the disclosure are administered at 0.8 mg/kg. In some embodiments, variant ActRIIB polypeptides of the disclosure are administered at 0.9 mg/kg. In some embodiments, variant ActRIIB polypeptides of the disclosure are administered at 1.0 mg/kg. In some embodiments, variant ActRIIB polypeptides of the disclosure are administered at 1.1 mg/kg. In some embodiments, variant ActRIIB polypeptides of the disclosure are administered at 1.2 mg/kg. In some embodiments, variant ActRIIB polypeptides of the disclosure are administered at 1.3 mg/kg. In some embodiments, variant ActRIIB polypeptides of the disclosure are administered at 1.4 mg/kg. In some embodiments, variant ActRIIB polypeptides of the disclosure are administered at 1.5 mg/kg. In some embodiments, variant ActRIIB polypeptides of the disclosure are administered at 1.6 mg/kg. In some embodiments, variant ActRIIB polypeptides of the disclosure are administered at 1.7 mg/kg. In some embodiments, variant ActRIIB polypeptides of the disclosure are administered at 1.8 mg/kg. In some embodiments, variant ActRIIB polypeptides of the disclosure are administered at 1.9 mg/kg. In some embodiments, variant ActRIIB polypeptides of the disclosure are administered at 2.0 mg/kg. The various factors include, but are not limited to, the patient's age, sex, and diet, the severity disease, time of administration, and other clinical factors. Optionally, the dosage may vary with the type of matrix used in the reconstitution and the types of compounds in the composition.

In some embodiments, a patient's hematologic parameters can be monitored by periodic assessments in order to determine if they have higher than normal red blood cell levels and/or hemoglobin levels (e.g., hemoglobin levels>16.0 g/dL or hemoglobin levels>18.0 g/dL). In some embodiments, patient's having higher than normal red blood cell levels and/or hemoglobin levels may receive a delayed or reduced dose until the levels have returned to a normal or acceptable level.

The probability of a patient having hemoglobin levels greater than 18 g/dL or increases in hemoglobin of greater than 2 g/dL may be higher during initial treatment with a variant ActRIIB polypeptide. In certain embodiments, a dosing regimen can be used to prevent, ameliorate, or decrease the adverse changes in hemoglobin levels. In some embodiments, variant ActRIIB polypeptides of the disclosure are administered using a dosing regimen. In some embodiments, the method comprises administering a dosing regimen of a therapeutically effective amount of a variant ActRIIB polypeptide as disclosed herein to a patient, comprising a first dose of between 0.1 mg/kg and 1.0 mg/kg of said polypeptide for a first period of time, and a second dose of between 0.1 mg/kg and 1.0 mg/kg of said polypeptide subsequently administered for a second period of time. In some embodiments, the method comprises administering a dosing regimen of therapeutically effective amount of a variant ActRIIB polypeptide as disclosed herein to a patient, comprising a first dose of between 0.1 mg/kg and 1.0 mg/kg of said polypeptide for a first period of time, a second dose of between 0.1 mg/kg and 1.0 mg/kg of said polypeptide administered for a second period of time, and a third dose of between 0.1 mg/kg and 1.0 mg/kg of said polypeptide subsequently administered for a third period of time. In some embodiments, the first dose of a variant ActRIIB polypeptide is administered to a patient in an amount from about 0.2 mg/kg to about 0.4 mg/kg. In some embodiments, the first dose of a variant ActRIIB polypeptide is administered to a patient at a dose of 0.3 mg/kg. In some embodiments, the second dose of a variant ActRIIB polypeptide is administered to a patient in an amount from about 0.5 mg/kg to about 0.8 mg/kg. In some embodiments, the second dose of a variant ActRIIB polypeptide is administered to a patient at a dose of 0.7 mg/kg. In some embodiments, the third dose of a variant ActRIIB polypeptide is administered to a patient in an amount from about 0.2 mg/kg to about 0.4 mg/kg. In some embodiments, the third dose of a variant ActRIIB polypeptide is administered to a patient at a dose of 0.3 mg/kg.

In some embodiments, the dosing regimen comprises administering a first dose of a variant ActRIIB polypeptide to a patient in an amount of 0.3 mg/kg followed by administration of a second dose of a variant ActRIIB polypeptide to the patient in an amount of 0.7 mg/kg. In some embodiments, the dosing regimen comprises administering a first dose of a variant ActRIIB polypeptide to a patient in an amount of 0.3 mg/kg, administering a second dose of a variant ActRIIB polypeptide to the patient in an amount of 0.7 mg/kg, and administering a third dose of a variant ActRIIB polypeptide to the patient in an amount of 0.3 mg/kg. In some embodiments, the second dose exceeds the first dose. In some embodiments, the first dose exceeds the second dose. In some embodiments, the third dose exceeds the second dose. In some embodiments, the second dose exceeds the third dose. In some embodiments, the first period of time is at least 3 weeks. In some embodiments, the second period of time is at least 3 weeks. In some embodiments, the third period of time is at least 3 weeks. In some embodiments, the second period of time is at least 21 weeks. In some embodiments, the second period of time is at least 45 weeks. In some embodiments, the second period of time exceeds the first period of time. In some embodiments, the third period of time exceeds the first period of time. In some embodiments, the third period of time exceeds the second period of time.

In some embodiments, the change in dosing between the first dose and the second dose is determined by the attending physician considering various factors (e.g., hemoglobin levels). In some embodiments, the change in dosing between the second dose and the third dose is determined by the attending physician considering various factors (e.g., hemoglobin levels). In some embodiments, the various factors include, but are not limited to, the patient's change in hematologic parameters over a period of time. In some embodiments, a patient's hematologic parameters are monitored in order to determine if they have higher than normal red blood cell levels and/or hemoglobin levels (e.g., hemoglobin levels>16.0 g/dL or hemoglobin levels>18.0 g/dL). In some embodiments, a patient's hematologic parameters are monitored in order to determine if they have a higher than normal increase in hemoglobin levels over a period of time (e.g., hemoglobin level increase of >2 g/dL in less than 3 weeks). In some embodiments, the patient's dose of a variant ActRIIB polypeptide as disclosed herein will be decreased (e.g., decrease in dose from 0.7 mg/kg to 0.3 mg/kg) if one or more of the patient's hematologic parameters before or during treatment is abnormal. In some embodiments, the patient's dose of a variant ActRIIB polypeptide as disclosed herein will be maintained (e.g., maintained at 0.3 mg/kg or 0.7 mg/kg) if one or more of the patient's hematologic parameters before or during treatment is abnormal.

In some embodiments, the dosing regimen prevents, ameliorates, or decreases adverse effects of the variant ActRIIB polypeptide. In some embodiments, administration of a variant ActRIIB polypeptide in accordance with the dosage regimen as provided herein results in decreased adverse side effects. In some embodiments, administration of a variant ActRIIB polypeptide in accordance with the dosage regimen as provided herein decreases the probability of having hemoglobin levels greater than 18 g/dL during the first period of time. In some embodiments, administration of a variant ActRIIB polypeptide in accordance with the dosage regimen as provided herein decreases the probability of having hemoglobin levels greater than 18 g/dL in the first 3 weeks of treatment. In some embodiments, administration of a variant ActRIIB polypeptide in accordance with the dosage regimen as provided herein decreases the probability of increasing hemoglobin levels by greater than 2 g/dL during the first period of time. In some embodiments, administration of a variant ActRIIB polypeptide in accordance with the dosage regimen as provided herein decreases the probability of increasing hemoglobin levels by greater than 2 g/dL in the first 3 weeks of treatment.

In certain embodiments, variant ActRIIB polypeptides of the disclosure are administered once a day. In certain embodiments, variant ActRIIB polypeptides of the disclosure are administered twice a day. In certain embodiments, variant ActRIIB polypeptides of the disclosure are administered once a week. In certain embodiments, variant ActRIIB polypeptides of the disclosure are administered twice a week. In certain embodiments, variant ActRIIB polypeptides of the disclosure are administered three times a week. In certain embodiments, variant ActRIIB polypeptides of the disclosure are administered every two weeks. In certain embodiments, variant ActRIIB polypeptides of the disclosure are administered every three weeks. In certain embodiments, variant ActRIIB polypeptides of the disclosure are administered every four weeks. In certain embodiments, variant ActRIIB polypeptides of the disclosure are administered every month.

In certain embodiments, the present invention also provides gene therapy for the in vivo production of variant ActRIIB polypeptides. Such therapy would achieve its therapeutic effect by introduction of the variant ActRIIB polypeptide polynucleotide sequences into cells or tissues having the disorders as listed above. Delivery of variant ActRIIB polypeptide polynucleotide sequences can be achieved using a recombinant expression vector such as a chimeric virus or a colloidal dispersion system. Preferred for therapeutic delivery of variant ActRIIB polypeptide polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the variant ActRIIB polypeptide. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for variant ActRIIB polypeptide polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### 8. Kits

The present disclosure provides a kit comprising a lyophilized polypeptide and an injection device. In certain embodiments, the lyophilized polypeptide comprises one or more variant ActRIIB polypeptides of the present disclosure (e.g., variant ActRIIB polypeptides in either homomeric or heteromeric forms), or fragments, functional variants, or modified forms thereof. In certain embodiments, the lyophilized polypeptide binds to one or more ligands selected from the group consisting of activin A, activin B, and GDF11. In certain such embodiments, the lyophilized polypeptide further binds to one or more ligands selected from the group consisting of BMP10, GDF8, and BMP6. In certain embodiments, the lyophilized polypeptide binds to activin and/or GDF11.

In certain embodiments, the lyophilized polypeptide is part of a homodimer polypeptide complex. In certain embodiments, the lyophilized polypeptide is part of a heteromultimer polypeptide complex. In certain embodiments, the heteromultimer is a heterodimer.

In certain embodiments, the polypeptide is glycosylated.

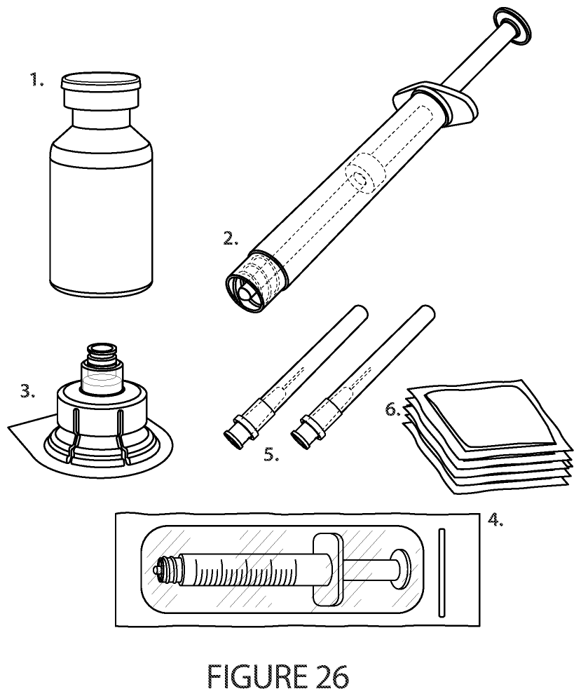

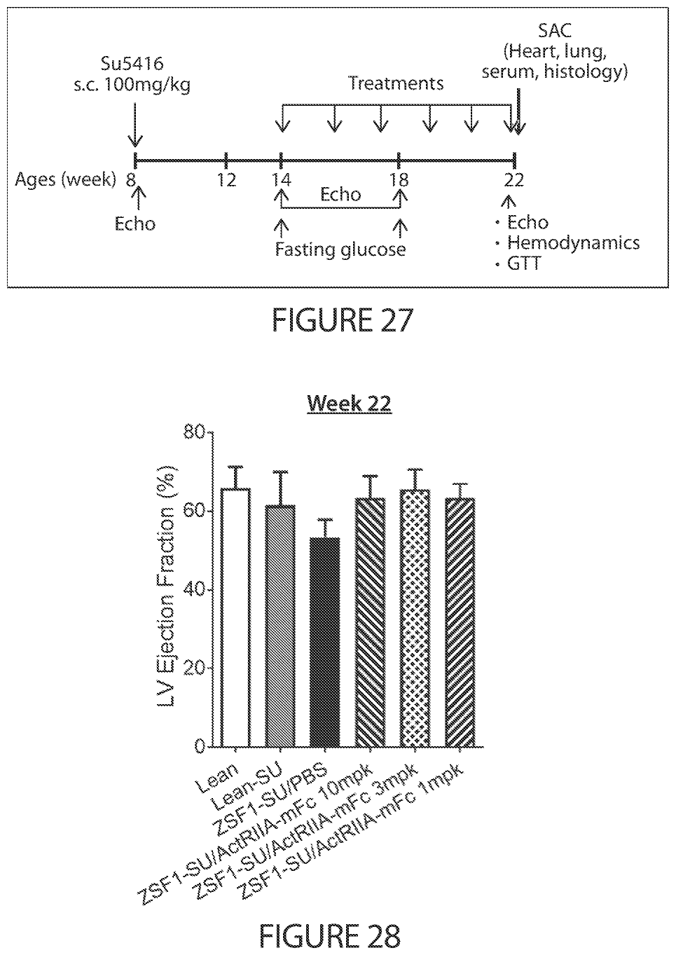

The present disclosure provides a kit comprising a sterile powder comprising a lyophilized polypeptide as disclosed herein and an injection device. In some embodiments of the kits disclosed herein, the sterile powder comprising a lyophilized polypeptide is pre-filled in one or more containers, such as one or more vials [FIG. 26 (1)].

In certain embodiments, the pH range for the sterile powder comprising a lyophilized polypeptide is from 7 to 8. In some embodiments, the sterile powder comprising a lyophilized polypeptide further comprises a buffering agent. In some embodiments, the buffering agent may be added in an amount of at least 10 mM. In some embodiments, the buffering agent may be added in an amount in the range of between about 10 mM to about 200 mM. In some embodiments, the buffering agent comprises citric acid monohydrate and/or trisodium citrate dehydrate.

In some embodiments, the sterile powder comprising a lyophilized polypeptide further comprises a surfactant. In some embodiments, the surfactant comprises a polysorbate. In some embodiments, the surfactant comprises polysorbate 80.

In some embodiments, the sterile powder comprising a lyophilized polypeptide further comprises a lyoprotectant. In some embodiments, the lyoprotectant comprises a sugar, such as disaccharides (e.g, sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the sterile powder comprises the lyoprotectant and lyophilized polypeptide in a weight ratio of at least 1:1 lyophilized polypeptide to lyoprotectant. In some embodiments, the sterile powder comprises the lyoprotectant and lyophilized polypeptide in a weight ratio of from 1:1 to 1:10 lyophilized polypeptide to lyoprotectant. In some embodiments, the sterile powder comprises the lyoprotectant and lyophilized polypeptide in a weight ratio of 1:1, 1:2, 1:3, 1:4, 1:5, 1:6, 1:7, 1:8, 1:9, or 1:10 lyophilized polypeptide to lyoprotectant. In some embodiments, the sterile powder comprises the lyoprotectant and lyophilized polypeptide in a weight ratio of 1:6 lyophilized polypeptide to lyoprotectant. In certain embodiments of the foregoing, the sterile powder comprises lyoprotectant in an amount sufficient to stabilize the lyophilized polypeptide.

In certain embodiments of the kits disclosed herein, the injection device comprises a syringe [FIG. 26 (2)]. In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises aqueous solutions such as water, physiologically buffered saline, or other solvents or vehicles such as glycols, glycerol, oils or injectable organic esters. In some embodiments, the pharmaceutically acceptable excipient comprises a pharmaceutically acceptable excipient selected from calcium phosphates, calcium carbonates, calcium sulfates, halites, metallic oxides, sugars, sugar alcohols, starch, glycols, povidones, mineral hydrocarbons, acrylic polymers, fatty alcohols, mineral stearates, glycerin, and/or lipids. In certain embodiments, the reconstitution solution comprises pharmaceutically acceptable sterile isotonic aqueous or nonaqueous solutions, dispersions, suspensions or emulsions. In certain such embodiments, the reconstitution solution comprises antioxidants, buffers, bacteriostats, and/or solutes which render the formulation isotonic with the blood of the intended recipient. In other embodiments, the reconstitution solution comprises suspending or thickening agents.

In certain embodiments of the kits disclosed herein, the kit further comprises a vial adapter [FIG. 26 (3)]. In some embodiments, the vial pre-filled with sterile powder comprising a lyophilized polypeptide attaches to one end of the vial adapter. In some embodiments, the syringe pre-filled with a reconstitution solution as disclosed herein attaches to an end of the vial adapter. In some embodiments, the syringe pre-filled with a reconstitution solution as disclosed herein and the vial pre-filled with sterile powder comprising a lyophilized polypeptide are attached to opposite ends of the vial adapter. In some embodiments, the reconstitution solution is transferred from the pre-filled syringe to the vial. In some embodiments, transfer of the reconstitution solution to the vial pre-filled with sterile powder comprising a lyophilized polypeptide reconstitutes the lyophilized polypeptide into a sterile injectable solution. In some embodiments, the lyophilized polypeptide is reconstituted into a sterile injectable solution. In some embodiments, the lyophilized polypeptide is reconstituted into a sterile injectable solution prior to use.

In other embodiments of the kits disclosed herein, the kit further comprises a pump apparatus. In certain embodiments, the pump apparatus comprises an electromechanical pumping assembly. In certain embodiments, the pump apparatus comprises a reservoir for holding a sterile injectable solution. In certain embodiments, the reservoir holds 1 mL of sterile injectable solution. In certain embodiments, the pump apparatus comprises one or more vials or cartridges comprising a sterile injectable solution. In certain embodiments, the vials or cartridges are prefilled with sterile injectable solution. In certain embodiments, the vials or cartridges comprise sterile injectable solution reconstituted from a lyophilized polypeptide. In certain embodiments, the reservoir is coupled to the vial or cartridge. In certain embodiments, the vial or cartridge holds 1-20 mL of sterile injectable solution. In certain embodiments, the electromechanical pumping assembly comprises a pump chamber. In certain embodiments, the electromechanical pumping assembly is coupled to the reservoir. In certain embodiments, the sterile injectable solution is received from the reservoir into the pump chamber. In some embodiments, the electromechanical pumping assembly comprises a plunger that is disposed such that sterile injectable solution in the pump chamber is in direct contact with the plunger. In certain embodiments, a sterile injectable solution is received from the reservoir into the pump chamber during a first pumping phase, and is delivered from the pump chamber to a subject during a second pumping phase. In certain embodiments, the electromechanical pumping assembly comprises control circuitry. In certain embodiments, control circuitry drives the plunger to (a) draw the sterile injectable solution into the pump chamber during the first pumping phase and (b) deliver the sterile injectable solution from the pump chamber in a plurality of discrete motions of the plunger during the second pumping phase, thereby delivering the therapeutic substance to the subject in a plurality of controlled and discrete dosages throughout the second pumping phase. In certain embodiments, a cycle of alternating the first and second pumping phases may be repeated until a desired dose is administered. In certain embodiments, the pump apparatus is coupled to a wearable patch. In certain embodiments, the pump apparatus is a wearable pump apparatus. In some embodiments, the pump apparatus administers a dose every 3 weeks. In some embodiments, the pump apparatus administers the dose via subcutaneous injection.

The present disclosure provides a kit used for reconstituting a lyophilized polypeptide into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution parenterally [FIG. 26 (1, 2, 3, 4, and 5)]. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose. In some embodiments, the weight based dose is 0.3 mg/kg. In some embodiments, the weight based dose is 0.7 mg/kg.

In some embodiments of the kits disclosed herein, the kit further comprises one or more vials or cartridges containing the lyophilized polypeptide. In some embodiments, the kit comprises at least two vials or cartridges containing the lyophilized polypeptide. In some embodiments, the kit comprises at least three vials or cartridges containing the lyophilized polypeptide. In some embodiments, the two vials can contain the same or different amounts of the lyophilized polypeptide. In some embodiments, the vials or cartridges comprise a vial or cartridge containing between 25 mg to 60 mg of lyophilized polypeptide. In some embodiments, at least one of the vials or cartridges comprise a vial or cartridge containing 60 mg of lyophilized polypeptide. In some embodiments, at least one of the vials or cartridges comprise a vial or cartridge containing 45 mg of lyophilized polypeptide. In some embodiments, at least one of the vials or cartridges comprise a vial or cartridge containing 30 mg of lyophilized polypeptide. In some embodiments, at least one of the vials or cartridges comprise a vial or cartridge containing 25 mg of lyophilized polypeptide. In some embodiments, a first vial or cartridge contains 45 mg of lyophilized polypeptide and a second vial or cartridge contains 60 mg of lyophilized polypeptide. In some embodiments, a first vial or cartridge contains 30 mg of lyophilized polypeptide and a second vial or cartridge contains 60 mg of lyophilized polypeptide. In some embodiments, a first vial or cartridge contains 30 mg of lyophilized poly peptide, a second vial or cartridge contains 45 mg of lyophilized poly peptide, and a third vial or cartridge contains 60 mg of lyophilized polypeptide. In some embodiments, a first vial or cartridge contains 25 mg of lyophilized polypeptide, a second vial or cartridge contains 45 mg of lyophilized polypeptide, and a third vial or cartridge contains 60 mg of lyophilized polypeptide. In some embodiments, the one or more vials or cartridges are refrigerated at 2-8° C.

### 9. Exemplification

The disclosure above will be more readily understood by reference to the following examples, which are included merely for purposes of illustration of certain embodiments of the present invention, and are not intended to be limiting.

### Example 1. Generation of an ActRIIB-Fc Fusion Polypeptide

Applicants constructed a soluble ActRIIB fusion polypeptide that has the extracellular domain of human ActRIIB fused to a human G1Fc domain with a linker (three glycine amino acids) in between. The construct is referred to as ActRIIB-G1Fc.

ActRIIB-G1Fc is shown below in SEQ ID NO: 5 (with the linker underlined) as purified from CHO cell lines:

(SEQ ID NO: 5)
GRGEAETRECIYYNANWELERTNQSGLERCEGEQDKRLHCYASWRNSS
GTIELVKKGCWLDDFNCYDRQECVATEENPQVYFCCCEGNFCNERFTH
LPEAGGPEVTYEPPPTAPTGGGTHTCPPCPAPELLGGPSVFLFPPKPK
DTLMISRTPEVTCVVVDVSHEDPEVKFNWYVDGVEVHNAKTKPREEQY
NSTYRVVSVLTVLHQDWINGKEYKCKVSNKALPAPIEKTISKAKGQPR
EPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKS
LSLSPGK

The ActRIIB-G1Fc polypeptide was expressed in CHO cell lines. Three different leader sequences were considered:

(i) Honey bee mellitin (HBML):
(SEQ ID NO: 7)
MKFLVNVALVFMVVYISYIYA
(ii) Tissue plasminogen activator (TPA):
(SEQ ID NO: 8)
MDAMKRGLCCVLLLCGAVFVSP
(iii) Native:
(SEQ ID NO: 9)
MTAPWVALALLWGSLCAG.

The selected form employs the TPA leader and has the following unprocessed amino acid sequence:

(SEQ ID NO: 6)

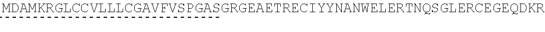

LHCYASWRNSSGTIELVKKGCWLDDFNCYDRQECVATEENPQVYFCCCEGNFCNERFTHLPE

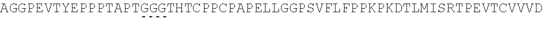

VSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK

This polypeptide is encoded by the following nucleic acid sequence (SEQ ID NO: 10):

(SEQ ID NO: 10)
ATGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GCTAGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG CCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA

N-terminal sequencing of the CHO-cell produced material revealed a major sequence of -GRGEAE (SEQ ID NO: 11). Notably, other constructs reported in the literature begin with an -SGR . . . sequence.

Applicants also constructed a soluble ActRIIB fusion polypeptide that has the extracellular domain of human ActRIIB fused to a murine IgG2a Fc domain with a linker in between. The construct is referred to as ActRIIB-mFc.

ActRIIB-mFc is shown below in SEQ ID NO: 56 (with the linker underlined) as follows:

| | |
|---|---|
| 1 | ILGRSETQEC LFFNANWEKD RTNQTGVEPC YGDKDKRRHC FATWKNISGS |
| 51 | IEIVKQGCWL DDINCYDRTD CVEKKDSPEV YFCCCEGNMC NEKFSYFPEM |
| 101 | EVTQPTSNPV TPKPPTGGGE PRVPITQNPC PPLKECPPCA APDLLGGPSV |
| 151 | FIFPPKIKDV LMISLSPMVT CVVVDVSEDD PDVQISWFVN NVEVHTAQTQ |
| 201 | THREDYNSTL RVVSALPIQH QDWMSGKEFK CKVNNRALPS PIEKTISKPR |
| 251 | GPVRAPQVYV LPPPAEEMTK KEFSLTCMIT GFLPAEIAVD WTSNGRTEQN |
| 301 | YKNTATVLDS DGSYFMYSKL RVQKSTWERG SLFACSVVHE GLHNHLTTKT |
| 351 | ISRSLGK |

In some embodiments ActRIIB-mFc may be provided with a lysine at the C-terminus. In some embodiments, ActRIIB-mFc may be provided without a lysine at the C-terminus. In some embodiments, ActRIIB-mFc polypeptide may comprise a mixture of polypeptides with a lysine at the C-terminus and polypeptides without a lysine at the C-terminus.

The unprocessed amino acid sequence of ActRIIB-mFc employs the TPA leader and has the following unprocessed amino acid sequence:

| (SEQ ID NO: 57) | |
|---|---|
| 1 | MDAMKRGLCC VLLLCGAVFV SPGAAILGRS ETQECLFFNA NWEKDRTNQT |
| 51 | GVEPCYGDKD KRRHCFATWK NISGSIEIVK QGCWLDDINC YDRTDCVEKK |
| 101 | DSPEVYFCCC EGNMCNEKFS YFPEMEVTQP TSNPVTPKPP TGGGEPRVPI |
| 151 | TQNPCPPLKE CPPCAAPDLL GGPSVFIFPP KIKDVLMISL SPMVTCVVVD |
| 201 | VSEDDPDVQI SWFVNNVEVH TAQTQTHRED YNSTLRVVSA LPIQHQDWMS |
| 251 | GKEFKCKVNN RALPSPIEKT ISKPRGPVRA PQVYVLPPPA EEMTKKEFSL |
| 301 | TCMITGFLPA EIAVDWTSNG RTEQNYKNTA TVLDSDGSYF MYSKLRVQKS |
| 351 | TWERGSLFAC SVVHEGLHNH LTTKTISRSL GK* |

The leader (signal) sequence and linker are underlined.

This polypeptide is encoded by the following nucleic acid sequence (SEQ ID NO: 58):

| | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCGCTATACT TGGTAGATCA GAAACTCAGG |
| 101 | AGTGTCTTTT CTTTAATGCT AATTGGGAAA AAGACAGAAC CAATCAAACT |
| 151 | GGTGTTGAAC CGTGTTATGG TGACAAAGAT AAACGGCGGC ATTGTTTTGC |
| 201 | TACCTGGAAG AATATTTCTG GTTCCATTGA AATAGTGAAA CAAGGTTGTT |
| 251 | GGCTGGATGA TATCAACTGC TATGACAGGA CTGATTGTGT AGAAAAAAAA |
| 301 | GACAGCCCTG AAGTATATTT CTGTTGCTGT GAGGGCAATA TGTGTAATGA |
| 351 | AAAGTTTTCT TATTTTCCGG AGATGGAAGT CACACAGCCC ACTTCAAATC |
| 401 | CAGTTACACC TAAGCCACCC ACCGGTGGGG GTGAGCCCAG AGTGCCCATA |
| 451 | ACACAGAACC CCTGTCCTCC ACTCAAAGAG TGTCCCCCAT GCGCAGCTCC |
| 501 | AGACCTCTTG GGTGGACCAT CCGTCTTCAT CTTCCCTCCA AAGATCAAGG |
| 551 | ATGTACTCAT GATCTCCCTG AGCCCCATGG TCACATGTGT GGTGGTGGAT |
| 601 | GTGAGCGAGG ATGACCCAGA CGTCCAGATC AGCTGGTTTG TGAACAACGT |
| 651 | GGAAGTACAC ACAGCTCAGA CACAAACCCA TAGAGAGGAT TACAACAGTA |
| 701 | CTCTCCGGGT GGTCAGTGCC CTCCCCATCC AGCACCAGGA CTGGATGAGT |
| 751 | GGCAAGGAGT TCAAATGCAA GGTCAACAAC AGAGCCCTCC CATCCCCCAT |
| 801 | CGAGAAAACC ATCTCAAAAC CCAGAGGGCC AGTAAGAGCT CCACAGGTAT |
| 851 | ATGTCTTGCC TCCACCAGCA GAAGAGATGA CTAAGAAAGA GTTCAGTCTG |
| 901 | ACCTGCATGA TCACAGGCTT CTTACCTGCC GAAATTGCTG TGGACTGGAC |
| 951 | CAGCAATGGG CGTACAGAGC AAAACTACAA GAACACCGCA ACAGTCCTGG |
| 1001 | ACTCTGATGG TTCTTACTTC ATGTACAGCA AGCTCAGAGT ACAAAAGAGC |
| 1051 | ACTTGGGAAA GAGGAAGTCT TTTCGCCTGC TCAGTGGTCC ACGAGGGTCT |
| 1101 | GCACAATCAC CTTACGACTA AGACCATCTC CCGGTCTCTG GGTAAATGA |

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

The ActRIIB-Fc fusion polypeptide was also expressed in HEK293 cells and COS cells. Although material from all cell lines and reasonable culture conditions provided polypeptide with muscle-building activity in vivo, variability in potency was observed perhaps relating to cell line selection and/or culture conditions.

### Example 2. Computational Methods

The Activin IIB receptor (ActRIIB) binds multiple TGFβ superfamily ligands, including activin A, activin B, GDF8, and GDF11, that stimulate Smad2/3 activation, as well as bone morphogenic proteins (BMPs), such as BMP9 and BMP10, that stimulate Smad1/5/8 activation. ActRIIB-Fc fusion polypeptides can function as ligand traps that bind to soluble ligands and block Smad activation by preventing ligands from binding to cell surface receptors. ActRIIB-Fc antagonism of BMP9-mediated Smad1/5/8 activation has been known to result in undesired side effects, including epistaxis and telangiectasias (Campbell, C. et al. Muscle Nerve 55: 458-464, 2017). In order to design mutations in ActRIIB that diminish BMP9 binding, while retaining binding to ligands that stimulate Smad2/3 activation, we compared the crystal structures of three ActRIIB ligand complexes: (1) BMP9:ActRIIB:Alk1, PDB ID=4fao, (2) ActRIIB:Activin A, PDB ID:1s4y, and (3)GDF11:ActRIIB:Alk5, PDB ID: 6mac (available from the Protein Data Bank (PDB) https://www.rcsb.org/). Comparison of contacts between ActRIIB and the three ligands based on the crystal structures revealed residues for mutational focus based on charge, polarity, and hydrophobicity differences of the ligand residues contacted by the same corresponding ActRIIB residue. After identifying residues to target for mutation, the Schrödinger Bioluminate biologics modeling software platform (version 2017-4: Bioluminate, Schrödinger, LLC, New York, NY) was used to computationally predict mutations in ActRIIB that would diminish binding to BMP9, while maintaining other ligand-binding activities.

All residues identified from the comparison of the crystal structures were considered for mutation. Residue Scanning Calculations were performed considering both stability and affinity of the molecules in the structural complex, producing a specified list of potential mutations and energies for each molecule (ligand and receptor) and complex structure, as well as energy differences for both the wild type and the mutant form. After analyzing affinity/stability/prime energy, etc. parameters, the top 5%-10% of the single mutations were identified. This analysis was followed by potential combination of these mutations. Selected single mutations and mutation combinations were structurally analyzed in order to understand structural differences and formed/lost contacts. Ultimately, 817 single mutations were screened for each complex (ActRIIB:ligand), and top hits were selected based on Δaffinity, and also taking into selective consideration Δstability (solvated) and Δprime energy. Other properties were also considered when regarding striking of outliers.

### Example 3. Generation of Variant ActRIIB-Fc Polypeptides

Based on the findings described in Example 1, Applicants generated a series of mutations (sequence variations) in the extracellular domain of ActRIIB and produced these variant polypeptides as soluble homodimeric fusion polypeptides comprising a variant ActRIIB extracellular domain and an Fc domain joined by an optional linker. The background ActRIIB-Fc fusion used for the generation of variant ActRIIB-Fc polypeptides was ActRIIB-G1Fc, and is shown in Example 1 above as SEQ ID NO: 5.

Various substitution mutations were introduced into the background ActRIIB-G1Fc polypeptide. Based on the data presented in Example 1, it is expected that these constructs, if expressed with a TPA leader, will lack the N-terminal serine. Thus, the majority of mature sequences may begin with a glycine (lacking the N-terminal serine) but some species may be present with the N-terminal serine. Mutations were generated in the ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNA was isolated. For murine constructs (mFc), a murine IgG2a was substituted for the human IgG1. All mutants were sequence verified.

The amino acid sequence of unprocessed ActRIIB(F82I-N83R)-G1Fc is shown below (SEQ ID NO: 276). The signal sequence and linker sequence are indicated by solid underline, and the F82I and N83R substitutions are indicated by double underline. The amino acid sequence of SEQ ID NO: 276 may optionally be provided with the lysine removed from the C-terminus.

| (SEQ ID NO: 276) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDIRC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(F82I-N83R)-G1Fc fusion polypeptide is encoded by the following nucleic acid sequence (SEQ ID NO: 277):

| (SEQ ID NO: 277) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CATCCGTTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCIGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(F82I-N83R)-G1Fc fusion polypeptide (SEQ ID NO: 278) is as follows and may optionally be provided with the lysine removed from the C-terminus.

| (SEQ ID NO: 278) | |
|---|---|
| 1 | GRGEAETREC IYYNANWELE RTNQSGLERC EGEQDKRLHC YASWRNSSGT |
| 51 | IELVKKGCWL DDIRCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGEYP SDIAVEWESN GQPENNYKTT PPVLDSDGSE |
| 301 | FLYSKLTVDK SRWQQGNVFS CSVMHEALHN HYTQKSLSLS PGK* |

The amino acid sequence of unprocessed ActRIIB(F82K-N83R)-G1Fc is shown below (SEQ ID NO: 279). The signal sequence and linker sequence are indicated by solid underline, and the F82K and N83R substitutions are indicated by double underline. The amino acid sequence of SEQ ID NO: 279 may optionally be provided with the lysine removed from the C-terminus.

| (SEQ ID NO: 279) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDKRC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(F82K-N83R)-G1Fc fusion polypeptide is encoded by the following nucleic acid sequence (SEQ ID NO: 331):

| (SEQ ID NO: 331) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGGG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CAAGCGTTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(F82K-N83R)-G1Fc fusion polypeptide (SEQ ID NO: 332) is as follows and may optionally be provided with the lysine removed from the C-terminus.

| (SEQ ID NO: 332) | |
|---|---|
| 1 | GRGEAETREC IYYNANWELE RTNQSGLERC EGEQDKRLHC YASWRNSSGT |
| 51 | IELVKKGCWL DDKRCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSE |
| 301 | FLYSKLTVDK SRWQQGNVFS CSVMHEALHN HYTQKSLSLS PGK* |

The amino acid sequence of unprocessed ActRIIB(F82T-N83R)-G1Fc is shown below (SEQ ID NO: 333). The signal sequence and linker sequence are indicated by solid underline, and the F82T and N83R substitutions are indicated by double underline. The amino acid sequence of SEQ ID NO: 333 may optionally be provided with the lysine removed from the C-terminus.

| (SEQ ID NO: 333) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDTRC YDRQECVATE |
| 101 | ENPQVYFCCC EGNFCNERFT HLPEAGGPEV TYEPPPTAPT GGGTHTCPPC |
| 151 | PAPELLGGPS VELFPPKPKD TLMISRTPEV TCVVVDVSHE DPEVKFNWYV |
| 201 | DGVEVHNAKT KPREEQYNST YRVVSVLIVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(F82T-N83R)-G1Fc fusion polypeptide is encoded by the following nucleic acid sequence (SEQ ID NO: 334):

| (SEQ ID NO: 334) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CACCCGTTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(F82T-N83R)-G1Fc fusion polypeptide (SEQ ID NO: 335) is as follows and may optionally be provided with the lysine removed from the C-terminus.

| (SEQ ID NO: 335) | |
|---|---|
| 1 | GRGEAETREC IYYNANWELE RTNQSGLERC EGEQDKRLHC YASWRNSSGT |
| 51 | IELVKKGCWL DDTRCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVES CSVMHEALHN HYTQKSLSLS PGK* |

The amino acid sequence of unprocessed ActRIIB(F82T)-G1Fc is shown below (SEQ ID NO: 336). The signal sequence and linker sequence are indicated by solid underline, and the F82T substitution is indicated by double underline. The amino acid sequence of SEQ ID NO: 336 may optionally be provided with the lysine removed from the C-terminus.

| (SEQ ID NO: 336) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDTNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(F82T)-G1Fc fusion polypeptide is encoded by the following nucleic acid sequence (SEQ ID NO: 337):

| (SEQ ID NO: 337) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CACCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(F82T)-G1Fc fusion polypeptide (SEQ ID NO: 338) is as follows and may optionally be provided with the lysine removed from the C-terminus.

| (SEQ ID NO: 338) | |
|---|---|
| 1 | GRGEAETREC IYYNANWELE RTNQSGLERC EGEQDKRLHC YASWRNSSGT |
| 51 | IELVKKGCWL DDTNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK* |

The amino acid sequence of unprocessed ActRIIB(L79H-F82I)-G1Fc is shown below (SEQ ID NO: 339). The signal sequence and linker sequence are indicated by solid underline, and the L79H and F82I substitutions are indicated by double underline. The amino acid sequence of SEQ ID NO: 339 may optionally be provided with the lysine removed from the C-terminus.

| (SEQ ID NO: 339) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWHDDING YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLIVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(L79H-F82I)-G1Fc fusion polypeptide is encoded by the following nucleic acid sequence (SEQ ID NO: 340):

| (SEQ ID NO: 340) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCACGATGA CATCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(L79H-F82I)-G1Fc fusion polypeptide (SEQ ID NO: 341) is as follows and may optionally be provided with the lysine removed from the C-terminus.

| (SEQ ID NO: 341) | |
|---|---|
| 1 | GRGEAETREC IYYNANWELE RTNQSGLERC EGEQDKRLHC YASWRNSSGT |
| 51 | IELVKKGCWH DDINCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVES CSVMHEALHN HYTQKSLSLS PGK* |

The amino acid sequence of unprocessed ActRIIB(L79H)-G1Fc is shown below (SEQ ID NO: 342). The signal sequence and linker sequence are indicated by solid underline, and the L79H substitution is indicated by double underline. The amino acid sequence of SEQ ID NO: 342 may optionally be provided with the lysine removed from the C-terminus.

| | (SEQ ID NO: 342) |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWHDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(L79H)-G1Fc fusion polypeptide is encoded by the following nucleic acid sequence (SEQ ID NO: 343):

| | (SEQ ID NO: 343) |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCACGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(L79H)-G1Fc fusion polypeptide (SEQ ID NO: 344) is as follows and may optionally be provided with the lysine removed from the C-terminus.

| | (SEQ ID NO: 344) |
|---|---|
| 1 | GRGEAETREC IYYNANWELE RTNQSGLERC EGEQDKRLHC YASWRNSSGT |
| 51 | IELVKKGCWH DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK* |

The amino acid sequence of unprocessed ActRIIB(L79H-F82K)-G1Fc is shown below (SEQ ID NO: 345). The signal sequence and linker sequence are indicated by solid underline, and the L79H and F82K substitutions are indicated by double underline. The amino acid sequence of SEQ ID NO: 345 may optionally be provided with the lysine removed from the C-terminus.

| (SEQ ID NO: 345) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWHDDKNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWINGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(L79H-F82K)-G1Fc fusion polypeptide is encoded by the following nucleic acid sequence (SEQ ID NO: 346):

| | (SEQ ID NO: 346) |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCACGATGA CAAGAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(L79H-F82K)-G1Fc fusion polypeptide (SEQ ID NO: 347) is as follows and may optionally be provided with the lysine removed from the C-terminus.

| | (SEQ ID NO: 347) |
|---|---|
| 1 | GRGEAETREC IYYNANWELE RTNQSGLERC EGEQDKRLHC YASWRNSSGT |
| 51 | IELVKKGCWH DDKNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK* |

The amino acid sequence of unprocessed ActRIIB(E50L)-G1Fc is shown below (SEQ ID NO: 348). The signal sequence and linker sequence are indicated by solid underline, and the E50L substitution is indicated by double underline. The amino acid sequence of SEQ ID NO: 348 may optionally be provided with the lysine removed from the C-terminus.

| | (SEQ ID NO: 348) |
|---|---|
| 1 | MDAMKRGLCC VLLLCGAVFV SPGASGRGEA ETRECIYYNA NWELERTNQS |
| 51 | GLERCLGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(E50L)-G1Fc fusion polypeptide is encoded by the following nucleic acid sequence (codon optimized) (SEQ ID NO: 349):

| | (SEQ ID NO: 349) |
|---|---|
| 1 | ATGGATGCGA TGAAACGCGG CCTGTGCTGC GTGCTGCTGC TGTGCGGCGC |
| 51 | GGTGTTTGTG AGCCCGGGCG CCAGCGGCCG CGGCGAAGCG GAAACCCGCG |
| 101 | AATGCATTTA TTATAACGCG AACTGGGAAC TGGAACGCAC CAACCAGAGC |
| 151 | GGCCTGGAAC GCTGCCTGGG CGAACAGGAT AAACGCCTGC ATTGCTATGC |
| 201 | GAGCTGGCGC AACAGCAGCG GCACCATTGA ACTGGTGAAA AAAGGCTGCT |
| 251 | GGCTGGATGA TTTTAACTGC TATGATCGCC AGGAATGCGT GGCGACCGAA |
| 301 | GAAAACCCGC AGGTGTATTT TTGCTGCTGC GAAGGCAACT TTTGCAACGA |
| 351 | ACGCTTTACC CATCTGCCGG AAGCGGGCGG CCCGGAAGTG ACCTATGAAC |
| 401 | CGCCGCCGAC CGCGCCGACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(E50L)-G1Fc fusion polypeptide (SEQ ID NO: 350) is as follows and may optionally be provided with the lysine removed from the C-terminus.

| (SEQ ID NO: 350) | |
|---|---|
| 1 | GRGEAETREC IYYNANWELE RTNQSGLERC LGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK* |

The amino acid sequence of unprocessed ActRIIB(L38N-L79R)-G1Fc is shown below (SEQ ID NO: 351). The signal sequence and linker sequence are indicated by solid underline, and the L38N and L79R substitutions are indicated by double underline. The amino acid sequence of SEQ ID NO: 351 may optionally be provided with the lysine removed from the C-terminus.

| | (SEQ ID NO: 351) |
|---|---|
| 1 | MDAMKRGLCC VLLLCGAVFV SPGASGRGEA ETRECIYYNA NWENERTNQS |
| 51 | GLERCEGEQD KRLHCYASWR NSSGTIELVK KGCWRDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(L38N-L79R)-G1Fc fusion polypeptide is encoded by the following nucleic acid sequence (SEQ ID NO: 352):

| | (SEQ ID NO: 352) |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGA ACGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCGCGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(L38N-L79R)-G1Fc fusion polypeptide (SEQ ID NO: 353) is as follows and may optionally be provided with the lysine removed from the C-terminus.

| | (SEQ ID NO: 353) |
|---|---|
| 1 | GRGEAETREC IYYNANWENE RTNQSGLERC EGEQDKRLHC YASWRNSSGT |
| 51 | IELVKKGCWR DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLIVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVES CSVMHEALHN HYTQKSLSLS PGK* |

The amino acid sequence of unprocessed ActRIIB(V99G)-G1Fc is shown below (SEQ ID NO: 354). The signal sequence and linker sequence are indicated by solid underline, and the V99G substitution is indicated by double underline. The amino acid sequence of SEQ ID NO: 354 may optionally be provided with the lysine removed from the C-terminus.

| (SEQ ID NO: 354) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQGYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK* |

This ActRIIB(V99G)-G1Fc fusion polypeptide is encoded by the following nucleic acid sequence (codon optimized) (SEQ ID NO: 355):

| | (SEQ ID NO: 355) |
|---|---|
| 1 | ATGGATGCGA TGAAACGCGG CCTGTGCTGC GTGCTGCTGC TGTGCGGCGC |
| 51 | GGTGTTTGTG AGCCCGGGCG CCAGCGGCCG CGGCGAAGCG GAAACCCGCG |
| 101 | AATGCATTTA TTATAACGCG AACTGGGAAC TGGAACGCAC CAACCAGAGC |
| 151 | GGCCTGGAAC GCTGCGAAGG CGAACAGGAT AAACGCCTGC ATTGCTATGC |
| 201 | GAGCTGGCGC AACAGCAGCG GCACCATTGA ACTGGTGAAA AAAGGCTGCT |
| 251 | GGCTGGATGA TTTTAACTGC TATGATCGCC AGGAATGCGT GGCGACCGAA |
| 301 | GAAAACCCGC AGGGCTATTT TTGCTGCTGC GAAGGCAACT TTTGCAACGA |
| 351 | ACGCTTTACC CATCTGCCGG AAGCGGGCGG CCCGGAAGTG ACCTATGAAC |
| 401 | CGCCGCCGAC CGCGCCGACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAATGA |

A mature ActRIIB(V99G)-G1Fc fusion polypeptide (SEQ ID NO: 356) is as follows and may optionally be provided with the lysine removed from the C-terminus.

| (SEQ ID NO: 356) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQG YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK* |

Constructs were expressed in COS or CHO cells by transient infection and purified by filtration and protein A chromatography. In some instances, assays were performed with conditioned medium rather than purified polypeptides. Purity of samples for reporter gene assays was evaluated by SDS-PAGE and analytical size exclusion chromatography.

Mutants were tested in binding assays and/or bioassays described below.

Alternatively, similar mutations could be introduced into an ActRIIB extracellular domain possessing an N-terminal truncation of five amino acids and a C-terminal truncation of three amino acids as shown below (SEQ ID NO: 357). This truncated ActRIIB extracellular domain is denoted ActRIIB(25-131) based on numbering in SEQ ID NO: 2.

| (SEQ ID NO: 357) | |
|---|---|
| 25 | ETRECIYYNA NWELERINQS GLERCEGEQD KRLHCYASWR NSSGTIELVK |
| 75 | KGCWLDDFNC YDRQECVATE ENPQVYFCCC EGNFCNERFT HLPEAGGPEV |
| 125 | TYEPPPT |

The corresponding background fusion polypeptide, ActRIIB(25-131)-G1Fc, is shown below (SEQ ID NO: 12).

| (SEQ ID NO: 12) | |
|---|---|
| 1 | ETRECIYYNA NWELERTNQS GLERCEGEQD KRLHCYASWR NSSGTIELVK |
| 51 | KGCWLDDFNC YDRQECVATE ENPQVYFCCC EGNFCNERFT HLPEAGGPEV |
| 101 | TYEPPPTGGG THTCPPCPAP ELLGGPSVFL FPPKPKDTLM ISRTPEVTCV |
| 151 | VVDVSHEDPE VKFNWYVDGV EVHNAKTKPR EEQYNSTYRV VSVLTVLHQD |
| 201 | WLNGKEYKCK VSNKALPAPI EKTISKAKGQ PREPQVYTLP PSREEMTKNQ |
| 251 | VSLTCLVKGF YPSDIAVEWE SNGQPENNYK TTPPVLDSDG SFFLYSKLTV |
| 301 | DKSRWQQGNV FSCSVMHEAL HNHYTQKSLS LSPGK |

### Example 4. Activity and Ligand Binding Profiles of Variant ActRIIB-Fc Polypeptides

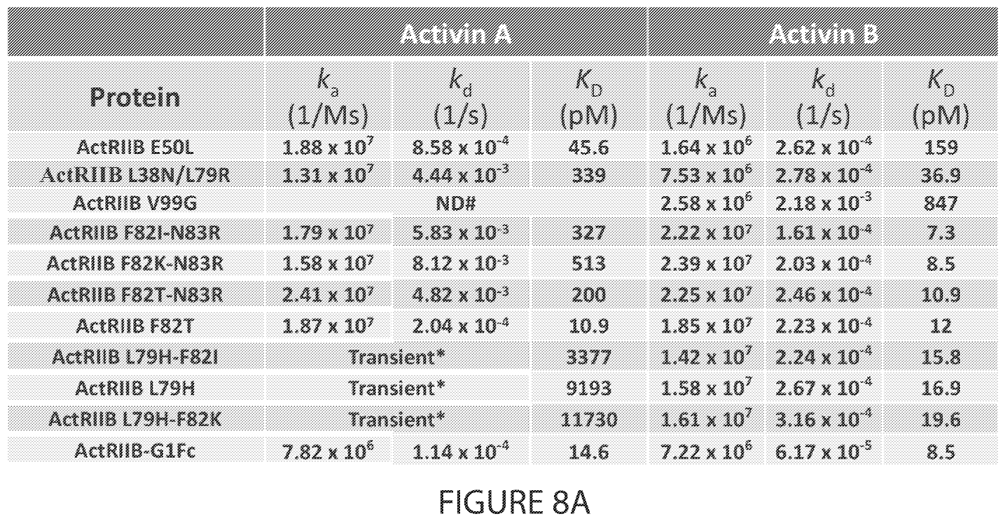

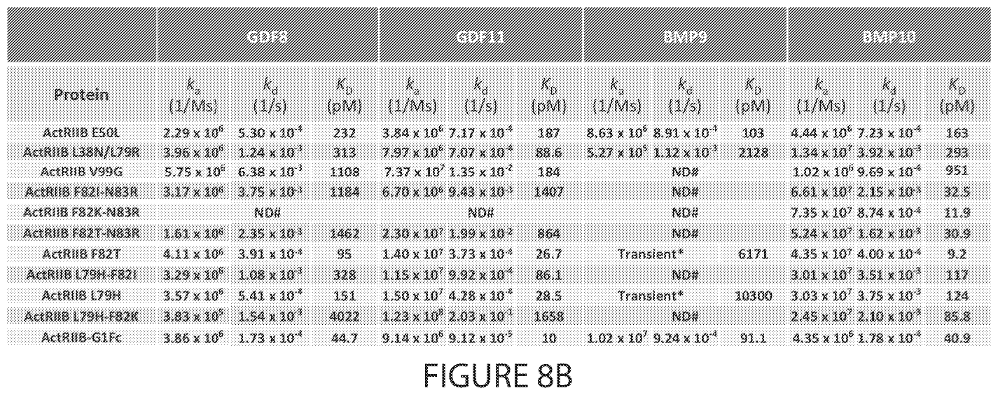

To determine ligand binding profiles of variant ActRIIB-Fc homodimers, a Biacore™-based binding assay was used to compare ligand binding kinetics of certain variant ActRIIB-Fc polypeptides. ActRIIB-Fc polypeptides to be tested were independently captured onto the system using an anti-Fc antibody. Ligands were then injected and allowed to flow over the captured receptor protein. Results of variant ActRIIB-Fc polypeptides analyzed at 37° C., are shown in FIGS. 8A and 8B. ActRIIB-G1Fc was used as the control polypeptide.

To determine activity of variant ActRIIB-Fc polypeptides, an A204 cell-based assay was used to compare effects among variant ActRIIB-Fc polypeptides on signaling by activin A, activin B, GDF8, GDF11, BMP9, and BMP10, in comparison to ActRIIB-G1Fc. In brief, this assay uses a human A204 rhabdomyosarcoma cell line (ATCC®: HTB-82™) derived from muscle and the reporter vector pGL3(CAGA)12 (Dennler et al., 1998, EMBO 17: 3091-3100) as well as a Renilla reporter plasmid (pRLCMV) to control for transfection efficiency. The CAGA12 motif is present in TGF-β responsive genes (e.g., PAI-1 gene), so this vector is of general use for ligands that can signal through Smad2/3, including activin A. GDF11, and BMP9.

On day 1. A204 cells were transferred into one or more 48-well plates. On day 2, these cells were transfected with 10 μg pGL3(CAGA)12 or pGL3(CAGA)12(10 μg)+pRLCMV (1 pg) and Fugene. On day 3, ligands diluted in medium containing 0.1% BSA were preincubated with ActRIIB-Fc polypeptides for 1 hr before addition to cells. Approximately six hour later, the cells were rinsed with PBS and lysed. Cell lysates were analyzed in a luciferase assay to determine the extent of Smad activation.

This assay was used to screen variant ActRIIB-Fc polypeptides for inhibitory effects on cell signaling by activin A, activin B. GDF8, GDF11. BMP9, and BMP10. Potencies of homodimeric Fc fusion polypeptides incorporating amino acid substitutions in the human ActRIIB extracellular domain were compared with that of an Fc fusion polypeptide comprising unmodified human ActRIIB extracellular domain, ActRIIB-G1Fc. For some variants tested, it was not possible to calculate an accurate IC₅₀, but signs of inhibition in the slope of the curves were detectable. For these variants, an estimate was included of the order of magnitude of the relative IC50, i.e. >10 nM or >100 nM instead of a definite number. Such data points are indicated by a (*) in Table 3 below. For some variants tested, there was no detectable inhibition in the slope of the curves over the concentration range tested, which is indicated by “ND” in Table 4.

TABLE 4
Inhibitory Potency of Homodimeric ActRIIB-Fc Constructs.
Inhibitory Potency of Homodimeric ActRIIB-Fc Constructs

| | IC₅₀ (nM) | | | | | |
|---|---|---|---|---|---|---|
| ActRIIB | | | Activin | Activin | | |
| polypeptide | GDF8 | GDF11 | A | B | BMP9 | BMP10 |
| ActRIIB- | 0.95 | 0.12 | 0.05 | 0.067 | 1.82 | 0.036 |
| G1Fc | | | | | | |
| F82I-N83R | ND | 9.95 | 1.67 | 0.08 | ND | 13.25 |
| F82K-N83R | ND | ND | 1.32 | 0.09 | ND | 0.53 |
| F82T-N83R | ND | 17.94 | 1.52 | 0.11 | ND | 12.57 |
| F82T | 2.17 | 0.27 | 0.10 | 0.09 | ND | 0.07 |
| L79H-F82I | >10* | 0.36 | >100* | 0.15 | ND | >100* |
| L79H | 5.7 | 0.24 | >10* | 0.07 | ND | >100* |
| L79H-F82K | ND | >100* | ND | 0.10 | ND | >100* |

ND: not detectable over concentration range tested
*estimate of the order of magnitude of the IC₅₀

As shown in Table 4 above as well as in FIGS. 8A and 8B, amino acid substitutions in the ActRIIB extracellular domain can alter the balance between ActRIIB:ligand binding and downstream signaling activities in various in vitro assay. In general, applicant achieved the goal of generating variants in the ActRIIB extracellular domain that exhibited decreased or non-detectable binding to BMP9, compared to a fusion polypeptide containing unmodified ActRIIB extracellular domain (ActRIIB-G1Fc), while retaining other ligand binding properties.

Additionally, variants ActRIIB (L79H-F82I), ActRIIB (L79H), and ActRIIB (L79H-F82K), while demonstrating a decrease in binding to BMP9, also exhibited a significant decrease in in activin A binding while retaining relatively high affinity for activin B, as compared to ActRIIB-G1Fc. IC50 values showing inhibitory potency in Table 4 are consistant with this ligand binding trend. Similarly, variants ActRIIB (F82K-N83R), ActRIIB (F82I-N83R), and ActRIIB (F82T-N83R) demonstrate a similar trend.

Furthermore, variants ActRIIB (F82K-N83R), ActRIIB (F82I-N83R), ActRIIB (F82T-N83R), and ActRIIB (L79H-F82K), while demonstrating a decrease in binding to BMP9 and retaining relatively high affinity for activin B, also exhibited a significant decrease in GDF8 and GDF11 binding, as compared to ActRIIB-G1Fc. IC₅₀ values showing inhibitory potency in Table 4 are consistent with this ligand binding trend.

It was further noted that, variants ActRIIB (L79H-F82I), ActRIIB (L79H), and ActRIIB (L79H-F82K), while demonstrating a decrease in binding to BMP9 and retaining relatively high affinity for activin B, also exhibited a decrease in BMP10 binding as compared to ActRIIB-G1Fc. IC₅₀ values showing inhibitory potency in Table 4 are consistent with this ligand binding trend.

Therefore, in addition to achieving the goal of producing ActRIIB variants that exhibit reduced to non-detectable binding to BMP9, Applicant has generated a diverse array of novel variant polypeptides, many of which are characterized in part by unique ligand binding/inhibition profiles. Accordingly, these variants may be more useful than ActRIIB-G1Fc in certain applications where such selective antagonism is advantageous. Examples include therapeutic applications where it is desirable to retain antagonism of activin B, while reducing antagonism of BMP9 and optionally one or more of activin A, GDF8, GDF11 and BMP10.

### Example 5. Generation of Variant ActRIIB-Fc Polypeptides

Applicants generated a series of mutations (sequence variations) in the extracellular domain of ActRIIB and produced these variant polypeptides as soluble homodimeric fusion polypeptides comprising a variant ActRIIB extracellular domain and an Fc domain joined by an optional linker. The background ActRIIB-Fc fusion was ActRIIB-G1Fc as shown in SEQ ID NO: 5.

Various substitution mutations were introduced into the background ActRIIB-Fc polypeptide. Based on the data presented in Example 1, it is expected that these constructs, if expressed with a TPA leader, will lack the N-terminal serine. Mutations were generated in the ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNA was isolated. For murine constructs (mFc), a murine IgG2a was substituted for the human IgG1. All mutants were sequence verified.

The amino acid sequence of unprocessed ActRIIB(K55A)-G1Fc is shown below (SEQ ID NO: 31). The signal sequence and linker sequence are indicated by solid underline, and the K55A substitution is indicated by double underline. The amino acid sequence of SEQ ID NO:31 may optionally be provided with the lysine removed from the C-terminus.

| (SEQ ID NO: 31) | |
|---|---|
| 1 | MDAMKRGLCC VLLLCGAVFV SPGASGRGEA ETRECIYYNA NWELERTNQS |
| 51 | GLERCEGEQD ARLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(K55A)-G1Fc fusion polypeptide is encoded by the following nucleic acid sequence (SEQ ID NO: 32):

| (SEQ ID NO: 32) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC GCCCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCCCCGGG |
| 1101 | TAAA |

The mature ActRIIB(K55A)-G1Fc fusion polypeptide (SEQ ID NO: 33) is as follows and may optionally be provided with the lysine removed from the C-terminus.

| (SEQ ID NO: 33) | |
|---|---|
| 1 | GRGEAETREC IYYNANWELE RTNQSGLERC EGEQDARLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(K55E)-G1Fc is shown below (SEQ ID NO: 34). The signal sequence and linker sequence are indicated by solid underline, and the K55E substitution is indicated by double underline. The amino acid sequence of SEQ ID NO:34 may optionally be provided with the lysine removed from the C-terminus.

| (SEQ ID NO: 34) | |
|---|---|
| 1 | MDAMKRGLCC VLLLCGAVFV SPGASGRGEA ETRECIYYNA NWELERTNQS |
| 51 | GLERCEGEQD ERLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(K55E)-G1Fc fusion polypeptide is encoded by the following nucleic acid sequence (SEQ ID NO: 35):

| (SEQ ID NO: 35) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC GAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCCCCGGG |
| 1101 | TAAA |

The mature ActRIIB(K55E)-G1Fc fusion polypeptide (SEQ ID NO: 36) is as follows and may optionally be provided with the lysine removed from the C-terminus.

| (SEQ ID NO: 36) | |
|---|---|
| 1 | GRGEAETREC IYYNANWELE RTNQSGLERC EGEQDERLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(F82I)-G1Fc is shown below (SEQ ID NO: 37). The signal sequence and linker sequence are indicated by solid underline, and the F82I substitution is indicated by double underline. The amino acid sequence of SEQ ID NO: may optionally be provided with the lysine removed from the C-terminus.

| (SEQ ID NO: 37) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDINC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(F82I)-G1Fc fusion polypeptide is encoded by the following nucleic acid sequence (SEQ ID NO: 38):

| (SEQ ID NO: 38) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CATCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

The mature ActRIIB(F82I)-G1Fc fusion polypeptide (SEQ ID NO: 39) is as follows and may optionally be provided with the lysine removed from the C-terminus.

| (SEQ ID NO: 39) | |
|---|---|
| 1 | GRGEAETREC IYYNANWELE RTNQSGLERC EGEQDKRLHC YASWRNSSGT |
| 51 | IELVKKGCWL DDINCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

The amino acid sequence of unprocessed ActRIIB(F82K)-G1Fc is shown below (SEQ ID NO: 40). The signal sequence and linker sequence are indicated by solid underline, and the F82K substitution is indicated by double underline. The amino acid sequence of SEQ ID NO: 40 may optionally be provided with the lysine removed from the C-terminus.

| (SEQ ID NO: 40) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDKNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPGK |

This ActRIIB(F82K)-G1Fc fusion polypeptide is encoded by the following nucleic acid sequence (SEQ ID NO: 41):

| (SEQ ID NO: 41) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CAAGAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGGAC TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

The mature ActRIIB(F82K)-G1Fc fusion polypeptide (SEQ ID NO: 42) is as follows and may optionally be provided with the lysine removed from the C-terminus.

| (SEQ ID NO: 42) | |
|---|---|
| 1 | GRGEAETREC IYYNANWELE RTNQSGLERC EGEQDKRLHC YASWRNSSGT |
| 51 | IELVKKGCWL DDKNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYKTT PPVLDSDGSF |
| 301 | FLYSKLTVDK SRWQQGNVFS CSVMHEALHN HYTQKSLSLS PGK |

Constructs were expressed in COS or CHO cells and purified by filtration and protein A chromatography. In some instances, assays were performed with conditioned medium rather than purified proteins. Purity of samples for reporter gene assays was evaluated by SDS-PAGE and Western blot analysis.

Mutants were tested in binding assays and/or bioassays described below.

Alternatively, similar mutations could be introduced into an ActRIIB extracelluar domain possessing an N-terminal truncation of five amino acids and a C-terminal truncation of three amino acids as shown below (SEQ ID NO: 53). This truncated ActRIIB extracellular domain is denoted ActRIIB(25-131) based on numbering in SEQ ID NO: 2.

| (SEQ ID NO: 53) | |
|---|---|
| 25 | ETRECIYYNA NWELERTNQS GLERCEGEQD KRLHCYASWR NSSGTIELVK |
| 75 | KGCWLDDFNC YDRQECVATE ENPQVYFCCC EGNFCNERFT HLPEAGGPEV |
| 125 | TYEPPPT |

The corresponding background fusion polypeptide, ActRIIB(25-131)-G1Fc, is shown below (SEQ ID NO: 12).

| (SEQ ID NO: 12) | |
|---|---|
| 1 | ETRECIYYNA NWELERTNQS GLERCEGEQD KRLHCYASWR NSSGTIELVK |
| 51 | KGCWLDDFNC YDRQECVATE ENPQVYFCCC EGNFCNERFT HLPEAGGPEV |
| 101 | TYEPPPTGGG THTCPPCPAP ELLGGPSVFL FPPKPKDTLM ISRTPEVTCV |
| 151 | VVDVSHEDPE VKFNWYVDGV EVHNAKTKPR EEQYNSTYRV VSVLTVLHQD |
| 201 | WLNGKEYKCK VSNKALPAPI EKTISKAKGQ PREPQVYTLP PSREEMTKNQ |
| 251 | VSLTCLVKGF YPSDIAVEWE SNGQPENNYK TTPPVLDSDG SFFLYSKLTV |
| 301 | DKSRWQQGNV FSCSVMHEAL HNHYTQKSLS LSPGK |

### Example 6. Ligand Binding Profiles of Variant ActRIIB-Fc Homodimers and Activity of Variant ActRIIB-Fc Polypeptides in a Cell-Based Assay

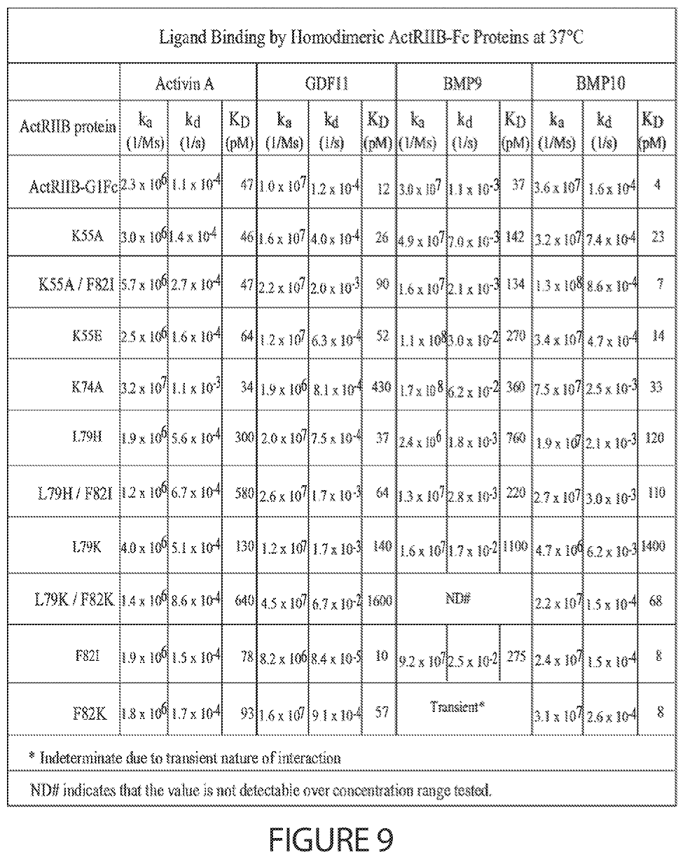

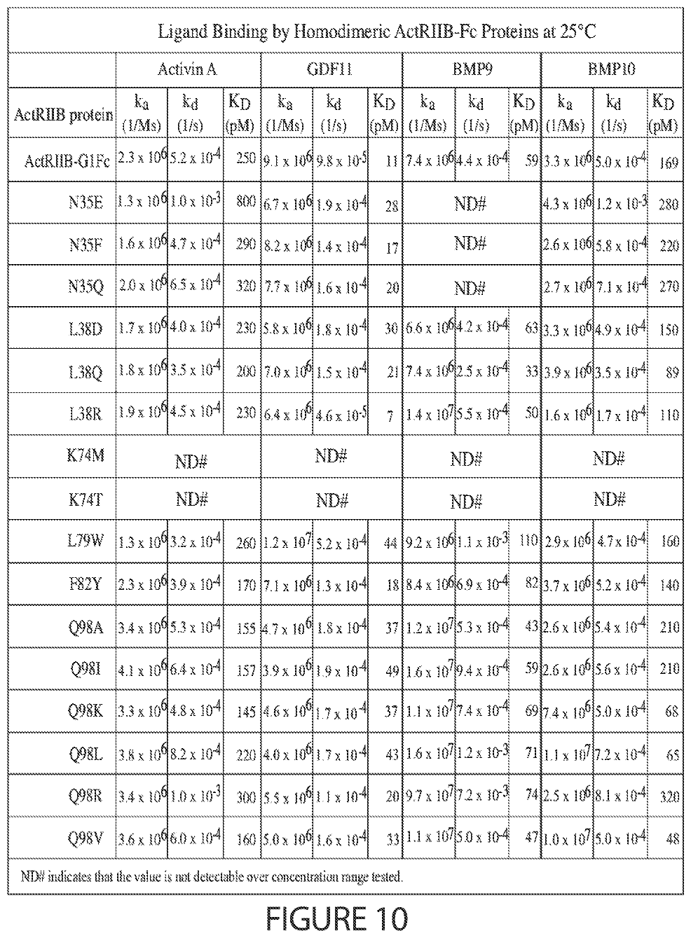

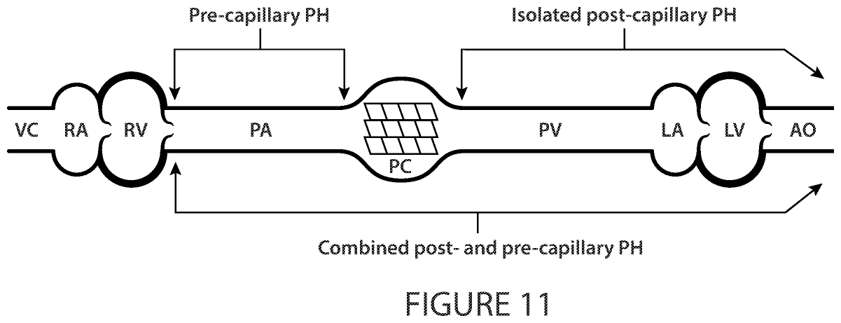

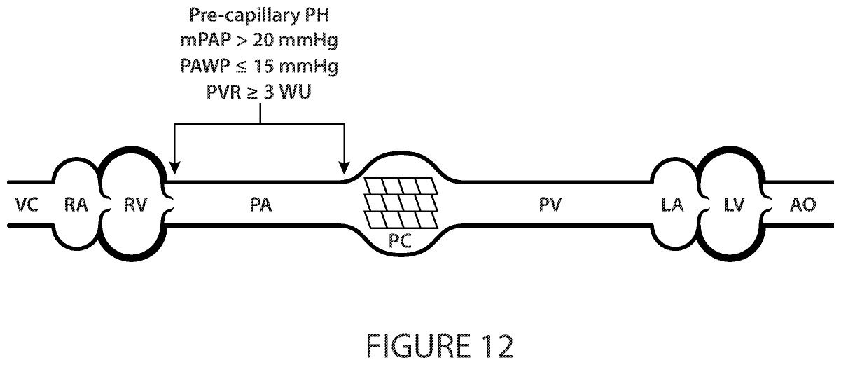

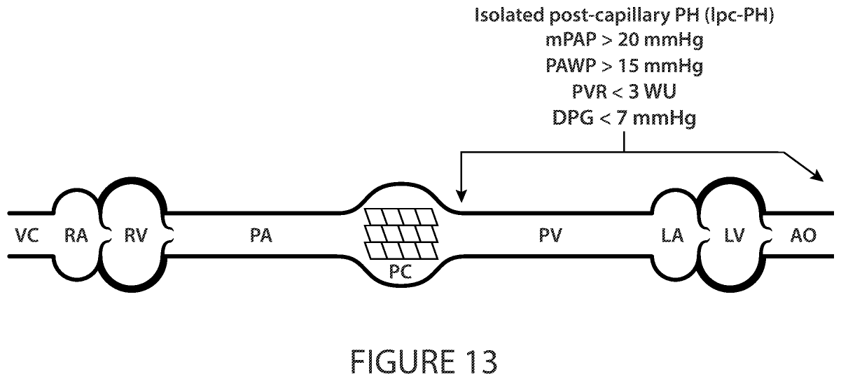

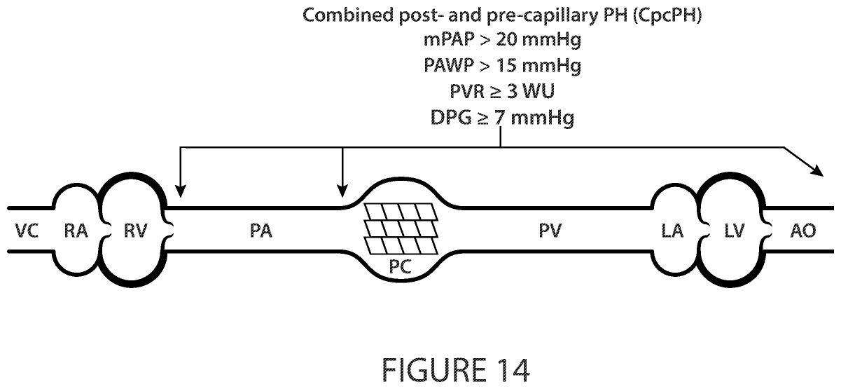

To determine ligand binding profiles of variant ActRIIB-Fc homodimers, a Biacore™-based binding assay was used to compare ligand binding kinetics of certain variant ActRIIB-Fc polypeptides. ActRIIB-Fc polypeptides to be tested were independently captured onto the system using an anti-Fc antibody. Ligands were then injected and allowed to flow over the captured receptor protein. Results of variant ActRIIB-Fc polypeptides analyzed at 37° C., are shown in FIG. 9. Compared to Fc-fusion polypeptide comprising unmodified ActRIIB extracellular domain, the variant polypeptides ActRIIB(K55A)-Fc, ActRIIB(K55E)-Fc, ActRIIB(F82I)-Fc, and ActRIIB(F82K)-Fc exhibited greater reduction in their affinity for BMP9 than for GDF11. Results of additional variant ActRIIB-Fc polypeptides analyzed at 25° C., are shown in FIG. 10.

These results confirm K55A, K55E, F82I, and F82K as substitutions that reduce ActRIIB binding affinity for BMP9 more than they reduce ActRIIB affinity for activin A or GDF11. Accordingly, these variant ActRIIB-Fc polypeptides may be more useful than unmodified ActRIIB-Fc polypeptide in certain applications where such selective antagonism is advantageous. Examples include therapeutic applications where it is desirable to retain antagonism of one or more of activin A, activin B. GDF8, and GDF11 while reducing antagonism of BMP9.

To determine activity of variant ActRIIB-Fc polypeptides, an A204 cell-based assay was used to compare effects among variant ActRIIB-Fc polypeptides on signaling by activin A, GDF11, and BMP9. In brief, this assay uses a human A204 rhabdomyosarcoma cell line (ATCC®: HTB-82™) derived from muscle and the reporter vector pGL3(CAGA)12 (Dennler el al., 1998, EMBO 17: 3091-3100) as well as a Renilla reporter plasmid (pRLCMV) to control for transfection efficiency. The CAGA12 motif is present in TGF-β responsive genes (e.g., PAI-1 gene), so this vector is of general use for ligands that can signal through Smad2/3, including activin A, GDF11, and BMP9.

On day 1, A-204 cells were transferred into one or more 48-well plates. On day 2, these cells were transfected with 10 μg pGL3(CAGA)12 or pGL3(CAGA)12(10 μg)+pRLCMV (1 μg) and Fugene. On day 3, ligands diluted in medium containing 0.1% BSA were preincubated with ActRIIB-Fc polypeptides for 1 hr before addition to cells. Approximately six hour later, the cells were rinsed with PBS and lysed. Cell lysates were analyzed in a luciferase assay to determine the extent of Smad activation.

This assay was used to screen variant ActRIIB-Fc polypeptides for inhibitory effects on cell signaling by activin A, GDF11, and BMP9. Potencies of homodimeric Fc fusion polypeptides incorporating amino acid substitutions in the human ActRIIB extracellular domain were compared with that of an Fc fusion polypeptide comprising unmodified human ActRIIB extracellular domain.

TABLE 5
Inhibitory Potency of
Homodimeric ActRIIB-Fc Constructs

| ActRIIB | IC₅₀ (ng/ml) | | |
|---|---|---|---|
| polypeptide | Activin A | GDF11 | BMP9 |
| Wild-type | 8 | 9 | 31 |
| A24N | 128 | 99 | 409 |
| R40A | — | 591 | 1210 |
| E50K | 132 | 180 | 721 |
| E50P | 756 | 638 | ~3000 |
| E52A | 198 | 71 | 359 |
| E52K | 762 | 296 | ~10000 |
| K55A | 15 | 11 | 122 |
| K55D | 396 | 365 | 5500 |
| K55E | 19 | 14 | 290 |
| K55R | 206 | 318 | 777 |
| Y60K | — | 414 | ND |
| Y60P | — | 544 | ND |
| K74R | — | 45 | 165 |
| K74Y | — | ND | ND |
| K74A/ | — | ND | ND |
| L79P | | | |
| L79K | — | 477 | ND |
| L79P | — | ND | ND |
| L79R | — | 234 | ND |
| D80A | — | ND | ND |
| F82I | 11 | 9 | 277 |
| F82K | 10 | 15 | ~5000 |
| F82W | — | 276 | ND |
| F82W/ | — | 389 | ~40000 |
| N83A | | | |
| V99E | — | ND | ND |
| V99K | — | ND | — |

ND: not detectable over concentration range tested
— Not tested

As shown in the table above, single amino acid substitutions in the ActRIIB extracellular domain can alter the balance between activin A or GDF11 inhibition and BMP9 inhibition in a cell-based reporter gene assay. Compared to a fusion polypeptide containing unmodified ActRIIB extracellular domain, the variants ActRIIB(K55A)-Fc, ActRIIB(K55E)-Fc, ActRIIB(F82I)-Fc, and ActRIIB(F82K)-Fc showed less potent inhibition of BMP9 (increased IC₅₀ values) while maintaining essentially undiminished inhibition of activin A and GDF11.

These results indicate that variant ActRIIB-Fc polypeptides such as ActRIIB(K55A)-Fc, ActRIIB(K55E)-Fc, ActRIIB(F82I)-Fc, and ActRIIB(F82K)-Fc are more selective antagonists of activin A and GDF11 compared to an Fc fusion polypeptide comprising unmodified ActRIIB extracellular domain. Accordingly, these variants may be more useful than ActRIIB-Fc in certain applications where such selective antagonism is advantageous. Examples include therapeutic applications where it is desirable to retain antagonism of one or more of activin A, GDF8, and GDF11 while reducing antagonism of BMP9 and potentially BMP10.

### Example 7. Generation of an ActRIIB-Fc:ActRIIB(L79E)-Fc Heterodimer

Applicants envision generation of a soluble ActRIIB-Fc:ActRIIB(L79E)-Fc heteromeric complex comprising the extracellular domains of unmodified human ActRIIB and human ActRIIB with a leucine-to-glutamate substitution at position 79, which are each separately fused to an G1Fc domain with a linker positioned between the extracellular domain and the G1Fc domain. The individual constructs are referred to as ActRIIB-Fc fusion polypeptide and ActRIIB(L79E)-Fc fusion polypeptide, respectively, and the sequences for each are provided below.

A methodology for promoting formation of ActRIIB-Fc:ActRIIB(L79E)-Fc heteromeric complexes, as opposed to the ActRIIB-Fc or ActRIIB(L79E)-Fc homodimeric complexes, is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the ActRIIB(L79E)-Fc and ActRIIB-Fc polypeptide sequences of SEQ ID NOs: 43-45 and 4648, respectively, one Fc domain can be altered to introduce cationic amino acids at the interaction face, while the other Fc domain can be altered to introduce anionic amino acids at the interaction face. The ActRIIB(L79E)-Fc fusion polypeptide and ActRIIB-Fc fusion polypeptide can each employ the TPA leader (SEQ ID NO: 8).

The ActRIIB(L79E)-Fc polypeptide sequence (SEQ ID NO: 43) is shown below:

| (SEQ ID NO: 43) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWEDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSREEMT KNQVSLTCLV KGFYPSDIAV |
| 301 | EWESNGQPEN NYDTTPPVLD SDGSFFLYSD LTVDKSRWQQ GNVFSCSVMH |
| 351 | EALHNHYTQK SLSLSPG |

The leader (signal) sequence and linker are underlined, and the L79E substitution is indicated by double underline. To promote formation of the ActRIIB-Fc:ActRIIB(L79E)-Fc heterodimer rather than either of the possible homodimeric complexes, two amino acid substitutions (replacing lysines with acidic amino acids) can be introduced into the Fc domain of the ActRIIB fusion polypeptide as indicated by double underline above. The amino acid sequence of SEQ ID NO: 43 may optionally be provided with lysine added to the C-terminus.

This ActRIIB(L79E)-Fc fusion polypeptide can be encoded by the following nucleic acid sequence (SEQ ID NO: 44):

| (SEQ ID NO: 44) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGGAAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGGA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACGACA CCACGCCTCC |
| 951 | CGTGCTGGAC TCCGACGGCT CCTTCTTCCT CTATAGCGAC CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | T |

The mature ActRIIB(L79E)-Fc fusion polypeptide (SEQ ID NO: 45) is as follows, and may optionally be provided with lysine added to the C-terminus.

| (SEQ ID NO: 45) | |
|---|---|
| 1 | GRGEAETREC IYYNANWELE RTNQSGLERC EGEQDKRLHC YASWRNSSGT |
| 51 | IELVKKGCWE DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | REEMTKNQVS LTCLVKGFYP SDIAVEWESN GQPENNYDTT PPVLDSDGSF |
| 301 | FLYSDLTVDK SRWQQGNVFS CSVMHEALHN HYTQKSLSLS PG |

The complementary form of ActRIIB-Fc fusion polypeptide (SEQ ID NO: 46) is as follows:

| (SEQ ID NO: 46) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPSRKEMT KNQVSLTCLV KGFYPSDIAV |
| 301 | EWESNGQPEN NYKTTPPVLK SDGSFFLYSK LTVDKSRWQQ GNVFSCSVMH |
| 351 | EALHNHYTQK SLSLSPGK |

The leader sequence and linker sequence are underlined. To guide heterodimer formation with the ActRIIB(L79E)-Fc fusion polypeptide of SEQ ID NOs: 43 and 45 above, two amino acid substitutions (replacing a glutamate and an aspartate with lysines) can be introduced into the Fc domain of the ActRIIB-Fc fusion polypeptide as indicated by double underline above. The amino acid sequence of SEQ ID NO: 46 may optionally be provided with lysine removed from the C-terminus.

This ActRIIB-Fc fusion polypeptide can be encoded by the following nucleic acid (SEQ ID NO: 47):

| (SEQ ID NO: 47) | |
|---|---|
| 1 | ATGGATGCAA TGAAGAGAGG GCTCTGCTGT GTGCTGCTGC TGTGTGGAGC |
| 51 | AGTCTTCGTT TCGCCCGGCG CCTCTGGGCG TGGGGAGGCT GAGACACGGG |
| 101 | AGTGCATCTA CTACAACGCC AACTGGGAGC TGGAGCGCAC CAACCAGAGC |
| 151 | GGCCTGGAGC GCTGCGAAGG CGAGCAGGAC AAGCGGCTGC ACTGCTACGC |
| 201 | CTCCTGGCGC AACAGCTCTG GCACCATCGA GCTCGTGAAG AAGGGCTGCT |
| 251 | GGCTAGATGA CTTCAACTGC TACGATAGGC AGGAGTGTGT GGCCACTGAG |
| 301 | GAGAACCCCC AGGTGTACTT CTGCTGCTGT GAAGGCAACT TCTGCAACGA |
| 351 | GCGCTTCACT CATTTGCCAG AGGCTGGGGG CCCGGAAGTC ACGTACGAGC |
| 401 | CACCCCCGAC AGCCCCCACC GGTGGTGGAA CTCACACATG CCCACCGTGC |
| 451 | CCAGCACCTG AACTCCTGGG GGGACCGTCA GTCTTCCTCT TCCCCCCAAA |
| 501 | ACCCAAGGAC ACCCTCATGA TCTCCCGGAC CCCTGAGGTC ACATGCGTGG |
| 551 | TGGTGGACGT GAGCCACGAA GACCCTGAGG TCAAGTTCAA CTGGTACGTG |
| 601 | GACGGCGTGG AGGTGCATAA TGCCAAGACA AAGCCGCGGG AGGAGCAGTA |
| 651 | CAACAGCACG TACCGTGTGG TCAGCGTCCT CACCGTCCTG CACCAGGACT |
| 701 | GGCTGAATGG CAAGGAGTAC AAGTGCAAGG TCTCCAACAA AGCCCTCCCA |
| 751 | GCCCCCATCG AGAAAACCAT CTCCAAAGCC AAAGGGCAGC CCCGAGAACC |
| 801 | ACAGGTGTAC ACCCTGCCCC CATCCCGGAA GGAGATGACC AAGAACCAGG |
| 851 | TCAGCCTGAC CTGCCTGGTC AAAGGCTTCT ATCCCAGCGA CATCGCCGTG |
| 901 | GAGTGGGAGA GCAATGGGCA GCCGGAGAAC AACTACAAGA CCACGCCTCC |
| 951 | CGTGCTGAAG TCCGACGGCT CCTTCTTCCT CTATAGCAAG CTCACCGTGG |
| 1001 | ACAAGAGCAG GTGGCAGCAG GGGAACGTCT TCTCATGCTC CGTGATGCAT |
| 1051 | GAGGCTCTGC ACAACCACTA CACGCAGAAG AGCCTCTCCC TGTCTCCGGG |
| 1101 | TAAA |

The mature ActRIIB-Fc fusion polypeptide sequence (SEQ ID NO: 48) is as follows and may optionally be provided with lysine removed from the C-terminus:

| (SEQ ID NO: 48) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPS |
| 251 | RKEMTKNQVS LTCLVKGFYP SDIAVEWESN GQPENNYKTT PPVLKSDGSF |
| 301 | FLYSKLTVDK SRWQQGNVFS CSVMHEALHN HYTQKSLSLS PGK |

The ActRIIB(L79E)-Fc and ActRIIB-Fc polypeptides of SEQ ID NO: 45 and SEQ ID NO: 48, respectively, may be co-expressed and purified from a CHO cell line, to give rise to a heteromeric polypeptide complex comprising ActRIIB-Fc:ActRIIB(L79E)-Fc.

In another approach to promote the formation of heteromultimer complexes using asymmetric Fc fusion polypeptides, the Fc domains can be altered to introduce complementary hydrophobic interactions and an additional intermolecular disulfide bond as illustrated in the ActRIIB(L79E)-Fc and ActRIIB-Fc polypeptide sequences of SEQ ID NOs: 49-50 and 51-52, respectively. The ActRIIB(L79E)-Fc fusion polypeptide and ActRIIB-Fc fusion polypeptide can each employ the TPA leader (SEQ ID NO: 8). ActRIIB(L79E)-Fc polypeptide sequence (SEQ ID NO: 49) is shown below:

| (SEQ ID NO: 49) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWEDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVY TLPPCREEMT KNQVSLWCLV KGFYPSDIAV |
| 301 | EWESNGQPEN NYKTTPPVLD SDGSFFLYSK LTVDKSRWQQ GNVFSCSVMH |
| 351 | EALHNHYTQK SLSLSPG |

The signal sequence and linker sequence are underlined, and the L79E substitution is indicated by double underline. To promote formation of the ActRIIB-Fc:ActRIIB(L79E)-Fc heterodimer rather than either of the possible homodimeric complexes, two amino acid substitutions (replacing a serine with a cysteine and a threonine with a trytophan) can be introduced into the Fc domain of the fusion polypeptide as indicated by be double underline above. The amino acid sequence of SEQ ID NO: 49 may optionally be provided with lysine added to the C-terminus.mature ActRIIB(L79E)-Fc fusion polypeptide (SEQ ID NO: 50) is as follows:

| (SEQ ID NO: 50) | |
|---|---|
| 1 | GRGEAETREC IYYNANWELE RTNQSGLERC EGEQDKRLHC YASWRNSSGT |
| 51 | IELVKKGCWE DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVYTLPPC |
| 251 | REEMTKNQVS LWCLVKGFYP SDIAVEWESN GQPENNYKTT PPVLDSDGSF |
| 301 | FLYSKLTVDK SRWQQGNVFS CSVMHEALHN HYTQKSLSLS PG |

The complementary form of ActRIIB-Fc fusion polypeptide (SEQ ID NO: 51) is as follows and may optionally be provided with lysine removed from the C-terminus.

| (SEQ ID NO: 51) | |
|---|---|
| 1 | MDAMKRGLCC VLLLCGAVFV SPGASGRGEA ETRECIYYNA NWELERTNQS |
| 51 | GLERCEGEQD KRLHCYASWR NSSGTIELVK KGCWLDDFNC YDRQECVATE |
| 101 | ENPQVYFCCC EGNFCNERFT HLPEAGGPEV TYEPPPTAPT GGGTHTCPPC |
| 151 | PAPELLGGPS VFLFPPKPKD TLMISRTPEV TCVVVDVSHE DPEVKFNWYV |
| 201 | DGVEVHNAKT KPREEQYNST YRVVSVLTVL HQDWLNGKEY KCKVSNKALP |
| 251 | APIEKTISKA KGQPREPQVC TLPPSREEMT KNQVSLSCAV KGFYPSDIAV |
| 301 | EWESNGQPEN NYKTTPPVLD SDGSFFLVSK LTVDKSRWQQ GNVFSCSVMH |
| 351 | EALHNHYTQK SLSLSPGK |

The leader sequence and linker are underlined. To guide heterodimer formation with the ActRIIB(L79E)-Fc fusion polypeptide of SEQ ID NOs: 49-50 above, four amino acid substitutions (replacement of tyrosine with cysteine, threonine with serine, leucine with alanine, and tyrosine with valine) can be introduced into the Fe domain of the ActRIIB-Fc fusion polypeptide as indicated by double underline. The amino acid sequence of SEQ ID NO: 51 may optionally be provided with lysine removed from the C-terminus.

The mature ActRIIB-Fc fusion polypeptide sequence is as follows and may optionally be provided with lysine removed from the C-terminus.

| (SEQ ID NO: 52) | |
|---|---|
| 1 | GRGEAETREC IYYNANWELE RTNQSGLERC EGEQDKRLHC YASWRNSSGT |
| 51 | IELVKKGCWL DDFNCYDRQE CVATEENPQV YFCCCEGNFC NERFTHLPEA |
| 101 | GGPEVTYEPP PTAPTGGGTH TCPPCPAPEL LGGPSVFLFP PKPKDTLMIS |
| 151 | RTPEVTCVVV DVSHEDPEVK FNWYVDGVEV HNAKTKPREE QYNSTYRVVS |
| 201 | VLTVLHQDWL NGKEYKCKVS NKALPAPIEK TISKAKGQPR EPQVCTLPPS |
| 251 | REEMTKNQVS LSCAVKGFYP SDIAVEWESN GQPENNYKTT PPVLDSDGSF |
| 301 | FLVSKLTVDK SRWQQGNVFS CSVMHEALHN HYTQKSLSLS PGK |

The ActRIIB(L79E)-Fc and ActRIIB-Fc polypeptides of SEQ ID NO: 50 and SEQ ID NO: 52, respectively, may be co-expressed and purified from a CHO cell line, to give rise to a heteromeric polypeptide complex comprising ActRIIB-Fc:ActRIIB(L79E)-Fc.

Purification of various ActRIIB-Fc:ActRIIB(L79E)-Fc complexes can be achieved by a series of column chromatography steps, including, for example, three or more of the following, in any order: protein A chromatography, Q sepharose chromatography, phenylsepharose chromatography, size exclusion chromatography, cation exchange chromatography, multimodal chromatography (e.g., with resin containing both electrostatic and hydrophobic ligands), and epitope-based affinity chromatography (e.g., with an antibody or functionally equivalent ligand directed against an epitope of ActRIIB). The purification can be completed with viral filtration and buffer exchange.

### Example 8. Ligand Binding Profile of ActRIIB-Fc:ActRIIB(L79E)-Fc Heteromer

A Biacore™-based binding assay was used to compare the ligand binding kinetics of an ActRIIB-Fc:ActRIIB(L79E)-Fc heterodimer with those of unmodified ActRIIB-Fc homodimer. Fusion proteins were captured onto the system using an anti-Fc antibody. Ligands were then injected and allowed to flow over the captured receptor protein at 37° C. Results are summarized in the table below, in which ligand off-rates (kd) most indicative of effective ligand traps are denoted in bold.

TABLE 6
Ligand binding of ActRIIB-Fc:ActRIIB(L79E)-Fc heterodimer
compared to ActRII-Fc homodimer at 37° C.

| | ActRIIB-Fc | | | ActRIIB-Fc:ActRIIB(L79E)-Fc | | |
|---|---|---|---|---|---|---|
| | homodimer | | | heterodimer | | |
| | kₐ | k_{d} | K_{D} | kₐ | k_{d} | K_{D} |
| Ligand | (1/Ms) | (1/s) | (pM) | (1/Ms) | (1/s) | (pM) |
| Activin A | 7.4 × 10⁶ | 1.9 × 10⁻⁴ | 25 | 8.8 × 10⁶ | 1.5 × 10⁻³ | 170 |
| Activin B | 8.1 × 10⁶ | 6.6 × 10⁻⁵ | 8 | 8.3 × 10⁶ | 2.1 × 10⁻⁴ | 25 |
| GDF3 | 1.4 × 10⁶ | 2.2 × 10⁻³ | 1500 | 5.8 × 10⁵ | 5.9 × 10⁻³ | 10000 |
| GDF8 | 3.8 × 10⁶ | 2.6 × 10⁻⁴ | 70 | 3.4 × 10⁶ | 5.0 × 10⁻⁴ | 150 |
| GDF11 | 4.1 × 10⁷ | 1.7 × 10⁻⁴ | 4 | 4.0 × 10⁷ | 3.6 × 10⁻⁴ | 9 |
| BMP6 | 1.3 × 10⁸ | 7.4 × 10⁻³ | 56 | 3.3 × 10⁸ | 1.8 × 10⁻² | 56 |
| BMP9 | 5.0 × 10⁶ | 1.3 × 10⁻³ | 250 | Transient* | | >2800 |
| BMP10 | 5.1 × 10⁷ | 2.0 × 10⁻⁴ | 4 | 4.8 × 10⁷ | 2.0 × 10⁻³ | 42 |

*Indeterminate due to transient nature of interaction

In this example, a single amino acid substitution in one of two ActRIIB polypeptide chains altered ligand binding selectivity of the Fc-fusion polypeptide relative to unmodified ActRIIB-Fc homodimer. Compared to ActRIIB-Fc homodimer, the ActRIIB(L79E)-Fc heterodimer largely retained high-affinity binding to activin B, GDF8, GDF11, and BMP6 but exhibited approximately ten-fold faster off-rates for activin A and BMP10 and an even greater reduction in the strength of binding to BMP9. Accordingly, a variant ActRIIB-Fc heteromer may be more useful than unmodified ActRIIB-Fc homodimer in certain applications where such selective antagonism is advantageous. Examples include therapeutic applications where it is desirable to retain antagonism of one or more of activin B, GDF8, GDF11, and BMP6, while reducing antagonism of activin A. BMP9, or BMP10.9. Generation of ActRIIB mutants:

A series of mutations in the extracellular domain of ActRIIB were generated and these mutant polypeptides were produced as soluble fusion polypeptides between extracellular ActRIIB and an Fe domain. A co-crystal structure of Activin and extracellular ActRIIB did not show any role for the final (C-terminal) 15 amino acids (referred to as the “tail” herein) of the extracellular domain in ligand binding. This sequence failed to resolve on the crystal structure, suggesting that these residues are present in a flexible loop that did not pack uniformly in the crystal. Thompson EMBO J. 2003 Apr. 1:22(7):1555-66. This sequence is also poorly conserved between ActRIIB and ActRIIA. Accordingly, these residues were omitted in the basic, or background, ActRIIB-Fc fusion construct. Additionally, in this example position 64 in the background form is occupied by an alanine. Thus, the background ActRIIB-Fc fusion in this example has the sequence (Fc portion underlined) (SEQ ID NO; 54):

SGRGEAETRECIYYNANWELERTNQSGLERCEGEQDKRLHCYASWANSSG
TIELVKKGCWLDDFNCYDRQECVATEENPQVYFCCCEGNFCNERFTHLPE
AGGGTHTCPPCPAPELLGGPSVFLFPPKPKDTIMISRTPEVTCVVVDVSH
EDPEVKFNWYVDGVEVHNAKTKPREEQYNSTYRVVSVLTVLHQDWLNGKE
YKCKVSNKALPVPIEKTISKAKGQPREPQVYTLPPSREEMTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

Surprisingly, as discussed below, the C-terminal tail was found to enhance activin and GDF-11 binding, thus a preferred version of ActRIIB-Fc has a sequence (Fc portion underlined) (SEQ ID NO: 55):

SGRGEAETRECIYYNANWELERTNQSGLERCEGEQDKRLHCYASWANSSG
TIELVKKGCWLDDFNCYDRQECVATEENPQVYFCCCEGNFCNERFTHLPE
AGGPEVTYEPPPTAPTGGGTHTCPPCPAPELLGGPSVFLFPPKPKDTIMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK

Various mutations were introduced into the background ActRIIB-Fc polypeptide. Mutations were generated in ActRIIB extracellular domain by PCR mutagenesis. After PCR, fragments were purified thru Qiagen column, digested with SfoI and AgeI and gel purified. These fragments were ligated into expression vector pAID4 such that upon ligation it created fusion chimera with human IgG1. DNAs were isolated. All of the mutants were produced in HEK293T cells by transient transfection. In summary, in a 500 ml spinner, HEK293T cells were set up at 6×10⁵ cells/ml in Freestyle (Invitrogen) media in 250 ml volume and grown overnight. Next day, these cells were treated with DNA:PEI (1:1) complex at 0.5 ug/ml final DNA concentration. After 4 hrs, 250 ml media was added and cells were grown for 7 days. Conditioned media was harvested by spinning down the cells and concentrated.

All the mutants were purified over protein A column and eluted with low pH (3.0) glycine buffer. After neutralization, these were dialyzed against PBS.

Mutants were also produced in CHO cells by similar methodology.

Mutants were tested in binding assays and bioassays described below. Proteins expressed in CHO cells and HEK293 cells were indistinguishable in the binding assays and bioassays.

### Example 9. Effects of an ActRIIA-mFc on Group 2 Pulmonary Hypertension in a Transverse Aortic Constriction (TAC) Induced PH Mouse Model

The effects of an ActRIIA-mFc fusion polypeptide (SEQ ID NO: 56) was examined in a mouse model of left ventricular systolic dysfunction (also referred to as HErEF) of pulmonary hypertension (PH). In this model, C57BL/6 mice underwent transverse aortic constriction (TAC) to induce left heart failure, and right heart and pulmonary remodeling. See, e.g., Xiong P Y, et al. Hypertension 2018, 71(1):34-55 and Chen Y, et al. Hypertension 2012, 59(6):1170-1178.

Twenty-six C57/B6 male mice (10 wks old) underwent TAC surgery and ten age-matched animals underwent a mock surgical procedure (Sham) at day 0. Two weeks after the surgery, TAC-PH mice were randomized into two groups. i) fourteen mice were injected subcutaneously with vehicle control (phosphate buffered saline (PBS)), twice weekly for 4 weeks starting from day 14 after surgery, “TAC-PH/PBS”: and a ii) twelve mice were injected subcutaneously with ActRIIA-mFc at a dose of 10 mg/kg twice weekly for 4 weeks starting from day 14 after TAC surgery, “TAC-PH/ActRII-mFc”. At the end of the study, echocardiography and pressure-volume catheter were performed to measure left and right ventricular remodeling and functional changes before animals were euthanized for heart and lung collection. Hearts and lungs of each mouse were weighed, fixed in 10% formalin, embedded in paraffin, and sectioned for Masson's trichrome stain to assess fibrosis.

Prior to euthanasia, in vivo cardiac function was assessed by transthoracic echocardiography (Acuson P300, 18 MHz transducer; Siemens) in conscious mice. From left ventricle (LV) short axis view, M-mode echocardiogram was acquired to measure left ventricle end diastolic diameter (LVEDD), and left ventricle end systolic diameter (LVESD). Fractional shortening (FS) was calculated from the end-diastolic diameter (EDD) and end-systolic diameter (ESD) using the following equation: FS=100%×[(EDD−ESD)/EDD]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Three to five beats were averaged for each mouse measurement. Tricuspid annular plane systolic excursion (TAPSE), a parameter of global right ventricular function, was also measured.

On day 42, mice were anesthetized by an intraperitoneal injection of ketamine/xylazine (100/5 mg/kg) to evaluate left and right ventricular function by Millar pressure-volume conductance catheter. The respiration was supported by a small animal ventilator. Thoracotomy was made through 4-5 intercostal space, and the heart was exposed. A pressure-volume catheter (1.0-Fr, PVR-1035, Millar Instruments, Houston, TX, USA) was inserted into the left ventricle and right ventricle from the apex. Ventricular pressure and volume were calculated with LabChart 7 software. Stroke work, ejection fraction, maximum and minimum rate of pressure development (+dp/dtm, −dp/dtm) were derived.

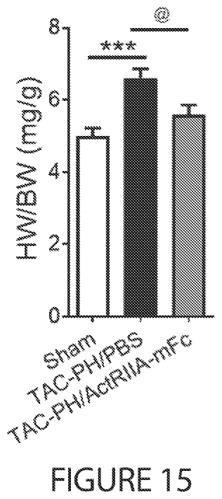

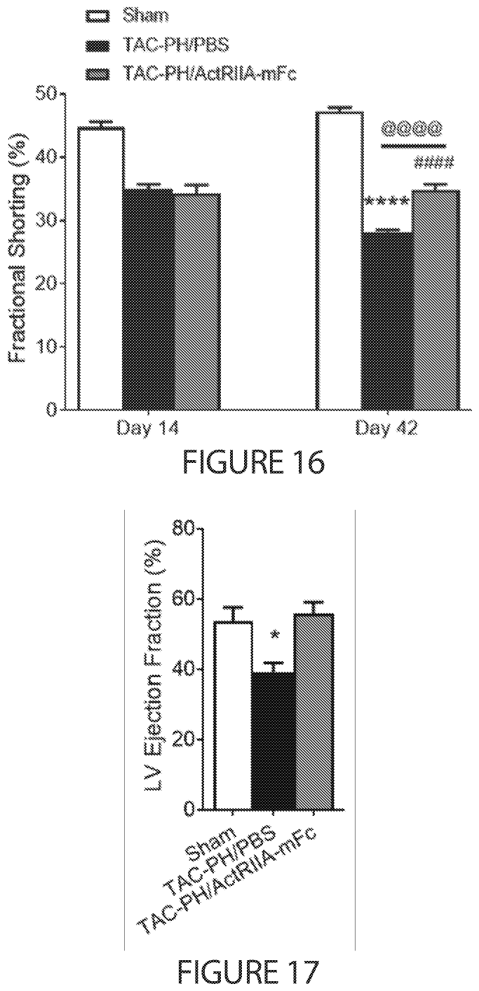

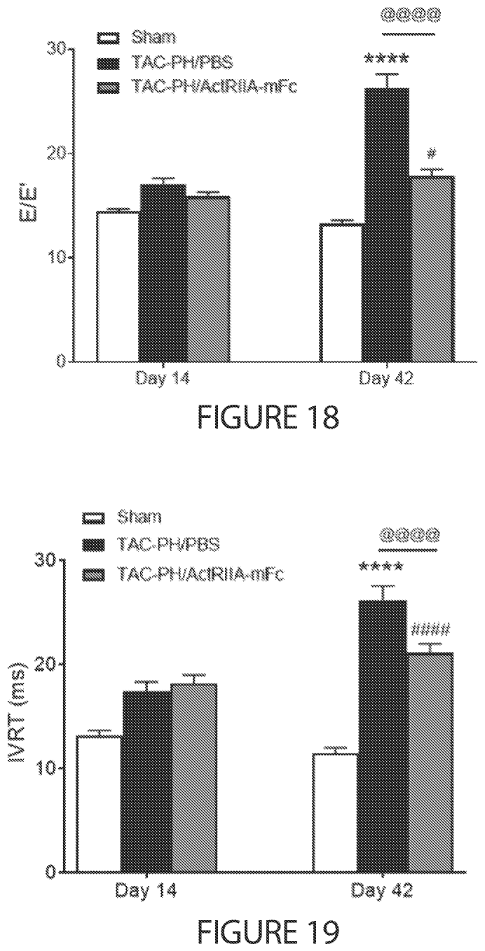

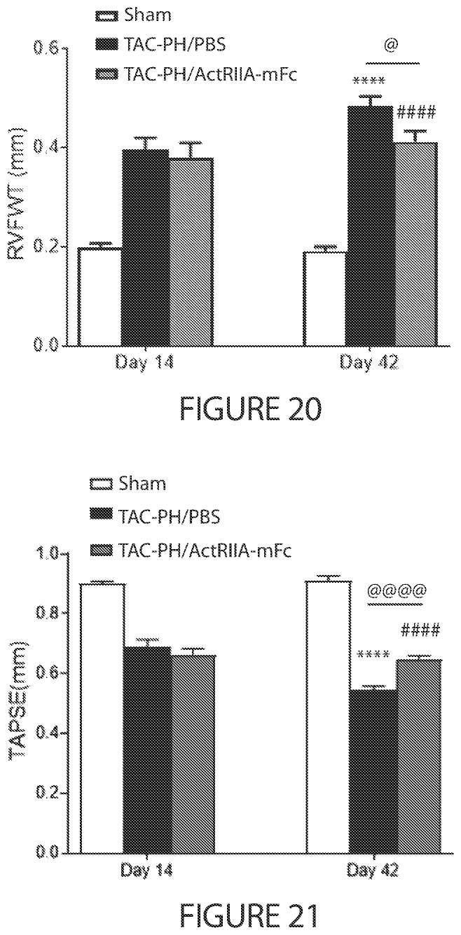

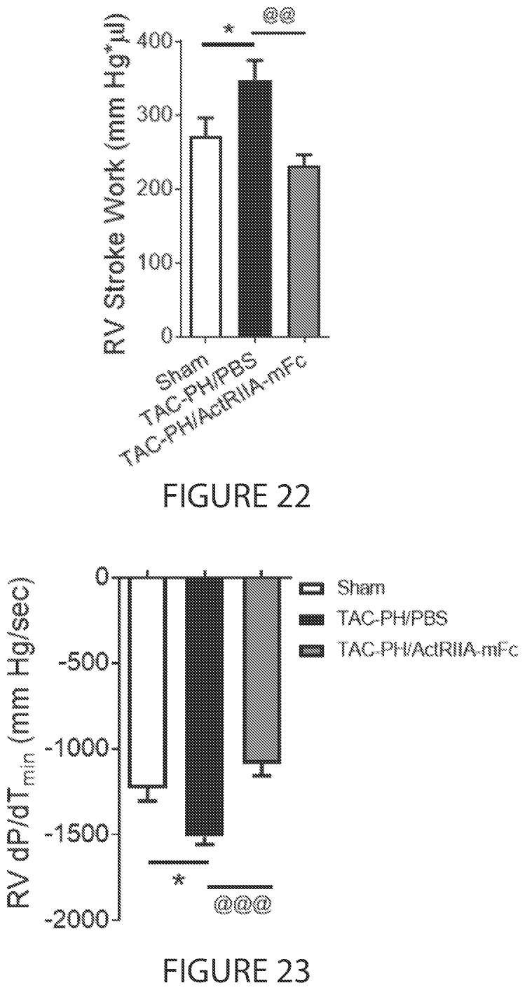

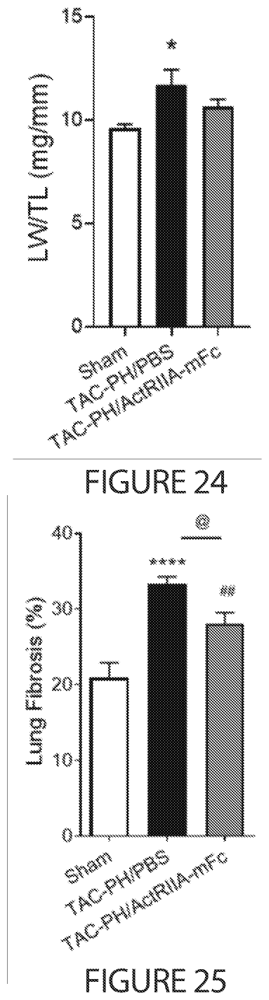

Compared to Sham control animals, TAC-PH mice in the PBS treatment group (TAC-PH/PBS) on day 42 were observed to have increased heart weight (HW/BW) (FIG. 15), reduced FS (FIG. 16), reduced LV ejection fraction (FIG. 17), increased E/E′ ratio (FIG. 18), and increased IVRT (FIG. 19), indicating cardiac hypertrophy and left heart failure. TAC mice also increased right ventricle free wall thickness (RVFWT) (FIG. 20), decreased TAPSE (FIG. 21), increased right ventricle (RV) stroke work (FIG. 22), and increased minimum rate of pressure development in RV (−dp/dTₘᵢₙ) (FIG. 23) compared to Sham control mice, suggesting the RV remodeling and RV dysfunction. In addition, increased lung weight (LW/TL) (FIG. 24) and lung fibrosis (FIG. 25) were observed in TAC-PH/PBS mice, indicating lung remodeling caused by TAC-induced left heart failure.

As shown in FIGS. 15-25, ActRIIA-mFc treatment (TAC-PH/ActRIIA-mFc) relative to PBS treatment (TAC-PH/PBS) on day 42 significantly reduced cardiac hypertrophy (FIG. 15), elevated FS (FIG. 16), restored LV ejection fraction (FIG. 17), reduced E/E′ ratio (FIG. 18), and reduced IVRT (FIG. 19). ActRIIA-mFc treatment (TAC-PH/ActRIIA-mFc) relative to PBS treatment (TAC-PH/PBS) on day 42 also significantly reduced elevated RVFWT (FIG. 20), increased reduced TAPSE (FIG. 21), reduced elevated RV stroke work (FIG. 22), and decreased increased RV−dp/dTₘᵢₙ (FIG. 23). ActRIIA-mFc treatment (TAC-PH/ActRII-mFc) relative to PBS treatment (TAC-PH/PBS) on day 42 decreased lung weight (FIG. 24) and significantly reduced lung fibrosis (FIG. 25).

Together, these data demonstrate that ActRIIA-mFc is effective in ameliorating various complications of Group 2 PH in a left heart failure-induced PH model (TAC-PH). In particular, ActRIIA-mFc had a significant effect in reducing cardiac hypertrophy, improving cardiac function, improving right heart remodeling and function, and reducing pulmonary remodeling and fibrosis.

### Example 10: Effects of an ActRIIA-mFc on Group 2 Pulmonary Hypertension in an HFPEF Induced PH Rat Model

The effects of an ActRIIA-mFc fusion polypeptide (SEQ ID NO: 56) was examined in a rat model of left ventricular diastolic dysfunction (also referred to as HEpEF) group 2 (subgroup 2.2) pulmonary hypertension (PH). In this model, ZSF1-Lepr^{fa}Lepr^{cp}/Crl rats were challenged with semaxanib to induce HFpEF-PH(l).

Forty ZSF1 Lepr^{fa}Lepr^{cp}/Crl male mice (8 wks old) and five lean rats were subcutaneously administered with a single dose of semaxanib (100 mg/kg) at day 0, and five lean rats were included as normal control. Six weeks after semaxanib (SU5416) treatment, Thirty-six ZSF1 Lepr^{fa}Lepr^{cp}/Crl rats were randomized into four groups: i) nine rats were injected subcutaneously with vehicle control (phosphate buffered saline (PBS)), twice weekly for 8 weeks starting from day 42 after semaxanib treatment, “ZSF1-SU/PBS”; a ii) ten rats were injected subcutaneously with ActRIIA-mFc at a dose of 1 mg/kg twice weekly for 8 weeks starting from day 42 after semaxanib treatment, “ZSF1-SU/ActRIIA-mFc 1 mpk”: a iii) nine rats were injected subcutaneously with ActRIIA-mFc at a dose of 3 mg/kg twice weekly for 8 weeks starting from day 42 after semaxanib treatment, “ZSF1-SU/ActRIIA-mFc 3 mpk”; and a iv) eight rats were injected subcutaneously with ActRIIA-mFc at a dose of 10 mg/kg twice weekly for 8 weeks starting from day 42 after semaxanib treatment, “ZSF1-SU/ActRIIA-mFc 10 mpk”. At the end of the study, echocardiography and pressure-volume catheter were performed to measure left and right ventricular remodeling and functional changes before animals were euthanized for heart and lung collection. Hearts and lungs of each rat were weighed, fixed in 10% formalin, embedded in paraffin, and sectioned for Masson's trichrome stain to assess fibrosis. Serum and urine samples were collected at the end of the study.

Rats were fasted overnight to measure fasting blood glucose levels at week 14 (before treatments started), week 18 (4 weeks after treatments), and week 22, and oral glucose tolerance test was performed at week 22. Blood glucose levels were measured with a glucometer after bleeding tail vein with a 27G needle. To prepare oral glucose tolerance test, 40% glucose stock solution run through a filter to sterilize it. After fasting overnight, rat body weight was measured. Blood glucose level was detected. Then 40% glucose solution was administered via oral gavage according to body weight (2 g/kg). Blood glucose levels were measured at 30, 60, 90, 120 minutes.

Prior to euthanization, in vivo cardiac function was assessed by transthoracic echocardiography (Acuson P300, 18 MHz linear transducer; Siemens) in lightly anesthetized rats as described (2). From left ventricle short axis view, M-mode echocardiogram was acquired to measure interventricular septal thickness at end diastole (IVSd), left ventricular posterior wall thickness at end diastole (LVPWd), left ventricular end diastolic diameter (LVEDD), and left ventricular end systolic diameter (LVESD). Left ventricular mass (LVM) was assessed by the equation: 1.05 [(LVEDD+LVPTD+IVSd)³-LVEDD³]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Pulmonary arterial acceleration time (PAAT), a parameter of right ventricular function, was also measured.

Fourteen weeks after semaxanib treatment, rats were anesthetized with ketamine (100 mg/kg) and xylazine (5 mg/kg) at the end of the experiment to evaluate cardiac and pulmonary hemodynamics. The respiration was supported by a small animal ventilator. Thoracotomy was made through 4-5 intercostal space, and the heart was exposed. A pressure-volume catheter (2.0-Fr, SPR-869, Millar Instruments, Houston, TX, USA) was be inserted into the left ventricle and right ventricle from the apex. Ventricular pressure and volume were calculated with LabChart 7 software. Stroke work, ejection fraction, and cardiac output were derived. After finishing left ventricular measurements, the catheter was advanced to the aorta, arterial blood systolic and diastolic pressure was detected. Then the catheter returned to the left ventricle and changed the direction laterally to enter the left atrium. Similarly, right atrial pressure was measured by moving the catheter from the right ventricle into atrium. To measure pulmonary arterial pressure, the sternum was cross-sectioned at the second inter-rib space. The right ventricular outflow tract was exposed. A hole was made with 27G needle, and then the catheter was inserted into the right ventricular outflow tract and advanced into the pulmonary artery.

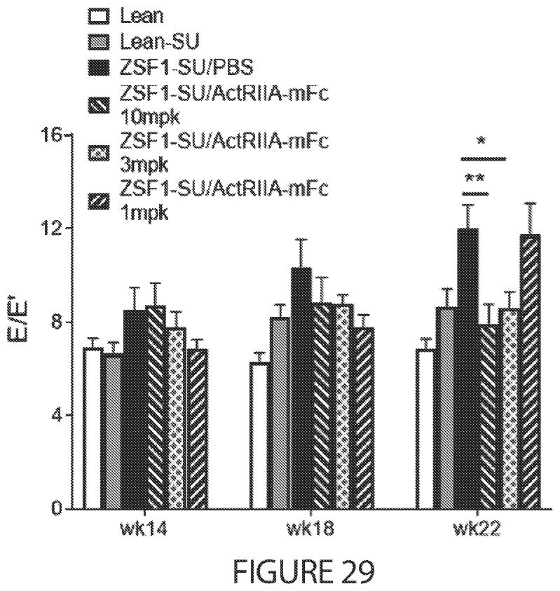

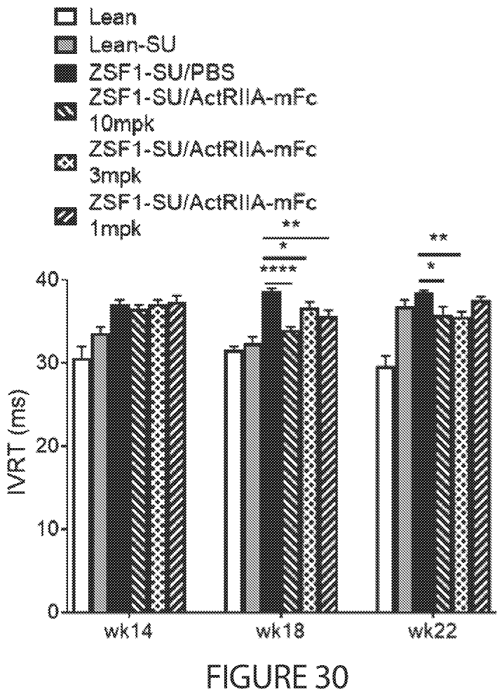

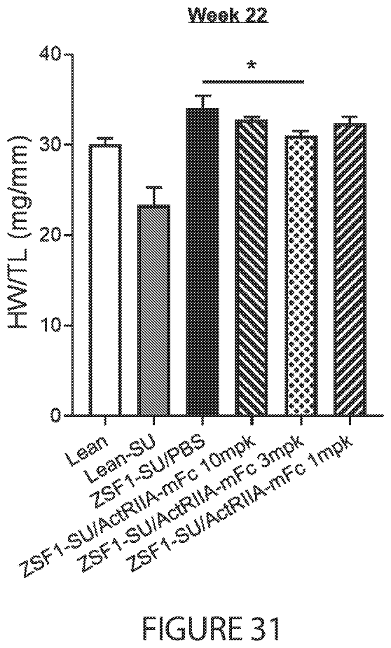

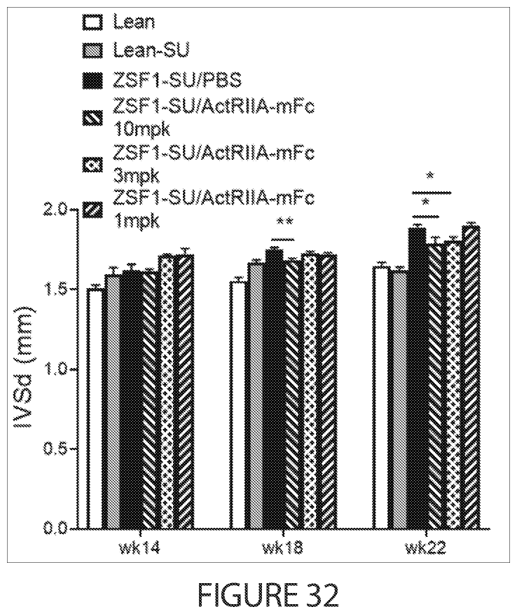

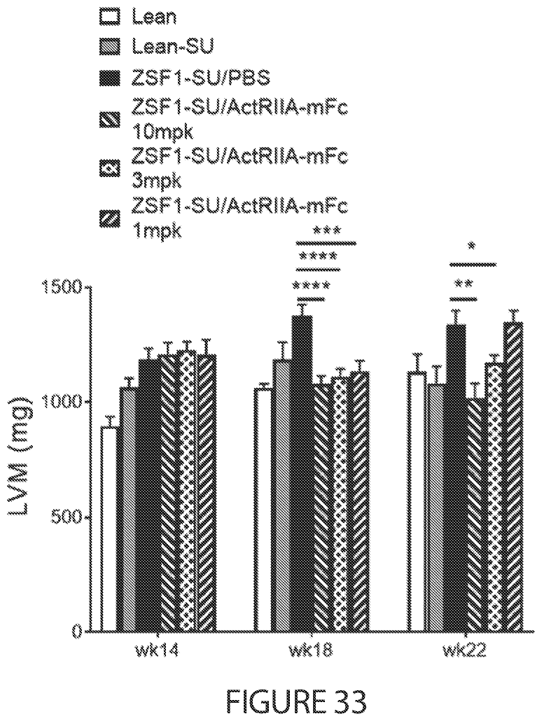

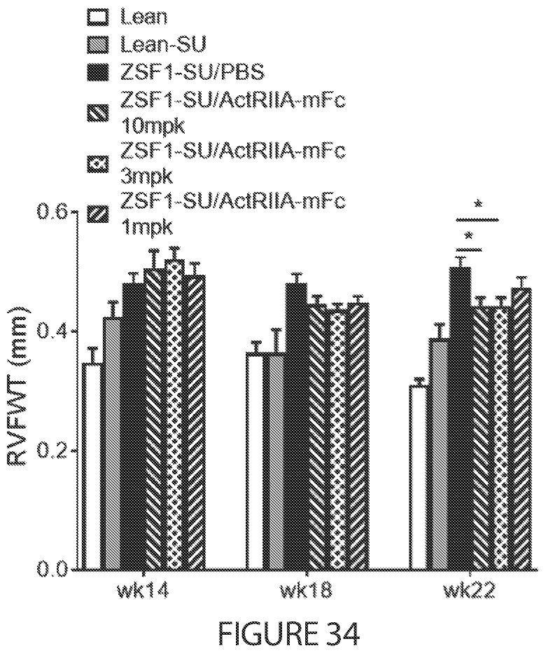

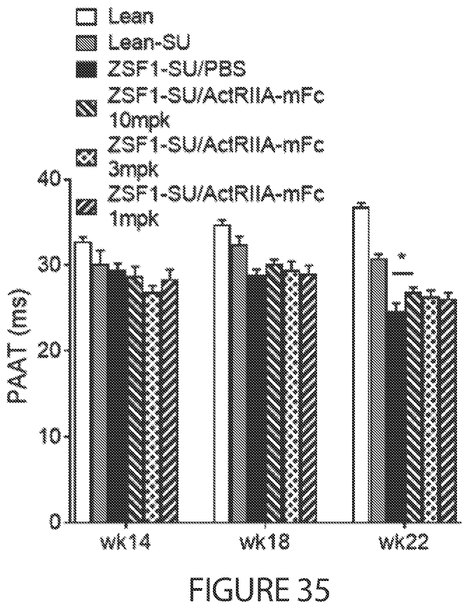

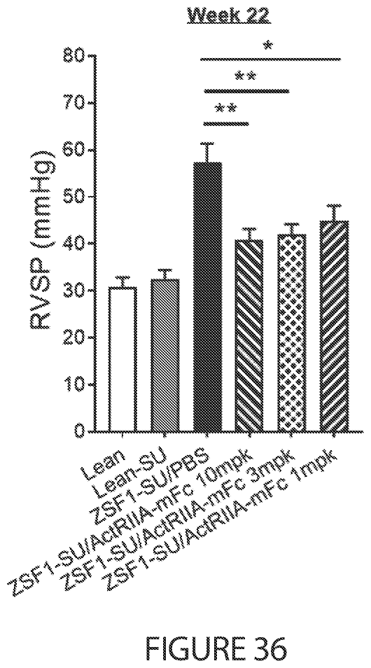

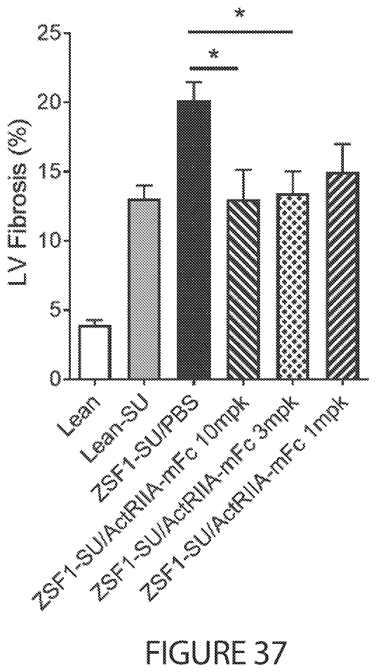

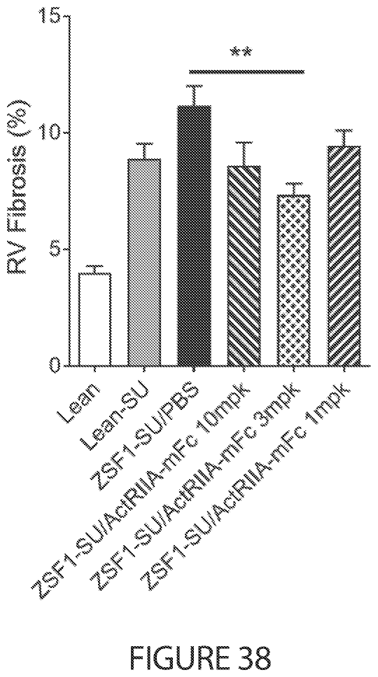

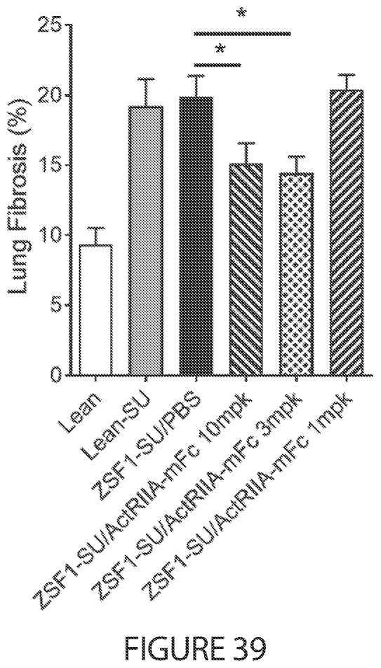

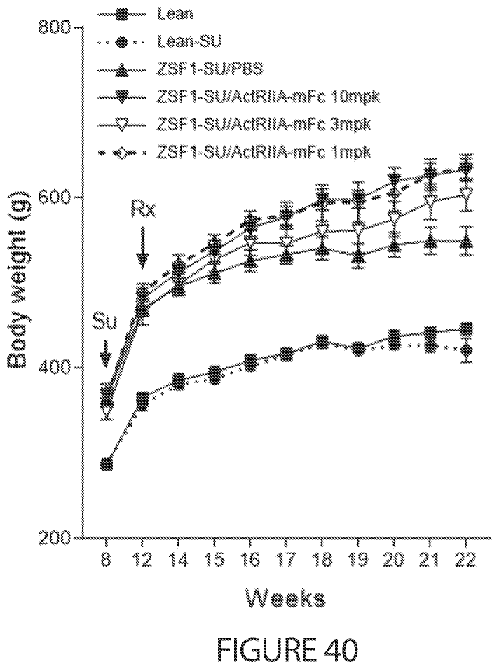

Compared to lean control animals, ZSF1-SU rats in the PBS treatment group (ZSF1-SU/PBS) 14-weeks after semaxanib treatment were observed to have increased heart weight (HWTL) (FIG. 31), increased IVSd (FIG. 32), and increased LVM (FIG. 33), preserved LV ejection fraction (FIG. 28), increased E/E′ ratio (FIG. 29), increased IVRT (FIG. 30), indicating cardiac hypertrophy and left ventricular diastolic dysfunction. ZSF1 rats also increased right ventricle free wall thickness (RVFWT) (FIG. 34), decreased PAAT (FIG. 35), and increased RVSP (FIG. 36), compared to lean control rats, suggesting the pulmonary hypertension and RV remodeling. In addition, increased fibrosis in LV, RV and lung (FIGS. 37-39) was observed in ZSF1-SU/PBS rats.

As shown in FIGS. 31 to 33, ActRIIA-mFc treatment (ZSF1-SU/ActRIIA-mFc) relative to PBS treatment (ZSF1-SU/PBS) both at 3 mpk and 10 mpk significantly reduced left heart remodeling (FIGS. 31-33), and reduced E/E′ ratio (FIG. 29), and decreased IVRT (FIG. 30). ActRIIA-mFc treatment (ZSF1-SU/ActRIIA-mFc) relative to PBS treatment (ZSF1-SU/PBS) both at 3 mpk and 10 mpk also significantly reduced elevated RVFWT (FIG. 34), reduced PAAT (FIG. 35), and reduced elevated RVSP (FIG. 36). ActRIIA-mFc treatment (ZSF1-SU/ActRIIA-mFc) relative to PBS treatment (ZSF1-SU/PBS) also significantly reduced the increased fibrosis in LV. RV and lung (FIGS. 37-39).

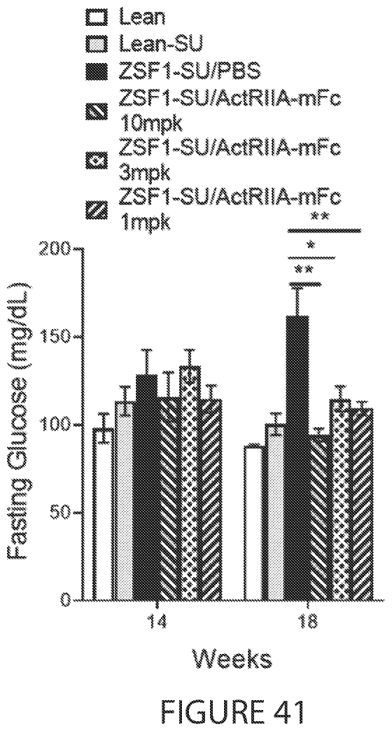

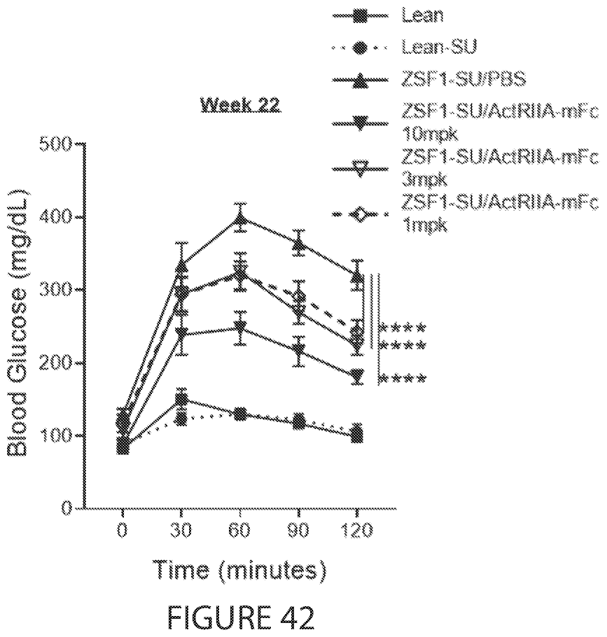

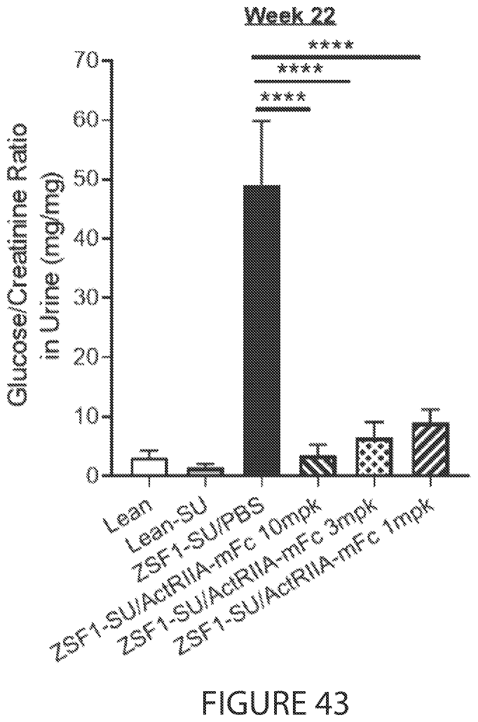

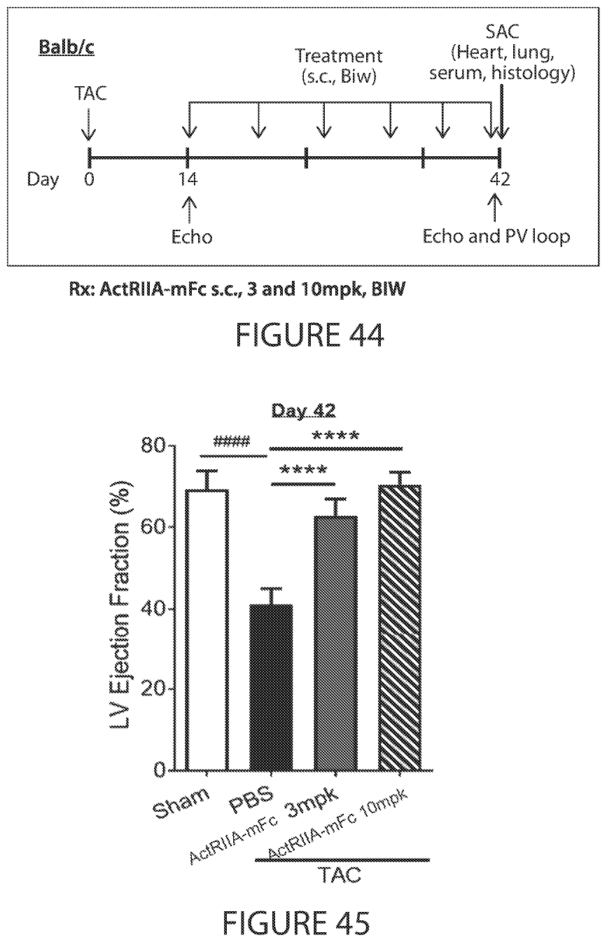

In addition, compared to lean control animals, ZSF1-SU rats in the PBS treatment group (ZSF1-SU/PBS) had elevated fasting blood glucose level and increased glucose level in urine, accompanied by glucose intolerance. ActRIIA-mFc treatment (ZSF1-SU/ActRIIA-mFc) relative to PBS treatment (ZSF1-SU/PBS) at 1 mpk, 3 mpk and 10 mpk significantly reduced fasting blood glucose, decreased glucose level in urine, and improved glucose tolerance (FIGS. 41-43).

Together, these data demonstrate that ActRIIA-mFc is effective in ameliorating various complications of Group 2 PH in a left heart failure-induced PH model (HFpEF-PH). In particular, ActRIIA-mFc had a significant effect in reducing cardiac hypertrophy, improving diastolic function, improving right heart remodeling and function, decreasing pulmonary hypertension, and reducing cardiac and pulmonary remodeling and fibrosis. Furthermore, ActRIIA-mFc had a robust effect in reducing glucose levels and improving glucose tolerance. The data indicate that other ActRII antagonists, particularly ones having activities similar to ActRIIA-mFc, may be useful in the treatment of Group 2 PH, particularly in preventing or reducing the severity various complications of Group 2 PH.

### Example 11: Effects of an ActRIIA-hFc Polypeptide in Patients with Cpc-PH Due to HFpEF

The effects of an an ActRIIA-mFc fusion polypeptide (SEQ ID NO: 56) are examined in a double-blind, randomized, placebo-controlled study to evaluate the effects of the ActRIIA-hFc fusion protein versus placebo for the treatment of combined pre- and postcapillary pulmonary hypertension (Cpc-PH) due to heart failure with preserved ejection fraction (HFpEF).

### Patients and Trial Design

Eligible patients will have confirmed Cpc-PH due to HFpEF, Functional Class 11 or III as assessed by the NYHA. Additionally, eligible patients are between 18 to 85 years of age and have a six minute walk distance greater than 100 meters repeated twice during screening and both values within 15% of each other, calculated from the highest value. Patients may be receiving stable medications for heart failure or any underlying condition for at least 30 days before and throughout the study. A planned interim analysis will occur when approximately 15 participants in each of the three treatment groups have completed 24 weeks on the study. Sensitivity analysis will be performed to account for any differences in background therapy. All patients will provide informed consent.

Initially, approximately 90 eligible patients will be randomly assigned in a 1:1:1 ratio to one of three treatment groups: (1) placebo; (2) ActRIIA-hFc fusion protein 0.3 mg/kg; or (3) ActRIIA-hFc fusion protein 0.3 mg/kg then escalating to 0.7 mg/kg. ActRIIA-hFc fusion protein or placebo (saline) will be given by subcutaneous injection every 21 days for a total of 24 weeks. Safety and efficacy will be assessed at screening and every 3 weeks for 24 weeks. See, e.g., Table 7 below. Adverse events are recorded from screening until the end of primary treatment study visit, 8 weeks after the last dose of study drug. An interim analysis will occur when approximately 15 participants in each of the 3 treatment groups have completed 24 weeks of treatment in the placebo-controlled treatment period.

Participants who have not discontinued early from the placebo-controlled treatment period and have had the 24-week PVR assessment will continue into the 18-month extension period and will be treated as follows: Placebo participants will be re-randomized in a 1:1 ratio to one of the two ActRIIA-hFc fusion protein treatment groups utilized in the placebo-controlled treatment period to receive either (1) ActRIIA-hFc fusion protein SC at a dose level of 0.3 mg/kg every 21 days for up to 18 months in the Extension Period or (2) ActRIIA-hFc fusion protein SC at a starting dose of 0.3 mg/kg plus background therapy, then escalate to 0.7 mg/kg at Visit 12 and every 21 days for up to 18 months in the Extension Period.

TABLE 7
Efficacy Endpoints

| Type | End points |
|---|---|
| Primary end | Change in pulmonary vascular resistance from baseline to 24 weeks |
| point | |
| Key | Change in 6-minute walk distance from baseline to 24 weeks |
| secondary | |
| end point | |
| Other | Clinical Worsening |
| secondary | Number of Clinical Worsening events, defined as follows, at 24 |
| end points | and 48 weeks: |
| | The occurrence of any 1 of the following clinical worsening |
| | events: hospitalization due to a cardiopulmonary indication |
| | (a non-elective hospitalization lasting at least 24 hours in |
| | duration caused by clinical conditions directly related to PH |
| | and/or heart failure), administration of IV diuretics, death (all |
| | causes), decrease in 6MWD > 15% from Baseline (or the |
| | subject was too ill to walk, and the cause was directly related |
| | to the disease under study) at 2 consecutive visits on different |
| | days (except Week 24) |
| | Number of Participants with first Clinical Worsening event, |
| | defined as above, at 24 and 48 weeks |
| | Time to Clinical Worsening, defined as above |
| | Change in dyspnea score (assessed by Borg CR10 scale ®) at Weck 24 |
| | from baseline |
| | Change in hemodynamic and ECHO parameters, including but not |
| | limited to mPAP, PCWP, TAPSE, RVFAC, and LVEF at 24 weeks |
| | from baseline |
| | Change in NT-proBNP at 24 weeks from baseline |
| | Change in NYHA FC at 24 weeks from baseline |
| | Change in 6MWD at 48 weeks from baseline |
| | Change in hemodynamic and ECHO parameters, including but not |
| | limited to PVR, mPAP, PCWP, TAPSE, RVFAC, and LVEF, at 48 |
| | weeks from baseline |
| | Change in NT-proBNP at 48 weeks from baseline |
| | Change in NYHA FC at 48 weeks from baseline |
| | Change in PVR, 6MWD and NYHA FC at week 48 from baseline in |
| | the extension in the Placebo-Crossed treatment group |
| | Change in PVR, 6MWD and NYHA FC from week 24 to week 48 in |
| | the extension in the Placebo-Crossed treatment group |
| Exploratory | Placebo-Controlled Treatment and Extension Periods |
| end points | Changes in Kansas City Cardiomyopathy Questionnaire (KCCQ) and |
| | EQ-5D-5L scores |
| | Change from baseline in disease-related biomarkers at 24 weeks and |
| | 48 weeks |
| | Correlation of clinical efficacy vs. genetic phenotype |

### Example 12: Effects of an ActRIIA-mFc on Group 2 Pulmonary Hypertension in a Transverse Aortic Constriction (TAC) Induced PH Mouse Model

The effects of an ActRIIA-mFc fusion polypeptide (SEQ ID NO: 56) was examined in a mouse model of left ventricular systolic dysfunction (also referred to as HErEF) of pulmonary hypertension (PH) and valvular heart disease. In this model, BALB/cJ mice underwent transverse aortic constriction (TAC) to induce left heart failure, and right heart and pulmonary remodeling. See, e.g., Xiong P Y. et al. Hypertension 2018, 71(1):34-55 and Chen Y. et al. Hypertension 2012, 59(6):1170-1178.

Forty-four BALB/cJ male mice (10 wks old) underwent TAC surgery and fourteen age-matched animals underwent a mock surgical procedure (Sham) at day 0. Two weeks after the surgery, TAC-PH mice were randomized into three groups. i) fourteen mice were injected subcutaneously with vehicle control (phosphate buffered saline (PBS)), twice weekly for 4 weeks starting from day 14 after surgery, “TAC PBS”; ii) fifteen mice were injected subcutaneously with ActRIIA-mFc at a dose of 3 mg/kg twice weekly for 4 weeks starting from day 14 after TAC surgery, “TAC ActRIIA-mFc 3 mpk”; and iii) fifteen mice were injected subcutaneously with ActRIIA-mFc at a dose of 10 mg/kg twice weekly for 4 weeks starting from day 14 after TAC surgery, “TAC ActRIIA-mFc 10 mpk.” At the end of the study, echocardiography and pressure-volume catheter were performed to measure left and right ventricular remodeling and functional changes before animals were euthanized for heart and lung collection. Hearts and lungs of each mouse were weighed, fixed in 10% formalin, embedded in paraffin, and sectioned for Masson's trichrome stain to assess fibrosis.

Prior to euthanasia, in vivo cardiac function was assessed by transthoracic echocardiography (Acuson P300, 18 MHz transducer; Siemens) in conscious mice. From left ventricle (LV) short axis view, M-mode echocardiogram was acquired to measure left ventricle end diastolic diameter (LVEDD), and left ventricle end systolic diameter (LVESD). Fractional shortening (FS) was calculated from the end-diastolic diameter (EDD) and end-systolic diameter (ESD) using the following equation: FS=100%×[(EDD−ESD)/EDD]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Three to five beats were averaged for each mouse measurement. RV free wall thickness (RVFWT) was measured using M-mode in a modified parasternal long-axis view through the aortic valve. Pulmonary artery acceleration time (PAAT) was measured as the time from start to peak velocity of blood flow in the lumen of the main pulmonary artery distal to the pulmonary valve as obtained from the pulse-wave doppler recording.

On day 42, mice were anesthetized by an intraperitoneal injection of ketamine/xylazine (100/5 mg/kg) to evaluate left and right ventricular function by Millar pressure-volume conductance catheter. The respiration was supported by a small animal ventilator.

Thoracotomy was made through 4-5 intercostal space, and the heart was exposed. A pressure-volume catheter (1.0-Fr, PVR-1035, Millar Instruments, Houston, TX, USA) was inserted into the left ventricle and right ventricle from the apex. Ventricular pressure and volume were calculated with LabChart 7 software. Ejection fraction was derived. Afterwards, animals were euthanized for heart and lung collection. Cardiac hypertrophy was measured by heart weight (HW) normalized by tibial length (TL). Left ventricle (LV), right ventricle (RV) and lung of each mouse were separated, fixed in 10% formalin, embedded in paraffin, and sectioned for Masson's trichrome stain to assess fibrosis. Serum and urine samples were collected at the end of the study.

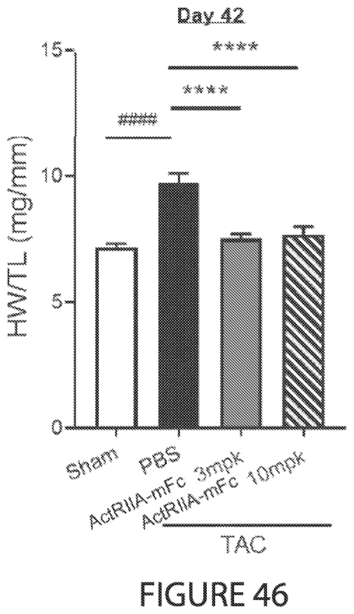

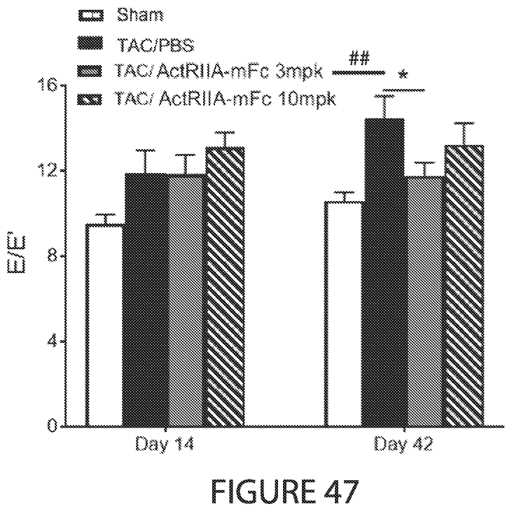

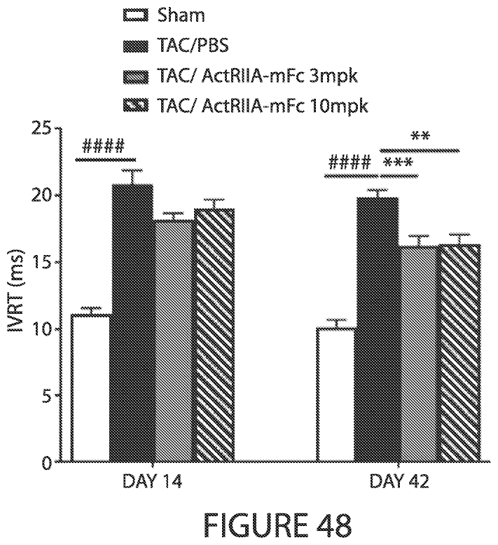

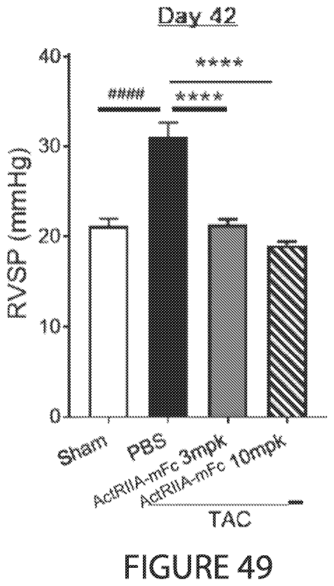

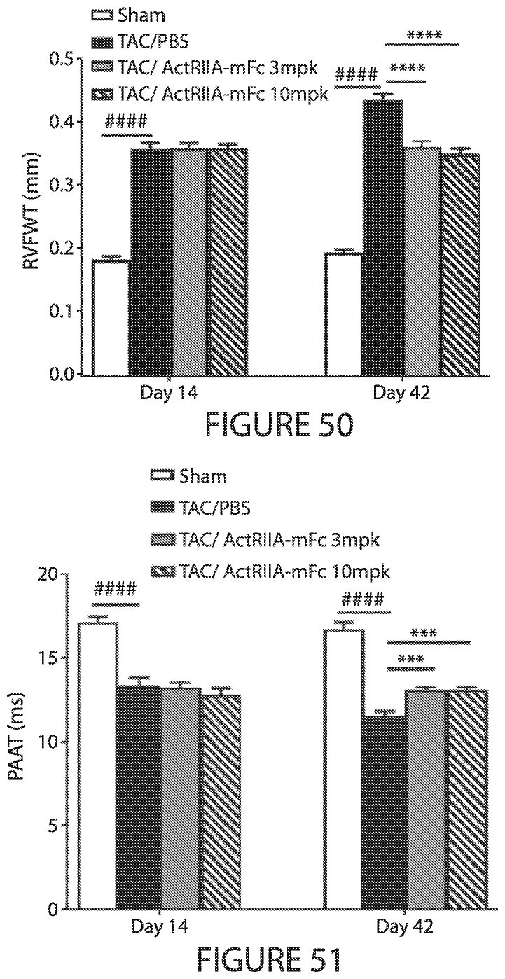

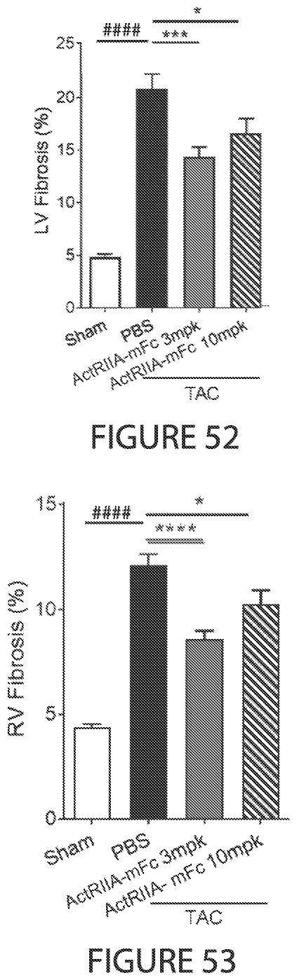

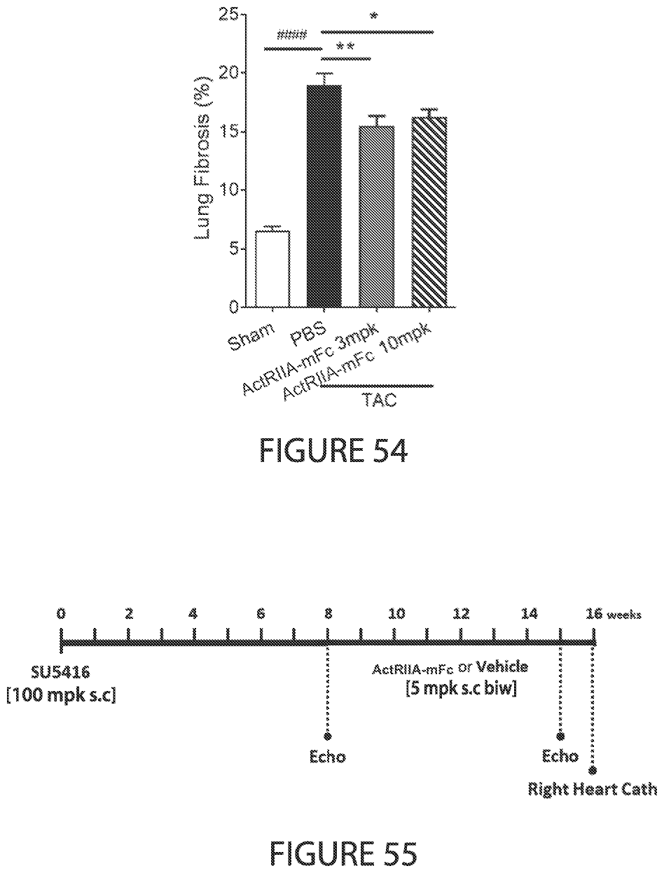

Compared to Sham control animals, TAC-PH mice in the PBS treatment group (TAC PBS) on day 42 were observed to have decreased left ventricle ejection fraction (FIG. 45), increased heart weight (HW/TL) (FIG. 46), increased E/E′ ratio (FIG. 47), increased isovolumic relaxation time (IVRT) (FIG. 48), and increased left ventricle fibrosis (FIG. 52), indicating cardiac hypertrophy and left heart failure. TAC mice also had increased right ventricle free wall thickness (RVFWT) (FIG. 50), decreased PAAT (FIG. 51), and increased right ventricle fibrosis (FIG. 53) compared to Sham control mice, suggesting the RV remodeling and RV dysfunction. In addition, increased RVSP (FIG. 49) and increased lung fibrosis (FIG. 54) were observed in TAC-PH/PBS mice, indicating pulmonary hypertension and lung remodeling caused by TAC-induced left heart failure.

As shown in FIGS. 45-54, ActRIIA-mFc treatment (TAC ActRIIA-mFc 3 mpk or TAC ActRIIA-mFc 10 mpk) relative to PBS treatment (TAC PBS) on day 42 significantly reduced cardiac hypertrophy (FIG. 46), restored LV ejection fraction (FIG. 45), reduced E/E′ ratio at 3 mpk (FIG. 47), and reduced IVRT (FIG. 48). ActRIIA-mFc treatment (TAC ActRIIA-mFc 3 mpk or TAC ActRIIA-mFc 10 mpk) relative to PBS treatment (TAC PBS) on day 42 also significantly reduced elevated RVFWT (FIG. 50), reduced RVSP (FIG. 49), and increased PAAT (FIG. 51). ActRIIA-mFc treatment (TAC ActRIIA-mFc 3 mpk or TAC ActRIIA-mFc 10 mpk) relative to PBS treatment (TAC PBS) on day 42 significantly reduced lung fibrosis (FIG. 54), LV fibrosis (FIG. 52), and RV fibrosis (FIG. 53).

Together, these data demonstrate that ActRIIA-mFc is effective in ameliorating various complications of Group 2 PH in a left heart failure-induced PH model (TAC PH). In particular, ActRIIA-mFc had a significant effect in reducing cardiac hypertrophy, improving cardiac function, improving right heart remodeling and function, improving LV function, and reducing pulmonary remodeling and fibrosis.

### Example 13: Effects of an ActRIIA-mFc on Group 2 Pulmonary Hypertension in an HFpEF Induced PH Rat Model

The effects of an ActRIIA-mFc fusion polypeptide (SEQ ID NO: 56) was examined in a rat model of left ventricular diastolic dysfunction (also referred to as HFpEF) group 2 (subgroup 2.2) pulmonary hypertension (PH). In this model, ZSF1-Lepr^{fa}Lepr^{cp}/Crl rats were challenged with semaxanib (SU5416) to induce HFpEF-PH.

Twenty ZSF1 Lepr^{fa}Lepr^{cp}/Crl male mice (8 wks old) and ten lean rats were subcutaneously administered with a single dose of semaxanib (100 mg/kg) at day 0, and ten lean rats were included as normal control. Eight weeks after semaxanib (SU5416) treatment, twenty ZSF1 Lepr^{fa}Lepr^{cp}/Crl rats were randomized into two groups: i) ten rats were injected subcutaneously with vehicle control (phosphate buffered saline (PBS)), twice weekly for 8 weeks starting from day 64 after semaxanib treatment, “Obese ZSF1 Veh”; and ii) ten rats were injected subcutaneously with ActRIIA-mFc at a dose of 5 mpk twice weekly for 8 weeks starting from day 64 after semaxanib treatment, “Obese ZSF1 ActRIIA-mFc.” At the end of the study, echocardiography and pressure-volume catheter were performed to measure left and right ventricular remodeling and functional changes before animals were euthanized for heart and lung collection.

Prior to euthanization, in vivo cardiac function was assessed by transthoracic echocardiography (Acuson P300, 18 MHz linear transducer: Siemens) in lightly anesthetized rats as described. Echocardiographic assessments were conducted at week 8 (before therapy with ActRIIA-mFc or vehicle) and week 15 (after therapy) in each rat. From left ventricle short axis view, M-mode echocardiogram was acquired to measure interventricular septal thickness at end diastole (IVSd), left ventricular posterior wall thickness at end diastole (LVPWd), left ventricular end diastolic diameter (LVEDD), and left ventricular end systolic diameter (LVESD). Left ventricular mass (LVM) was assessed by the equation: 1.05 [(LVEDD+LVPTD+IVSd)³-LVEDD³]. Early diastolic filling peak velocity (E), early diastolic mitral annular velocity (E′), and isovolumetric relaxation time (IVRT) were measured from the medial or septal wall at the mitral valve level from tissue Doppler image. LV diastolic function was assessed by measuring the E/E′ ratio and IVRT. Pulmonary arterial acceleration time (PAAT), a parameter of right ventricular function, was measured. Tricuspid annular plane systolic excursion (TAPSE), a parameter of global right ventricular function, was also measured. RV free wall thickness (RVFWT) was measured using M-mode in a modified parasternal long-axis view through the aortic valve. Pulmonary artery acceleration time (PAAT) was measured as the time from start to peak velocity of blood flow in the lumen of the main pulmonary artery distal to the pulmonary valve as obtained from the pulse-wave doppler recording.

Sixteen weeks after semaxanib treatment, rats were anesthetized with ketamine (100 mg/kg) and xylazine (5 mg/kg) at the end of the experiment to evaluate cardiac and pulmonary hemodynamics. The respiration was supported by a small animal ventilator. Thoracotomy was made through 4-5 intercostal space, and the heart was exposed. A pressure-volume catheter (2.0-Fr, SPR-869, Millar Instruments, Houston, TX, USA) was inserted into the left ventricle and right ventricle from the apex. Ventricular pressure and volume were calculated with LabChart 7 software. Stroke work, ejection fraction, and cardiac output were derived. After finishing left ventricular measurements, the catheter was advanced to the aorta, arterial blood systolic and diastolic pressure was detected. Then the catheter returned to the left ventricle and changed the direction laterally to enter the left atrium. Similarly, right atrial pressure was measured by moving the catheter from the right ventricle into atrium. To measure pulmonary arterial pressure, the sternum was cross-sectioned at the second inter-rib space. The right ventricular outflow tract was exposed. A hole was made with 27G needle, and then the catheter was inserted into the right ventricular outflow tract and advanced into the pulmonary artery.

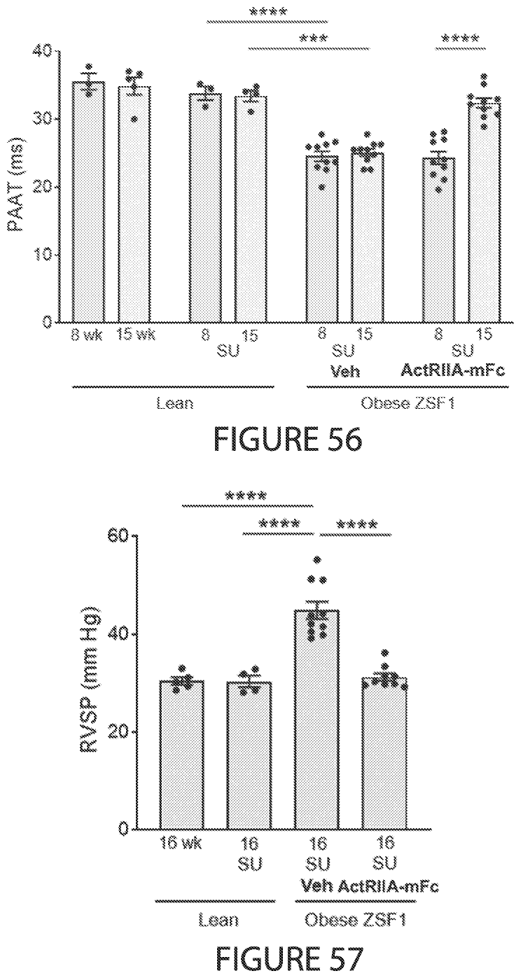

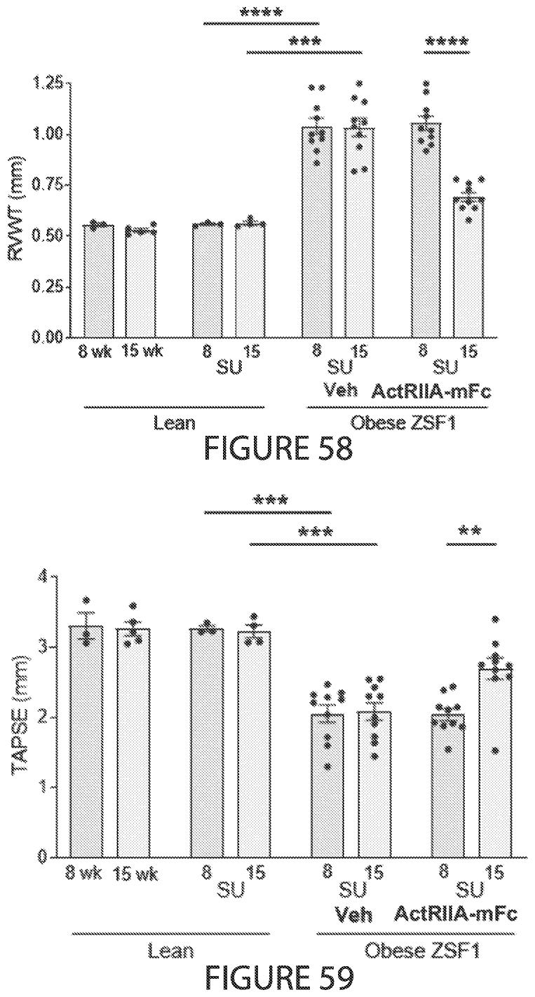

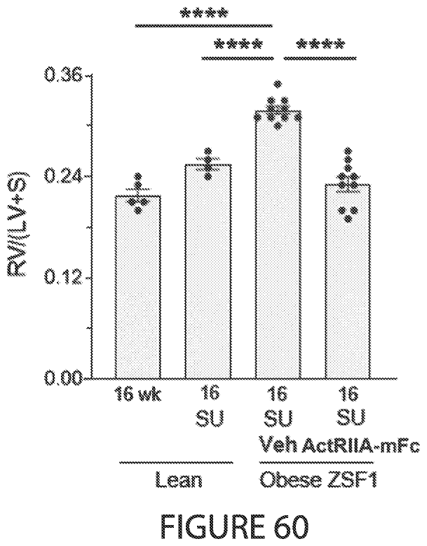

Compared to lean control animals, Obese ZSF1-SU rats in the PBS treatment group (Obese ZSF1 SU/Veh) 16-weeks after semaxanib treatment were observed to have decreased pulmonary artery acceleration time (PAAT) (FIG. 56), increased RVSP (FIG. 57), increased right ventricle free wall thickness (RVFWT) (FIG. 58), decreased tricuspid annular plane systolic excursion (TAPSE) (FIG. 59), and increased Fulton Index, calculated as the ratio of right ventricular weight (RV) to weight of the combined left ventricle and septum (LV+S) (FIG. 60).

As shown in FIGS. 56 and 57, ActRIIA-mFc treatment (Obese ZSF1-SU/ActRIIA-mFc) relative to PBS treatment (Obese ZSF1-SU/Veh) at 5 mpk normalized cardiopulmonary function as shown by the significantly increased PAAT (FIG. 56) and significantly reduced right ventricular systolic pressure (RVSP) (FIG. 57). ActRIIA-mFc treatment (Obese ZSF1-SU/ActRIIA-mFc) relative to PBS treatment (Obese ZSF1-SU/Veh) at 5 mpk also normalized right ventricular structure and function as shown by the significantly reduced elevated RVWT (FIG. 58), increased TAPSE (FIG. 59), and the decreased Fulton index (FIG. 60).

Together, these data demonstrate that ActRIIA-mFc is effective in ameliorating various complications of Group 2 PH in a left heart failure-induced PH model (HFpEF-PH). In particular, ActRIIA-mFc had a significant effect in normalizing cardiopulmonary function and in normalizing right ventricular structure and function.

1-177. (canceled)

178. A method of treating post-capillary pulmonary hypertension (PcPH), comprising administering to a patient in need thereof an effective amount of a fusion protein comprising:
(i) a variant ActRIIB polypeptide comprising an amino acid sequence that is at least 90% identical to an amino acid sequence that begins at any one of amino acids 20-29 of SEQ ID NO: 2 and ends at any one of amino acids 109-134 of SEQ ID NO: 2, and wherein the polypeptide comprises one or more amino acid substitutions at a position of SEQ ID NO: 2 selected from the group consisting of: A24, S26, N35, E37, L38, R40, S44, L46, E50, E52, Q53, D54, K55, R56, L57, Y60, R64, N65, S67, G68, K74, W78, L79, D80, F82, N83, T93, E94, Q98, V99, E105, F108, E111, R112, A119, G120, E123, P129, P130, and A132;
(ii) an Fc domain of an immunoglobulin;
(iii) a linker domain positioned between the ActRIM polypeptide domain and the one or more heterologous domains or Fc domain.

179. The method of claim 178, wherein the PcPH is isolated post-capillary pulmonary hypertension (IpcPH).

180. The method of claim 178, wherein the PcPH is combined post- and pre-capillary PH (CpcPH).

181. The method of claim 178, wherein the Fc domain of the immunoglobulin is at least 90% identical to the amino acid sequence of SEQ ID NO: 13, 14, 15, 16, and 17.

182. The method of claim 178, wherein the Fc domain of the immunoglobulin is at least 95% identical to the amino acid sequence of SEQ ID NO: 13, 14, 15, 16, and 17.

183. The method of claim 178, wherein the Fc domain of the immunoglobulin is at least 99% identical to the amino acid sequence of SEQ ID NO: 13, 14, 15, 16, and 17.

184. The method of claim 178, wherein the linker domain is selected from the group consisting of: TGGG, TGGGG, SGGGG, GGGGS, GGG, GGGG, SGGG, and GGGGS.

185. The method of claim 178, wherein the ActR1113 polypeptide comprises an amino acid sequence that is at least 95% identical to amino acids 29-109 of SEQ ID NO: 2.

186. The method of claim 178, wherein the ActR1113 polypeptide comprises an amino acid sequence that is at least 95% identical to SEQ ID NO: 53.

187. The method of claim 178, wherein the fusion protein comprises an amino acid sequence that is at least 95% identical to SEQ ID NO: 5.

188. The method of claim 178, wherein the ActR1113 polypeptide comprises an amino acid sequence that is at least 95% identical to SEQ ID NO: 12.

189. The method of claim 178, wherein the ActR1113 polypeptide comprises one or more amino acid substitution with respect to the amino acid sequence of SEQ ID NO: 2 selected from the group consisting of: A24N, S26T, N35E, E37A, E37D, L38N, R40A, R40K, S44T, L46V, L461, L46F, L46A, E50K, ESOP, E50L, E52A, E52D, E52G, E52H, E52K, E52N, E52P, E52R, E52S, E52T, E52Y, Q53R, Q53K, Q53N, Q53H, D54A, K55A, K55D, K55E, K55R, R56A, L57E, L57I, L57R, L57T, L57V, Y60D, Y60F, Y60K, Y60P, R64A, R64H, R64K, R64N, N65A, S67N, S67T, G68R, K74A, K74E, K74F, K74I, K74R, K74Y, W78A, W78Y, L79A, L79D, L79E, L79F, L79H, L79K, L79P, L79R, L79S, L79T, L79W, D80A, D80F, D80G, D80I, D80K, D80M, D80N, D80R, F82A, F82D, F82E, F82I, F82K, F82L, F82S, F82T, F82W, F82Y, N83A, N83R, T93D, T93E, T93G, T93H, T93K, T93P, T93R, T93S, T93Y, E94K, Q98D, Q98E, Q98K, Q98R, V99E, V99G, V99K, E105N, F1081, F108L, F108V, F108Y, E111D, E111H, E111K, 111N, E111Q, E111R, R112H, R112K, R112N, R112S, R112T, A119P, A119V, G120N, E123N, P129N, P129S, P130A, P130R, and A132N.

190. The method of claim 178, wherein the fusion protein comprises an amino acid sequence that is at least 95% identical to SEQ ID NO: 276, 278, 279, 332, 333, 335, 336, 338, 339, 341, 342, 344, 345, 347, 348, 350, 351, 353, 354, or 356.

191. The method of claim 178, wherein the fusion protein comprises an amino acid sequence that is at least 99% identical to SEQ ID NO: 276, 278, 279, 332, 333, 335, 336, 338, 339, 341, 342, 344, 345, 347, 348, 350, 351, 353, 354, or 356.

192. The method of claim 178, wherein the patient has Group 2 pulmonary hypertension as recognized by the World Health Organization (WHO).

193. The method of claim 178, wherein the patient has pulmonary hypertension due to heart failure with preserved left ventricular ejection fraction (LVEF).

194. The method of claim 178, wherein the method decreases pulmonary vascular resistance (PVR) in the patient.

195. The method of claim 178, wherein the method prevents the progression of IpcPH to CpcPH.

196. The method of claim 178, wherein the method reduces the development of a pre-capillary component of PH.

197. The method of claim 178, wherein the method increases the patient's 6-minute walk distance.

198. The method of claim 178, wherein the method delays clinical worsening of PcPH in accordance with the World Health Organization's functional classification system for pulmonary hypertension.

199. The method of claim 178, wherein the method reduces the risk of hospitalization for one or more complications associated with PcPH.
